SEQUENCE LISTING (83 sequences)

SEQ ID NO: 1 - protein, 359 aa, Homo Sapiens
MQVPNSTGPDNATLQMLRNPAIAVALPVVYSLVAAVSIPGNLFSLWVLCRRMGPRSPSVIFMINLSVTDLMLASVLPFQIYYHCNRHHWVFGVLLCNVVTVAFYANMYSSILTMTCISVERFLGVLYPLSSKRWRRRRYAVAACAGTWLLLLTALSPLARTDLTYPVHALGIITCFDVLKWTMLPSVAMWAVFLFTIFILLFLIPFVITVACYTATILKLLRTEEAHGREQRSAAVGLAAVVLLAFVTCFAPNNFVLLAHIVSRLFYGKSYYHVYKLTLCLSCLNNCLDPFVYYFASREFQLRLREYLGCRRVPRDTLDTRRESLFSARTTSVRSEAGAHPEGMEGATRPGLQRQESVF

SEQ ID NO: 2 - DNA, 1955 bp, Homo Sapiens
  misc_feature (1)...(1955): n = A,T,C or G
cccaagctaaaattaaccctcactaaagggaataagcttgcggccgcctttgcaaggttgctggacagatggaactggaagggcagccgtctgccgcccacgaacaccttctcaagcactttgagtgaccacggcttgcaagctggtggctggccccccgagtcccgggctctgaggcacggccgtcgacttaagcgttgcatcctgttacctggagaccctctgagctctcacctgctacttctgccgctgcttctgcacagagcccgggcgaggacccctccaggatgcaggtcccgaacagcaccggcccggacaacgcgacgctgcagatgctgcggaacccggcgatcgcggtggccctgcccgtggtgtactcgctggtggcggcggtcagcatcccgggcaacctcttctctctgtgggtgctgtgccggcgcatggggcccagatccccgtcggtcatcttcatgatcaacctgagcgtcacggacctgatgctggccagcgtgttgcctttccaaatctactaccattgcaaccgccaccactgggtattcggggtgctgctttgcaacgtggtgaccgtggccttttacgcaaacatgtattccagcatcctcaccatgacctgtatcagcgtggagcgcttcctgggggtcctgtacccgctcagctccaagcgctggcgccgccgtcgttacgcggtggccgcgtgtgcagggacctggctgctgctcctgaccgccctgtccccgctggcgcgcaccgatctcacctacccggtgcacgccctgggcatcatcacctgcttcgacgtcctcaagtggacgatgctccccagcgtggccatgtgggccgtgttcctcttcaccatcttcatcctgctgttcctcatcccgttcgtgatcaccgtggcttgttacacggccaccatcctcaagctgttgcgcacggaggaggcgcacggccgggagcagcggagcgccgcggtgggcctggccgcggtggtcttgctggcctttgtcacctgcttcgcccccaacaacttcgtgctcctggcgcacatcgtgagccgcctgttctacggcaagagctactaccacgtgtacaagctcacgctgtgtctcagctgcctcaacaactgtctggacccgtttgtttattactttgcgtcccgggaattccagctgcgcctgcgggaatatttgggctgccgccgggtgcccagagacaccctggacacgcgccgcgagagcctcttctccgccaggaccacgtccgtgcgctccgaggccggtgcgcaccctgaagggatggagggagccaccaggcccggcctccagaggcaggagagtgtgttctgagtcccgggggcgcagcttggagagccgggggcgcagcttggagatccaggggcgcatggagaggccacggtgccagaggttcagggagaacagctgcgttgctcccaggcactgcagaggcccggtggggaagggtctccaggctttattcctcccaggcactgcagaggcaccggtgaggaagggtctccaggcttcactcagggtagagaaacaagcaaagcccagcagcgcacagggtgcttgttatcctgcagagggtgcctctgcctcttcaccacgcccggctaatttttgtattttttttagtagagctgggctgtcacccccgagctccttagacactcctcacacctgtccatacccgaggatggatattcaaccagccccaccgcctacccactcggtttctggatatcctctgtgggcgaactgcgagccccattcccagctcttctccctgctgacatcgtcccttagtttggtgggttcttggccttctccattctctcmaaggggttctggncyttcgagcccccggtgcacgcccaaattttctgggttattttcactcagggcactttgtgg

SEQ ID NO: 3 - protein, 269 aa, Artificial Sequence
  Transmembrane Receptor of the Rhodopsin Family
GNILVIWVICRYRRMRTPMNYFIVNLAVADLLFSLFTMPFWMVYYVMGGRWPFGDFMCRIWMYFDYMNMYASIFFLTCISIDRYLWAICHPMRYMRWMTPRHRAWVMIIIIWVMSFLISMPPFLMFRWSTYRDENEWNMTWCMIYDWPEWMWRWYVILMTIIMGFYIPMIIMLFCYWRIYRIARLWMRMIPSWQRRRRMSMRRERRIVKMLIIIMVVFIICWLPYFIVMFMDTLMMWWFCEFCIWRRLWMYIFEWLAYVNCPCINPIIY

SEQ ID NO: 4 - protein, 319 aa, Homo Sapiens
MSQQNTSGDCLFDGVNELMKTLQFAVHIPTFVLGLLLNLLAIHGFSTFLKNRWPDYAATSIYMINLAVFDLLLVLSLPFKMVLSQVQSPFPSLCTLVECLYFVSMYGSVFTICFISMDRFLAIRYPLLVSHLRSPRKIFGICCTIWVLVWTGSIPIYSFHGKVEKYMCFHNMSDDTWSAKVFFPLEVFGFLLPMGIMGFCCSRSIHILLGRRDHTQDWVQQKACIYSIAASLAVFVVSFLPVHLGFFLQFLVRNSFIVECRAKQSISFFLQLSMCFSNVNCCLDVFCYYFVIKEFRMNIRAHRPSRVQLVLQDTTISRG

SEQ ID NO: 5 - DNA, 1617 bp, Homo Sapiens
  CDS (206)...(1165)
aggtacagcctttggccattagagaactaaggcaggaacctccaacctgaccttgctcttgtggactgcagttgtgattcaatgggcatgaattgctgtgtgatgctgggaaggtgtttgtgattcttgacaaagtcatttgaatccatcacttcaagagagtgaaaggagccccgtctgatctgttggtgttgtaggaagaaacatgagtcagcaaaacaccagtggggactgcctgtttgacggtgtcaacgagctgatgaaaaccctacagtttgcagtccacatccccaccttcgtcctgggcctgctcctcaacctgctggccatccatggctttagcaccttccttaagaacaggtggcccgattatgctgccacctccatctacatgatcaacctggcagtctttgacctgctgctggtgctctccctcccattcaagatggtcctgtcccaggtacagtcccccttcccgtccctgtgcaccctggtggagtgcctttacttcgtcagcatgtacggaagcgtcttcaccatctgcttcatcagcatggaccggttcttggccatccgttacccgctactggtgagccacctccggtcccccaggaagatctttgggatctgctgcaccatctgggtcctggtgtggaccggaagcatccctatctacagtttccatgggaaagtggaaaaatacatgtgcttccacaacatgtctgatgatacctggagcgccaaggtcttcttcccgctggaggtgtttggcttcctccttcccatgggcatcatgggcttctgctgctccaggagcatccacatcctgctgggccgccgagaccacacccaggactgggtgcagcagaaagcctgcatctacagcatcgcagccagcctggctgtcttcgtggtctccttcctcccagtccacctggggttcttcctgcagttcctggtgagaaacagctttatcgtagagtgcagagccaagcagagcatcagcttcttcttgcaattgtccatgtgtttctccaacgtcaactgctgcctggatgttttctgctactactttgtcatcaaagaattccgcatgaacatcagggcccaccggccttccagggtccagctggtcctgcaggacaccacgatctcccggggctaacggaaggacatcctgttcaggggaagaaagccctggccctgaattctggtaacggatttcgcgttccagggttttgatgtggtgggatgatccgcaccatcttcactgatgtgcttccctttgatgcccattgagtgccagctttgctcattataccccaaagaccttttttccactgcccagacagcttataccacccagtgttcagggatctctgaagaacccacagaccaggtgaattactgatttctaagtccaaaaactatagagcagaagaattgagaaagagaatgagaccatgtcaacaaggctgtttccaactctccccattttcctgttcgactgggaggttctggaaagaaagagagagagagaaaagaggtaaaggagggagccaagagagtcagttattggggagagtgtcttgggcagaggtggggtggtagggatga

SEQ ID NO: 6 - protein, 337 aa, Homo Sapiens
MDETGNLTVSSATCHDTIDDFRNQVYSTLYSMISVVGFFGNGFVLYVLIKTYHKKSAFQVYMINLAVADLLCVCTLPLRVVYYVHKGIWLFGDFLCRLSTYALYVNLYCSIFFMTAMSFFRCIAIVFPVQNINLVTQKKARFVCVGIWIFVILTSSPFLMAKPQKDEKNNTKCFEPPQDNQTKNHVLVLHYVSLVGGFIIPFVIIIVCYTMIILTLLKKSMKKNLSSHKKAIGMIMVVTAAFLVSFMPYHIQRTIHLHFLHNETKPCDSVLRMQKSVVITLSLAASNCCFDPLLYFFSGGNFRKRLSTFRKHSLSSVTYVPRKKASLPEKGEEICKV

SEQ ID NO: 7 - DNA, 1358 bp, Homo Sapiens
  CDS (230)...(1243)
actttcaggccagaattcggcacgaggctggtagatcgaatttactgaagacttggagcttgcttctgagaacaaacgcaaaaggacagtaaactgtggaccttgaagttagcagcgtgggcttcctctaatattacaccgtaaaaggcattgatcaccataagaaggaacatttgtgaaggtactccagtgccagaaagaggcacaaagcagacattcgtagagaaacatggatgaaacaggaaatctgacagtatcttctgccacatgccatgacactattgatgacttccgcaatcaagtgtattccaccttgtactctatgatctctgttgtaggcttctttggcaatggctttgtgctctatgtcctcataaaaacctatcacaagaagtcagccttccaagtatacatgattaatttagcagtagcagatctactttgtgtgtgcacactgcctctccgtgtggtctattatgttcacaaaggcatttggctctttggtgacttcttgtgccgcctcagcacctatgctttgtatgtcaacctctattgtagcatcttctttatgacagccatgagctttttccggtgcattgcaattgtttttccagtccagaacattaatttggttacacagaaaaaagccaggtttgtgtgtgtaggtatttggatttttgtgattttgaccagttctccatttctaatggccaaaccacaaaaagatgagaaaaataataccaagtgctttgagcccccacaagacaatcaaactaaaaatcatgttttggtcttgcattatgtgtcattggttgttggctttatcatcccttttgttattataattgtctgttacacaatgatcattttgaccttactaaaaaaatcaatgaaaaaaaatctgtcaagtcataaaaaggctataggaatgatcatggtcgtgaccgctgcctttttagtcagtttcatgccatatcatattcaacgtaccattcaccttcattttttacacaatgaaactaaaccctgtgattctgtccttagaatgcagaagtccgtggtcataaccttgtctctggctgcatccaattgttgctttgaccctctcctatatttcttttctgggggtaactttaggaaaaggctgtctacatttagaaagcattctttgtccagcgtgacttatgtacccagaaagaaggcctctttgccagaaaaaggagaagaaatatgtaaagtatagtttaaacccatttccagtccaaaccaatgaaaatagtttcccaaataagtattttgtcaaatcatttacaaaaaaaataaaaattttacttaaaaaaaaaaaaaaaaaaggaaaa

SEQ ID NO: 8 - protein, 372 aa, Homo Sapiens
MLANSSSTNSSVLPCPDYRPTHRLHLVVYSLVLAAGLPLNALALWVFLRALRVHSVVSVYMCNLAASDLLFTLSLPVRLSYYALHHWPFPDLLCQTTGAIFQMNMYGSCIFLMLINVDRYAGIVHPLRLRHLRRARVARLLCLGVWALILVFAVPAARVHRPSRCRYRDLEVRLCFESFSDELWKGRLLPLVLLAEALGFLLPLAAVVYSSGRVFWTLARPDATQSQRRRKTVRLLLANLVIFLLCFVPYNSTLAVYGLLRSKLVAASVPARDRVRGVLMVMVLLAGANCVLDPLVYYFSAEGFRNTLRGLGTPHRARTSATNGTRAALAQSERSAVTTDATRPDAASQGLLRPSDSHSLSSFTQCPQDSAL

SEQ ID NO: 9 - DNA, 2559 bp, Homo Sapiens
  CDS (137)...(1255)
ccatgacctccctctgcttgttttgggaccatgtctgtacagcctctaggccccagccccggaggtgaatgccatgccatgattctggtgtgctccatggcatccccagcctagctcccaatcccactttggcacgatgttagccaacagctcctcaaccaacagttctgttctcccgtgtcctgactaccgacctacccaccgcctgcacttggtggtctacagcttggtgctggctgccgggctccccctcaacgcgctagccctctgggtcttcctgcgcgcgctgcgcgtgcactcggtggtgagcgtgtacatgtgtaacctggcggccagcgacctgctcttcaccctctcgctgcccgttcgtctctcctactacgcactgcaccactggcccttccccgacctcctgtgccagacgacgggcgccatcttccagatgaacatgtacggcagctgcatcttcctgatgctcatcaacgtggaccgctacgccggcatcgtgcacccgctgcgactgcgccacctgcggcgggcccgcgtggcgcggctgctctgcctgggcgtgtgggcgctcatcctggtgtttgccgtgcccgccgcccgcgtgcacaggccctcgcgttgccgctaccgggacctcgaggtgcgcctatgcttcgagagcttcagcgacgagctgtggaaaggcaggctgctgcccctcgtgctgctggccgaggcgctgggcttcctgctgcccctggcggcggtggtctactcgtcgggccgagtcttctggacgctggcgcgccccgacgccacgcagagccagcggcggcggaagaccgtgcgcctcctgctggctaacctcgtcatcttcctgctgtgcttcgtgccctacaacagcacgctggcggtctacgggctgctgcggagcaagctggtggcggccagcgtgcctgcccgcgatcgcgtgcgcggggtgctgatggtgatggtgctgctggccggcgccaactgcgtgctggacccgctggtgtactactttagcgccgagggcttccgcaacaccctgcgcggcctgggcactccgcaccgggccaggacctcggccaccaacgggacgcgggcggcgctcgcgcaatccgaaaggtccgccgtcaccaccgacgccaccaggccggatgccgccagtcaggggctgctccgaccctccgactcccactctctgtcttccttcacacagtgtccccaggattccgccctctgaacacacatgccattgcgctgtccgtgcccgactcccaacgcctctcgttctgggaggcttacagggtgtacacacaagaaggtgggctgggcacttggacctttgggtggcaattccagcttagcaacgcagaagagtacaaagtgtggaagccagggcccagggaaggcagtgctgctggaaatggcttctttaaactgtgagcacgcagagcaccccttctccagcggtgggaagtgatgcagaaagcccacccgtgcagagggcagaagaggacgaaatgcctttgggtgggcagggcattaaactgctaaaagctggttagatggaccagaaaatgggcattctggattttaacccgccacaggggcttgagagttgaagagcaccaggtttggtggacaaagctactgagatgcctgttcatctgctgacttctgtctaggctcatggatgccaccccctttcattttggcctaggcttcccctgctcaccactgaggcctaatacaagagttcctatggacagaactacattctttctcgcatagtgacttgtgacaatttagacttggcatccagcatgggatagttggggcaaggcaaaactaacttagagtttccccctcaacaacatccaagtccaaaccctttttaggttatcctttcttccatcacatccccttttccaggcctcctccattttaggtccttaatattctttctttttctctctctctcgtttctctcttctctctcctctcctctctcttctcctcttctctctctctccctctctctccttgtccagagtaaggataaattctttctactaaagcactggttctcaaactttttggtctcagaccccactcttagaaattgaggatctcaaagagctttgcttatattttgttcttttgatacttaccatactagaaattaaagcgaatacatttttaaaataaatacacatgcacacattacattagccatgggagcaataatgtcaccacacacacttcatgaagcctctggaaaactctacagtatacttgtgagagaatgagagtgaaagggacaaataacatctgtgtagcagtattatgaaaatagcttgacctcgtggacttcctcagagggttggtccctggatcacactttgagaaccatacttgtcctgaagtattggagttcatgtctaacttcttcccagggcattatgtacagtgctttttattactgtggggagagggcagtgctaaataaattaatcactactgatagtcaaaaaaaaaaaaaaa

SEQ ID NO: 10 - protein, 269 aa, Artificial Sequence; identical to SEQ ID NO: 3
  Rhodopsin family transmembrane receptor

SEQ ID NO: 11 - protein, 398 aa, Homo Sapiens
MESGLLRPAPVSEVIVLHYNYTGKLRGARYQPGAGLRADAVVCLAVCAFIVLENLAVLLVLGRHPRFHAPMFLLLGSLTLSDLLAGAAYAANILLSGPLTLKLSPALWFAREGGVFVALTASVLSLLAIALERSLTMARRGPAPVSSRGRTLAMAAAAWGVSLLLGLLPALGWNCLGRLDACSTVLPLYAKAYVLFCVLAFVGILAAICALYARIYCQIRANARRLPARPGTAGTTSTRARRKPRSLALLRTLSVVLLAFVACWGPLFLLLLLDVACPARTCPVLLQADPFLGLAMANSLLNPIIYTLTNRDLRHALLRLVCCGRHSCGRDPSGSQQSASAAEASGGLRRCLPPGLDGSFSGSERSSPQRDGLDTSGSTGSPGAPTAARTLVSEPAAD

SEQ ID NO: 12 - DNA, 1901 bp, Homo Sapiens
  misc_feature (1)...(1901): n = A,T,C or G
cccacgcgtccggggagaggactcaggctaaggtggcccccactgaagactcctgctaagcaacccactgaagacccctccgaatcatcgacggggcgtccttggggtgcagcccaggaagctcagttcacagccttggggcgcgcggcccatggagtcggggctgctgcggccggcgccggtgagcgaggtcatcgtcctgcattacaactacaccggcaagctccgcggtgcgcgctaccagccgggtgccggcctgcgcgccgacgccgtggtgtgcctggcggtgtgcgccttcatcgtgctagagaatctagccgtgttgttggtgctcggacgccacccgcgcttccacgctcccatgttcctgctcctgggcagcctcacgttgtcggatctgctggcaggcgccgcctacgccgccaacatcctactgtcggggccgctcacgctgaaactgtcccccgcgctctggttcgcacgggagggaggcgtcttcgtggcactcactgcgtccgtgctgagcctcctggccatcgcgctggagcgcagcctcaccatggcgcgcagggggcccgcgcccgtctccagtcgggggcgcacgctggcgatggcagccgcggcctggggcgtgtcgctgctcctcgggctcctgccagcgctgggctggaattgcctgggtcgcctggacgcttgctccactgtcttgccgctctacgccaaggcctacgtgctcttctgcgtgctcgccttcgtgggcatcctggccgcgatctgtgcactctacgcgcgcatctactgccagatacgcgccaacgcgcggcgcctgccggcacggcccgggactgcggggaccacctcgacccgggcgcgtcgcaagccgcgctcgctggccttgctgcgcacgctcagcgtggtgctcctggcctttgtggcatgttggggccccctcttcctgctgctgttgctcgacgtggcgtgcccggcgcgcacctgtcctgtactcctgcaggccgatcccttcctgggactggccatggccaactcacttctgaaccccatcatctacacgctcaccaaccgcgacctgcgccacgcgctcctgcgcctggtctgctgcggacgccactcctgcggcagagacccgagtggctcccagcagtcggcgagcgcggctgaggcttccgggggcctgcgccgctgcctgcccccgggccttgatgggagcttcagcggctcggagcgctcatcgccccagcgcgacgggctggacaccagcggctccacaggcagccccggtgcacccacagccgcccggactctggtatcagaaccggctgcagactgacaccctcggcccacgactgtcttcccaagttttacagacttgttctttttacataaaggaatttgtaggaaatgcagccaaaggtgcagtcggaaaagatgcaggggaaatgtatttatgcagcgacaccccacaatgtgaacaaacagacaaaaaatctgtgccctcgtggaattgacgttctgcttgggaacacagaaaagaactcggtgatgaaataatggagatgattccagtgacaaacgacagagatggtgatggtggtcagggaagacctctctgcagaggtagtgacttgtgatgtgagctgagacctctgtcctgggaagaccaaaagaaaagcatttcaggatgaggggaatggcatgcgcaaaggccctgaggctgaaaatgtccccattgtgttctaagaaatgcagcatgcttggtgktgcctggagcangggacgarggggagatggggaaggagacaaggactgaaggranttagttcccgagnacttntgggtgatttagangatttccttttgtnctggttnagggtgggagcctt

SEQ ID NO: 13 - protein, 269 aa, Artificial Sequence; identical to SEQ ID NO: 3
  Transmembrane Receptor of the Rhodopsin Superfamily

SEQ ID NO: 14 - protein, 314 aa, Homo Sapiens
MDGTNGSTQTHFILLGFSDRPHLERILFVVILIAYLLTLVGNTTIILVSRLDPHLHTPMYFFLAHLSFLDLSFTTSSIPQLLYNLNGHDKTISYMGCAIQLFLFLGLGGVECLLLAVMAYDWCVAICKPLHYMVIMNPRLCRGLVSVTWGCGVANSLAMSPVTLRLPRCGHHEVDHFLREMPALIRMACVSTVAIEGTVFVLAVGVVLSPLVFILLSYSYIVRAVLQIRSASGRQKAFGTCGSHLTVVSLFYGNIIYMYMQPGASSSQDQGKFLTLFYNIVTPLLNPLIYTLRNREVKGALGRLLLGKRELGKE

SEQ ID NO: 15 - DNA, 1629 bp, Homo Sapiens
  CDS (56)...(1000)
tatagggagtcgacccacgcgtccggattgatatttctgttcagctgcagtagagatggatggaaccaatggcagcacccaaacccatttcatcctactgggattctctgaccgaccccatctggagaggatcctctttgtggtcatcctgatcgcgtacctcctgaccctcgtaggcaacaccaccatcatcctggtgtcccggctggacccccacctccacacccccatgtacttcttcctcgcccacctttccttcctggacctcagtttcaccaccagctccatcccccagctgctctacaaccttaatggacatgacaagaccatcagctacatgggctgtgccatccagctcttcctgttcctgggtctgggtggtgtggagtgcctgcttctggctgtcatggcctatgactggtgtgtggctatctgcaagcccctgcactacatggtgatcatgaaccccaggctctgccggggcttggtgtcagtgacctggggctgtggggtggccaactccttggccatgtctcctgtgaccctgcgcttaccccgctgtgggcaccacgaggtggaccacttcctgcgtgagatgcccgccctgatccggatggcctgcgtcagcactgtggccatcgaaggcaccgtctttgtcctggcggtgggtgttgtgctgtcccccttggtgtttatcctgctctcttacagctacattgtgagggctgtgttacaaattcggtcagcatcaggaaggcagaaggcctttggcacctgcggctcccatctcactgtggtctcccttttctatggaaacatcatctacatgtacatgcagccaggagccagttcttcccaggaccagggcaagttcctcacgctcttctacaacattgtcacccccctcctcaatcctctcatctacaccctcagaaacagagaggtgaagggggcactgggaaggttgcttctggggaagagagagctaggaaaggagtaaaggcatctccacctgacttcacctccatccagggccactggcagcatctggaacggctgaattccagctgatattagcccacgactcccaacttgcctttttctggacttttgtgaggctgtttcagttctgacattatgtgtttttgttgttgctcttaaaattgagacggggtctcactctgtcacctagggtggagtgcagtggtgccaccatagctccttcgactattgggcttaagcgatcctcccccacctcagccttccaagtaactgggactacaggtgtgcatcactggcagtgggaattgtggcttttctgtcttctatggagacggggtcttgctgtgttgaccaggctggtcccaaactcctggcctcatgtgatcctcctgccatggcctcctaaagttctgggattacacgtgtgagtcactgtgactggccaacattatgtgatttatgtgtgaactatataacacaaatcatccccaaaacccatcatgatctgtaaagcagctgcaaagaatgaagtgagagaaacagttgtaaagatgagtttccacctacttataccagagtgctaagaggaaataactcttctcaatcagaaaaaaaaaaaaaaaaagg

SEQ ID NO: 16 - protein, 337 aa, Homo Sapiens
MNEPLDYLANASDFPDYAAAFGNCTDENIPLKMHYLPVIYGIIFLVGFPGNAVVISTYIFKMRPWKSSTIIMLNLACTDLLYLTSLPFLIHYYASGENWIFGDFMCKFIRFSFHFNLYSSILFLTCFSIFRYCVIIHPMSCFSIHKTRCAVVACAVVWIISLVAVIPMTFLITSTNRTNRSACLDLTSSDELNTIKWYNLILTATTFCLPLVIVTLCYTTIIHTLTHGLQTDSCLKQKARRLTILLLLAFYVCFLPFHILRVIRIESRLLSISCSIENQIHEAYIVSGPLAALNTFGNLLLYVVVSDNFQQAVCSTVRCKVSGNLEQAKKISYSNNP

SEQ ID NO: 17 - DNA, 1729 bp, Homo Sapiens
  CDS (294)...(1307)
ccttttttttttttttttaacttttatatttttattagatgcatttagtaacttgcctcatagtcattttcttggaaattcaatttcttctccacagggtctcttttgagattaaagagagagaagtggcaaatttaggatgttagaataattttcatttaaaagtagatccttgtttttattaccctatcattaatgttttctgttttcctttatcagcgagttactgctcatttgattcatattgccaaactgaactctcttgttttcttgcaagatgaaaggagacaaccatgaatgagccactagactatttagcaaatgcttctgatttccccgattatgcagctgcttttggaaattgcactgatgaaaacatcccactcaagatgcactacctccctgttatttatggcattatcttcctcgtgggatttccaggcaatgcagtagtgatatccacttacattttcaaaatgagaccttggaagagcagcaccatcattatgctgaacctggcctgcacagatctgctgtatctgaccagcctccccttcctgattcactactatgccagtggcgaaaactggatctttggagatttcatgtgtaagtttatccgcttcagcttccatttcaacctgtatagcagcatcctcttcctcacctgtttcagcatcttccgctactgtgtgatcattcacccaatgagctgcttttccattcacaaaactcgatgtgcagttgtagcctgtgctgtggtgtggatcatttcactggtagctgtcattccgatgaccttcttgatcacatcaaccaacaggaccaacagatcagcctgtctcgacctcaccagttcggatgaactcaatactattaagtggtacaacctgattttgactgcaactactttctgcctccccttggtgatagtgacactttgctataccacgattatccacactctgacccatggactgcaaactgacagctgccttaagcagaaagcacgaaggctaaccattctgctactccttgcattttacgtatgttttttacccttccatatcttgagggtcattcggatcgaatctcgcctgctttcaatcagttgttccattgagaatcagatccatgaagcttacatcgtttctggaccattagctgctctgaacacctttggtaacctgttactatatgtggtggtcagcgacaactttcagcaggctgtctgctcaacagtgagatgcaaagtaagcgggaaccttgagcaagcaaagaaaattagttactcaaacaacccttgaaatatttcatttacttaaccaaaaacaaatacttgctgatactttacctagcatcctaagatgttcaggatgtctccctcaatggaactcctggtaaatactgtgtattcaagtaatcatgtgccaaagccagggcagagcttctagttctttgcaatccctttattgagctcctccactggggagatataagaatgggatgcatgtatatcagcaaagtattcagacatagtattacaagctattggaactcagaggcatcttagagaacatctgttcccaccaacttactatatatacacggaaaccaatttcttacccttgccctagattgctcagtaaatttgtgccaagataggagaaaaccaatcttttcactcatcatttcatgcttctctgcactctgggcctatttgtattga

SEQ ID NO: 18 - protein, 337 aa, Homo Sapiens [sequence omitted]

SEQ ID NO: 19 - DNA, 1334 bp, Homo Sapiens
  CDS (67)...(1080)
gtccgacgtgctggacaaatcttaactcctcaaggactcccaaaaccagagacaccaggagcctgaatggggaacgattctgtcagctacgagtatggggattacagcgacctctcggaccgccctgtggactgcctggatggcgcctgcctggccatcgacccgctgcgcgtggccccgctcccactgtatgccgccatcttcctggtgggggtgccgggcaatgccatggtggcctgggtggctgggaaggtggcccgccggagggtgggtgccacctggttgctccacctggccgtggcggatttgctgtgctgtttgtctctgcccatcctggcagtgcccattgcccgtggaggccactggccgtatggtgcagtgggctgtcgggcgctgccctccatcatcctgctgaccatgtatgccagcgtcctgctcctggcagctctcagtgccgacctctgcttcctggctctcgggcctgcctggtggtctacggttcagcgggcgtgcggggtgcaggtggcctgtggggcagcctggacactggccttgctgctcaccgtgccctccgccatctaccgccggctgcaccaggagcacttcccagcccggctgcagtgtgtggtggactacggcggctcctccagcaccgagaatgcggtgactgccatccggtttctttttggcttcctggggcccctggtggccgtggccagctgccacagtgccctcctgtgctgggcagcccgacgctgccggccgctgggcacagccattgtggtggggttttttgtctgctgggcaccctaccacctgctggggctggtgctcactgtggcggccccgaactccgcactcctggccagggccctgcgggctgaacccctcatcgtgggccttgccctcgctcacagctgcctcaatcccatgctcttcctgtattttgggagggctcaactccgccggtcactgccagctgcctgtcactgggccctgagggagtcccagggccaggacgaaagtgtggacagcaagaaatccaccagccatgacctggtctcggagatggaggtgtaggctggagagacattgtgggtgtgtatcttcttatctcatttcacaagactggcttcaggcatagctggatccaggagctcaatgatgtcttcattttattccttccttcattcaacagatatccatcatgcacttgctatgtgcaaggcctttttaggcactagagatatagcagtgaccaaaacagacacaaatcctgccctcagggagctgatattcttctagtggaggaagacagactataaacaaagata

SEQ ID NO: 20 - protein, 450 aa, Homo Sapiens
MGETMSKRVRLHLGGEAEMEERAFVNPFPDYEAAAGALLASGAAEETGCVRPPATTDEPGLPFHQDGKIIHNFIRRIQTKIKDLLQQMEEGLKTADPHDCSAYTGWTGIALLYLQLYRVTCDQTYLLRSLDYVKRTLRNLNGRRVTFLCGDAGPLAVGAVIYHKLRSDCESQECVTKLLQLQRSVVCQESDLPDELLYGRAGYLYALLYLNTEIGPGTVCESAIKEVVNAIIESGKTLSREERKTERCPLLYQWHRKQYVGAAHGMAGIYYMLMQPAAKVDQETLTEMVKPSIDYVRHKKFRSGNYPSSLSNETDRLVHWCHGAPGVIHMLMQAYKVFKEEKYLKEAMECSDVIWQRGLLRKGYGICHGTAGHGYSFLSLYRLTQDKKYLYRACKFAEWCLDYGAHGCRIPDRPYSLFEGMAGAIHFLSDVLGPETSRFPAFELDSSKRD

SEQ ID NO: 21 - DNA, 1743 bp, Homo Sapiens
  CDS (162)...(1514)
ggcagtgcacgctcagacgccccgctcctcccgccagcgcgcggcctcgctcctcctagaggacgctctctgcgcgggccctcggaggaggcggcggcggggcgagctgcagcgccgggacaggaggtttgtccccgcccgcgcgccgtaccgcggcggagatgggcgagaccatgtcaaaacgcgtccggctccacctgggaggggaggcagaaatggaggaacgggcgttcgtcaaccccttcccggactacgaggccgccgccggggcgctgctcgcctccggagcggccgaagagacaggctgtgttcgtcccccggcgaccacggatgagcccggcctcccttttcatcaggacgggaagatcattcataatttcataagacggatccagaccaaaattaaagatcttctgcagcaaatggaagaagggctgaagacagctgatccccatgactgctctgcttatactggctggacaggcatagcccttttgtacctgcagttgtaccgggtcacatgtgaccaaacctacctgctccgatccctggattacgtaaaaagaacacttcggaatctgaatggccgcagggtcaccttcctctgtggggatgctggccccctggctgttggagctgtgatttatcacaaactcagaagtgactgtgagtcccaggaatgtgtcacaaaacttttgcagctccagagatcggttgtctgccaagaatcagaccttcctgatgagctgctttatggacgggcaggttatctgtatgccttactgtacctgaacacagagataggtccaggcaccgtgtgtgagtcagctattaaagaggtagtcaatgctattattgaatcgggtaagactttgtcaagggaagaaagaaaaacggagcgctgcccgctgttgtaccagtggcaccggaagcagtacgttggagcagcccatggcatggctggaatttactatatgttaatgcagccggcagcaaaagtggaccaagaaaccttgacagaaatggtgaaacccagtattgattatgtgcgccacaaaaaattccgatctgggaattacccatcatcattaagcaatgaaacagaccggctggtgcactggtgccacggcgccccgggggtcatccacatgctcatgcaggcgtacaaggtctttaaggaggagaagtacttgaaagaggccatggagtgtagcgatgtgatttggcagcgaggtttgctgcggaagggctacgggatatgccatgggactgctggccacggctattccttcctgtccctttaccgtctcacgcaggataagaagtacctctaccgagcttgcaagtttgcagagtggtgtctagattacggagcacacgggtgccgcattcctgacagaccctattcgctctttgaaggcatggctggcgctattcactttctctctgatgtcctgggaccagagacatcacggtttccagcatttgaacttgactcttcgaagagggattaaaaggtgcaaaaagacaactaaaatacccatttggaccaaaagccgccagattgcttagtgcctgacacagaaacaactgggaatcctgaaagagaagcagacaccgtcacaggcccctctggttagactagcatgagtgaccgaagccatccatcaacattttctaacagcaccctcatcaatataaaatatgacttcttcacatacaaaaaaaaaaaaaaaagggcgg

SEQ ID NO: 22 - protein, 486 aa, Homo Sapiens
MRGRGSQQQQPTRRQGQKLPSPSPAGKYESAQPGGTQPEPGLGARMAIHKALVMCLGLPLFLFPGAWAQGHVPPGCSQGLNPLYYNLCDRSGAWGIVLEAVAGAGIVTTFVLTIILVASLPFVQDTKKRSLLGTQVFFLLGTLGLFCLVFACVVKPDFSTCASRRFLFGVLFAICFSCLAAHVFALNFLARKNHGPRGWVIFTVALLLTLVEVIINTEWLIITLVRGSGEGGPQGNSSAGWAVASPCAIANMDFVMALIYVMLLLLGAFLGAWPALCGRYKRWRKHGVFVLLTTATSVAIWVVWIVMYTYGNKQHNSPTWDDPTLAIALAANAWAFVLFYVIPEVSQVTKSSPEQSYQGDMYPTRGVGYETILKEQKGQSMFVENKAFSMDEPVAAKRPVSPYSGYNGQLLTSVYQPTEMALMHKVPSEGAYDIILPRATANSQVMGSANSTLRAEDMYSAQSHQAATPPKDGKNSQVFRNPYVWD

SEQ ID NO: 23 - DNA, 2025 bp, Homo Sapiens
  CDS (208)...(1668)
  misc_feature (1)...(2025): n = A,T,C or G
caccncgcgtccgacgggagggcctggacaaaggtgacaaaggctaggtgtcccccacggagacgcgccaaggtagccccgcgcgtgtccgtaggcgcgctctctggaagacgcggtggggggtgcgcagggctgcaccctcacaccaattgccccggcgaaggccgagcccagaaagtgagtgcgcgtgagtgtgcgcgcgcccgcatgcgggggcgtggcagtcaacagcaacaacccacacgccggcagggccagaaactcccatctccctcaccagccggaaagtacgagtcggctcagcctggagggacccaaccagagcctggcctgggagccaggatggccatccacaaagccttggtgatgtgcctgggactgcctctcttcctgttcccaggggcctgggcccagggccatgtcccacccggctgcagccaaggcctcaaccccctgtactacaacctgtgtgaccgctctggggcgtggggcatcgtcctggaggccgtggctggggcgggcattgtcaccacgtttgtgctcaccatcatcctggtggccagcctcccctttgtgcaggacaccaagaaacggagcctgctggggacccaggtattcttccttctggggaccctgggcctcttctgcctcgtgtttgcctgtgtggtgaagcccgacttctccacctgtgcctctcggcgcttcctctttggggttctgttcgccatctgcttctcttgtctggcggctcacgtctttgccctcaacttcctggcccggaagaaccacgggccccggggctgggtgatcttcactgtggctctgctgctgaccctggtagaggtcatcatcaatacagagtggctgatcatcaccctggttcggggcagtggcgagggcggccctcagggcaacagcagcgcaggctgggccgtggcctccccctgtgccatcgccaacatggactttgtcatggcactcatctacgtcatgctgctgctgctgggtgccttcctgggggcctggcccgccctgtgtggccgctacaagcgctggcgtaagcatggggtctttgtgctcctcaccacagccacctccgttgccatatgggtggtgtggatcgtcatgtatacttacggcaacaagcagcacaacagtcccacctgggatgaccccacgctggccatcgccctcgccgccaatgcctgggccttcgtcctcttctacgtcatccccgaggtctcccaggtgaccaagtccagcccagagcaaagctaccagggggacatgtaccccacccggggcgtgggctatgagaccatcctgaaagagcagaagggtcagagcatgttcgtggagaacaaggccttttccatggatgagccggttgcagctaagaggccggtgtcaccatacagcgggtacaatgggcagctgctgaccagtgtgtaccagcccactgagatggccctgatgcacaaagttccgtccgaaggagcttacgacatcatcctcccacgggccaccgccaacagccaggtgatgggcagtgccaactcgaccctgcgggctgaagacatgtactcggcccagagccaccaggcggccacaccgccgaaagacggcaagaactctcaggtctttagaaacccctacgtgtgggactgagtcagcggtggcgaggagaggcggtcggatttggggagggccctgaggacctggccccgggcaagggactctccaggctcctcctccccctggcaggcccagcaacatgtgccccagatgtggaagggcctccctctctgccagtgtttgggtgggtgtcatggggtgtccccacccactcctcaatgtttgtgggaagtcgaggagccaaccccagccttctgcaaggatcacctcggngggtcacacttcaaccaaatagtgttctcggggtnggtggctgggcagcgcctattgtttctctggaagattnctgcaacctcaagaantttccaagcgctcaagcctgggatcttg

SEQ ID NO: 24 - protein, 6 aa, Artificial Sequence
  Amino Acid Fragment
SILTLT

SEQ ID NO: 25 - protein, 7 aa, Artificial Sequence
  Amino Acid Fragment
SILFLTC

SEQ ID NO: 26 - protein, 11 aa, Artificial Sequence
  Amino Acid Fragment
NLYSSILFLTC

SEQ ID NO: 27 - protein, 7 aa, Artificial Sequence
  Amino Acid Fragment
LAVADLL

SEQ ID NO: 28 - protein, 6 aa, Artificial Sequence
  Amino Acid Fragment
LALLLT

SEQ ID NO: 29 - protein, 6 aa, Artificial Sequence
  Amino Acid Fragment
LRRSLP

SEQ ID NO: 30 - protein, 9 aa, Artificial Sequence
  Amino Acid Fragment
FLVGDPGNA

SEQ ID NO: 31 - protein, 5 aa, Artificial Sequence
  Amino Acid Fragment
GNAMV

SEQ ID NO: 32 - protein, 5 aa, Artificial Sequence
  Amino Acid Fragment
LAVAD

SEQ ID NO: 33 - protein, 9 aa, Artificial Sequence
  Amino Acid Fragment
FLVGVPGNA

SEQ ID NO: 34 - protein, 5 aa, Artificial Sequence
  Amino Acid Fragment
ALLLT

SEQ ID NO: 35 - protein, 10 aa, Artificial Sequence
  Amino Acid Fragment
ADLLCCLSLP

SEQ ID NO: 36 - protein, 7 aa, Artificial Sequence
  Amino Acid Fragment
YVGAAHG

SEQ ID NO: 37 - protein, 12 aa, Artificial Sequence
  Amino Acid Fragment
LVHWCHGAPGVI

SEQ ID NO: 38 - protein, 6 aa, Artificial Sequence
  Amino Acid Fragment
QAYKVF

SEQ ID NO: 39 - protein, 5 aa, Artificial Sequence
  Amino Acid Fragment
EEKYL

SEQ ID NO: 40 - protein, 8 aa, Artificial Sequence
  Amino Acid Fragment
SLFEGMAG

SEQ ID NO: 41 - protein, 7 aa, Artificial Sequence
  Amino Acid Fragment
RFPAFEL

SEQ ID NO: 42 - protein, 6 aa, Artificial Sequence
  Amino Acid Fragment
LLQQME

SEQ ID NO: 43 - protein, 12 aa, Artificial Sequence
  Amino Acid Fragment
TFLCGDAGPLAV

SEQ ID NO: 44 - protein, 5 aa, Artificial Sequence
  Amino Acid Fragment
AGIYY

SEQ ID NO: 45 - protein, 5 aa, Artificial Sequence
  Amino Acid Fragment
SGNYP

SEQ ID NO: 46 - protein, 9 aa, Artificial Sequence
  Amino Acid Fragment
QAYKVFKEE

SEQ ID NO: 47 - protein, 5 aa, Artificial Sequence
  Amino Acid Fragment
DVIWQ

SEQ ID NO: 48 - protein, 17 aa, Artificial Sequence
  Amino Acid Fragment
KYLYRACKFAEWCLDYG

SEQ ID NO: 49 - protein, 6 aa, Artificial Sequence
  Amino Acid Fragment
ELLYGR

SEQ ID NO: 50 - protein, 7 aa, Artificial Sequence
  Amino Acid Fragment
PYSLFEG

SEQ ID NO: 51 - protein, 6 aa, Artificial Sequence
  Amino Acid Fragment
VTFLCG

SEQ ID NO: 52 - protein, 469 aa, Homo Sapiens
MAFLMHLLVCVFGMGSWVTINGLWVELPLLVMELPEGWYLPSYLTVVIQLANIGPLLVTLLHHFRPSCLSEVPIIFTLLGVGTVTCIIFAFLWNMTSWVLDGHHSIAFLVLTFFLALVDCTSSVTFLPFMSRLPTYYLTTFFVGEGLSGLLPALVALAQGSGLTTCVNVTEISDSVPSPVPTRETDIAQGVPRALVSALPGMEAPLSHLESRYLPAHFSPLVFFLLLSIMMACCLVAFFVLQRQPRCWEASVEDLLNDQVTLHSIRPREENDLGPAGTVDSSQGQGYLEEKAAPCCPAHLAFIYTLVAFVNALTNGMLPSVQTYSCLSYGPVAYHLAATLSIVANPLASLVSMFLPNRSLLFLGVLSVLGTCFGGYNMAMAVMSPCPLLQGHWGGEVLIVASWVLFSGCLSYVKVMLGVVLRDLSRSALLWCGAAVQLGSLLGALLMFPLVNVLRLFSSADFCNLHCPA

SEQ ID NO: 53 - DNA, 1859 bp, Homo Sapiens
  CDS (218)...(1627)
aacactgcagcgtgaactgaggagtcccggacaggccgcttgctgcagaggatccagtccagatcccaggagagcccctctgccccttcggacctcgtctcccatctacaaaacgtgaagattggcccagttggcgtgtctctacaaaaaggtgcatataccactgccccgctgcaggctgatctgagaaagcctctggcccagggcagataccgccatggccttcctgatgcacctgctggtctgcgtcttcggaatgggctcctgggtgaccatcaatgggctctgggtagagctgcccctgctggtgatggagctgcccgagggctggtacctgccctcctacctcacggtggtcatccagctggccaacatcgggcccctcctggtcaccctgctccatcacttccggcccagctgcctttccgaagtgcccatcatcttcaccctgctgggcgtgggaaccgtcacctgcatcatctttgccttcctctggaatatgacctcctgggtgctggacggccaccacagcatcgccttcttggtcctcaccttcttcctggccctggtggactgcacctcttcagtgaccttcctgccgttcatgagccggctgcccacctactacctcaccaccttctttgtgggtgaaggactcagcggcctcttgcccgccctggtggctcttgcccagggctccggtctcactacctgcgtcaatgtcactgagatatcagacagcgtaccaagccctgtacccacgagggagactgacatcgcacagggagttcccagagctttggtgtccgccctccccggaatggaagcacccttgtcccacctggagagccgctaccttcccgcccacttctcacccctggtcttcttcctcctcctatccatcatgatggcctgctgcctcgtggcgttctttgtcctccagcgtcaacccaggtgctgggaggcttccgtggaagaccttctcaatgaccaggtcaccctccactccatccggccgcgggaagagaatgacttgggccctgcaggcacggtggacagcagccagggccaggggtatctagaggagaaagcagccccctgctgcccggcgcacctggccttcatctataccctggtggccttcgtcaacgcgctcaccaacggcatgctgccctctgtgcagacctactcctgcctgtcctatgggccagttgcctaccacctggctgccaccctcagcattgtggccaaccctcttgcctcgttggtctccatgttcctgcctaacaggtctctgctgttcctgggggtcctctccgtgcttgggacctgctttgggggctacaacatggccatggcggtgatgagcccctgccccctcttgcagggccactggggtggggaagtcctcattgtggcctcgtgggtgcttttcagcggctgcctcagctacgtcaaggtgatgctgggcgtggtcctgcgcgacctcagccgcagcgccctcttgtggtgcggggcggcggtgcagctgggctcgctgctcggagcgctgctcatgttccctctggtcaacgtgctgcggctcttctcgtccgcggacttctgcaatctgcactgtccagcctaggcaggccgccgaccccgcccccatcgctcacggacggaactggggtccagagaggccaggtcacagagcaaggggcaggaacagagagacagagcctgagtaattgaatcatgaacgcaagtgcccactggggactgtggggaagatggcacctggaaatgcaaggtgcggctctatccccaactctgtgtcacactacctgtgacgaccagctcagatctcctttgctttg

SEQ ID NO: 54 - DNA, 1536 bp, Homo Sapiens
  CDS (229)...(1299)
  misc_feature (1)...(1536): n = A,T,C or G
gccacgcgtccgtggagttaaagactgcagcgtgaactgaggagtcccggacaggccgcttgctgcagaggatccagtccagatcccaggagagcccctctgccccttcggacctcgtctcccatctacaaaacgcgaagattggcccagttagcgtgtctctacaaaaaggtgcatataccactgccccgctgcaggctgatctgagaaagcctctggcccaccgccatggccttcctgatgcacctgctggtttgcgtcttcggaatgggctcctgggtgaccatcaatgggctctgggtagagctgcccctgctggtgatggagctgcccgagggctggtacctgccctcctacctcacggtggtcatccagctggccaacatcgggcccctcctggtcaccctgctccatcacttccggcccagctgcctttccgaagtgcccatcatcttcaccctgctgggcgtgggaaccgtcacctgcatcatctttgccttcctctggaatatgacctcctgggtgctggacggccaccacagcatcgccttcttggtcctcaccttcttcctggccctggtggactgcacctcttcagtgaccttcctgccgttcatgagccggctgcccacctactacctcaccaccttctttgtgggtgaaggactcagcggcctcttgcccgccctggtggctcttgcccagggctccggtctcactacctgcgtcaatgtcactgagatatcagacagcgtaccaagccctgtacccacgagggagactgacatcgcacagggagttcccagagctttggtgtccgccctccccggaatggaagcacccttgtcccacctggagagccgctaccttcccgcccacttctcacccctggtcttcttcctcctcctatccatcatgatggcctgctgcctcgtggcgttctttgtcctccagcgtcaacccaggtgctgggaggcttccgtggaagaccttctcaatgaccaggtcaccctccactccatccggccgcgggaagagaatgacttgggccctgcaggcacggtggacagcaagccagggccaggggtatctagaggagaaagcagccccctgctgcccggcgcacctggccttcatctataccctggtggccttcgtcaacgcgctcaccaacggcatgctgccctctgtgcagacctactcctgcctgtcctatgggccagttgcctaccacctggctgccaccctcagcattgtggccaaccctcttgcctcgttggtctccatgttcctgcctaacaggtctctgctgttcctgggggtcctctccgtgcttgggacctgctttgggggctacaacatggccatggcggtgatgagcccctgccccctcttgcagggccactggggtggggaagtcctcattgtggcctcgngggtgctttttcagcggcttgcctcagctacgtcaaggtgatgctgggcgtggtcctgcgcgacctcagccgnagcgccctcttgtggtgccggggcggc

SEQ ID NO: 55 - protein, 356 aa, Homo Sapiens
MAFLMHLLVCVFGMGSWVTINGLWVELPLLVMELPEGWYLPSYLTVVIQLANIGPLLVTLLHHFRPSCLSEVPIIFTLLGVGTVTCIIFAFLWNMTSWVLDGHHSIAFLVLTFFLALVDCTSSVTFLPFMSRLPTYYLTTFFVGEGLSGLLPALVALAQGSGLTTCVNVTEISDSVPSPVPTRETDIAQGVPRALVSALPGMEAPLSHLESRYLPAHFSPLVFFLLLSIMMACCLVAFFVLQRQPRCWEASVEDLLNDQVTLHSIRPREENDLGPAGTVDSKPGPGVSRGESSPLLPGAPGLHLYPGGLRQRAHQRHAALCADLLLPVLWASCLPPGCHPQHCGQPSCLVGLHVPA

SEQ ID NO: 56 - protein, 384 aa, Homo Sapiens
MTNSSSTSTSSTTGGSLLLLCEEEESWAGRRIPVSLLYSGLAIGGTLANGMVIYLVSSFRKLQTTSNAFIVNGCAADLSVCALWMPQEAVLGLLPTGSAEPPADWDGAGGSYRLLRGGLLGLGLTVSLLSHCLVALTRYLLITRAPATYQALYQRRHTAGMLALSWALALGLVLLLPPWAPRPGAAPPRVHYPALLAAAALLAQTALLLHCYLGIVRRVRVSVKRVSVLNFHLLHQLPGCAAAAAAFPGAQHAPGPGGAAHPAQAQPLPPALHPRRAQRRLSGLSVLLLCCVFLLATQPLVWVSLASGFSLPVPWGVQAASWLLCCALSALNPLLYTWRNEEFRRSVRSVLPGVGDAAAAAVAATAVPAVSQAQLGTRAAGQHW

SEQ ID NO: 57 - DNA, 2040 bp, Homo Sapiens
  CDS (189)...(1343)
  misc_feature (1)...(2040): n = A,T,C or G
cacccccgtcctcctcctcgagctccctttctccccctcccccagcccattattctgcttcagtcttttgtgtcagyggcagarggctgaggggatggatttgcccttctggcaggcaggacagtgtcaggatggaccgcgctgccagaagccgacgctagcgagggaggtgtgaagagttggccagaatgaccaactcctcctccacatccacctcctccaccaccggtggctcgctgctgctgctctgcgaggaagaggagtcgtgggcgggccggcgcatcccggtgtcactcctgtattcgggcctggccatcgggggcacgctggccaacggcatggtcatctatctcgtgtcgtccttccgaaagctgcagaccaccagcaacgccttcatcgtgaacggctgcgccgccgacctcagcgtctgcgccctctggatgccgcaggaggcggtgctcgggctcctgcccaccggctctgcggagccccccgcagactgggacggcgctgggggcagctaccgcctgctacggggtgggctgctgggcctcggactcacggtgtccctcctctcccactgcctcgtggccctgacccgctacctgctcatcacccgggcgcccgccacctaccaggcgctgtaccagaggcgccacacggcgggcatgctggcgctgtcctgggcgctcgccctgggcctcgtgctgctgctcccgccctgggcaccgcggcccggcgccgcgccaccgcgagtccactacccggcgctgctggccgccgcggcgctgctggcgcagacagctctgctgctgcactgctacctgggcatcgtgcgccgcgtgcgtgtcagcgtcaagcgggtcagcgtgctcaacttccacctgctgcaccagttgcccggctgcgccgccgccgccgccgccttcccgggcgcccagcacgcgccgggccccggtggcgccgcgcacccggcgcaggcccagcccctgccgcccgcgctgcacccgcggcgggcacagcggcgtctcagcggcctgtcggtgctgctgctctgctgcgtcttcctgctggccacgcagccactggtgtgggtgagcctggccagcggcttctcgctgccggtgccctggggagtgcaggcggccagctggctcctgtgctgcgccctgtccgcgctcaatccgctgctctacacgtggaggaacgaggagttccgccgctccgtgcgctcagtcctgccgggcgtcggcgacgcggcggccgctgccgttgccgccacagccgtgcccgcagtgtcccaggcgcaactgggcacccgcgccgcgggccagcactggtaacctagccggggcccgagggaagcggagatccccggcttccgacgtccttgggcaccgtcgcctccttccctcctagggcatcccctgcctgaacgaagacttccgccgcgaagcccgatagatcgggggaaaatggggccttcgaccccagcgggctacctgaaccaaggcgtctctctaagtggggcgcccgaagtcattttggacggccacctgatttttaccctttgtttctgtgttttagaggaatcctaaagacagaacaccagagacttgaagaacttgcaaactggcgttttaaaataaccggttaatttatttccacacagtttgtttttgaaaaagagctttcataatgtataaccctttccactttcatcgtcttatatatgaagcgccttgagtgtgcatgaaccaaaggaaataacattgaagaaggaaaacaatatgtagaaagtattyttagaaagtaacctgtctttgatgatgcttctcttaccatttagntntttgtatattaccctggggcagttgaagccctaggtgtgcccaccagtatgagttgccattaagacctcaagccctttattcttaaaagggtttytaataaaagtctttctcaaatgaggtagaatcttagccaagtngagaaaaaaaaattattttatgctccttttttttcgcacctcttaaagac

SEQ ID NO: 58 - protein, 121 aa, Artificial Sequence
  Consensus Sequence For Seven Transmembrane Receptors
GNLLVILVILRTKKLRTPTNIFILNLAVADLLFLLTLPPWALYYLVGGSEDWPFGSALCKLVTALDVVNMYASILLLTAISIDRYLAIVHPLRYRRRRTSPRRAKVVILLVWVLALLLSLP

SEQ ID NO: 59 - protein, 14 aa, Artificial Sequence
  Consensus Sequence For Seven Transmembrane Receptors
WLAYVNSCLNPIIY

SEQ ID NO: 60 - DNA, 1347 bp, Homo Sapiens
  CDS (176)...(886)
  misc_feature (1)...(1347): n = A,T,C or G
tycttagggagtcgacccacgcgtccggcggggccctacacaaacccgytggtagcgctgggccgactcgcccagcctggacccattcagtcagaggcagccagcgggacctgcttcaccgagcgcagcgaagccgagacccgggctggcccctctgctgcccccggagcgggccatgccgccgcgggagctgagcgaggccgagccgcccccgctccgggccccgacccctcccccgcggcggcgtagcgcgcccccagagctgggcatcaagtgcgtgctggtgggcgacggcgccgtgggcaagagcagcctcatcgtcagctacacctgcaatgggtaccccgcgcgctaccggcccactgcgctggacaccttctctgtgcaagtcctggtggatggagctccggtgcgcattgagctctgggacacagcgggacaggaggattttgaccgacttcgttccctttgctacccggataccgatgtcttcctggcgtgcttcagcgtggtgcagcccagctcctttcaaaacatcacagagaaatggctgcccgagatccgcacgcacaacccccaggcgcctgtgctgctggtgggcacccaggccgacctgagggacgatgtcaacgtactaattcagctggaccaggggggccgggagggccccgtgccccaaccccaggctcagggtctggccgagaagatccgagcctgctgctaccttgagtgctcagccttgacgcagaagaacttgaaggaagtatttgactcggctattctcagtgccattgagcacaaagcccggctggagaagaaactgaatgccaaaggtgtgcgcaccctctcccgctgccgctggaagaagttcttctgcttcgtttgagcagctatggctgcatagcaagtagtaggcaggaggccaaagacttctgagacctggggcacccgggcctttgcggcagctactggcagggcctggccacctcataggactcagttcccttctgaacactcgggggacatgggcctctaactgcccactctgatatgcctgggtgagcctaggagggaaggctctgatttggatttctccagtcaaagctcacagaaaaaaacctggcactttgattttcatgggatggtcctaacagggtcaagtcacctccgagcagtttgggaacccagtttcttgtcctgggccctcaggtcagcctggctgaattaggacccttncttggcacargggtgagaaagaacttggggaacgcttggcattatgganggctggaaaggggctyaaccccgatttggaaaaaagtttgggaatggaattggccaaaaaat

SEQ ID NO: 61 - protein, 236 aa, Homo Sapiens
MPPRELSEAEPPPLRAPTPPPRRRSAPPELGIKCVLVGDGAVGKSSLIVSYTCNGYPARYRPTALDTFSVQVLVDGAPVRIELWDTAGQEDFDRLRSLCYPDTDVFLACFSVVQPSSFQNITEKWLPEIRTHNPQAPVLLVGTQADLRDDVNVLIQLDQGGREGPVPQPQAQGLAEKIRACCYLECSALTQKNLKEVFDSAILSAIEHKARLEKKLNAKGVRTLSRCRWKKFFCFV

SEQ ID NO: 62 - DNA, 1023 bp, Homo Sapiens
  CDS (245)...(886)
  misc_feature (1)...(1023): n = A,T,C or G
gtcgacccacgcgtccggtcagagtgcgtggtgctgatgctgctgccatttcatcacctttgcgagcgcagcatccatccctccgctctcccggcgcctgggcctacccagcttcgggctcccaggccagcgatgcgctcgcggctgagctagatcctgccgagccgcgctctctgaggcgtcggcggggcgccccctcccgccgtccccggtccgggccaaggagacctgcagagccgcggccatggaggccatctggctgtaccagttccggctcattgtcatcggggattccacagtgggcaagtcctgcctgatccgccgcttcaccgagggtcgctttgcccaggtttctgaccccaccgtgggggtggattttttctcccgcttggtggagatcgagccaggaaaacgcatcaagctccagatctgggataccgcgggtcaagagaggttcagatccatcactcgcgcctactacaggaactcagtaggtggtcttctcttatttgacattaccaaccgcaggtccttccagaatgtccatgagtggttagaagagaccaaagtacacgttcagccctaccaaattgtatttgttctggtgggtcacaagtgtgacctggatacacagaggcaagtgactcgccacgaggccgagaaactggctgctgcatacggcatgaagtacattgaaacgtcagcccgagatgccattaatgtggagaaagccttcacagacctgacaagagacatatatgagctggttaaaaggggggagattacaatccaggagggctgggaaggggtgaagagtggatttgtaccaaatgtggttcactcttcagaagaggttgtcaaatcagagaggagatgtttgtgctagtcagttcttttatttccaaaacatgctctcctacttgaactgaaaagtaagagaaataaatagaatctttgtgtnaaaaaaaaaaaaaaaaaaaaaaaaaaaaaaaaaaaaaaaaagggcggccgctagacnagtct

SEQ ID NO: 63 - protein, 213 aa, Homo Sapiens
MEAIWLYQFRLIVIGDSTVGKSCLIRRFTEGRFAQVSDPTVGVDFFSRLVEIEPGKRIKLQIWDTAGQERFRSITRAYYRNSVGGLLLFDITNRRSFQNVHEWLEETKVHVQPYQIVFVLVGHKCDLDTQRQVTRHEAEKLAAAYGMKYIETSARDAINVEKAFTDLTRDIYELVKRGEITIQEGWEGVKSGFVPNVVHSSEEVVKSERRCLC

SEQ ID NO: 64 - DNA, 1161 bp, Homo Sapiens
  CDS (18)...(641)
cacgcgtccgcgagaagatggcgaagacgtacgattatctcttcaagctcctgctgatcggcgactcgggggtaggcaagacctgcctcctgttccgcttctcagaggacgccttcaacaccaccttcatctccaccatcggaattgattttaaaattagaacgatagaactagatggaaagaaaattaagcttcagatatgggacacagcgggtcaggaaagattccgaacaatcacgacagcgtactacagaggagccatgggcattatgctggtctatgacatcacaaatgaaaaatcctttgacaatattaaaaattggatcagaaacattgaagagcatgcctcttccgatgtcgaaagaatgatcctgggtaacaaatgtgatatgaatgacaaaagacaagtgtcaaaagaaagaggggagaagctagcaattgactatgggattaaattcttggagacaagcgcaaaatccagtgcaaatgtagaagaggcattttttacacttgcacgagatataatgacaaaactcaacagaaaaatgaatgacagcaattcagcaggagcaggtggaccagtgaaaataacagaaaaccgatcaaagaagaccagtttctttcgttgctcgctactttgatgaactctttctgagagactgcagcacacctagagggccctttcctgcttctctgaaagcacaggtcacccagcctcagaatcacacctcccggctgctgctgagagcaccactgaacttagacctctcaacacagtatgccaagtggattccagcctcatggcctagcaaaagaacagactccctttttcaaacatggaagcaatgaagtggagacacatgcaggacctaactcgttttttccttgttttattacctgttgcagaagcggttatctttctttttttactttgcacatcagtgttagcctttccctatttcagcacaatcttagactcatatttgcacacttttgtgtcgtgaagttctagacaaatttgtacatgtggcaatgttaaaagagcatttacagcagaggttaatatactaaaattaaagggtatttggtctggttcatatggtcaaatattactgccttggtagcatttatttaagggctttttcttaaataagaatcatt

SEQ ID NO: 65 - protein, 207 aa, Homo Sapiens
MAKTYDYLFKLLLIGDSGVGKTCLLFRFSEDAFNTTFISTIGIDFKIRTIELDGKKIKLQIWDTAGQERFRTITTAYYRGAMGIMLVYDITNEKSFDNIKNWIRNIEEHASSDVERMILGNKCDMNDKRQVSKERGEKLAIDYGIKFLETSAKSSANVEEAFFTLARDIMTKLNRKMNDSNSAGAGGPVKITENRSKKTSFFRCSLL

SEQ ID NO: 66 - DNA, 1199 bp, Homo Sapiens
  CDS (193)...(744)
gtcgacccacgcgtccggatcacgtgggcagctccgggcgcggcgcttgttttggtttccttctaacttgcccacggcagcttcggggtgagcgactttcctgcaccagctgccgcgcctgctcacaccctgacctcgttttcgggctctctgagcccgcagttccgcaagcccctggggcgggctcctgccatgccgctagtccgctacaggaaggtggtcatcctcggataccgctgtgtagggaagacatctttggcacatcaatttgtggaaggcgagttctcggaaggctacgatcctacagtggagaatacttacagcaagatagtgactcttggcaaagatgagtttcacctacatctggtggacacagcagggcaggatgagtacagcattctgccctattcattcatcattggggtccatggttatgtgcttgtgtattctgtcacctctctgcatagcttccaagtcattgagagtctgtaccaaaagctacatgaaggccatgggaaaacccgggtgccagtggttctagtggggaacaaggcagatctctctccagagagagaggtacaggcagttgaaggaaagaagctggcagagtcctggggtgcgacatttatggagtcatctgctcgagagaatcagctgactcaaggcatcttcaccaaagtcatccaggagattgcccgtgtggagaattcctatgggcaagagcgtcgctgccatctcatgtgagcccttgggtgtggggtaactgccttgcttctgcccccggcacttgccatgttccagtggggggcagatcctcaggacttcacgggtatggttgccagctgtgttcctggcccctggacacacagtgtggcatcctcatgtttgcacactttccccaggctccagtggcctggatgtcaatgtttacaaaggggcaaggacctctcatggacactggcctctagccctctgtttttgtttgatgaattctgttataacctatggggtcaggatatgagtcctgggcattatttatccaggacccatcctcttgggtgggttttgggtgttggctgggtaaggggagccggggacttctgaaatagagctggctccctggggtgacaatgtatatatgcaaataaattgagaaatctttaaaaaaaaaaaaaaaaaaaaaaaaaaagggcggccgc

SEQ ID NO: 67 - protein, 183 aa, Homo Sapiens
MPLVRYRKVVILGYRCVGKTSLAHQFVEGEFSEGYDPTVENTYSKIVTLGKDEFHLHLVDTAGQDEYSILPYSFIIGVHGYVLVYSVTSLHSFQVIESLYQKLHEGHGKTRVPVVLVGNKADLSPEREVQAVEGKKLAESWGATFMESSARENQLTQGIFTKVIQEIARVENSYGQERRCHLM

SEQ ID NO: 68 - DNA, 1116 bp, Homo Sapiens
  CDS (124)...(699)
ctcctttggggagtcgacccacgcgtccggacgggcacgccaggcgccgttgccacccgggatggcgaggcccccgagcgctccccgccctgcagtccgagctacgacctcacgggcaaggtgatgcttctgggagacacaggcgtcggcaaaacatgtttcctgatccaattcaaagacggggccttcctgtccggaaccttcatagccaccgtcggcatagacttcaggaacaaggtggtgactgtggatggcgtgagagtgaagctgcagatctgggacaccgctgggcaggaacggttccgaagcgtcacccatgcttattacagagatgctcaggccttgcttctgctgtatgacatcaccaacaaatcttctttcgacaacatcagggcctggctcactgagattcatgagtatgcccagagggacgtggtgatcatgctgctaggcaacaaggcggatatgagcagcgaaagagtgatccgttccgaagacggagagaccttggccagggagtacggtgttcccttcctggagaccagcgccaagactggcatgaatgtggagttagcctttctggccatcgccaaggaactgaaataccgggccgggcatcaggcggatgagcccagcttccagatccgagactatgtagagtcccagaagaagcgctccagctgctgctccttcatgtgaatcccagggggcagagaggaggctctggaggcacacaggatgcagccttccccctcccaggcctggcttattccaagaggctgagccaatggggagaaagatggaggactcactgcacagccgcttcctagcagggagctatactccaactcctacttgagttcctgcggtctccccgcatccacagggagggtaaaacacttagcttttattttaatagtacataatttaataccaaaaaaggcgcctggatccccaaaaaaccgaggctgggagctagtggcccttttgctttctaggacttggggggccggccctccctcctaagcataacaaaggtggtgttgctccagctcagccccaggggacacagatgcactttgggggtgagggcaagtaatgactccatcgcaccct

SEQ ID NO: 69 - protein, 191 aa, Homo Sapiens
MLLGDTGVGKTCFLIQFKDGAFLSGTFIATVGIDFRNKVVTVDGVRVKLQIWDTAGQERFRSVTHAYYRDAQALLLLYDITNKSSFDNIRAWLTEIHEYAQRDVVIMLLGNKADMSSERVIRSEDGETLAREYGVPFLETSAKTGMNVELAFLAIAKELKYRAGHQADEPSFQIRDYVESQKKRSSCCSFM

SEQ ID NO: 70 - protein, 198 aa, Artificial Sequence
  Pfam accession number PF00071
KLVLIGDSGVGKSSLLIRFTDNKFVEEYIPTIGVDFYTKTVEVDGKTVKLQIWDTAGQERFRALRPAYYRGAQGFLLVYDITSRDSFENVKKWLEEILRHADKDENVPIVLVGNKCDLEDDEDLELTEGQKRVVSTEEGEALAKELGALPFMETSAKTNTNVEEAFEELAREILKKVSEVNVNLDQPAKKKKSKCCIL

SEQ ID NO: 71 - protein, 373 aa, Homo Sapiens
MANTTGEPEEVSGALSPPSASAYVKLVLLGLIMCVSLAGNAILSLLVLKERALHKAPYYFLLDLCLADGIRSAVCFPFVLASVRHGSSWTFSALSCKIVAFMAVLFCFHAAFMLFCISVTRYMAIAHHRFYAKRMTLWTCAAVICTAWTLSVAMAFPPVFDVGTYKFIRGEDQCIFEHRYFKANDTLGFMLMLAVLMAATHAVYGKLLLFEYRHRKMKPVQMVPAISQNWTFHGPGATGQAAANWIAGFGRGPMPPTLLGIRQNGHAASRRLLGMDEVKGEKQLGRMFYAITLLFLLLWSPYIVACYWRVFVKACAVPHRYLATAVWMSFAQAAVNPIVCFLLNKDLKKCLRTHAPCWGTGGAPAPREPYCVM

SEQ ID NO: 72 - DNA, 2548 bp, Homo Sapiens
  CDS (733)...(1854)
tgtgaaacttttggttgagctgtgtgtgtgcgtgcatgtatgtgtagcgggttgtgatgtaaaatgtatttttttactgtgggtggcagtaaaaaaagtctgaacacaaccttagagctttgcaaaaggggagaagagctgcaccaacatccctgccccacagatccaccagtggaggaaacagataaccaaagacatccaaagaaatgcagcatcctcacctgacaaggagcgggtaggagcaggagtggcccaggggcagggcctggcaccagccagggcataattggggagggctcgtagacacactaaccctaccctttctgtttcctcctcatctttcctttccatctgtttctcatggactcctgtctgtctctctctccctcccctctttctctctcctcgctctttctcatcccctccatttctgtgtcaatctcaatccatttatatcggtggccacttttctatctctttgttctatctctctctctctctttcccactttgtctctgcacgcctgttgtgtttttctgcctgtctctctcttgccctcatctctctgtctctctcttgccctcatctctctgtctctctgtgtctgtgtctcccccgctcattcccatttgcaggtgcaatgtagcaggacaactcatggagcccccccgggcccatcgagtaccggactggctgaccccctagggttggcagtagcccctgacccccagtatggccaacactaccggagagcctgaggaggtgagcggcgctctgtccccaccgtccgcatcagcttatgtgaagctggtactgctgggactgattatgtgcgtgagcctggcgggtaacgccatcttgtccctgctggtgctcaaggagcgtgccctgcacaaggctccttactacttcctgctggacctgtgcctggccgatggcatacgctctgccgtctgcttcccctttgtgctggcttctgtgcgccacggctcttcatggaccttcagtgcactcagctgcaagattgtggcctttatggccgtgctcttttgcttccatgcggccttcatgctgttctgcatcagcgtcacccgctacatggccatcgcccaccaccgcttctacgccaagcgcatgacactctggacatgcgcggctgtcatctgcacggcctggaccctgtctgtggccatggccttcccacctgtctttgacgtgggcacctacaagtttattcggggggaggaccagtgcatctttgagcatcgctacttcaaggccaatgacacgctgggcttcatgcttatgttggctgtgctcatggcagctacccatgctgtctacggcaagctgctcctcttcgagtatcgtcaccgcaagatgaagccagtgcagatggtgccagccatcagccagaactggacattccatggtcccggggccaccggccaggctgctgccaactggatcgccggctttggccgtgggcccatgccaccaaccctgctgggtatccggcagaatgggcatgcagccagccggcggctactgggcatggacgaggtcaagggtgaaaagcagctgggccgcatgttctacgcgatcacactgctctttctgctcctctggtcaccctacatcgtggcctgctactggcgagtgtttgtgaaagcctgtgctgtgccccaccgctacctggccactgctgtttggatgagcttcgcccaggctgccgtcaacccaattgtctgcttcctgctcaacaaggacctcaagaagtgcctgaggactcacgccccctgctggggcacaggaggtgccccggctcccagagaaccctactgtgtcatgtgaagcaggctggtaggcagacaggcagagagaaggtcatggccaccgtgatggggccaacagcaagggaggggtaggggcccatacaggagtcctcctttctgagctccagccccagcccctcgaaccacctgtaatctaggcacctttgccaacaccttccaaggatggaggactgggcgagggactgggaaagaggcatatttagttttgtggggcctgtctccgctgcctccttctccacttctacaatctcattctctctctctctctctctgtctctctctctctctctctctctctctcagaagtgacaattcagaaaaagaaaagaacactgagaatgcaggtttttctactaacagctgaggagacaggctttcttactttaatgtctctccttctgacactgtccagaagggggaatttgtcctgtaaaatagacttccagagctcttttgccccctctgctcccatgcaccctccccttccaagtcctttagcaaggcctgggatgtttagggagaaagtggtccaaggctgctgacaagagggaccaaaggggggtgctgggttcccagggagcaggggatgtttaattactatgtcatgtgcaatgttgttttaggccaacccttgccccaagcccagtctcttctccctccccaccatgtccagacctccaaaatggttcttg

SEQ ID NO: 73 - protein, 188 aa, Artificial Sequence
  Seven Transmembrane Segment Rhodopsin Superfamily
GNLLVILVILRTKKLRTPTNIFILNLAVADLLFLLTLPPWALYYLVGGSEDWPFGSALCKLVTALDVVNMYASILLLTAISIDRYLAIVHPLRYRRRRTSPRRAKVVILLVWVLALLLSLPPLLKTLLVVVVVFVLCWLPYFIVLLLDTLCLSIIMSSTCELERVLPTALLVTLWLAYVNSCLNPIIY

SEQ ID NO: 74 - protein, 6 aa, Artificial Sequence
  Amino Acid Fragment
SLLAIA

SEQ ID NO: 75 - protein, 6 aa, Artificial Sequence
  Amino Acid Fragment
DPTLAI

SEQ ID NO: 76 - protein, 7 aa, Artificial Sequence
  Amino Acid Fragment
AWGIVLE

SEQ ID NO: 77 - protein, 9 aa, Artificial Sequence
  Amino Acid Fragment
FLLGTLGLF

SEQ ID NO: 78 - protein, 6 aa, Artificial Sequence
  Amino Acid Fragment
ICFSCL

SEQ ID NO: 79 - protein, 8 aa, Artificial Sequence
  Amino Acid Fragment
VYQPTEMA

SEQ ID NO: 80 - protein, 7 aa, Artificial Sequence
  Amino Acid Fragment
EAVAGAG

SEQ ID NO: 81 - protein, 9 aa, Artificial Sequence
  Amino Acid Fragment
MDFVMALIY

SEQ ID NO: 82 - protein, 9 aa, Artificial Sequence
  Amino Acid Fragment
ENKAFSMDE

SEQ ID NO: 83 - protein, 59 aa, Artificial Sequence
  Amino Acid Fragment
MYTYGNKQHNSPTWDDPTLAIALAANAWAFVLFYVIPEVSQVTKSSPEQSYQGDMYPTR

(19) United States
(12) Patent Application Publication
(10) Pub. No.: US 2003/0224417 A1
(43) Pub. Date: Dec. 4, 2003

(54) 14400, 2838, 14618, 15334, 14274, 32164, 39404, 38911, 26904, 31237, 18057, 16405, 32705, 23224, 27423, 32700, 32712 and 12216, novel seven-transmembrane proteins/G-protein coupled receptors

(75) Inventors: Maria A. Glucksmann, Lexington, MA (US); Nadine S. Weich, Brookline, MA (US); John Joseph Hunter, Somerville, MA (US); David White, Braintree, MA (US); Kyle J. MacBeth, Boston, MA (US); Mark J. Williamson, Saugus, MA (US); Rachel E. Meyers, Newton, MA (US); Miyoung Chun, Belmont, MA (US)

Correspondence Address: Jean M. Silveri, Millennium Pharmaceuticals, Inc., 75 Sidney Street, Cambridge, MA, 02139, US

(73) Assignee: Millennium Pharmaceuticals, Inc.

(21) Appl. No.: 10/400,991

(22) Filed: Mar. 27, 2003

Related U.S. Application Data
(63) Continuation-in-part of application No. 10/190,469, filed on Jul. 5, 2002.
Application No. 10/190,469 is a continuation of application No. 09/439,159, filed on Nov. 12, 1999, now abandoned.
Application No. 09/439,159 is a division of application No. 09/137,063, filed on Aug. 20, 1998, now abandoned.
(63) Continuation-in-part of application No. 10/167,192, filed on Jun. 11, 2002.
Application No. 10/167,192 is a division of application No. 09/420,187, filed on Oct. 18, 1999, now abandoned.
Application No. 09/420,187 is a continuation-in-part of application No. 09/173,869, filed on Oct. 16, 1998, now abandoned.
(63) Continuation-in-part of application No. 10/339,056, filed on Jan. 9, 2003.
Application No. 10/339,056 is a continuation of application No. 09/377,429, filed on Aug. 19, 1999, now abandoned.
Application No. 09/377,429 is a continuation-in-part of application No. 09/136,726, filed on Aug. 19, 1998, now abandoned.
(63) Continuation-in-part of application No. 09/911,583, filed on Jul. 24, 2001, now abandoned.
Application No. 09/911,583 is a continuation-in-part of application No. 09/476,287, filed on Dec. 30, 1999, now abandoned.
(63) Continuation-in-part of application No. 09/475,790, filed on Dec. 30, 1999, now abandoned.
(63) Continuation-in-part of application No. 09/779,448, filed on Feb. 8, 2001, now abandoned.
(63) Continuation-in-part of application No. 09/347,094, filed on Jul. 2, 1999, now abandoned.
(63) Continuation-in-part of application No. 09/794,257, filed on Feb. 27, 2001.
(63) Continuation-in-part of application No. 09/448,687, filed on Nov. 24, 1999.
Application No. 09/448,687 is a continuation-in-part of application No. 09/200,302, filed on Nov. 25, 1998, now abandoned.
(60) Provisional application No. 60/180,986, filed on Feb. 8, 2000.
(60) Provisional application No. 60/185,606, filed on Feb. 29, 2000.

(51) Int. Cl. C12Q 1/68; G01N 33/53; C07K 14/705; C12P 21/02; C12N 5/06; A61K 38/17

(52) U.S. Cl. 435/6; 435/7.1; 435/69.1; 435/320.1; 435/325; 530/350; 536/23.5; 514/12

(57) ABSTRACT

The present invention relates to a newly identified receptor belonging to the superfamily of G-protein-coupled receptors. The invention also relates to polynucleotides encoding the receptor. The invention further relates to methods using the receptor polypeptides and polynucleotides as a target for diagnosis and treatment in receptor-mediated disorders. The invention further relates to drug-screening methods using the receptor polypeptides and polynucleotides to identify agonists and antagonists for diagnosis and treatment. The invention further encompasses agonists and antagonists based on the receptor polypeptides and polynucleotides. The invention further relates to procedures for producing the receptor polypeptides and polynucleotides.

### RELATED APPLICATIONS

The present application is a continuation-in-part of U.S. patent application Ser. No. 10/190,469, filed Jul. 05, 2002 (pending), which is a continuation of U.S. patent application Ser. No. 09/439,159, filed Nov. 12, 1999, which is a divisional of U.S. patent application Ser. No. 09/137,063, filed Aug. 20, 1998. The present application is also a continuation-in-part of U.S. patent application Ser. No. 10/167,192, filed Jun. 11, 2002 (pending), which is a divisional of U.S. patent application Ser. No. 09/420,187, filed Oct. 18, 1999, which is a continuation-in-part of U.S. patent application Ser. No. 09/173,869, filed Oct. 16, 1998. The present application is also a continuation-in-part of U.S. patent application Ser. No. 10/339,056, filed on Jan. 9, 2003 (pending), which is a continuation of U.S. patent application Ser. No. 09/377,429, filed Aug. 19, 1999, which is a continuation-in-part of U.S. patent application Ser. No. 09/136,726, filed Aug. 19, 1998. The present application is also a continuation-in-part of U.S. patent application Ser. No. 09/911,583, filed Jul. 24, 2001 (pending), which is a continuation-in-part of U.S. patent application Ser. No. 09/476,287, filed Dec. 30, 1999. The present application is also a continuation-in-part of U.S. patent application Ser. No. 09/475,790, filed Dec. 30, 1999 (pending). The present application is also a continuation-in-part of U.S. patent application Ser. No. 09/779,448, filed Feb. 8, 2001 (pending), which claims the benefit of U.S. Provisional Application Serial No. 60/180,986, filed Feb. 8, 2000. The present application is also a continuation-in-part of U.S. patent application Ser. No. 09/347,094, filed Jul. 02, 1999 (pending). The present application is also a continuation-in-part of U.S. patent application Ser. No. 09/794,257, filed Feb. 27, 2001 (pending), which claims the benefit of U.S. Provisional Application Serial No. 60/185,606, filed Feb. 29, 2000. The present application is also a continuation-in-part of U.S. patent application Ser. No. 09/448,687, filed Nov. 24, 1999 (pending), which is a continuation-in-part of U.S. patent application Ser. No. 09/200,302, filed Nov. 25, 1998. The entire contents of each of the above-referenced patent applications are incorporated herein by this reference.

### FIELD OF THE INVENTION

The present invention relates to newly identified receptors belonging to the superfamily of G-protein-coupled receptors. The invention also relates to polynucleotides encoding the receptors. The invention further relates to methods of using the receptor polypeptides and polynucleotides as targets for diagnosis and treatment in receptor-mediated disorders. The invention further relates to drug-screening methods using the receptor polypeptides and polynucleotides to identify agonists and antagonists for diagnosis and treatment. The invention further encompasses agonists and antagonists based on the receptor polypeptides and polynucleotides. The invention further relates to procedures for producing the receptor polypeptides and polynucleotides.

### BACKGROUND OF THE INVENTION

### G-protein Coupled Receptors

G-protein coupled receptors (GPCRs) constitute a major class of proteins responsible for transducing a signal within a cell. GPCRs have three structural domains: an amino terminal extracellular domain, a transmembrane domain containing seven transmembrane segments, three extracellular loops, and three intracellular loops, and a carboxy terminal intracellular domain. Upon binding of a ligand to an extracellular portion of a GPCR, a signal is transduced within the cell that results in a change in a biological or physiological property of the cell. GPCRs, along with G-proteins and effectors (intracellular enzymes and channels modulated by G-proteins), are the components of a modular signaling system that connects the state of intracellular second messengers to extracellular inputs.

GPCR genes and gene-products are potential causative agents of disease (Spiegel et al. (1993) J. Clin. Invest. 92:1119-1125; McKusick et al. (1993) J. Med. Genet. 30:1-26). Specific defects in the rhodopsin gene and the V2 vasopressin receptor gene have been shown to cause various forms of retinitis pigmentosum (Nathans et al. (1992) Annu. Rev. Genet. 26:403-424), and nephrogenic diabetes insipidus (Holtzman et al. (1993) Hum. Mol. Genet. 2:1201-1204). These receptors are of critical importance to both the central nervous system and peripheral physiological processes. Evolutionary analyses suggest that the ancestor of these proteins originally developed in concert with complex body plans and nervous systems.

The GPCR protein superfamily can be divided into five families: Family I, receptors typified by rhodopsin and the β2-adrenergic receptor and currently represented by over 200 unique members (Dohlman et al. (1991) Annu. Rev. Biochem. 60:653-688); Family II, the parathyroid hormone/calcitonin/secretin receptor family (Juppner et al. (1991) Science 254:1024-1026; Lin et al. (1991) Science 254:1022-1024; Family III, the metabotropic glutamate receptor family (Nakanishi (1992) Science 258 597:603); Family IV, the cAMP receptor family, important in the chemotaxis and development of D. discoideum (Klein et al. (1988) Science 241:1467-1472); and Family V, the fungal mating pheromone receptors such as STE2 (Kurjan (1992) Annu. Rev. Biochem. 61:1097-1129).

There are also a small number of other proteins which present seven putative hydrophobic segments and appear to be unrelated to GPCRs; they have not been shown to couple to G-proteins. Drosophila expresses a photoreceptor-specific protein, bride of sevenless (boss), a seven-transmembrane-segment protein which has been extensively studied and does not show evidence of being a GPCR (Hart et al. (1993) Proc. Natl. Acad. Sci. USA 90:5047-5051). The gene frizzled (fz) in Drosophila is also thought to be a protein with seven transmembrane segments. Like boss, fz has not been shown to couple to G-proteins (Vinson et al. (1989) Nature 338:263-264).

G proteins represent a family of heterotrimeric proteins composed of α, β and γ subunits, that bind guanine nucleotides. These proteins are usually linked to cell surface receptors, e.g., receptors containing seven transmembrane segments. Following ligand binding to the GPCR, a conformational change is transmitted to the G protein, which causes the α-subunit to exchange a bound GDP molecule for a GTP molecule and to dissociate, from the βγ-subunits. The GTP-bound form of the α-subunit typically functions as an effector-modulating moiety, leading to the production of second messengers, such as cAMP (e.g., by activation of adenyl cyclase), diacylglycerol or inositol phosphates. Greater than 20 different types of α-subunits are known in humans. These subunits associate with a smaller pool of β and γ subunits. Examples of mammalian G proteins include Gi, Go, Gq, Gs and Gt. G proteins are described extensively in Lodish et al. Molecular Cell Biology, (Scientific American Books Inc., New York, N.Y., 1995), the contents of which are incorporated herein by reference. GPCRs, G proteins and G protein-linked effector and second messenger systems have been reviewed in The G-Protein Linked Receptor Fact Book, Watson et al. eds., Academic Press (1994).

### Lipid Ligands for GPCRs

Lysophospholipids have been shown to act on distinct G-protein-coupled receptors to serve a variety of overlapping biological functions. Lysophosphatidic acid (LPA) is an extracellular phospholipid that produces multiple cellular responses including cellular proliferation, inhibition of differentiation, cell surface fibronectin binding, tumor cell invasion, chemotaxis, Cl⁻ mediated membrane depolarization, increased tight junction permeability, myoblast differentiation, stimulation of fibroblast chemotaxis, acute loss of gap junctional communication, platelet aggregation, smooth muscle contraction, neurotransmitter release, stress fiber formation, cell rounding, and neurite retraction, among others. See, Moolenaar, W. H. et al., Curr. Opin. Cell Biol. 9:168-173 (1997). LPA acts through G-protein-coupled receptors to evoke the multiple cellular responses. It is generated from activated platelets and can, also be generated from microvesicles shed from blood cells challenged with inflammatory stimuli. It is one of the major mitogens found in blood serum. LPA has been shown to serve as an EDG family ligand (for EDG-2). This is consistent with a general role for this receptor family in proliferation-related signal transduction (see below herein).

The N1E-115 neuronal cell line shows morphological responses to LPA. LPA induces retraction of developing neurites and rounding of the cell body, changes driven by contraction of the actomyosin system, regulated by the GTP binding protein Rho. See, Postma, EMBO J. 15:2388-2395 (1996).

In Xenopus oocytes, LPA elicits oscillatory Cl⁻ currents. Expression depends upon a high affinity LPA receptor having features common to members of the rhodopsin seven transmembrane receptor superfamily. An antisense oligonucleotide derived from the first 5-11 amino acids selectively inhibited expression of this receptor. See, Guo et al., Proc. Nat'l. Acad. Sci. U.S.A. 93:14367-14372 (1996).

The intracellular biochemical signaling events that mediate the effects of LPA include stimulation of phospholipase C and consequent increases in cytoplasmic calcium concentration, inhibition of adenyl cyclase, and activation of phosphatidylinositol-3-kinase, the Ras-Raf-MAP kinase cascade and Rho GTPase and Rho-dependent kinases. The Ras-Raf-MAP kinase and Rho pathways stimulate the transcription factors ternary complex factor and serum response factor, respectively. Ternary complex factors and serum response factors synergistically activate transcription of growth-related immediate early genes such as c-fos by binding to serum response element (SRE) in the promoters (Hill et al., Cell 81:1159-1170 (1995)).

LPA receptors in fibroblasts couple to at least three distinct G-proteins: G_{q}, Gᵢ, and G₁₂₋₁₃. Activation of G_{q} stimulates phospholipase C and consequent mobilization of intracellular calcium. Activation of Gᵢ inhibits adenyl cyclase and stimulates the Ras-Raf-MAP kinase pathway leading to transcriptional activation mediated by ternary complex factors. Activation of G₁₂₋₁₃ stimulates Rho which leads to actin-based cytoskeleton changes and transcriptional activation mediated by serum response factor. The Gᵢ and Rho-activated pathways synergistically stimulate transcription of many growth-related genes containing serum response elements in their promoters (An, et al., J. Biol. Chem. 273:7906-7910 (1998)).

It has been reported that serum albumin contains about a dozen as yet unidentified lipids (methanol soluble) with LPA-like biological activity. See Postma, cited above.

Sphingolipids have also been reported to be involved in cell signaling. Ceramide (N-acyl-sphingosine), sphingosine and sphingosine-1-phosphate (S1P) are second messengers involved in various biological functions. Ceramide is involved in apoptosis. S1P is a platelet-derived lysosphingolipid that acts on cognate G-protein-coupled receptors to evoke multiple cellular responses, such as cellular proliferation and tumor metastasis. See Moolenaar, cited above, and Meyer et al. (FEBS. Lett. 410:34-38 (1997)) for a review. Typical receptor-mediated responses to S1P (and LPA) include stimulation of phospholipase C and consequent calcium mobilization, inhibition of adenylate cyclase, mitogen activated protein (MAP) kinase activation, DNA synthesis, mitogenesis and cytoskeletal changes, such as cell rounding and neurite retraction (Zondag, cited above), microfilament reorganization, cell migration, stress fiber formation, membrane depolarization, and fibroblast proliferation.

S1P has been shown to act on neuronal N1E-115 cells by means of a high affinity receptor, to remodel the actin cytoskeleton in a Rho-dependent manner. See, Postma, et al., cited above. Like LPA, S1P induces neurite retraction and cell rounding in differentiated PC12 cells. See, Sato, et al., Biochem. Biophys. Res. Comm. 240:329-334 (1997).

S1P acts by activating a G-protein-coupled receptor distinct from the LPA receptor. Recently, S1P has been demonstrated to act as a ligand for three members of the EDG subfamily of GPCRs, EDG-1, EDG-3, and H218.

A distinct receptor is also activated by another lysosphingolipid, sphingosylphosphorylcholine (SPC or lysosphingomyelin). It is a strong mitogen and evokes biochemical responses similar to those by LPA, except by a distinct receptor (in some cells, however, SPC and S1P might act on the same receptor). See, Moolenaar, cited above. SPC has also been shown to mediate fibroblast mitogenesis, platelet activation, and neurite retraction. It has been shown to activate MAP kinases. See, An, et al., FEBS Lett. 417:279-282 (1997). S1P and SPC also activate pathways involving Gᵢ, Ras-Raf-ERK and Rho GTPases (An, et al., FEBS Lett.).

Since S1P and LPA are both released from activated platelets, they may play a role in wound healing and tissue remodeling, including during traumatic injury of the nervous system. Because LPA can also be generated from blood cells challenged with inflammatory stimuli, LPA may stimulate responses not only at the site of injury but also at sites of inflammation.

### EDG (Endothelial Differentiation Gene) Receptors

Hecht et al. (J. Cell Biol. 135:1071-1083 (1996)) cloned a cDNA from mouse neocortical cell lines. This gene, termed ventricular zone gene-1 (vzg-1) was shown to be 96% identical to an unpublished sheep sequence designated EDG-2 (GenBank Accession No. U18405) and identified as an LPA receptor. This cDNA was also isolated as an orphan receptor by Macrae et al. (Mol. Brain Res. 42:245-254 (1996)) who designated it Rec1.3. EDG-2 is closely homologous to a Gᵢ-linked orphan receptor EDG-1 (37% homology). A cDNA homologous to that encoding sheep EDG-2 protein was cloned from a human lung cDNA library (An et al., Biochem. Biophys. Res. Comm. 231:619-622 (1997)). A search of GenBank showed that EDG-2 cDNA from mouse and cow had also been cloned and sequenced. The human EDG-2 protein was shown to be a receptor for LPA. The cDNA was expressed in mammalian cells (HEK293 and CHO) using a reporter gene assay quantifying the transcriptional activation of a serum response element-containing promoter. This assay can sensitively measure the G-protein-activated signaling pathways linked to LPA receptors. The mouse EDG-2 (Vzg-1) showed 96% identity to the human EDG-2 (Hecht et al., J. Cell Biol. 135:1071-1083 (1996)). EDG-2 was demonstrated to mediate inhibition of adenyl cyclase by Gᵢ and cell morphological changes via Rho-related GTPases (An et al., J. Biol. Chem. 273:7906-7910 (1998)).

Human EDG-1 cDNA was cloned from a human cDNA library of human umbilical vein endothelial cells exposed to fluid sheer stress (Takada et al., Biochem. Biophys. Res. Comm. 240:737-741 (1997)). EDG-1 mRNA levels in endothelial cells increased markedly in response to fluid flow. This suggested that EDG-1 is a receptor gene that could function to regulate endothelial function under physiological blood flow conditions. Recently, it was shown that the EDG-1 receptor is capable of mediating a subset of early responses to sphingosine 1-phosphate (S1P), notably, inhibition of adenylate cyclase and activation of the G₁-MAP kinase pathway, but not activation of the PLC-Ca²⁺ signaling pathway. (Zondag, G. C. et al., Bio. Chem. J. 330:605-609 (1998)).

The overexpression of EDG-1 receptors has been shown to induce exaggerated cell-cell aggregation, enhanced expression of cadherins, and formation of well-developed adherens junction, dependent upon S1P. The third intracellular loop has been shown to interact with G-a-i-1 and G-a-i-3 in a ligand-independent manner.

In the study of Zondag, the results indicated that EDG-1 but not EDG-2 was capable of mediating the specific subset of cellular actions induced by S1P. However, these responses were specific in that LPA failed to mimic S1P.

Another study (Fukushima et al., Proc. Natl. Acad. Sci. USA 95:6151-6156 (1998)) showed that the human EDG-2 mediates multiple cellular responses to LPA. At least six biological responses to LPA were reported, including the production of LPA membrane binding sites, LPA dependent G-protein activation, stress fiber formation, neurite retraction, transcriptional serum response element activation and increased DNA synthesis. EDG-1 and EDG-2 were shown to signal through at least two distinct pathways, a Gᵢ/Gₒ pathway and a PTX insensitive pathway that involves Rho activation. It was demonstrated that Gᵢ coupled directly with Vzg-1 (EDG-2) after LPA exposure. At the same time it was shown that Vzg-1 mediates actin-based cytoskeletal changes that operate through a Rho-sensitive pathway. See Fukushima, cited above. The results were consistent with a model in which EDG-2 transduces LPA signals onto the same DNA target through two separate pathways. Activation of serum response element-dependent transcription can be effected through stimulation of the Ras-Raf-MAP kinase cascade (by a ternary complex factor) and through a Rho-mediated pathway. An important response related to the serum response element activation is progression through the cell cycle.

Using the cDNA sequence of the EDG-2 human LPA receptor to perform a sequence-based search for lysosphingolipid receptors, An et al. (FEBS. Lett. 417:279-282 (1997)) found two closely related G-protein-coupled receptors, designated rat H218 and human EDG-3. Both of these, when overexpressed in Jurkat cells, mobilized calcium and activated serum response element-driven transcriptional reporter gene (which requires activation of Rho and Ras GTPases) in response to S1P, dihydro-S1P, and sphingosylphosphorylcholine, but not to LPA. Expressed in Xenopus oocytes, the genes conferred responsiveness to S1P in agonist-triggered calcium efflux.

EDG-2 was also used for a sequence-based search for new genes encoding novel subtypes of LPA receptors. A human cDNA encoding a G-protein-coupled receptor designated EDG-4 was identified by searching GenBank for homologies with the EDG-2 LPA receptor. When overexpressed in Jurkat cells, this protein mediates LPA-induced activation of a serum response element reporter gene with LPA concentration-dependence and specificity (An et al., J. Biol. Chem. 273:7906-7910 (1998)). Jurkat cells are a preferred assay system because they lack background responses to LPA in the serum response element reporter gene assay. EDG4 was shown to mediate activation of serum response element-driven transcription in Jurkat cells involving Gᵢ and Rho GTPase.

### Purinoceptors

Purines, and especially adenosine and adenine nucleotides, have a broad range of pharmacological effects mediated through cell-surface receptors. For a general review, see Adenosine and Adenine Nucleotides in The G-Protein Linked Receptor Facts Book, Watson et al. (Eds.) Academic Press 1994, pp. 19-31.

Some effects of ATP include the regulation of smooth muscle activity, stimulation of the relaxation of intestinal smooth muscle and bladder contraction, stimulation of platelet activation by ADP when released from vascular endothelium, and excitatory effects in the central nervous system. Some effects of adenosine include vasodilation, bronchoconstriction, immunosuppression, inhibition of platelet aggregation, cardiac depression, stimulation of nociceptive afferants, inhibition of neurotransmitter release, pre- and postsynaptic depressant action, reducing motor activity, depressing respiration, inducing sleep, relieving anxiety, and inhibition of release of factors, such as hormones.

Distinct receptors exist for adenosine and adenine nucleotides. Clinical actions of such analogs as methylxanthines, for example, theophylline and caffeine, are thought to achieve their effects by antagonizing adenosine receptors. Adenosine has a low affinity for adenine nucleotide receptors, while adenine nucleotides have a low affinity for adenosine receptors.

There are four accepted subtypes of adenosine receptors, designated A₁, A_{2A}, A_{2B}, and A₃. In addition, an A₄ receptor has been proposed based on labeling by 2-phenylaminoadenosine (Cornfield et al. (1992) Mol. Pharmacol. 42:552-561).

P_{2X} receptors are ATP-gated cation channels (See Neuropharmacology 36 (1977)). The proposed topology for P_{2X} receptors is two transmembrane regions, a large extracellular loop, and intracellular N and C-termini.

Numerous cloned receptors designated P_{2Y} have been proposed to be members of the G-protein coupled family. UDP, UTP, ADP, and ATP have been identified as agonists. To date, P_{2Y1-7} have been characterized although it has been proposed that P_{2Y7} may be a leukotriene B4 receptor (Yokomizo et al. (1997) Nature 387:620-624). It is widely accepted, however, that P_{2Y 1, 2, 4,} and ₆ are members of the G-protein coupled family of P_{2Y} receptors.

At least three P₂ purinoceptors from the hematopoietic cell line HEL have been identified by intracellular calcium mobilization and by photoaffinity labeling (Akbar et al. (1996) J. Biochem. 271:18363-18567).

The A₁ adenosine receptor was designated in view of its ability to inhibit adenylcyclase. The receptors are distributed in many peripheral tissues such as heart, adipose, kidney, stomach and pancreas. They are also found in peripheral nerves, for example intestine and vas deferens. They are present in high levels in the central nervous system, including cerebral cortex, hippocampus, cerebellum, thalamus, and striatum, as well as in several cell lines. Agonists and antagonists can be found on page 22 of The G-Protein Linked Receptor Facts Book cited above, herein incorporated by reference. These receptors are reported to inhibit adenylcyclase and voltage-dependent calcium channels and to activate potassium channels through a pertussis-toxin-sensitive G-protein suggested to be of the Gᵢ/Gₒ class. A₁ receptors have also been reported to induce activation of phospholipase C and to potentiate the ability of other receptors to activate this pathway.

The A_{2A} adenosine receptor has been found in brain, such as striatum, olfactory tubercle and nucleus accumbens. In the periphery, A₂ receptors mediate vasodilation, immunosuppression, inhibition of platelet aggregation, and gluconeogenesis. Agonists and antagonists are found in The G-Protein Linked Receptor Facts Book cited above on page 25, herein incorporated by reference. This receptor mediates activation of adenylcyclase through G₈.

The A_{2B} receptor has been shown to be present in human brain and in rat intestine and urinary bladder. Agonists and antagonists are discussed on page 27 of The G-Protein Linked Receptor Facts Book cited above, herein incorporated by reference. This receptor mediates the stimulation of cAMP through G₈.

The A₃ adenosine receptor is expressed in testes, lung, kidney, heart, central nervous system, including cerebral cortex, striatum, and olfactory bulb. A discussion of agonists and antagonists can be found on page 28 of The G-Protein Linked Receptor Facts Book cited above, herein incorporated by reference. The receptor mediates the inhibition of adenylcyclase through a pertussis-toxin-sensitive G-protein, suggested to be of the Gᵢ/Gₒ class.

The P_{2Y} purinoceptor shows a similar affinity for ATP and ADP with a lower affinity for AMP. The receptor has been found in smooth muscle, for example, taeni caeci and in vascular tissue where it induces vasodilation through endothelium-dependent release of nitric oxide. It has also been shown in avian erythrocytes. Agonists and antagonists are discussed on page 30 of The G-Protein Linked Receptor Facts Book cited above, herein incorporated by reference. The receptor function through activation of phosphoinositide metabolism through a pertussis-toxin-insensitive G-protein, suggested to be of the Gᵢ/Gₒ class.

### Receptor for Human C5a Anaphylatoxin

Chemotaxis of phagocytic cells is a key event in host defense and inflammatory responses. The C5a receptor mediates the pro-inflammatory and chemotaxis actions of the complement anaphylatoxin C5a. This receptor stimulates chemotaxis granule enzyme release, superoxide anion production, and upregulates expression and activity of the adhesion molecule MAC-1 and of CR-1, and mediates a decrease in cell surface glycoprotein 100, MEL-14, in anaphylaxis and in septic shock. This receptor is a member of the rhodopsin superfamily of receptors. In contrast to other receptors of this family (adrenergic, serotoninergic, dopaminergic, FSHILH, substance P and substance K), the C5a receptor functions in a concentration gradient of ligand and internalizes bound receptor during chemotaxis.

### The Ras Superfamily of GTPases

Proteins regulating Ras and its relatives have been reviewed in Boguski et al. (Nature 366:643-654 (1993)), summarized below. Ras proteins and their relatives are key in the control of normal and transformed cell growth. Small GTPases related to Ras control a wide variety of cellular processes which include aspects of growth and differentiation, control of the cytoskeleton and regulation of cellular traffic between membrane bound compartments. These proteins cycle between active and inactive states bound to GTP and GDP. This cycling is influenced by three classes of proteins that switch the GTPase on, switch it off, and prevent it from switching. Further, the intracellular location of the GTPase can be controlled by another class of regulatory protein. The GTP-bound form of the GTPase is converted to the GDP-bound form by an intrinsic capacity to hydrolyze GTP. This process is accelerated by a GTPase-activating protein (GAP). Activation involves the replacement of GDP with GTP. This event is mediated by proteins designated guanine nucleotide exchange factors (GEF) or guanine nucleotide releasing protein (GNRP) and guanine nucleotide dissociation stimulator (GDS). The process is inhibited by guanine nucleotide dissociation inhibitors (GDI). Further, membrane anchoring of the GTPase is critical for proper function and is regulated, among other enzymes, by prenyltransferases.

The Ras superfamily of GTPases can be roughly divided into three main families. The first family is the “true” Ras protein, each of which has the ability to function as an oncogene following mutational activation. These proteins transmit signals from tyrosine kinases at the plasma membrane to a cascade of serine/threonine kinases, which deliver signals to the cell nucleus. Constitutive activation of the pathway contributes to malignant transformation. The second group is the Rho/Rac protein subgroup, involved in organizing the cytoskeleton. Rac is required for membrane ruffling induced by growth factors and the formation of actin stress fibers requires Rho. In yeast, the CDC42 product controls cell polarity, another process in which actin is involved. In addition, Rac proteins are components of the NADPH oxidase system that generates superoxide in phagocytes. A third family is the Rab protein family. Members of this group regulate membrane trafficking, i.e., transport of vesicles between different intracellular compartments.

In addition to the three major families, further subgroups exist, exemplified by Ran and Arf. Ran proteins are nuclear GTPases involved in mitosis. Arf (ADP-ribosylation factor) proteins are necessary for ADP-ribosylation of Gₛₐ (the GTPase subunit of s-type heterotrimeric G-proteins) by cholera toxin and are thought to be involved in membrane vesicle fusion and transport.

Ras GEFs are proteins that activate Ras proteins by exchanging bound GDP for free GTP. These include Ras GRF, MmSosI, DnSoS, Ste6, Cdc25, Scd25, Lte1, and BUD5. The loss of GEF function can be complemented by mutations that constitutively activate the Ras proteins or, in some cases, by a loss of GAP activity. GEFs first associate with the GDP-bound form of the GTPase. GDP dissociates from this complex at an increased rate leaving the GEF bound to the empty GTPase. GTP then binds immediately, effecting GEF dissociation and leaving the GTPase in active form. Accordingly, a stable complex can exist between GEF and GTPase in the absence of nucleotide. Thus, GEFs recognize both GDP and GTP-bound forms of Ras in vitro and in vivo.

Dominant negative Ras mutants exist that block normal Ras activation. These have reduced affinity for GTP and may be defective in the final step of the exchange process, i.e. displacement of GEF by GTP. Accordingly, these mutants sequester GEF into a dead-end complex and are useful to remove GEF activity from cells so that activation of endogenous Ras proteins cannot occur. However, Ras may also be activated by inhibiting GAP activity without the need for GEF.

GEFs also include ral GEF. It is 20-fold more active on Ral A and Ral B than on members of the Ras, Rho/Rac and Rab GTPase families.

GEFs also include rap GEF. Cell polarity and budding in yeast involve GTPases of the Rap and Rho subgroup. A GEF specific for mammalian Rap proteins remains to be identified. Rap has the ability to interfere with Ras signaling by blocking activation of RAF and the serine/threonine kinase cascade.

GEFs also include Rho/Rac GEFs. GEFs specific for Rac and Rho proteins include, but are not limited to, Cdc24, Dbl, Vav, Bcr, Ras GRF, and ect 2. The human Dbl has been shown to act as a GEF for CDC42Hs (the human homolog of CDC42 is known as G25K) and on Rho. Further, Dbl binds several Rac/Rho-like proteins in vitro.

smg GDS (small GTP-binding protein) was originally described as a GEF for mammalian Rap proteins. It also promotes nucleotide exchange on Rho and Rac proteins. The protein works efficiently only on isoprenylated proteins. Ras and Rho/Rac proteins are modified by different isoprenoid moieties. Rho/Rac proteins receive 20-carbon geranylgeranyl groups.

Guanine nucleotide dissociation inhibitors (GDIs) include rab GDI. The protein affects the rate of GDP dissociation from Rab proteins. It inhibits GDP/GTP exchange and prevents the GDP-bound form from binding to membranes. These activities depend on the C-terminal geranylgeranyl group, at least of Rab3A.

Rho GDI was first identified as a factor capable of inhibiting dissociation of GDP from post-translationally modified Rho proteins. It has the ability to remove Rho proteins from cellular membranes in cell-free systems. This indicates that it could regulate the available Rho proteins associated with membranes or facilitate movement of Rho from one membrane compartment to another. Rac proteins bound to Rho GDI have also been identified as components of the NADPH oxidase system that generates oxygen radicals in activated phagocytes. Rac and Rho GDI form a heterodimer required for oxidase stimulation in vitro. Along with two other cytosolic factors, the components assemble into a membrane-bound complex which uses electrons from NADPH to generate superoxide anions. Recombinant Rac proteins in their GDP-bound state can replace the requirement for Rac and Rho GDI in this system. This indicates that Rho GDI can recognize the GTP-bound form of Rac and protect it from Rac GAPs.

GTPase-activating proteins are disclosed within Table 1 in Boguski, et al., above. These include Ras GAP proteins. These proteins have low intrinsic GTPase activity and their inactivation is dependent on GAP in vivo. Of the Ras GAPs, neurofibromin, p120 GAP, Ira1, and Ira2 also have specificity for Rac. Of the rap GAP family, Rap1GAP also has specificity for Rac. Rho/Rac GAPs with specificity for Rac include Bcr, N-chimerin, rotund, p190, GRB-1/p85a, and 3BP-1.

Ras-like GTPases are targeted to membranes where they act by the post-translational attachment of isoprenoid lipids (or prenyl groups). Prenylation involves the covalent thioether linkage of farnesyl (15-carbon) or geranylgeranyl (20-carbon) groups to cysteine residues near the C-terminus. These reactions are catalyzed by prenyltransferases that differ in their isoprenoid substrates and protein targets. Type 1 geranylgeranyl transferase recognizes a CAAX motif but prefers a leucine residue in the X-position. Substrates include members of Rho/Rac families.

p21-activated protein kinases (PAKs) are activated through direct interaction with the GTPases Rac and Cdc42Hs. These GTPases are implicated in the control of mitogen-activated protein kinase (MAP) kinase c-Jun N-terminal kinase (JNK) and the reorganization of the actin cytoskeleton. Recently, Aronheim et al. (Current Biology 8:1125-1128 (1998)) reported on the biological role of PAK2 and identified its molecular targets. A two-hybrid system, “the Ras recruitment system” was used to detect protein-protein interactions at the inner surface of the plasma membranes. The PAK2 regulatory domain was fused at the carboxy terminus of a Ras mutant protein and screened against a cDNA library. Four clones were identified that interacted specifically with PAK regulatory region and were shown to encode a homolog of the GTPase Cdc42Hs. This protein, designated Chp, showed an overall sequence identity to Cdc42Hs of approximately 52%. Results from microinjection of this protein into cells implicated it in the induction of lamellipodia and showed that it activates the JNK MAP kinase cascade.

Accordingly, GPCRs, GTPases, EDG receptors and purinoceptors, are major targets for drug action and development. Accordingly, it is valuable to the field of pharmaceutical development to identify and characterize previously unknown GPCRs, GTPases, EDG receptors and purinoceptors. The present invention advances the state of the art by providing previously unidentified human GPCRs, GTPases, EDG receptors and purinoceptors, commonly referred to herein as GPCRs.

### SUMMARY OF THE INVENTION

It is an object of the invention to identify novel GPCRs.

It is a further object of the invention to provide novel GPCR polypeptides that are useful as reagents or targets in receptor assays applicable to treatment and diagnosis of GPCR-mediated disorders.

It is a further object of the invention to provide polynucleotides corresponding to the novel GPCR receptor polypeptides that are useful as targets and reagents in receptor assays applicable to treatment and diagnosis of GPCR-mediated disorders and useful for producing novel receptor polypeptides by recombinant methods.

A specific object of the invention is to identify compounds that act as agonists and antagonists and modulate the expression of the novel receptors.

A further specific object of the invention is to provide compounds that modulate expression of the receptors for treatment and diagnosis of GPCR-related disorders.

The invention is thus based on the identification of novel GPCRs, designated 14400, 2838, 14618, 15334, 14274, 32164, 39404, 38911, 26904, 31237, 18057, 16405, 32705, 23224, 27423, 32700, 32712 and 12216 (refer to table 1 below).

TABLE 1
Sequences of the invention

| Gene | Protein | cDNA | ATCC Accession |
|---|---|---|---|
| Name | SEQ ID NO: | SEQ ID NO: | Number and Deposit Date |
| 14400 | 1 | 2 | N/A |
| 2838 | 4 | 5 | N/A |
| 14618 | 6 | 7 | N/A |
| 15334 | 8 | 9 | PTA-1658 (Deposited on Apr. |
| | | | 6, 2000) |
| 14274 | 11 | 12 | N/A |
| 32164 | 14 | 15 | PTA-1650 (deposited on Apr. |
| | | | 6, 2000) |
| 39404 | 16 | 17 | N/A |
| 38911 | 18 | 19 | N/A |
| 26904 | 20 | 21 | N/A |
| 31237 | 22 | 23 | N/A |
| 18057 | 52 | 53 | N/A |
| 16405 | 56 | 57 | N/A |
| 32705 | 61 | 60 | N/A |
| 23224 | 63 | 62 | N/A |
| 27423 | 65 | 64 | N/A |
| 32700 | 67 | 66 | N/A |
| 32712 | 69 | 68 | N/A |
| 12216 | 71 | 72 | N/A |

The invention provides isolated 14400, 2838, 14618, 15334, 14274, 32164, 39404, 38911, 26904, 31237, 18057, 16405, 32705, 23224, 27423, 32700, 32712 or 12216 receptor polypeptides including a polypeptide having the amino acid sequence shown in SEQ ID NO:1, 4, 6, 8, 11, 14, 16, 18, 20, 22, 52, 56, 61, 63, 65, 67, 69 or 71, or the amino acid sequence encoded by the cDNA deposited as ATCC Patent Deposit No. PTA-1658 or PTA-1650, both deposited on Apr. 6, 2000 (“the deposited cDNAs”).

The invention also provides isolated 14400, 2838, 14618, 15334, 14274, 32164, 39404, 38911, 26904, 31237, 18057, 16405, 32705, 23224, 27423, 32700, 32712 or 12216 receptor nucleic acid molecules having the sequence shown in SEQ ID NO:2, 5, 7, 9, 12, 15, 17, 19, 21, 23, 53, 57, 60, 62, 64, 66, 68 or 72 or in the deposited cDNAs.

The invention also provides variant polypeptides having an amino acid sequence that is substantially homologous to the amino acid sequence shown in SEQ ID NO:1, 4, 6, 8, 11, 14, 16, 18, 20, 22, 52, 56, 61, 63, 65, 67, 69 or 71 or encoded by the deposited cDNAs.

The invention also provides variant nucleic acid sequences that are substantially homologous to the nucleotide sequence shown in SEQ ID NO:2, 5, 7, 9, 12, 15, 17, 19, 21, 23, 53, 57, 60, 62, 64, 66, 68 or 72 or in the deposited cDNAs.

The invention also provides fragments of the polypeptide shown in SEQ ID NO:1, 4, 6, 8, 11, 14, 16, 18, 20, 22, 52, 56, 61, 63, 65, 67, 69 or 71 and nucleotide sequence shown in SEQ ID NO:2, 5, 7, 9, 12, 15, 17, 19, 21, 23, 53, 57, 60, 62, 64, 66, 68 or 72, as well as substantially homologous fragments of the polypeptide or nucleic acid.

The invention further provides nucleic acid constructs comprising the nucleic acid molecules described above. In a preferred embodiment, the nucleic acid molecules of the invention are operatively linked to a regulatory sequence.

The invention also provides vectors and host cells for expressing the receptor nucleic acid molecules and polypeptides and particularly recombinant vectors and host cells.

The invention also provides methods of making the vectors and host cells and methods for using them to produce the receptor nucleic acid molecules and polypeptides.

The invention also provides antibodies or antigen-binding fragments thereof that selectively bind the receptor polypeptides and fragments.

The invention also provides methods of screening for compounds that modulate expression or activity of the receptor polypeptides or nucleic acid (RNA or DNA).

The invention also provides a process for modulating receptor polypeptide or nucleic acid expression or activity, especially using the screened compounds. Modulation may be used to treat conditions related to aberrant activity or expression of the receptor polypeptides or nucleic acids.

The invention also provides assays for determining the presence or absence of and level of the receptor polypeptides or nucleic acid molecules in a biological sample, including for disease diagnosis.

The invention also provides assays for determining the presence of a mutation in the receptor polypeptides or nucleic acid molecules, including for disease diagnosis.

In still a further embodiment, the invention provides a computer readable means containing the nucleotide and/or amino acid sequences of the nucleic acids and polypeptides of the invention, respectively.

### DETAILED DESCRIPTION OF THE INVENTION

### Receptor Function/Signal Pathway

The 14400, 2838, 14618, 15334, 14274, 32164, 39404, 31237, 16405, 32705, 23224, 27423, 32700, 32712 or 12216 receptor proteins are GPCRs that participates in signaling pathways. The 38911, 26904 and 18057 seven-transmembrane proteins are putative GPCRs that participate in signaling pathways. As used herein, a “signaling pathway” refers to the modulation (e.g., stimulation or inhibition) of a cellular function/activity upon the binding of a ligand to the GPCR (14400, 2838, 14618, 15334, 14274, 32164, 39404, 38911, 26904, 31237, 18057, 16405, 32705, 23224, 27423, 32700, 32712 or 12216 protein). Examples of such functions include mobilization of intracellular molecules that participate in a signal transduction pathway, e.g., phosphatidylinositol 4,5-bisphosphate (PIP₂), inositol 1,4,5-triphosphate (IP₃) and adenylate cyclase; polarization of the plasma membrane; production or secretion of molecules; alteration in the structure of a cellular component; cell proliferation, e.g., synthesis of DNA; cell migration; cell differentiation; and cell survival.

Since the 14400 receptor protein is expressed in spleen, thymus, prostate, testes, uterus, small intestine, colon, peripheral blood lymphocytes, heart, brain, placenta, lung, liver, skeletal muscle, kidney, and pancreas, cells participating in a 14400 receptor protein signaling pathway include, but are not limited to cells derived from these tissues.

Since the 2838 receptor protein is expressed in thymus, lymph node, spleen, testes, colon, and peripheral blood lymphocytes including but not limited to activated T helper cells-1, activated T-helper cells-2, CD3(both CD4 and CD8), activated B cells, and granulocytes, cells participating in a 2838 receptor protein signaling pathway include, but are not limited to, these tissues and cells.

Since the 14618 receptor protein is expressed in breast, skeletal muscle, thyroid, lymph node, spleen, and peripheral blood lymphocytes including, but not limited to, CD34⁺ cells, resting B cells, and megakaryocytes, cells participating in a 14618 receptor protein signaling pathway include, but are not limited to, cells derived from these tissues and cells.

Since the 15334 receptor protein is expressed in colon, pancreas, tonsil, lymph node, spleen, thymus, adrenal gland, heart, peripheral blood cells, megakaryocytes, and erythroblasts, cells participating in a 15334 receptor protein signaling pathway include, but are not limited to, cells derived from these tissues and cells.

The 14274 receptor shows very high expression in brain and high expression in spleen, bone marrow, lung, resting T-cells compared to activated T-cells, and CD8 T-cells. There is also significant 14274 expression in lung carcinoma samples, colon carcinaoma samples, samples of liver metastases from colon, GCSF-treated mPB leukocytes and CD3 T cells. The expression in CD34⁻ suggests that the gene is expressed in nonprogenitor marrow cells. The expression of the gene in nonactivated lymphocytes (more specifically, CD3 T-cells) suggests that the gene functions in the central nervous system. Finally, based on cellular expression, the 14274 receptor may function in inflammation and hematopoetic contexts (relatively high expression in resting T-cells as compared to activated T-cells). Expression of the 14274 receptor is particularly pronounced in lung carcinoma, and particularly squamous cell carcinoma. The gene also shows increased expression in colon carcinoma. The gene also shows a significant decrease in expression in breast carcinoma. Since the 14274 receptor protein is expressed in these tissues, cells participating in a 14274 receptor protein signaling pathway include, but are not limited to cells derived from these tissues.

32164 expression is detected at high levels in hematopoietic progenitor CD34+ cells, especially erythroid lineages, and 32164 expression increases as bone marrow/blood cell differentiation proceeds. The 32164 expression pattern supports a role for the encoded GPCR in the development of cells of the erythroid lineage.

The 39404 protein is expressed at high levels in the brain, kidney, aortic intimal proliferations, and internal mammary artery. 39404 is also moderately expressed in the breast, skeletal muscle, colon, testes, thyroid, fetal kidney, fetal liver and saphenous veins. Therefore, cells participating in a 39404 protein signaling pathway include, but are not limited to, cells derived from these tissues, especially those tissues in which the gene is highly expressed, such as brain, kidney, aortic intimal proliferations, and internal mammary artery.

Since the 38911 protein is expressed at high levels in osteoclasts, spleen, liver, kidney, tonsils, and testis, and at moderate levels in the breast, skeletal muscle, lung, adipose and lymph nodes, cells participating in a 38911 protein signaling pathway include, but are not limited to, cells derived from these tissues, especially those cells or tissues in which the gene is highly expressed, such as osteoclasts, spleen, liver, kidney, tonsils, and testis. 38911 is also expressed in CD4⁺ cells (T- lymphocytes), in peripheral blood monocytes, and in neutrophils.

Since the 26904 protein is expressed in brain, cells participating in a 26904 protein signaling pathway include, but are not limited to, cells derived from this tissue.

Since the 31237 protein is expressed in colon, cells participating in a 31237 protein signaling pathway include, but not are limited to, cells derived from this tissue.

Since the 18057 receptor protein is expressed in the various tissues including, but not limited to testes, vein, small intestine, kidney, colon, brain, aorta, and prostate, cells participating in a 18057 receptor protein signaling pathway may include, but are not limited to cells derived from these tissues. In one embodiment, cells are derived from testes.

Since the 16405 receptor protein is expressed in spleen, brain, glioblastoma, and sclerotic lesions (derived from atherosclerotic tissue), cells participating in a 16405 receptor protein signaling pathway include, but are not limited to cells derived from these tissues.

Since the 32705 G-protein is expressed in brain, lung, ganglia and virus-infected hepatocytes, cells participating in a receptor protein signaling pathway in which this protein is involved may include, but are not limited to, cells derived from these tissues. In one embodiment, cells are derived from hepatocytes infected with hepatitis B virus, and specifically the HepG2 cell line.

Since the 12216 receptor protein is highly expressed in brain, skeletal muscle, colon, mobilized peripheral blood cells, and human embryonic kidney cells, cells participating in a 12216 receptor protein signaling pathway include, but are not limited to cells derived from these tissues. Since the gene is also expressed in normal endothelial cells and, in atherosclerosis, is expressed in other atherogenic cell types, including but not limited to smooth muscle and macrophages, cells participating in a 12216 receptor protein signaling pathway include, but are not limited to, these cells as well.

The response mediated by the receptor protein depends on the type of cell. For example, in some cells, binding of a ligand to the receptor protein may stimulate an activity such as release of compounds, gating of a channel, cellular adhesion, migration, differentiation, etc., through phosphatidylinositol or cyclic AMP metabolism and turnover while in other cells, the binding of the ligand will produce a different result. Regardless of the cellular activity/response modulated by the receptor protein, it is universal that the protein is a GPCR and interacts with G proteins to produce one or more secondary signals, in a variety of intracellular signal transduction pathways, e.g., through phosphatidylinositol or cyclic AMP metabolism and turnover, in a cell.

As used herein, “phosphatidylinositol turnover and metabolism” refers to the molecules involved in the turnover and metabolism of phosphatidylinositol 4,5-bisphosphate (PIP₂) as well as to the activities of these molecules. PIP₂ is a phospholipid found in the cytosolic leaflet of the plasma membrane. Binding of ligand to the receptor activates, in some cells, the plasma-membrane enzyme phospholipase C that in turn can hydrolyze PIP₂ to produce 1,2-diacylglycerol (DAG) and inositol 1,4,5-triphosphate (IP₃). Once formed IP₃ can diffuse to the endoplasmic reticulum surface where it can bind an IP₃ receptor, e.g., a calcium channel protein containing an IP₃ binding site. IP₃ binding can induce opening of the channel, allowing calcium ions to be released into the cytoplasm. IP₃ can also be phosphorylated by a specific kinase to form inositol 1,3,4,5-tetraphosphate (IP₄), a molecule which can cause calcium entry into the cytoplasm from the extracellular medium. IP₃ and IP₄ can subsequently be hydrolyzed very rapidly to the inactive products inositol 1,4-biphosphate (IP₂) and inositol 1,3,4-triphosphate, respectively. These inactive products can be recycled by the cell to synthesize PIP₂. The other second messenger produced by the hydrolysis of PIP₂, namely 1,2-diacylglycerol (DAG), remains in the cell membrane where it can serve to activate the enzyme protein kinase C. Protein kinase C is usually found soluble in the cytoplasm of the cell, but upon an increase in the intracellular calcium concentration, this enzyme can move to the plasma membrane where it can be activated by DAG. The activation of protein kinase C in different cells results in various cellular responses such as the phosphorylation of glycogen synthase, or the phosphorylation of various transcription factors, e.g., NF-KB. The language “phosphatidylinositol activity”, as used herein, refers to an activity of PIP₂ or one of its metabolites.

Another signaling pathway in which the receptor may participate is the cAMP turnover pathway. As used herein, “cyclic AMP turnover and metabolism” refers to the molecules involved in the turnover and metabolism of cyclic AMP (cAMP) as well as to the activities of these molecules. Cyclic AMP is a second messenger produced in response to ligand-induced stimulation of certain G protein coupled receptors. In the cAMP signaling pathway, binding of a ligand to a GPCR can lead to the activation of the enzyme adenyl cyclase, which catalyzes the synthesis of cAMP. The newly synthesized cAMP can in turn activate a cAMP-dependent protein kinase. This activated kinase can phosphorylate a voltage-gated potassium channel protein, or an associated protein, and lead to the inability of the potassium channel to open during an action potential. The inability of the potassium channel to open results in a decrease in the outward flow of potassium, which normally repolarizes the membrane of a neuron, leading to prolonged membrane depolarization.

### 14400 Polypeptide

The invention is based, in part, on the discovery of a novel G- protein coupled receptor identified herein as 14400. Specifically, an expressed sequence tag (EST) was selected based on homology to G-protein-coupled receptor sequences. This EST was used to design primers based on sequences that it contains and used to identify a cDNA from a B cell cDNA library. Positive clones were sequenced and the overlapping fragments were assembled. Analysis of the assembled sequence revealed that the cloned cDNA molecule encodes a G-protein coupled receptor.

The invention thus relates to a novel 1440 GPCR having the deduced amino acid sequence shown in SEQ ID NO:1 or having the amino acid sequence encoded by the deposited cDNAs, ATCC No.______.

This deposit will be maintained under the terms of the Budapest Treaty on the International Recognition of the Deposit of Microorganisms. The deposit is provided as a convenience to those of skill in the art and is not an admission that a deposit is required under 35 U.S.C. §112. This deposited sequence, as well as the polypeptide encoded by this sequence, is incorporated herein by reference and controls in the event of any conflict, such as a sequencing error, with description in this application.

The “14400 receptor polypeptide” or “14400 receptor protein” refers to the polypeptide in SEQ ID NO:1 or encoded by the deposited cDNA. The term “receptor protein” or “receptor polypeptide”, however, further includes the numerous variants described herein, as well as fragments derived from the full length 14400 polypeptide and variants.

The present invention thus provides an isolated or purified 14400 receptor polypeptide and variants and fragments thereof.

The 14400 polypeptide is a 359 residue protein exhibiting three main structural domains. The amino terminal extracellular domain is identified to be within residues 1 to about 23 in SEQ ID NO:1. The transmembrane domain is identified to be within residues from about 24 to about 296 in SEQ ID NO:1. The carboxy terminal intracellular domain is identified to be within residues from about 297 to 359 in SEQ ID NO:1. The transmembrane domain contains seven segments that span the membrane. The transmembrane segments are found from about amino acid 24 to about amino acid 48, from about amino acid 59 to about amino acid 78, from about amino acid 89 to about amino acid 105, from about amino acid 139 to about amino acid 159, from about amino acid 189 to about amino acid 213, from about amino acid 234 to about amino acid 251, and from about amino acid 277 to about amino acid 296 of SEQ ID NO:1. Within the region spanning the entire transmembrane domain are three intracellular and three extracellular loops. The three intracellular loops are found from about amino acid 49 to about amino acid 58, from about amino acid 106 to about amino acid 138, and from about amino acid 214 to about amino acid 233 of SEQ ID NO:1. The three extracellular loops are found at from about amino acid 79 to about amino acid 88, from about amino acid 160 to about amino acid 188, and from about amino acid 252 to about amino acid 276 of SEQ ID NO:1.

An analysis of the 14400 open reading frame for amino acids corresponding to specific functional sites revealed one glycosylation site at amino acids 5 to 8 of SEQ ID NO:1 (which corresponds to the amino terminal extracellular domain); a second glycosylation site at amino acids 11 to 14 of SEQ ID NO:1 (which also corresponds to the amino terminal extracellular domain); a third glycosylation site at amino acids 64 to 67 of SEQ ID NO:1 (which corresponds to the second transmembrane segment); a cyclic AMP or cyclic GMP-dependent protein kinase phosphorylation site at amino acids 321 to 324 of SEQ ID NO:1 (which is in the carboxy terminal intracellular domain); a protein kinase C phosphorylation site at amino acids 130 to 132 of SEQ ID NO:1 (which is in the second intracellular loop); three other protein kinase C phosphorylation sites in the carboxy terminal intracellular domain at amino acids 320 to 322, 327 to 329, and 332 to 334 of SEQ ID NO:1; a casein kinase II phosphorylation site at amino acids 7 to 10 of SEQ ID NO:1 (in the amino terminal extracellular loop); a second casein kinase II phosphorylation site at amino acids 66 to 69 of SEQ ID NO:1 (which is in the second transmembrane segment); a third casein kinase II phosphorylation site at amino acids 174 to 177 of SEQ ID NO:1 (which is in the second extracellular loop); a fourth casein kinase II phosphorylation site at amino acids 320 to 323 of SEQ ID NO:1 (which is in the carboxy terminal intracellular domain); and four N-myristoylation sites at amino acids 40 to 45, 92 to 97, 171 to 176, and 343 to 348 of SEQ ID NO:1 (which are in the first transmembrane segment, the second transmembrane segment, the second extracellular loop, and the carboxy terminal intracellular domain, respectively).

A hydropathy plot of human 14400 was performed. Polypeptides of the invention include fragments which include: all or part of a hydrophobic sequence, e.g., the sequence from about amino acid 20 to 40, from about 60 to 80, from about 95 to 125, from about 145 to 155, from about 170 to 215 and from about 245 to 260 of SEQ ID NO:1; all or part of a hydrophilic sequence, e.g., the sequence from about amino acid 126 to 144, from about 216 to 240, and from about 300 to 359 of SEQ ID NO:1; a sequence which includes a Cys, or a glycosylation site.

Based on a BLAST search performed on 14400, highest homology to 14400 was shown to thrombin receptors.

A comparison of the 14400 receptor against the Prosite database of protein patterns specifically shows a high score against the Seven Transmembrane Segment Rhodopsin Superfamily (SEQ ID NO:3). The most commonly conserved sequence is an aspartate, arginine, tyrosine (DRY) triplet. DRY is implicated in signal transduction. Arginine is invariant. Aspartate is conservatively placed in several GPCRs. In the present case, the arginine is found in the sequence ERF at residues 120-122 of in SEQ ID NO:1, which matches the position of DRY or invariant arginine at residue 121 of SEQ ID NO:1 in GPCRs of the rhodopsin superfamily of receptors.

As assessed by TaqMan analysis, the 14400 receptor protein is expressed in spleen, thymus, prostate, testes, uterus, small intestine, colon, peripheral blood lymphocytes, heart, brain, placenta, lung, liver, skeletal muscle, kidney, and pancreas.

### 2838, 14618 and 15334 Polypeptides

The invention is based, in part, on the discovery of novel G-coupled protein receptors, identified herein as 2838, 14618 and 15334. Specifically, an expressed sequence tag (EST) was selected based on homology to G-protein-coupled receptor sequences. This EST was used to design primers based on sequences that it contains and used to identify a 2838 cDNA from a B cell cDNA library, a 14618 cDNA from a liver and spleen cDNA library, and a 15334 cDNA from a spleen cDNA library. Positive clones were sequenced and the overlapping fragments were assembled. Analysis of the assembled sequences revealed that the three cloned cDNA molecules encode G-protein coupled receptors.

The invention thus relates to a novel 2838 GPCR having the deduced amino acid sequence shown in SEQ ID NO:4 or having the amino acid sequence encoded by the deposited cDNA, ATCC No.______.

The invention also thus relates to a novel 14618 GPCR having the deduced amino acid sequence shown in SEQ ID NO:6 or having the amino acid sequence encoded by the deposited cDNA, ATCC No.______.

The invention also thus relates to a novel 15334 GPCR having the deduced amino acid sequence shown in SEQ ID NO:8 or having the amino acid sequence encoded by the deposited cDNA, ATCC Patent Deposit No. PTA-1658.

The deposits will be maintained under the terms of the Budapest Treaty on the International Recognition of the Deposit of Microorganisms. The deposits are provided as a convenience to those of skill in the art and is not an admission that a deposit is required under 35 U.S.C. §112. The deposited sequences, as well as the polypeptides encoded by the sequences, are incorporated herein by reference and control in the event of any conflict, such as a sequencing error, with description in this application.

The “2838 receptor polypeptide” or “2838 receptor protein” refers to the polypeptide in SEQ ID NO:4 or encoded by the deposited cDNA. The “14618 receptor polypeptide” or “14618 receptor protein” refers to the polypeptide in SEQ ID NO:6 or encoded by the deposited cDNA. The “15334 receptor polypeptide” or “15334 receptor protein” refers to the polypeptide in SEQ ID NO:8 or encoded by the deposited cDNA. The term “receptor protein” or “receptor polypeptide”, however, further includes the numerous variants of 2838, 14618, or 15334 polypeptides described herein, as well as fragments derived from the full length 2838, 14618, or 15334 polypeptides and variants.

The present invention thus provides isolated or purified 2838, 14618, and 15334 receptor polypeptides and variants and fragments thereof.

The 2838 polypeptide is a 319 residue protein exhibiting three main structural domains. The amino terminal extracellular domain is identified to be within residues 1 to about 24 in SEQ ID NO:4. The transmembrane domain is identified to be within residues from about 25 to about 292 in SEQ ID NO:4. The carboxy terminal intracellular domain is identified to be within residues from about 293 to 319 in SEQ ID NO:4. The transmembrane domain contains seven segments that span the membrane. The transmembrane segments are found from about amino acid 25 to about amino acid 49, from about amino acid 56 to about amino acid 79, from about amino acid 100 to about amino acid 117, from about amino acid 138 to about amino acid 159, from about amino acid 187 to about amino acid 210, from about amino acid 224 to about amino acid 248, and from about amino acid 268 to about amino acid 292 of SEQ ID NO:4. Within the region spanning the entire transmembrane domain are three intracellular and three extracellular loops. The three intracellular loops are found from about amino acid 50 to about amino acid 55, from about amino acid 118 to about amino acid 137, and from about amino acid 211 to about amino acid 223 of SEQ ID NO:4. The three extracellular loops are found from about amino acid 80 to about amino acid 99, from about amino acid 160 to about amino acid 186, and from about amino acid 249 to about amino acid 267 of SEQ ID NO:4.

An analysis of the 2838 open reading frame for amino acids corresponding to specific functional sites revealed a glycosylation site at amino acids 5 to 8 of SEQ ID NO:4 (which corresponds to the amino terminal extracellular domain); a second glycosylation site at amino acids 171 to 174 of SEQ ID NO:4 (which corresponds to the second extracellular loop); a protein kinase C phosphorylation site at amino acids 134 to 136 of SEQ ID NO:4 (which is in the second intracellular loop); a second protein kinase C phosphorylation site at amino acids 178 to 180 of SEQ ID NO:4 (which is in the second extracellular loop); a casein kinase II phosphorylation site at amino acids 6 to 9 of SEQ ID NO:4 (which is in the carboxy terminal intracellular domain), a second casein kinase II phosphorylation site at amino acids 95 to 98 of SEQ ID NO:4 (which is in the first extracellular loop); an N-myristoylation site at amino acids 34 to 39 of SEQ ID NO:4 (which is in the first transmembrane segment); a second N-myristoylation site at amino acids 107 to 112 of SEQ ID NO:4 (which is in the third transmembrane segment); a third N-myristoylation site at amino acids 140 to 145 of SEQ ID NO:4 (which is in the fourth transmembrane segment); and an amidation site at amino acids 209 to 212 of SEQ ID NO:4 (which spans the fifth transmembrane segment and third intracellular loop).

The transmembrane domain of 2838 includes a GPCR signal transduction signature, DRF, at residues 118-120 of SEQ ID NO:4. The sequence includes an arginine at residue 119, an invariant amino acid in GPCRs.

A hydropathy plot of human 2838 was performed. Polypeptides of the invention include fragments which include: all or part of a hydrophobic sequence, e.g., the sequence from about amino acid 21 to 41, from about 62 to 82, from about 95 to 125, from about 132 to 151, from about 183 to 201, from about 225 to 245 and from about 265 to 285 of SEQ ID NO:4; all or part of a hydrophilic sequence, e.g., the sequence from about amino acid 51 to 61 and from about 211 to 221 of SEQ ID NO:4; a sequence which includes a Cys, or a glycosylation site.

Based on a BLAST search performed on 2838, highest homology was shown to purinoceptors.

A comparison of the 2838 receptor against the Prosite database of protein patterns specifically shows a high score against the Seven Transmembrane Segment Rhodopsin Superfamily conserved sequence (SEQ ID NO:10). The most commonly conserved sequence is an aspartate, arginine, tyrosine (DRY) triplet. DRY is implicated in signal transduction. Arginine is invariant. Aspartate is conservatively placed in several GPCRs. In the present case, the arginine is found in the sequence DRF, which matches the position of DRY or invariant arginine in GPCRs of the rhodopsin superfamily of receptors.

As assessed by TaqMan analysis, the 2838 receptor protein is expressed in lymph node, thymus, spleen, testes, colon, and peripheral blood lymphocytes, and in activated T-helper cells (1 and 2), hypoxic Hep 3B cells, CD3 cells (both CD4 and CD8), activated B cells, Jurkat cells, granulocytes, among others.

The 14618 polypeptide is a 337 residue protein exhibiting three main structural domains. The amino terminal extracellular domain is identified to be within residues 1 to about 28 of SEQ ID NO:6. The transmembrane domain is identified to be within residues from about 29 to about 297 of SEQ ID NO:6. The carboxy terminal intracellular domain is identified to be within residues from about 298 to 337 of SEQ ID NO:6. The transmembrane domain contains seven segments that span the membrane. The transmembrane segments are found from about amino acid 29 to about amino acid 49, from about amino acid 84 to about amino acid 60, from about amino acid 103 to about amino acid 127, from about amino acid 142 to about amino acid 161, from about amino acid 194 to about amino acid 217, from about amino acid 231 to about amino acid 247, and from about amino acid 276 to about amino acid 297 of SEQ ID NO:6. Within the region spanning the entire transmembrane domain are three intracellular and three extracellular loops. The three intracellular loops are found from about amino acid 50 to about amino acid 59, from about amino acid 128 to about amino acid 141, and from about amino acid 218 to about amino acid 230 of SEQ ID NO:6. The three extracellular loops are found at from about amino acid 85 to about amino acid 102, from about amino acid 162 to about amino acid 193, and from about amino acid 248 to about amino acid 275 of SEQ ID NO:6.

An analysis of the 14618 open reading frame for amino acids corresponding to specific functional sites revealed a glycosylation site at amino acids 6 to 9 of SEQ ID NO:6 (which corresponds to the amino terminal extracellular domain); a second glycosylation site at amino acids 169 to 172 of SEQ ID NO:6 (which corresponds to the second extracellular loop); a third glycosylation site at amino acids 180 to 183 of SEQ ID NO:6 (which also corresponds to the second extracellular loop); a fourth glycosylation site at amino acids 224 to 227 of SEQ ID NO:6 (which corresponds to the third intracellular loop); a fifth glycosylation site at amino acids 262 to 265 of SEQ ID NO:6 (which corresponds to the third extracellular loop);, three cAMP- and cGMP-dependent protein kinase phosphorylation sites at amino acids 304 to 307, 310 to 313, and 323 to 326 of SEQ ID NO:6 (all in the carboxy terminal intracellular domain); a protein kinase C phosphorylation site at amino acids 136 to. 138 of SEQ ID NO:6 (which corresponds to the second intracellular loop); a second and third protein kinase C phosphorylation sites at amino acids 220 to 222 and 227 to 229 of SEQ ID NO:6 (both corresponding to the third intracellular loop); a fourth protein kinase C phosphorylation site at amino acids 308 to 310 of SEQ ID NO:6 (corresponding to the carboxy terminal intracellular domain); two Casein kinase II phosphorylation sites at amino acids 13 to 16 and 17 to 20 of SEQ ID NO:6 (both in the amino terminal extracellular domain); a third casein kinase II phosphorylation site at amino acids 326 to 329 of SEQ ID NO:6 (corresponding to the carboxy terminal intracellular domain); and a microbodies C-terminal targeting signal at amino acids 335 to 338 of SEQ ID NO:6 (corresponding to the carboxy terminal intracellular domain).

The transmembrane domain of 14618 includes a GPCR signal transduction signature, FRC, at residues 121-123 of SEQ ID NO:6. The sequence includes an arginine at residue 122, an invariant amino acid in GPCRs.

A hydropathy plot of human 14618 was performed. Polypeptides of the invention include fragments which include: all or part of a hydrophobic sequence, e.g., the sequence from about amino acid 25 to 45, from about 65 to 125, from about 140 to 160, from about 185 to 215, from about 231 to 241 and from about 275 to 285 of SEQ ID NO:6; all or part of a hydrophilic sequence, e.g., the sequence from about amino acid 161 to 181 of SEQ ID NO:6; a sequence which includes a Cys, or a glycosylation site.

Based on a BLAST search performed on 14618, highest homology was shown to purinoceptors.

A comparison of the 14618 receptor against the Prosite database of protein patterns specifically shows a high score against the Seven Transmembrane Segment Rhodopsin Superfamily conserved sequence (SEQ ID NO:10). The most commonly conserved sequence is an aspartate, arginine, tyrosine (DRY) triplet. DRY is implicated in signal transduction. Arginine is invariant. Aspartate is conservatively placed in several GPCRs. In the present case, the arginine is found in the sequence DRF, which matches the position of DRY or invariant arginine in GPCRs of the rhodopsin superfamily of receptors.

As assessed by TaqMan anlysis, the 14618 receptor protein is expressed in breast, skeletal muscle, lymph node, spleen and blood peripheral lymphocytes, as well as CD34⁺ cells and megakaryocytes.

The 15334 polypeptide is a 372 residue protein exhibiting three main structural domains. The amino terminal extracellular domain is identified to be within residues 1 to about 25 of SEQ ID NO:8. The transmembrane domain is identified to be within residues from about 26 to about 299 of SEQ ID NO:8. The carboxy terminal intracellular domain is identified to be within residues from about 300 to 372 of SEQ ID NO:8. The transmembrane domain contains seven segments that span the membrane. The transmembrane segments are found from about amino acid 26 to about amino acid 48, from about amino acid 56 to about amino acid 77, from about amino acid 99 to about amino acid 115, from about amino acid 140 to about amino acid 157, from about amino acid 188 to about amino acid 209, from about amino acid 235 to about amino acid 259, and from about amino acid 277 to about amino acid 299 of SEQ ID NO:8. Within the region spanning the entire transmembrane domain are three intracellular and three extracellular loops. The three intracellular loops are found from about amino acid 49 to about amino acid 55, from about amino acid 116 to about amino acid 139, and from about amino acid 210 to about amino acid 234 of SEQ ID NO:8. The three extracellular loops are found at from about amino acid 78 to about amino acid 98, from about amino acid 158 to about amino acid 187, and from about amino acid 260 to about amino acid 276 of SEQ ID NO:8.

An analysis of the 15334 open reading frame for amino acids corresponding to specific functional sites revealed two glycosylation sites at amino acids 4 to 7 and 9 to 12 of SEQ ID NO:8 (which are in the amino terminal extracellular domain); a third glycosylation site at amino acids 251 to 254 of SEQ ID NO:8 (which is in the sixth transmembrane segment); a fourth glycosylation site at amino acids 323 to 326 of SEQ ID NO:8 (which is in the carboxy terminal domain); a cAMP- and cGMP-dependent protein kinase phosphorylation site at amino acids 229 to 232 of SEQ ID NO:8 (which is in the third intracellular loop); six protein kinase C phosphorylation sites from amino acids 21 to 23 of SEQ ID NO:8 (which corresponds to the amino terminal domain), from 211 to 213, 226 to 228, and 232 to 234 of SEQ ID NO:8 (which corresponds to the third intracellular loop), and from 307 to 309 and 332 to 334 of SEQ ID NO:8 (which corresponds to the carboxy terminal intracellular domain); two casein kinase II phosphorylation sites from amino acids 178 to 181 of SEQ ID NO:8 (which is in the second extracellular loop); and from 342 to 345 of SEQ ID NO:8 (which is in the carboxy terminal intracellular domain); and three N-myristoylation sites at amino acids 36 to 41 of SEQ ID NO:8 (which is in the amino terminal extracellular domain), from 258 to 263 of SEQ ID NO:8 (which spans the sixth transmembrane segment and third extracellular loop), and from 324 to 329 of SEQ ID NO:8 (which corresponds to the carboxy terminal intracellular domain.

The transmembrane domain of 15334 includes a GPCR signal transduction signature, DRY, at residues 118-120 in SEQ ID NO:8. The sequence includes an arginine at residue 119, an invariant amino acid in GPCRs.

A hydropathy plot of human 15334 was performed. Polypeptides of the invention include fragments which include: all or part of a, hydrophobic sequence, e.g., the sequence from about amino acid 25 to 71, from about 101 to 111, from about 135 to 150, from about 185 to 205, from about 231 to 245 and from about 281 to 295 of SEQ ID NO:8; all or part of a hydrophilic sequence, e.g., the sequence from about amino acid 151 to 165 and from about 215 to 225 of SEQ ID NO:8; a sequence which includes a Cys, or a glycosylation site.

Based on a BLAST search performed on 15334, highest homology was shown to purinoceptors.

A comparison of the 15334 receptor against the Prosite database of protein patterns specifically shows a high score against the Seven Transmembrane Segment Rhodopsin Superfamily conserved sequence (SEQ ID NO:10). The most commonly conserved sequence is an aspartate, arginine, tyrosine (DRY) triplet. DRY is implicated in signal transduction. Arginine is invariant. Aspartate is conservatively placed in several GPCRs. In the present case, the arginine is found in the sequence DRY, which matches the position of DRY or invariant arginine in GPCRs of the rhodopsin superfamily of receptors.

As assessed by TaqMan analysis, the 15334 receptor protein is expressed colon, placenta, pancreas, tonsil, lymph node, spleen, peripheral blood cells, thymus, adrenal gland and heart, as well as K562 cells, erythroblasts, and megakaryocytes.

### 14274 Polypeptides

The invention is based, in part, on the discovery of a novel G-coupled protein receptor, identified herein as 14274. Specifically, an expressed sequence tag (EST) was selected based on homology to G-protein-coupled receptor sequences. This EST was used to design primers based on sequences that it contains and used to identify a cDNA from a natural killer T-cell cDNA library. Positive clones were sequenced and the overlapping fragments were assembled. Analysis of the assembled sequence revealed that the cloned cDNA molecule encodes a G-protein coupled receptor showing a high homology score against the seven transmembrane segment rhodopsin superfamily, also with high homology to the EDG receptor family. The 14274 receptor has been shown to have high homology with the EDG-1 family of the EDG receptor family. Accordingly, its ligand is likely to be S1P. Highest homology was shown against the mouse EDG-1. The third intracellular loop, having a high degree of identity with other EDG-1 sequences, contains a stretch of 18 arginine-rich amino acids that appears unique to the 14274 receptor. Similar identity is observed in the second intracellular domain. A motif of six amino acids (SLLAIA (SEQ ID NO:74)) is identified in this region. This six amino acid domain is conserved in adenosine AA2 and AA3 and melanocortin-5 receptors (human, mouse, rat, and dog) and is characterized by means of Prosite analysis to be a GPCR signature.

The invention thus relates to a novel 14274 GPCR having the deduced amino acid sequence shown in SEQ ID NO:11 or having the amino acid sequence encoded by the deposited cDNA, ATCC No.______.

The deposit will be maintained under the terms of the Budapest Treaty on the International Recognition of the Deposit of Microorganisms. The deposit is provided as a convenience to those of skill in the art and is not an admission that a deposit is required under 35 U.S.C. §112. The deposited sequence, as well as the polypeptide encoded by the sequence, is incorporated herein by reference and controls in the event of any conflict, such as a sequencing error, with description in this application.

The “14274 receptor polypeptide” or “14274 receptor protein” refers to the polypeptide in SEQ ID NO:11 or encoded by the deposited cDNA. The term “receptor protein” or “receptor polypeptide”, however, further includes the numerous variants described herein, as well as fragments derived from the full length 14274 polypeptide and variants.

The present invention, thus provides an isolated or purified 14274 receptor polypeptide and variants and fragments thereof.

The 14274 polypeptide is a 398 residue protein exhibiting three main structural domains. The amino terminal extracellular domain is identified to be within residues 1 to about 39 of SEQ ID NO:11. The region spanning the entire transmembrane domain is identified to be within residues from about 40 to about 308 of SEQ ID NO:11. Discrete transmembrane segments are estimated to be from about amino acid 40-62, 71-95, 114-131, 152-173, 192-213, 253-273, and 291-308 of SEQ ID NO:11. Accordingly, the six extracellular and intracellular loops correspond to about amino acids 63-70, 96-113, 132-151, 174-191, 214-252, and 274-290 of SEQ ID NO:11. The carboxy terminal intracellular domain is identified to be within residues from about 309 to about 398 of SEQ ID NO:11. The transmembrane domain includes the invariant arginine of a GPCR signal transduction signature, ERS, at residues 132-134 of SEQ ID NO:11.

An analysis of the 14274 open reading frame for amino acids corresponding to specific functional sites revealed one N-glycosylation site at about amino acids 20 to 23 of SEQ ID NO:11; six protein kinase C phosphorylation sites at about amino acids 22 to 24, 100 to 102, 146 to 148, 237 to 239, 309 to 311 and 363 to 365 of SEQ ID NO:11; four casein kinase II phosphorylation sites at amino acids 79 to 82, 309 to 312, 340 to 343. and 361 to 364 of SEQ ID NO:11; twelve N-myristoylation sites at about amino acids 86 to 91, 114 to 119, 166 to 171, 203 to 208, 231 to 236, 293 to 298, 334 to 339, 347 to 352, 355 to 360, 362 to 367, 372 to 377 and 383 to 388 of SEQ ID NO:11; and one G-protein-coupled receptor signature represented by ERS in the sequence at about amino acids 121 to 137 of SEQ ID NO:11.

A comparison of the 14274 receptor against the Prosite database of protein patterns specifically shows a high score against the Seven Transmembrane Segment Rhodopsin Superfamily conserved sequence (SEQ ID NO:13). The 14274 polypeptide contains an area showing a GPCR signature. The most commonly conserved intracellular sequence is the aspartate, arginine, tyrosine (DRY) triplet. Arginine is invariant. Aspartate is conservatively placed in several GPCRs. DRY is implicated in signal transduction. In the present case, the arginine is found in the sequence ERS, which matches the position of the DRY or invariant arginine for a rhodopsin family seven transmembrane receptor.

A hydropathy plot of human 14274 was performed. Polypeptides of the invention include fragments which include: all or part of a hydrophobic sequence, e.g., the sequence from about amino acid 40 to 65, from about 75 to 105, from about 115 to 130, from about 155 to 215 and from about 255 to 300 of SEQ ID NO:11; all or part of a hydrophilic sequence, e.g., the sequence from about amino acid 220 to 250 and from about 325 to 398 of SEQ ID NO:11; a sequence which includes a Cys, or a glycosylation site.

The 14274 amino acid sequence showed approximately 35% identity with EDG-4, 35% identity with EDG-2, 46% identity with EDG-3, and 50% identity with EDG-1. Approximate percent identity among various EDG family members as follows: EDG1-EDG2:40%; EDG1-EDG4:40%; EDG1-EDG3:55%; EDG2-EDG4:57%; EDG2-EDG3:39%; and EDG3-EDG4:32%.

As assessed by TaqMan analysis, the 14274 receptor protein is expressed in CD34⁻ bone marrow cells, peripheral blood cells, such as CD3 and CD8 T-cells, brain, spleen, lung, lung carcinoma, colon carcinoma, liver metastases from colon, GCSF-treated mPB leukocytes, and placenta, among others.

### 32164 Polypeptides

The invention is based, in part, on the identification of a novel human seven transmembrane protein, potentially a novel human G-coupled protein receptor, identified herein as 32164. Specifically, an expressed sequence tag (EST) was selected based on homology to G-protein-coupled receptor sequences. This EST was used to design primers based on primary sequences that it contains and used to identify a cDNA from a human spleen cDNA library. Positive clones were sequenced and the overlapping fragments were assembled. Analysis of the assembled sequence revealed that the cloned cDNA molecule encodes a seven transmembrane protein, potentially a G-protein coupled receptor, with homology to the rhodopsin family of GPCRs.

The invention thus relates to a novel seven transmembrane protein having the deduced amino acid sequence shown in SEQ ID NO:14 or having the amino acid sequence encoded by the cDNA insert of the plasmid deposited with American Type Culture Collection (ATCC), 10801 University Boulevard, Manassas Va. 20110-2209 as Patent Deposit No. PTA-1650.

The deposit will be maintained under the terms of the Budapest Treaty on the International Recognition of the Deposit of Microorganisms. The deposit is provided as a convenience to those of skill in the art and is not an admission that a deposit is required under 35 U.S.C. §112. The deposited sequence, as well as the polypeptide encoded by the sequence, is incorporated herein by reference and controls in the event of any conflict, such as a sequencing error, with description in this application.

The “32164 polypeptide” or “32164 protein” refers to the polypeptide in SEQ ID NO:14 or encoded by the deposited cDNA. The terms, however, further include the numerous variants described herein, as well as fragments derived from the full length 32164 polypeptide and variants.

The present invention thus provides an isolated or purified 32164 polypeptide and variants and fragments thereof.

The 32164 polypeptide is a 314 residue protein exhibiting three main structural domains, an amino terminal extracellular domain, a transmembrane domain, and a carboxy terminal intracellular domain. The transmembrane domain contains seven segments that span the membrane. Within the region spanning the entire transmembrane domain are three intracellular and three extracellular loops.

An analysis of the 32164 open reading frame for amino acids corresponding to specific functional sites revealed two glycosylation sites from about amino acid 5 to 8 and 42 to 45 of SEQ ID NO:14; four protein kinase C phosphorylation sites from about amino acid 18 to 20, 163 to 165, 232 to 234 and 291 to 293 of SEQ ID NO:14; three casein kinase II phosphorylation sites from about amino acid 49 to 52, 67 to 70 and 266 to 269 of SEQ ID NO:14; five N-myristoylation sites from about amino acid 3 to 8, 108 to 113, 150 to 155, 239 to 244 and 263 to 268 of SEQ ID NO:14; and one amidation site from about amino acid 306 to 309 of SEQ ID NO:14. In the case of glycosylation, the actual modified residue is the first amino acid. In the case of protein kinase C phosphorylation, casein kinase II phosphorylation, and N-myristoylation, the actual modified residue is the first amino acid. It is predicted that amino acids 1-25 of SEQ ID NO:14 constitute the amino terminal extracellular domain, amino acids 26-292 of SEQ ID NO:14 constitute the region spanning the transmembrane domain, and amino acids 293-314 of SEQ ID NO:14 constitute the carboxy terminal intracellular domain.

The transmembrane domain contains seven transmembrane segments, three extracellular loops and three intracellular loops. The transmembrane segments are found from about amino acid 26 to about amino acid 48, from about amino acid 59 to about amino acid 78, from about amino acid 101 to about amino acid 120, from about amino acid 143 to about amino acid 159, from about amino acid 199 to about amino acid 222, from about amino acid 237 to about amino acid 260, and from about amino acid 273 to about amino acid 292 of SEQ ID NO:14. Within the region spanning the entire transmembrane domain are three intracellular and three extracellular loops. The three intracellular loops are found from about amino acid 49 to about amino acid 58, from about amino acid 121 to about amino acid 142, and from about amino acid 223 to about amino acid 236 of SEQ ID NO:14. The three extracellular loops are found at from about amino acid 79 to about amino acid 100, from about amino acid 160 to about amino acid 198, and from about amino acid 261 to about amino acid 272 of SEQ ID NO:14.

Based on a BLAST search performed on 32164, homology was shown to human and other mammalian olfactory receptors of the rhodopsin family of GPCRs.

A hydropathy plot of human 32164 was performed. Polypeptides of the invention include fragments which include: all or part of a hydrophobic sequence, e.g., the sequence from about amino acid 25 to 45, from about 100 to 130, from about 140 to 160, from about 185 to 225, from about 241 to 261 and from about 275 to 285 of SEQ ID NO:14; all or part of a hydrophilic sequence, e.g., the sequence from about amino acid 165 to 180 and from about 226 to 235 of SEQ ID NO:14; a sequence which includes a Cys, or a glycosylation site.

Expression of 32164 is highly specific for hematopoietic cells. Hematopoietic progenitor CD34+ cells show significant expression of 32164 message. High level expression was also detected in fetal liver containing hematopoietic islands, and in erythroid lineage cells. Expression was regulated during both in vivo and in vitro generation of erythroid cells. Megakaryotes generated in vitro from CD34+ cells treated with Steel factor and thrombopoietin (which has previously been shown to induce the expression of erythroid-specific genes) showed high level expression of 32164.

### 39404, 38911, 26904 and 31237 Polypeptides

The invention is based, in part, on the identification of novel seven-transmembrane proteins/G-protein coupled receptors, identified herein as 39404, 38911, 26904 and 31237. Specifically, an expressed sequence tag (EST) was selected based on homology to G-protein-coupled receptor sequences or motifs (e.g., seven-transmembrane domains). This EST was used to design primers based on sequences that it contains and used to identify a 39404 cDNA from a human colon cDNA library, a 38911 cDNA from a human bone marrow cDNA library, a 26904 cDNA from a human brain cDNA library, a 31237 cDNA from a human colon cDNA library. Positive clones were sequenced and the overlapping fragments were assembled. Analysis of the assembled sequences revealed that the cloned cDNA molecules encode G-protein coupled receptors (39404, 31237) or putative G-protein coupled receptors (38911, 26904).

The invention thus relates to a novel 39404 GPCR having the deduced amino acid sequence shown in SEQ ID NO:16 or having the amino acid sequence encoded by the deposited cDNA, ATCC No.______.

The invention also thus relates to a novel putative 38911 GPCR having the deduced amino acid sequence shown in SEQ ID NO:18 or having the amino acid sequence encoded by the deposited cDNA, ATCC No.______.

The invention also thus relates to a novel putative 26904 GPCR having the deduced amino acid sequence shown in SEQ ID NO:20 or having the amino acid sequence encoded by the deposited cDNA, ATCC No.______.

The invention also thus relates to a novel 31237 GPCR having the deduced amino acid sequence shown in SEQ ID NO:22 or having the amino acid sequence encoded by the deposited cDNA, ATCC No.______.

The deposits will be maintained under the terms of the Budapest Treaty on the International Recognition of the Deposit of Microorganisms. The deposits are provided as a convenience to those of skill in the art and is not an admission that a deposit is required under 35 U.S.C. §112. The deposited sequences, as well as the polypeptides encoded by the sequences, are incorporated herein by reference and control in the event of any conflict, such as a sequencing error, with description in this application.

The “39404 polypeptide” or “39404 protein” refers to the polypeptide in SEQ ID NO:16 or encoded by the deposited cDNA. The “38911 polypeptide” or “38911 protein” refers to the polypeptide in SEQ ID NO:18 or encoded by the deposited cDNA. The “26904 polypeptide” or “26904 protein” refers to the polypeptide in SEQ ID NO:20 or encoded by the deposited cDNA. The “31237 polypeptide” or “31237 protein” refers to the polypeptide in SEQ ID NO:22 or encoded by the deposited cDNA. The term “protein” or “polypeptide”, however, further includes the numerous variants of 39404, 38911, 26904, or 31237 polypeptides described herein, as well as fragments derived from the full length 39404, 38911, 26904 or 31237 polypeptides and variants.

The present invention thus provides isolated or purified 39404, 38911, 26904, and 31237 polypeptides and variants and fragments thereof.

The 39404 polypeptide is a 337 residue protein exhibiting three main structural domains, an amino terminal extracellular domain, transmembrane domain, and carboxy terminal intracellular domain. 39404 also exhibits three glycosylation sites at amino acids about 10 to 13, 23 to 26 and 176 to 179 of SEQ ID NO:16; two cAMP- and cGMP-dependent protein kinase phosphorylation sites at about amino acids 240 to 243 and 329 to 332 of SEQ ID NO:16; four protein kinase C phosphorylation sites at about amino acids 175 to 177, 178 to 180, 194 to 196 and 316 to 318 of SEQ ID NO:16; and one casein kinase II phosphorylation site at about amino acids 187 to 190 of SEQ ID NO:16. In addition, amino acids corresponding in position to the GPCR signature and containing the invariant arginine are found in the sequence FRY at amino acids 130-132 of SEQ ID NO:16.

Additionally, transmembrane segments predicted by MEMSAT for the predicted entire coding sequence, predicted that amino acids 1 to about 37 of SEQ ID NO:16 constitute the amino terminal extracellular domain, amino acids about 38-305 of SEQ ID NO:16 constitute the region spanning the transmembrane domain, and amino acids about 306-337 of SEQ ID NO:16 constitute the carboxy terminal intracellular domain. The transmembrane domain contains seven transmembrane segments, three extracellular loops and three intracellular loops. The transmembrane segments are found from about amino acid 38 to about amino acid 60, from about amino acid 70 to about amino acid 90, from about amino acid 117 to about amino acid 136, from about amino acid 149 to about amino acid 172, from about amino acid 200 to about amino acid 222, from about amino acid 242 to about amino acid 260, and from about amino acid 283 to about amino acid 305 of SEQ ID NO:16. Within the region spanning the entire transmembrane domain are three intracellular and three extracellular loops. The three intracellular loops are found from about amino acid 61 to about amino acid 69, from about amino acid 137 to about amino acid 148, and from about amino acid 223 to about amino acid 241 of SEQ ID NO:16. The three extracellular loops are found at from about amino acid 91 to about amino acid 116, from about amino acid 173 to about amino acid 199, and from about amino acid 261 to about amino acid 282 of SEQ ID NO:16. Based on a BLAST search, highest homology was shown to purinoceptors (rhodopsin superfamily).

A hydropathy plot of human 39404 was performed. Polypeptides of the invention include fragments which include: all or part of a hydrophobic sequence, e.g., the sequence from about amino acid 40 to 51, from about 65 to 91, from about 121 to 141, from about 151 to 171, from about 205 to 221, from about 245 to 261 and from about 285 to 301 of SEQ ID NO:16; all or part of a hydrophilic sequence, e.g., the sequence from about amino acid 225 to 241 and from about 321 to 337 of SEQ ID NO:16; a sequence which includes a Cys, or a glycosylation site.

As assessed by TaqMan analysis, the 39404 protein is expressed at high levels in brain, kidney, fetal kidney and fetal liver and in moderate levels in breast, vein, fetal kidney and fetal liver. High expression was also onserved in aortic intimal proliferations and internal mammary artery.

The 38911 polypeptide is a 337 residue protein exhibiting three main structural domains, the amino terminal extracellular domain, transmembrane domain, and carboxy terminal intracellular domain. 38911 also exhibits one glycosylation site at about amino acids 3 to 6 of SEQ ID NO:18; one cAMP- and cGMP-dependent protein kinase phosphorylation site at about amino acids 324 to 327 of SEQ ID NO:18; two protein kinase C phosphorylation sites at about amino acids 17 to 19 and 323 to 325 of SEQ ID NO:18; three casein kinase II phosphorylation sites at about amino acids 194 to 197, 327 to 330, and 333 to 336 of SEQ ID NO:18; and nine N-myristoylation sites at about amino acids 26 to 31, 49 to 54, 103 to 108, 150 to 155, 156 to 161, 191 to 196, 253 to 258, 278 to 283, and 316 to 321 of SEQ ID NO:18. For the cAMP and cGMP dependent protein kinase phosphorylation, the actual modified residue is the last amino acid. For protein kinase C phosphorylation, the actual modified residue is the first amino acid. For casein kinase II phosphorylation, the actual modified residue is the first amino acid. For N-myristoylation, the actual modified residue is the first amino acid.

Additionally, transmembrane segments predicted by MEMSAT for the predicted entire coding sequence of 38911, predicted that amino acids 1 to about 40 of SEQ ID NO:18 constitute the amino terminal extracellular domain, amino acids about 41-294 of SEQ ID NO:18 constitute the region spanning the transmembrane domain, and amino acids about 259-337 of SEQ ID NO:18 constitute the carboxy terminal intracellular domain. The transmembrane domain contains seven transmembrane segments, three extracellular loops and three intracellular loops. The transmembrane segments are found from about amino acid 41 to about amino acid 60, from about amino acid 68 to about amino acid 92, from about amino acid 113 to about amino acid 137, from about amino acid 153 to about amino acid 172, from about amino acid 205 to about amino acid 228, from about amino acid 237 to about amino acid 260, and from about amino acid 275 to about amino acid 294 of SEQ ID NO:18. Within the region spanning the entire transmembrane domain are three intracellular and three extracellular loops. The three intracellular loops are found from about amino acid 61 to about amino acid 67, from about amino acid 138 to about amino acid 152, and from about amino acid 229 to about amino acid 236 of SEQ ID NO:18. The three extracellular loops are found at from about amino acid 93 to about amino acid 112, from about amino acid 173 to about amino acid 204, and from about amino acid 261 to about amino acid 274 of SEQ ID NO:18.

Based on a BLAST search performed on 38911, highest homology to 38911 was shown to the C5a anaphylatoxin receptor (G-protein Linked Receptor Facts Book, Watson and Arkinstall, Editors, Academic Press (1994) New York, pgs. 71-73, incorporated herein by reference for its teachings regarding this receptor).

A hydropathy plot of human 38911 was performed. Polypeptides of the invention include fragments which include: all or part of a hydrophobic sequence, e.g., the sequence from about amino acid 25 to 61, from about 65 to 95, from about 111 to 135, from about 145 to 165, from about 205 to 221, from about 231 to 265 and from about 275 to 291 of SEQ ID NO:18; all or part of a hydrophilic sequence, e.g., the sequence from about amino acid 305 to 337 of SEQ ID NO:18; a sequence which includes a Cys, or a glycosylation site.

As assessed by TaqMan analysis, the 38911 protein is expressed in osteoclasts, spleen, tonsils, liver, kidney, and testis.

The 26904 polypeptide is a 450 residue protein exhibiting three main structural domains, the amino terminal extracellular domain, transmembrane domain, and carboxy terminal intracellular domain. 26904 also exhibits one glycosylation site at about amino acids 312 to 315 of SEQ ID NO:20; one cAMP- and cGMP-dependent protein kinase phosphorylation site at about amino acids 143 to 146 of SEQ ID NO:20; seven protein kinase C phosphorylation sites at about amino acids 6 to 8, 136 to 138, 234 to 236, 245 to 247, 314 to 316, 436 to 438, and 446 to 448 of SEQ ID NO:20; seven casein kinase II phosphorylation sites at about amino acids 55 to 58, 167 to 170, 218 to 221, 239 to 242, 284 to 287, 416 to 419, and 447 to 450 of SEQ ID NO:20; four tyrosine kinase phosphorylation sites at about amino acids 118 to 125, 336 to 343, 382 to 389, and 409 to 415 of SEQ ID NO:20; seven N-myristoylation sites at about amino acids 36 to 41, 91 to 96, 261 to 266, 304 to 309, 365 to 370, 404 to 409, and 420 to 425 of SEQ ID NO:20; one amidation site at about amino acids 141 to 144 of SEQ ID NO:20; and one ATP/GTP-binding site motif A (P-loop) at about amino acids 230 to 237 of SEQ ID NO:20. In the case of protein kinase C phosphorylation, the actual modified residue is the first amino acid. In the case of casein kinase II phosphorylation, the actual modified residue is the first amino acid. In the case of the tyrosine kinase phosphorylation, the modified amino acid is the last amino acid. In the case of N-myristoylation, the modified amino acid is the first amino acid.

Additionally, transmembrane segments predicted by MEMSAT for the predicted entire coding sequence of 26904, predicted that amino acids 1 to about 30 of SEQ ID NO:20 constitute the amino terminal extracellular domain, amino acids about 30-435 of SEQ ID NO:20 constitute the region spanning the transmembrane domain, and amino acids about 435-450 of SEQ ID NO:20 constitute the carboxy terminal intracellular domain. The transmembrane domain contains seven transmembrane segments, three extracellular loops and three intracellular loops. The transmembrane segments are found from about amino acid 30 to about amino acid 50, from about amino acid 100 to about amino acid 120, from about amino acid 140 to about amino acid 165, from about amino acid 200 to about amino acid 240, from about amino acid 305 to about amino acid 340, from about amino acid 360 to about amino acid 380, and from about amino acid 410 to about amino acid 450 of SEQ ID NO:20. Within this region spanning the entire transmembrane domain are three intracellular and three extracellular loops.

A hydropathy plot of human 26904 was performed. Polypeptides of the invention include fragments which include: all or part of a hydrophobic sequence, e.g., the sequence from about amino acid 31 to 41, from about 105to 115, from about 145 to 155 and from about 415 to 431 of SEQ ID NO:20; all or part of a hydrophilic sequence, e.g., the sequence from about amino acid 51 to 65, from about 75 to 101, from about 131 to 141, from about 160 to 170, from about 231 to 245 and from about 291 to 315 of SEQ ID NO:20; a sequence which includes a Cys, or a glycosylation site.

As assessed by TaqMan analysis, the isolated 26904 protein is expressed in brain samples.

The 31237 polypeptide is a 486 residue protein exhibiting three main structural domains, an amino terminal extracellular domain, transmembrane domain, and carboxy terminal intracellular domain. PFAM signature analysis shows that the 31237 receptor has the highest homology to the metabotropic family of G-protein coupled receptors.

As assessed by TaqMan analysis, the isolated 31237 protein is expressed in colon samples.

### 18057 Polypeptides

The invention is based, in part, on the identification of a novel human seven transmembrane protein, potentially a novel human G-coupled protein receptor, identified herein as 18057. Specifically, an expressed sequence tag (EST) was selected based on homology to G-protein-coupled receptor sequences. This EST was used to design primers based on primary sequences that it contains and used to identify a cDNA from a human cDNA library. Positive clones were sequenced and the overlapping fragments were assembled. Analysis of the assembled sequence revealed that the cloned cDNA molecule encodes a seven transmembrane protein, potentially a G-protein coupled receptor.

The invention thus relates to a novel seven transmembrane protein having the deduced amino acid sequence shown in SEQ ID NO:52.

The “18057 polypeptide” or “18057 protein” refers to the polypeptide in SEQ ID NO:52. The terms, however, further include the numerous variants described herein, as well as fragments derived from the full length 18057 polypeptide and variants.

The present invention thus provides an isolated or purified 18057 polypeptide and variants and fragments thereof.

The 18057 polypeptide is a 469 residue protein exhibiting three main structural domains, an amino terminal extracellular domain, a transmembrane domain, and a carboxy terminal intracellular domain. The transmembrane domain contains seven segments that span the membrane. Within the region spanning the entire transmembrane domain are three intracellular and three extracellular loops. 18057 also exhibits three glycosylation sites from about amino acid 94 to 97, 168 to 171, and 357 to 360 of SEQ ID NO:52; one protein kinase C phosphorylation site from about amino acid 264 to 266 of SEQ ID NO:52; three casein kinase II phosphorylation sites from about amino acid 207 to 210, 251 to 254, and 458 to 461 of SEQ ID NO 52; and nine N-myristoylation sites from about amino acid 15 to 20, 82 to 87, 149 to 154, 160 to 165, 190 to 195, 277 to 282, 316 to 321, 370 to 375, and 439 to 444 of SEQ ID NO:52. In the case of glycosylation, the actual modified residue is the first amino acid. In the case of protein kinase C phosphorylation, casein kinase II phosphorylation, and N-myristoylation, the actual modified residue is the first amino acid. Predicted transmembrane segments for the deduced 18057 amino acid sequence (SEQ ID NO:52) include from about amino acid 7 to 25, 38 to 61, 72 to 93, 106 to 127, 136 to 158, 221 to 241, 292 to 310, 332 to 351, 360 to 383, 397 to 421 and 428 to 451 of SEQ ID NO:52.

A hydropathy plot of human 18057 was performed. Polypeptides of the invention include fragments which include: all or part of a hydrophobic sequence, e.g., the sequence from about amino acid 1 to 25, from about 65 to 81, from about 101 to 111, from about 131 to 145, from about 215 to 235, from about 295 to 305, from about 331 to 351, from about 360 to 380, from about 385 to 411 and from about 425 to 469 of SEQ ID NO:52; all or part of a hydrophilic sequence, e.g., the sequence from about amino acid 255 to 265 and from about 275 to 285 of SEQ ID NO:52; a sequence which includes a Cys, or a glycosylation site.

Based on a BLAST search performed on 18057, homology of 18057 was shown to GPCRs.

TaqMan analyses demonstrate that the 18057 nucleic acid is highly expressed in tissues or cells that include, but are not limited to human testes. The gene also shows expression in various other normal human tissues including, but not limited to, aorta, brain, breast, cervix, colon, esophagus, heart, kidney, liver, lung, lymph, muscle, ovary, placenta, prostate, small intestine, spleen, testes, thymus, thyroid, vein, pancreas, spinal cord, and astrocytes. Additional TaqMan analyses using oncology panels demonstrate 18057 expression in breast tumor, lung tumor, ovary tumor, colon tumor, prostate tumor, brain tumor, and metastatic liver cells.

### 16405 Polypeptides

The invention is based, in part, on the discovery of a novel G-coupled protein receptor, identified herein as 16405. Specifically, an expressed sequence tag (EST) was selected based on homology to G-protein-coupled receptor sequences. This EST was used to design primers based on sequences that it contains and used to identify a cDNA from a human spleen cDNA library. Positive clones were sequenced and the overlapping fragments were assembled. Analysis of the assembled sequence revealed that the cloned cDNA molecule encodes a G-protein coupled receptor.

The invention thus relates to a novel 16405 GPCR having the deduced amino acid sequence shown in SEQ ID NO:56 or having the amino acid sequence encoded by the deposited cDNA, ATCC No.______.

The deposit will be maintained under the terms of the Budapest Treaty on the International Recognition of the Deposit of Microorganisms. The deposit is provided as a convenience to those of skill in the art and is not an admission that a deposit is required under 35 U.S.C. §112. The deposited sequence, as well as the polypeptide encoded by the sequence, is incorporated herein by reference and controls in the event of any conflict, such as a sequencing error, with description in this application.

The “16405 receptor polypeptide” or “16405 receptor protein” refers to the polypeptide in SEQ ID NO:56 or encoded by the deposited cDNA. The term “receptor protein” or “receptor polypeptide”, however, further includes the numerous variants described herein, as well as fragments derived from the full length polypeptide and variants.

The present invention thus provides an isolated or purified receptor polypeptide and variants and fragments thereof.

The 16405 polypeptide is a 384 residue protein exhibiting three main structural domains, an amino terminal extracellular domain, a transmembrane domain, and a carboxy terminal intracellular domain. The transmembrane domain contains seven segments that span the membrane. Within the region spanning the entire transmembrane domain are three intracellular and three extracellular loops. An analysis of the 16405 open reading frame for amino acids corresponding to specific functional sites revealed that 16405 contains one glycosylation site from about amino acid 3 to about amino acid 6 of SEQ ID NO:56; three cAMP and cGMP-dependent protein kinase phosphorylation sites from about amino acids 155 to 158, 224 to 227, and 279 to 282 of SEQ ID NO:56; five protein kinase C phosphorylation sites from about amino acids 58 to 60, 111 to 113, 222 to 224, 337 to 339, and 346 to 348 of SEQ ID NO:56; nine N-myristoylation sites from about amino acids 40 to 45, 92 to 97, 107 to 112, 117 to 122, 123 to 128, 239 to 244, 316 to 321, 353 to 358, and 376 to 381 of SEQ ID NO:56; one amidation site from about amino acid 28 to 31 of SEQ ID NO:56; and one leucine zipper pattern from about amino acid 115 to 136 of SEQ ID NO:56. It is predicted that amino acids 1-31 of SEQ ID NO:56 constitute the amino terminal extracellular domain, amino acids 32-338 of SEQ ID NO:56 constitute the region spanning the transmembrane domain, and amino acids 339-383 of SEQ ID NO:56 constitute the carboxy terminal intracellular domain. The transmembrane domain contains seven transmembrane segments, three extracellular loops and three intracellular loops. The transmembrane segments are found from about amino acid 32 to about amino acid 56, from about amino acid 68 to about amino acid 85, from about amino acid 118 to about amino acid 1136, from about amino acid 159 to about amino acid 176, from about amino acid 194 to about amino acid 216, from about amino acid 281 to about amino acid 305, and from about amino acid 319 to about amino acid 338 of SEQ ID NO:56. Within the region spanning the entire transmembrane domain are three intracellular and three extracellular loops. The three intracellular loops are found from about amino acid 57 to about amino acid 67, from about amino acid 137 to about amino acid 158 and from about amino acid 217 to about amino acid 280 of SEQ ID NO:56. The three extracellular loops are found at from about amino acid 86 to about amino acid 117, from about amino acid 177 to about amino acid 193, and from about amino acid 304 to about amino acid 318 of SEQ ID NO:56.

A comparison of the 16405 receptor against the Prosite database of protein patterns specifically shows a high score against the Seven Transmembrane Segment Rhodopsin Superfamily consensus sequence (SEQ ID NO:58 and 59). The most commonly conserved sequence is an aspartate, arginine, tyrosine (DRY) triplet. DRY is implicated in signal transduction. Arginine is invariant. Aspartate is conservatively placed in several GPCRs. In the present case, the arginine is found in the sequence RYL at residues 137-139 of SEQ ID NO:56, which corresponds to DRY or the invariant arginine in GPCRs of the rhodopsin superfamily of receptors.

A hydropathy plot of human 16405 was performed. Polypeptides of the invention include fragments which include: all or part of a hydrophobic sequence, e.g., the sequence from about amino acid 35 to 55, from about 70 to 95, from about 120 to 140, from about 160 to 180, from about 190 to 215, from about 225 to 245, from about 290 to 330 and from about 355 to 370 of SEQ ID NO:56; all or part of a hydrophilic sequence, e.g., the sequence from about amino acid 100 to 115, from about 145 to 155, from about 250 to 280 and from about 335 to 345 of SEQ ID NO:56; a sequence which includes a Cys, or a glycosylation site.

As assessed by TaqMan analysis, the 16405 receptor protein is expressed in spleen, glioblastoma, and sclerotic lesion samples.

### 32705, 23224, 27423. 32700 and 32712 Polypeptides

The invention is based, in part, on the identification of novel human G-proteins, potentially having GTPase activity, identified herein as 32705, 23224, 27423, 32700 or 32712. Specifically, an expressed sequence tag (EST) was selected based on homology to G-protein sequences. This EST was used to design primers based on primary sequences that it contains and used to identify a cDNA from human cDNA libraries. Positive clones were sequenced and the overlapping fragments were assembled. Analysis of the assembled sequence revealed that the cloned cDNA molecules encode small G-proteins, potentially with GTPase activity.

The invention thus relates to novel 32705, 23224, 27423, 32700 or 32712 G-proteins having the deduced amino acid sequence shown in SEQ ID NO:61, SEQ ID NO:63, SEQ ID NO:65, SEQ ID NO:67, and SEQ ID NO:69, respectively.

The “G-protein polypeptide” or “G-protein” refers to a polypeptide in SEQ ID NO:61, SEQ ID NO:63, SEQ ID NO:65, SEQ ID NO:67, and SEQ ID NO:69. The terms, however, further include the numerous variants described herein, as well as fragments derived from the full length G-protein polypeptide and variants.

The present invention thus provides an isolated or purified G-protein polypeptide and variants and fragments thereof.

Based on a BLAST search of the 32705 sequence, homology was shown to human Ras-like proteins, and in particular GTP-binding proteins, for example, Rac1 (GenBank Accession No. AAA67040), and also having homology to the Rac Chp homolog (GenBank Accession No. AAC69198). Homology has also been shown to the human Rac3 gene (GenBank Accession No. AF097887). A search for complete domains of 32705 in PFAM detected a Ras family domain at amino acids 33 to 228 of SEQ ID NO:61. Analysis of the 23224 sequence in PFAM showed the highest scores with the Rab subgroup and the Ras family at amino acids 10 to 213 of SEQ ID NO:63. Homology analysis of the 27423 G-protein also showed the highest scores with Rab and the Ras family at amino acids 10 to 207 of SEQ ID NO:65. Homology analysis of the 32700 G-protein showed the highest scores with Rab and the Ras family at amino acids 8 to 183 of SEQ ID NO:67. Homology analysis of the 32712 G-protein showed the highest scores with Rab and the Ras family amino acids 2 to 191 of SEQ ID NO:69. The Pfam consensus amino acid sequence of the Ras family domain which was identified in the 32705, 23224, 27423, 32700 and 32712 polypeptides corresponds to SEQ ID NO:70.

An analysis of the open reading frame for the 32705 amino acid sequence (SEQ ID NO:61) corresponding to predicted functional sites revealed an N-glycosylation site at about amino acids 120 to 123 of SEQ ID NO:61; one cAMP and cGMP-dependent protein kinase phosphorylation site at about amino acids 22 to 25 of SEQ ID NO:61; two protein kinase C phosphorylation sites at about amino acids 122 to 124 and 190 to 192 of SEQ ID NO:61; four casein kinase II phosphorylation sites at about amino acids 7 to 10, 63 to 66, 143 to 146 and 204 to 207 of SEQ ID NO:61; and one ATP/GTP-binding site motif at amino acid residues 38 to 45 of SEQ ID NO:61. For the N-glycosylation site, the actual modified residue is the first amino acid. For the cAMP-and cGMP-dependent protein kinase phosphorylation site, the actual modified residue is the last amino acid. For the protein kinase C phosphorylation sites, the actual modified residue is the first amino acid. For the casein kinase II phosphorylation sites, the actual modified residue is the first amino acid.

A hydropathy plot of human 32705 was performed. Polypeptides of the invention include fragments which include: all or part of a hydrophobic sequence, e.g., the sequence from about amino acid 32 to 40, from about 42 to 49, from about 65 to 79, from about 101 to 111, from about 132 to 141, from about 181 to 191 and from about 195 to 205 of SEQ ID NO:61; all or part of a hydrophilic sequence, e.g., the sequence from about amino acid 1 to 31, from about 51 to 61, from about 81 to 91, from about 112 to 131, from about 155 to 171 and from about 206 to 225 of SEQ ID NO:61; a sequence which includes a Cys, or a glycosylation site.

An analysis of the open reading frame for the 23224 amino acid sequence (SEQ ID NO:63) corresponding to predicted functional sites revealed one cAMP and cGMP-dependent protein kinase phosphorylation site at about amino acids 26 to 29 of SEQ ID NO:63; four protein kinase C phosphorylation sites at about amino acids 92 to 94, 129 to 131, 153 to 155 and 207 to 209 of SEQ ID NO:63; four casein kinase II phosphorylation sites at about amino acids 134 to 137, 153 to 156, 181 to 184 and 200 to 203 of SEQ ID NO:63; one N-myristoylation site at about amino acids 188 to 193 of SEQ ID NO:63; one amidation site at about amino acids 54 to 57 of SEQ ID NO:63; and one ATP/GTP-binding site motif at amino acid residues 15 to 22 of SEQ ID NO:63. For the cAMP-and cGMP-dependent protein kinase phosphorylation site, the actual modified residue is the first amino acid. For the protein kinase C phosphorylation sites, the actual modified residue is the first amino acid. For the casein kinase II phosphorylation sites, the actual modified residue is the first amino acid.

A hydropathy plot of human 23224 was performed. Polypeptides of the invention include fragments which include: all or part of a hydrophobic sequence, e.g., the sequence from about amino acid 1 to 21, from about 42 to 52, from about 82 to 89 and from about 115 to 120 of SEQ ID NO:63; all or part of a hydrophilic sequence, e.g., the sequence from about amino acid 25 to 35, from about 65 to 81, from about 91 to 111 and from about 125 to 141 of SEQ ID NO:63; a sequence which includes a Cys, or a glycosylation site.

An analysis of the open reading frame for the 27423 amino acid sequence (SEQ ID NO:65) corresponding to predicted functional sites revealed three N-glycosylation sites at about amino acids 34 to 37, 178 to 181 and 194 to 197 of SEQ ID NO:65; one glycosaminoglycan attachment site at about amino acids 17 to 20 of SEQ ID NO:65; one cAMP and cGMP-dependent protein kinase phosphorylation site at about amino acids 197 to 200 of SEQ ID NO:65; two protein kinase C phosphorylation sites at about amino acids 151 to 153 and 196 to 198 of SEQ ID NO:65; one casein kinase II phosphorylation site at about amino acids 112 to 115 of SEQ if) NO:65; one N-myristoylation site at about amino acids 18 to 23 of SEQ ID NO:65; one amidation site at about amino acids 53 to 56 of SEQ ID NO:65; one prenyl group binding site at about amino acids 204 to 207 of SEQ ID NO:65; and one ATP/GTP-binding site motif at amino acid residues 15 to 22 of SEQ ID NO:65. For the N-glycosylation site, the actual modified residue is the first amino acid. For the cAMP-and cGMP-dependent protein kinase phosphorylation site, the actual modified residue is the last amino acid. For the protein kinase C phosphorylation sites, the actual modified residue is the first amino acid. For the casein kinase II phosphorylation sites, the actual modified residue is the first amino acid.

A hydropathy plot of human 27423 was performed. Polypeptides of the invention include fragments which include: all or part of a hydrophobic sequence, e.g., the sequence from about amino acid 11 to 19, from about 21 to 29, from about 35 to 45 and from about 83 to 90 of SEQ ID NO:65; all or part of a hydrophilic sequence, e.g., the sequence from about amino acid 65 to 73, from about 91 to 115, from about 121 to 135, from about 165 to 175 and from about 185 to 195 of SEQ ID NO:65; a sequence which includes a Cys, or a glycosylation site.

An analysis of the 32700 open reading frame for amino acids (SEQ ID NO:67) corresponding to predicted functional sites revealed a protein kinase phosphorylation site at about amino acids 149 to 151 of SEQ ID NO:67; two casein kinase II phosphorylation sites at about amino acids 144 to 147 and 149 to 152 of SEQ if) NO:67; one amidation site at about amino acids 133 to 136 of SEQ ID NO:67; one prenyl group binding site at about amino acids 180 to 183 of SEQ ID NO:67; and one ATP/GTP-binding site motif at amino acid residues 13 to 20 of SEQ ID NO:67. For the protein kinase C phosphorylation site, the actual modified residue is the first amino acid. For the casein kinase II phosphorylation sites, the actual modified residue is the first amino acid.

A hydropathy plot of human 32700 was performed. Polypeptides of the invention include fragments which include: all or part of a hydrophobic sequence, e.g., the sequence from about amino acid 10 to 19, from about 71 to 85 and from about 112 to 117 of SEQ ID NO:67; all or part of a hydrophilic sequence, e.g., the sequence from about amino acid 31 to 41, from about 45 to 53, from about 59 to 65, from about 101 to 111, from about 121 to 135, from about 149 to 155 and from about 171 to 183 of SEQ ID NO:67; a sequence which includes a Cys, or a glycosylation site.

A hydropathy plot of human 32712 was performed. Polypeptides of the invention include fragments which include: all or part of a hydrophobic sequence, e.g., the sequence from about amino acid 11 to 19, from about 22 to 34, from about 71 to 79, from about 102 to 110 and from about 145 to 155 of SEQ ID NO:69; all or part of a hydrophilic sequence, e.g., the sequence from about amino acid 55 to 69, from about 112 to 131 and from about 159 to 191 of SEQ ID NO:69; a sequence which includes a Cys, or a glycosylation site.

As assessed by TaqMan analysis, 32705 is highly expressed in tissues or cells that include, but are not limited to lung, brain, pancreas, skeletal muscle, nerve, normal skin, static HUVEC (Human Umbilical Vein Endothelial Cells), ganglia and virus-infected hepatocytes. Expression of 32705 is particularly high in brain. Differential expression of 32705 is shown in hepatitis B virus-infected HepG2 cells. 23224 is expressed in tissues and cells that include, but are not limited to kidney, pancreas, spinal cord, brain cortex, brain hypothalamus, erythroid and dorsal root ganglia. 32700 is expressed in tissues and cells that include, but are not limited to, HUVEC (Human Umbilical Vein Endothelial Cells), hemangioma, skeletal muscle, brain cortex (normal), brain hypothalamus (normal), DRG (Dorsal Root Ganglion), ovary (tumor) and erythroid cells. 32712 is expressed in tissues and cell types including, but not limited to, kidney, primary osteoblasts, spinal cord (normal), brain cortex (normal), brain hypothalamus (normal), DRG (Dorsal Root Ganglion), prostate (normal), prostate (tumor), liver (normal), liver fibrosis, spleen (normal), tonsil (normal), lymph node (normal), BM-MNC (Bone Marrow Mononuclear Cells), neutrophils, megakaryocytes and erythroid cells.

### 12216 Polypeptides

The invention is based, in part, on the discovery of a novel G-coupled protein receptor, identified herein as 12216. Specifically, an expressed sequence tag (EST) was selected based on homology to G-protein-coupled receptor sequences. This EST was used to design primers based on sequences that it contains and used to identify a cDNA from a prostate fibroblast cDNA library. Positive clones were sequenced and the overlapping fragments were assembled. Analysis of the assembled sequence revealed that the cloned cDNA molecule encodes a G-protein coupled receptor.

The invention thus relates to a novel 12216 GPCR having the deduced amino acid sequence shown in SEQ ID NO:71 or having the amino acid sequence encoded by the deposited cDNA, ATCC No.______.

The deposit will be maintained under the terms of the Budapest Treaty on the International Recognition of the Deposit of Microorganisms. The deposit is provided as a convenience to those of skill in the art and is not an admission that a deposit is required under 35 U.S.C. §112. The deposited sequence, as well as the polypeptide encoded by the sequence, is incorporated herein by reference and controls in the event of any conflict, such as a sequencing error, with description in this application.

The “12216 receptor polypeptide” or “12216 receptor protein” refers to the polypeptide in SEQ ID NO:71 or encoded by the deposited cDNA. The term “receptor protein” or “receptor polypeptide”, however, further includes the numerous variants described herein, as well as fragments derived from the full length 12216 polypeptide and variants.

The present invention thus provides an isolated or purified 12216 receptor polypeptide and variants and fragments thereof.

The 12216 polypeptide is a 373 residue protein exhibiting three main structural domains. The amino terminal extracellular domain is identified to be within residues 1 to about 25 in SEQ ID NO:71. The transmembrane domain is identified to be within residues from about 26 to about 343 in SEQ ID NO:71. The carboxy terminal intracellular domain is identified to be within residues from about 344 to 373 in SEQ ID NO:71. The transmembrane domain contains seven segments that span the membrane. The transmembrane segments are found from about amino acid 26 to about amino acid 48, from about amino acid 59 to about amino acid 83, from about amino acid 98 to about amino acid 119, from about amino acid 137 to about amino acid 156, from about amino acid 187 to about amino acid 204, from about amino acid 287 to about amino acid 308, and from about amino acid 321 to about amino acid 343 in SEQ ID NO:71. Within the region spanning the entire transmembrane domain are three intracellular and three extracellular loops. The three intracellular loops are found from about amino acid 49 to about amino acid 58, from about amino acid 120 to about amino acid 136, and from about amino acid 205 to about amino acid 286 in SEQ ID NO:71. The three extracellular loops are found at from about amino acid 84 to about amino acid 97, from about amino acid 157 to about amino acid 186, and from about amino acid 309 to about amino acid 320 in SEQ ID NO:71.

An analysis of the 12216 open reading frame for amino acids corresponding to specific functional sites revealed three N-glycosylation sites, from about amino acid 3 to 6, 184 to 187, and 229 to 232 of SEQ ID NO:71; one cyclic AMP/cyclic GMP-dependent protein kinase phosphorylation site at about amino acids 133 to 136 of SEQ ID NO:71; four protein kinase C phosphorylation sites at about amino acid 82 to 84, 95 to 97, 164 to 166, and 269 to 271 of SEQ ID NO:71; one casein kinase II phosphorylation site at about amino acid 4 to 7 of SEQ ID NO:71; five N-myristoylation sites from about amino acid 30 to 35, 69 to 74, 86 to 91, 239 to 244, and 260 to 265 of SEQ ID NO:71. Finally, the protein is also predicted to contain a prenylation site (prenyl group binding site/CAAX box) at about amino acid 371 to 374 of SEQ ID NO:71.

A comparison of the 12216 receptor against the Prosite database of protein patterns specifically shows a high score against the Seven Transmembrane Segment Rhodopsin Superfamily (SEQ ID NO:73). The most commonly conserved sequence is an aspartate, arginine, tyrosine (DRY) triplet. DRY is implicated in signal transduction. Arginine is invariant. Aspartate is conservatively placed in several GPCRs. In the present case, the arginine is found in the sequence TRY at residues 120 to 122 in SEQ ID NO:71, which matches the position of DRY or invariant arginine in GPCRs of the rhodopsin superfamily of receptors.

A hydropathy plot of human 12216 was performed. Polypeptides of the invention include fragments which include: all or part of a hydrophobic sequence, e.g., the sequence from about amino acid 22 to 50, from about 60 to 82, from about 92 to 122, from about 135 to 160, from about 187 to 208 and from about 290 to 345 of SEQ ID NO:71; all or part of a hydrophilic sequence, e.g., the sequence from about amino acid 123 to 133, from about 165 to 184, from about 210 to 220, from about 227 to 240, from about 260 to 285 and from about 348 to 374 of SEQ ID NO:71; a sequence which includes a Cys, or a glycosylation site.

As assessed by TaqMan analysis, the 12216 receptor protein is expressed in brain, skeletal muscle, colon, heart CHF samples, mobilized peripheral blood CD34⁺ cells, human embryonic kidney cell lines, aorta, kidney, and monkey coronary, femoral, and renal arterial tissue, among others.

### POLYPEPTIDES OF THE PRESENT INVENTION

As used herein, a polypeptide is said to be “isolated” or “purified” when it is substantially free of cellular material when it is isolated from recombinant and non-recombinant cells, or free of chemical precursors or other chemicals when it is chemically synthesized. A polypeptide, however, can be joined to another polypeptide with which it is not normally associated in a cell and still be considered “isolated” or “purified.”

The receptor polypeptides can be purified to homogeneity. It is understood, however, that preparations in which the polypeptide is not purified to homogeneity are useful and considered to contain an isolated form of the polypeptide. The critical feature is that the preparation allows for the desired function of the polypeptide, even in the presence of considerable amounts of other components. Thus, the invention encompasses various degrees of purity.

In one embodiment, the language “substantially free of cellular material” includes preparations of the receptor polypeptide having less than about 30% (by dry weight) other proteins (i.e., contaminating protein), less than about 20% other proteins, less than about 10% other proteins, or less than about 5% other proteins. When the receptor polypeptide is recombinantly produced, it can also be substantially free of culture medium, i.e., culture medium represents less than about 20%, less than about 10%, or less than about 5% of the volume of the protein preparation.

A receptor polypeptide is also considered to be isolated when it is part of a membrane preparation or is purified and then reconstituted with membrane vesicles or liposomes.

The language “substantially free of chemical precursors or other chemicals” includes preparations of the receptor polypeptide in which it is separated from chemical precursors or other chemicals that are involved in its synthesis. In one embodiment, the language “substantially free of chemical precursors or other chemicals” includes preparations of the polypeptide having less than about 30% (by dry weight) chemical precursors or other chemicals, less than about 20% chemical precursors or other chemicals, less than about 10% chemical precursors or other chemicals, or less than about 5% chemical precursors or other chemicals.

In one embodiment, the receptor polypeptide comprises the amino acid sequence shown in SEQ ID NO:1, 4, 6, 8, 11, 14, 16, 18, 20, 22, 52, 56, 61, 63, 65, 67, 69 or 71. However, the invention also encompasses sequence variants.

Variants include a substantially homologous protein encoded by the same genetic locus in an organism, i.e., an allelic variant, as for 14400, in proximity to marker SHGC-5431, on the Y chromosome. The 16405 receptor has been mapped to chromosome 1, in proximity to the AFM297zg1 marker. The 2838 receptor maps to chromosome 2, in close proximity to WI-7921. The 15334 receptor has been mapped to chromosome 12 in close proximity to SHGC-30262. The 12216 receptor has been mapped to the X chromosome, in proximity to the SHGG-31766 marker. Variants also encompass proteins derived from other genetic loci in an organism, but having substantial homology to the 14400, 2838, 14618, 15334, 14274, 32164, 39404, 38911, 26904, 31237, 18057, 16405, 32705, 23224, 27423, 32700, 32712 or 12216 receptor protein of SEQ ID NO:1, 4, 6, 8, 11, 14, 16, 18, 20, 22, 52, 56, 61, 63, 65, 67, 69 or 71. Variants also include proteins substantially homologous to the 14400, 2838, 14618, 15334, 14274, 32164, 39404, 38911, 26904, 31237, 18057, 16405, 32705, 23224, 27423, 32700, 32712 or 12216 receptor protein but derived from another organism, i.e., an ortholog. Variants also include proteins that are substantially homologous to the 14400, 2838, 14618, 15334, 14274, 32164, 39404, 38911, 26904, 31237, 18057, 16405, 32705, 23224, 27423, 32700, 32712 or 12216 receptor protein that are produced by chemical synthesis. Variants also include proteins that are substantially homologous to the 14400, 2838, 14618, 15334, 14274, 32164, 39404, 38911, 26904, 31237, 18057, 16405, 32705, 23224, 27423, 32700, 32712 or 12216 receptor protein that are produced by recombinant methods. It is understood, however, that variants exclude any amino acid sequences disclosed prior to the invention.

As used herein, two proteins (or a region of the proteins) are substantially homologous when the amino acid sequences are at least about 50-55%, 55-60%, 60-65%, 65-70%, typically at least about 70-75%, more typically at least about 80-85%, and most typically at least about 90-95% or more homologous. A substantially homologous amino acid sequence, according to the present invention, will be encoded by a nucleic acid sequence hybridizing to the nucleic acid sequence, or portion thereof, of the sequence shown in SEQ ID NO:2, 5, 7, 9, 12, 15, 17, 19, 21, 23, 53, 57, 60, 62, 64, 66, 68 or 72 under stringent conditions as more fully described below.

To determine the percent homology of two amino acid sequences, or of two nucleic acids, the sequences are aligned for optimal comparison purposes (e.g., gaps can be introduced in the sequence of one protein or nucleic acid for optimal alignment with the other protein or nucleic acid). The amino acid residues or nucleotides at corresponding amino acid positions or nucleotide positions are then compared. When a position in one sequence is occupied by the same amino acid residue or nucleotide as the corresponding position in the other sequence, then the molecules are homologous at that position. As used herein, amino acid or nucleic acid “homology” is equivalent to amino acid or nucleic acid “identity”. The percent homology between the two sequences is a function of the number of identical positions shared by the sequences (i.e., per cent homology equals the number of identical positions/total number of positions times 100).

The invention also encompasses polypeptides having a lower degree of identity but having sufficient similarity so as to perform one or more of the same functions performed by the 14400, 2838, 14618, 15334, 14274, 32164, 39404, 38911, 26904, 31237, 18057, 16405, 32705, 23224, 27423, 32700, 32712 or 12216 polypeptide. Similarity is determined by conserved amino acid substitution. Such substitutions are those that substitute a given amino acid in a polypeptide by another amino acid of like characteristics. Conservative substitutions are likely to be phenotypically silent. Typically seen as conservative substitutions are the replacements, one for another, among the aliphatic amino acids Ala, Val, Leu, and Ile; interchange of the hydroxyl residues Ser and Thr, exchange of the acidic residues Asp and Glu, substitution between the amide residues Asn and Gln, exchange of the basic residues Lys and Arg and replacements among the aromatic residues Phe, Tyr. Guidance concerning which amino acid changes are likely to be phenotypically silent are found in Bowie et al., Science 247:1306-1310 (1990).

TABLE 2
Conservative Amino Acid Substitutions.

| | |
|---|---|
| Aromatic | Phenylalanine |
| | Tryptophan |
| | Tyrosine |
| Hydrophobic | Leucine |
| | Isoleucine |
| | Valine |
| Polar | Glutamine |
| | Asparagine |
| Basic | Arginine |
| | Lysine |
| | Histidine |
| Acidic | Aspartic Acid |
| | Glutamic Acid |
| Small | Alanine |
| | Serine |
| | Threonine |
| | Methionine |
| | Glycine |

Both identity and similarity can be readily calculated (Computational Molecular Biology, Lesk, A. M., ed., Oxford University Press, New York, 1988; Biocomputing: Informatics and Genome Projects, Smith, D. W., ed., Academic Press, New York, 1993; Computer Analysis of Sequence Data, Part 1, Griffin, A. M., and Griffin, H. G., eds., Humana Press, New Jersey, 1994; Sequence Analysis in Molecular Biology, von Heinje, G., Academic Press, 1987; and Sequence Analysis Primer, Gribskov, M. and Devereux, J., eds., M Stockton Press, New York, 1991).

The comparison of sequences and determination of percent identity and similarity between two sequences can be accomplished using a mathematical algorithm. (Computational Molecular Biology, Lesk, A. M., ed., Oxford University Press, New York, 1988; Biocomputing: Informatics and Genome Projects, Smith, D. W., ed., Academic Press, New York, 1993; Computer Analysis of Sequence Data, Part 1, Griffin, A. M., and Griffin, H. G., eds., Humana Press, New Jersey, 1994; Sequence Analysis in Molecular Biology, von Heinje, G., Academic Press, 1987; and Sequence Analysis Primer, Gribskov, M. and Devereux, J., eds., M Stockton Press, New York, 1991). A preferred, non-limiting example of such a mathematical algorithm is described in Karlin et al. (1993) Proc. Natl. Acad. Sci. USA 90:5873-5877. Such an algorithm is incorporated into the NBLAST and XBLAST programs (version 2.0) as described in Altschul et al. (1997) Nucleic Acids Res. 25:3389-3402. When utilizing BLAST and Gapped BLAST programs, the default parameters of the respective programs (e.g., NBLAST) can be used. In one embodiment, parameters for sequence comparison can be set at score=100, wordlength=12, or can be varied (e.g., W=5 or W=20).

In a preferred embodiment, the percent identity between two amino acid sequences is determined using the Needleman et al. (1970) (J. Mol. Biol. 48:444-453) algorithm which has been incorporated into the GAP program in the GCG software package, using either a BLOSUM 62 matrix or a PAM250 matrix, and a gap weight of 16, 14, 12, 10, 8, 6, or 4 and a length weight of 1, 2, 3, 4, 5, or 6. In yet another preferred embodiment, the percent identity between two nucleotide sequences is determined using the GAP program in the GCG software package (Devereux et al. (1984) Nucleic Acids Res. 12(1):387), using a NWSgapdna.CMP matrix and a gap weight of 40, 50, 60, 70, or 80 and a length weight of 1, 2, 3, 4, 5, or 6.

Another preferred, non-limiting example of a mathematical algorithm utilized for the comparison of sequences is the algorithm of Myers and Miller, CABIOS (1989). Such an algorithm is incorporated into the ALIGN program (version 2.0) which is part of the CGC sequence alignment software package. When utilizing the ALIGN program for comparing amino acid sequences, a PAM120 weight residue table, a gap length penalty of 12, and a gap penalty of 4 can be used. Additional algorithms for sequence analysis are known in the art and include ADVANCE and ADAM as described in Torellis et al (1994) Comput. Appl. Biosci. 10:3-5; and FASTA described in Pearson et al. (1988) PNAS 85:2444-8.

The protein sequence of the present invention can be used as a “query sequence” to perform a search against public databases to, for example, identify other family members or related sequences. Such searches can be performed using the NBLAST and XBLAST programs (version 2.0) of Altschul, et al. (1990) J. Mol. Biol. 215:403-10. BLAST nucleotide searches can be performed with the NBLAST program, score=100, wordlength=12 to obtain nucleotide sequences homologous to the nucleic acid molecules of the invention. BLAST protein searches can be performed with the XBLAST program, score=50, wordlength=3 to obtain amino acid sequences homologous to the proteins of the invention. To obtain gapped alignments for comparison purposes, Gapped BLAST can be utilized as described in Altschul et al., (1997) Nucleic Acids Res. 25(17):3389-3402. When utilizing BLAST and Gapped BLAST programs, the default parameters of the respective programs (e.g., XBLAST and NBLAST) can be used.

A variant polypeptide can differ in amino acid sequence by one or more substitutions, deletions, insertions, inversions, fusions, and truncations or a combination of any of these.

Variant polypeptides can be fully functional or can lack function in one or more activities. Thus, in the present case, variations can affect the function, for example, of one or more of the regions corresponding to ligand binding, membrane association, G-protein binding and signal transduction.

Fully functional variants typically contain only conservative variation or variation in non-critical residues or in non-critical regions. Functional variants can also contain substitution of similar amino acids which result in no change or an insignificant change in function. Alternatively, such substitutions may positively or negatively affect function to some degree.

Non-functional variants typically contain one or more non-conservative amino acid substitutions, deletions, insertions, inversions, or truncation or a substitution, insertion, inversion, or deletion in a critical residue or critical region.

As indicated, variants can be naturally-occurring or can be made by recombinant means or chemical synthesis to provide useful and novel characteristics for the receptor polypeptide. This includes preventing immunogenicity from pharmaceutical formulations by preventing protein aggregation.

Useful variations further include alteration of ligand binding characteristics. For example, one embodiment involves a variation at the binding site that results in binding but not release, or slower release, of ligand. A further useful variation at the same sites can result in a higher affinity for ligand. Useful variations also include changes that provide for affinity for another ligand. Another useful variation includes one that allows binding but which prevents activation by the ligand. Another useful variation includes variation in the transmembrane G-protein-binding/signal transduction domain that provides for reduced or increased binding by the appropriate G-protein or for binding by a different G-protein than the one with which the receptor is normally associated. Another useful variation provides a fusion protein in which one or more domains or subregions is operationally fused to one or more domains or subregions from another G-protein coupled receptor.

Amino acids that are essential for function can be identified by methods known in the art, such as site-directed mutagenesis or alanine-scanning mutagenesis (Cunningham et al., Science 244:1081-1085 (1989)). The latter procedure introduces single alanine mutations at every residue in the molecule. The resulting mutant molecules are then tested for biological activity such as receptor binding or in vitro, or in vitro proliferative activity. Sites that are critical for ligand-receptor binding can also be determined by structural analysis such as crystallization, nuclear magnetic resonance or photoaffinity labeling (Smith et al., J. Mol. Biol. 224:899-904 (1992); de Vos et al. Science 255:306-312 (1992)).

Substantial homology can be to the entire nucleic acid or amino acid sequence or to fragments of these sequences.

The invention thus also includes polypeptide fragments of the 14400, 2838, 14618, 15334, 14274, 32164, 39404, 38911, 26904, 31237, 18057, 16405, 32705, 23224, 27423, 32700, 32712 or 12216 receptor protein. Fragments can be derived from the amino acid sequence shown in SEQ ID NO:1, 4, 6, 8, 11, 14, 16, 18, 20, 22, 52, 56, 61, 63, 65, 67, 69 or 71. However, the invention also encompasses fragments of the variants of the 14400, 2838, 14618, 15334, 14274, 32164, 39404, 38911, 26904, 31237, 18057, 16405, 32705, 23224, 27423, 32700, 32712 or 12216 receptor protein as described herein.

The fragments to which the invention pertains, however, are not to be construed as encompassing fragments that may be disclosed prior to the present invention.

Fragments can retain one or more of the biological activities of the protein, for example the ability to bind to a G-protein or ligand. Fragments can also be useful as an immunogen to generate receptor antibodies.

Biologically active fragments of 14400, for example, can comprise a domain or motif, e.g., an extracellular or intracellular domain or loop, one or more transmembrane segments, or parts thereof, G-protein binding site, or GPCR signature, glycosylation sites, cAMP or a GMP-dependent, or casein kinase II phosphorylation sites, and myristoylation sites. Such peptides can be, for example, 7, 10, 15, 20, 30, 35, 36, 37, 38, 39, 40, 50, 100 or more amino acids in length.

Possible fragments of 14400 include, but are not limited to: 1) soluble peptides comprising the entire amino terminal extracellular domain about amino acid 1 to about amino acid 23 of SEQ ID NO:1, or parts thereof; 2) peptides comprising the entire carboxy terminal intracellular domain from about amino acid 297 to amino acid 359 of SEQ ID NO:1, or parts thereof; 3) peptides comprising the region spanning the entire transmembrane domain from about amino acid 24 to about amino acid 296 of SEQ ID NO:1, or parts thereof; 4) any of the specific transmembrane segments, or parts thereof, from about amino acid 24 to about amino acid 48, from about amino acid 59 to about amino acid 78, from about amino acid 89 to about amino acid 105, from about amino acid 139 to about amino acid 159, from about amino acid 189 to about amino acid 213, from about amino acid 234 to about amino acid 251, and from about amino acid 277 to about amino acid 296 of SEQ ID NO:1; 5) any of the three intracellular or three extracellular loops, or parts thereof, from about amino acid 49 to about amino acid 58, from about amino acid 79 to about amino acid 88, from about amino acid 106 to about amino acid 138, from about amino acid 160 to about amino acid 188, from about amino acid 214 to about amino acid 233, and from about amino acid 252 to about amino acid 276 of SEQ ID NO:1. Fragments further include combinations of the above fragments, such as an amino terminal domain combined with one or more transmembrane segments and the attendant extra or intracellular loops or one or more transmembrane segments, and the attendant intra or extracellular loops, plus the carboxy terminal domain. Thus, any of the above fragments can be combined. Other fragments include the mature protein from about amino acid 6 to 359 of SEQ ID NO:1. Other fragments contain the various functional sites described herein, such as phosphorylation sites, glycosylation sites, and myristoylation sites and a sequence containing the GPCR signature sequence. Fragments, for example, can extend in one or both directions from the functional site to encompass 5, 10, 15, 20, 30, 40, 50, or up to 100 amino acids. Further, fragments can include sub-fragments of the specific domains mentioned above, which sub-fragments retain the function of the domain from which they are derived. Fragments also include amino acid sequences greater than 107 amino acids. Fragments also include antigenic fragments and specifically those shown to have a high antigenic index. Further specific fragments include a fragment from about 107 to 359 of SEQ ID NO:1 and sub-fragments thereof, from about 120 to 359 of SEQ ID NO:1 and sub-fragments thereof, from about 123 to 359 of SEQ ID NO:1 and sub-fragments thereof, and from about 150 to 359 of SEQ ID NO:1 and sub-fragments thereof. Further fragments include a fragment including any amino acid sequences from 1-108 of SEQ ID NO:1 but extending beyond amino acid 108, a fragment including any amino acid sequences from 1-120 of SEQ ID NO:1 but extending beyond amino acid 120, a fragment including any amino acid sequences from 1-123 of SEQ ID NO:1 but extending beyond amino acid 123, and a fragment including any amino acid sequences from 1-150 of SEQ ID NO:1 but extending beyond amino acid 150.

Accordingly, possible 14400 fragments include fragments defining a ligand-binding site, fragments defining a glycosylation site, fragments defining membrane association, fragments defining phosphorylation sites, fragments defining interaction with G proteins and signal transduction, and fragments defining myristoylation sites. By this is intended a discrete fragment that provides the relevant function or allows the relevant function to be identified. In a preferred embodiment, the fragment contains the ligand-binding site.

Biologically active fragments of 2838 receptor (peptides which are, for example, 8, 12, 15, 20, 30, 35, 36, 37, 38, 39, 40, 50, 100 or more amino acids in length) can comprise a domain or motif, e.g., an extracellular or intracellular domain or loop, one or more transmembrane segments, or parts thereof, G-protein binding site, or GPCR signature, glycosylation sites, protein kinase C and casein kinase II phosphorylation sites, N-myristoylation and amidation sites. Such domains or motifs can be identified by means of routine computerized homology searching procedures.

Possible 2838 fragments include, but are not limited to: 1) soluble peptides comprising the entire amino terminal extracellular domain from amino acid 1 to about amino acid 24 of SEQ ID NO:4, or parts thereof; 2) peptides comprising the entire carboxy terminal intracellular domain from about amino acid 293 to amino acid 319 of SEQ ID NO:4, or parts thereof; 3) peptides comprising the region spanning the entire transmembrane domain from about amino acid 25 to about amino acid 292 of SEQ ID NO:4, or parts thereof; 4) any of the specific transmembrane segments, or parts thereof, from about amino acid 25 to about amino acid 49, from about amino acid 56 to about amino acid 79, from about amino acid 100 to about amino acid 117, from about amino acid 138 to about amino acid 159, from about amino acid 187 to about amino acid 210, from about amino acid 224 to about amino acid 248, and from about amino acid 268 to about amino acid 292 of SEQ ID NO:4; 5) any of the three intracellular or three extracellular loops, or parts thereof, from about amino acid 50 to about amino acid 55, from about amino acid 118 to about amino acid 137, from about amino acid 211 to about amino acid 223, from about amino acid 80 to about amino acid 99, from about amino acid 160 to about amino acid 186, and from about amino acid 249 to about amino acid 267 of SEQ ID NO:4. Fragments further include combinations of the above fragments, such as an amino terminal domain combined with one or more transmembrane segments and the attendant extra or intracellular loops or one or more transmembrane segments, and the attendant intra or extracellular loops, plus the carboxy terminal domain. Thus, any of the above fragments can be combined. Other fragments include the mature protein from about amino acid 6 to 319 of SEQ ID NO:4. Other fragments contain the various functional sites described herein, such as phosphorylation sites, glycosylation sites, and myristoylation sites and a sequence containing the GPCR signature sequence. Fragments, for example, can extend in one or both directions from the functional site to encompass 5, 10, 15, 20, 30, 40, 50, or up to 100 amino acids. Further, fragments can include sub-fragments of the specific domains mentioned above, which sub-fragments retain the function of the domain from which they are derived. Fragments also include amino acid sequences greater than 7 amino acids from amino acid 1 to about amino acid 264 of SEQ ID NO:4. Fragments also include antigenic fragments and specifically those shown to have a high antigenic index.

Accordingly, possible 2838 fragments include fragments defining a ligand-binding site, fragments defining a glycosylation site, fragments defining membrane association, fragments defining phosphorylation sites, fragments defining interaction with G proteins and signal transduction, and fragments defining myristoylation sites. By this is intended a discrete fragment that provides the relevant function or allows the relevant function to be identified. In a preferred embodiment, the fragment contains the ligand-binding site.

Biologically active fragments of 14618 receptor (peptides which are, for example, 9, 12, 15, 20, 30, 35, 36, 37, 38, 39, 40, 50, 100 or more amino acids in length) can comprise a domain or motif, e.g., an extracellular or intracellular domain or loop, one or more transmembrane segments, or parts thereof, G-protein binding site, or GPCR signature, glycosylation sites, and cAMP- and cGMP-dependent, protein kinase C, and casein kinase II phosphorylation sites.

Possible 14618 fragments include, but are not limited to: 1) soluble peptides comprising the entire amino terminal extracellular domain about amino acid 1 to about amino acid 28 of SEQ ID NO:6, or parts thereof; 2) peptides comprising the entire carboxy terminal intracellular domain from about amino acid 298 to amino acid 337 of SEQ ID NO:6, or parts thereof; 3) peptides comprising the region spanning the entire transmembrane domain from about amino acid 29 to about amino acid 297 of SEQ ID NO:6, or parts thereof; 4) any of the specific transmembrane segments, or parts thereof, from about amino acid 29 to about amino acid 49, from about amino acid 60 to about amino acid 84, from about amino acid 103 to about amino acid 127, from about amino acid 142 to about amino acid 161, from about amino acid 194 to about amino acid 217, from about amino acid 231 to about amino acid 247, and from about amino acid 276 to about amino acid 297 of SEQ ID NO:6; 5) any of the three intracellular or three extracellular loops, or parts thereof, from about amino acid 50 to about amino acid 59, from about amino acid 128 to about amino acid 141, from about amino acid 218 to about amino acid 230, from about amino acid 85 to about amino acid 102, from about amino acid 162 to about amino acid 193, and from about amino acid 248 to about amino acid 275 of SEQ ID NO:6. Fragments further include combinations of the above fragments, such as an amino terminal domain combined with one or more transmembrane segments and the attendant extra or intracellular loops or one or more transmembrane segments, and the attendant intra or extracellular loops, plus the carboxy terminal domain. Thus, any of the above fragments can be combined. Other fragments include the mature protein from about amino acid 6 to 337 of SEQ ID NO:6. Other fragments contain the various functional sites described herein, such as phosphorylation sites, glycosylation sites, and a sequence containing the GPCR signature sequence. Fragments, for example, can extend in one or both directions from the functional site to encompass 5, 10, 15, 20, 30, 40, 50, or up to 100 amino acids. Further, fragments can include sub-fragments of the specific domains mentioned above, which sub-fragments retain the function of the domain from which they are derived. Fragments also include amino acid sequences greater than 8 amino acids from amino acid 1 to about amino acid 244 of SEQ ID NO:6. Fragments also include antigenic fragments and specifically those shown to have a high antigenic index.

Accordingly, possible 14618 fragments include fragments defining a ligand-binding site, fragments defining a glycosylation site, fragments defining membrane association, fragments defining phosphorylation sites, fragments defining interaction with G proteins and signal transduction, and fragments defining myristoylation sites. By this is intended a discrete fragment that provides the relevant function or allows the relevant function to be identified. In a preferred embodiment, the fragment contains the ligand-binding site.

Biologically active 15334 fragments (peptides which are, for example, 8, 12, 15, 20, 30, 35, 36, 37, 38, 39, 40, 50, 100 or more amino acids in length) can comprise a domain or motif, e.g., an extracellular or intracellular domain or loop, one or more transmembrane segments, or parts thereof, G-protein binding site, or GPCR signature, glycosylation sites, cAMP, cGMP, protein kinase C, and casein kinase II phosphorylation sites, and N-myristoylation sites.

Possible 15334 fragments include, but are not limited to: 1) soluble peptides comprising the entire amino terminal extracellular domain about amino acid 1 to about amino acid 23 of SEQ ID NO:8, or parts thereof; 2) peptides comprising the entire carboxy terminal intracellular domain from about amino acid 300 to amino acid 372 of SEQ ID NO:8, or parts thereof; 3) peptides comprising the region spanning the entire transmembrane domain from about amino acid 26 to about amino acid 299 of SEQ ID NO:8, or parts thereof; 4) any of the specific transmembrane segments, or parts thereof, from about amino acid 26 to about amino acid 48, from about amino acid 56 to about amino acid 77, from about amino acid 99 to about amino acid 115, from about amino acid 140 to about amino acid 157, from about amino acid 188 to about amino acid 209, from about amino acid 235 to about amino acid 259, and from about amino acid 277 to about amino acid 299 of SEQ ID NO:8; 5) any of the three intracellular or three extracellular loops, or parts thereof, from about amino acid 49 to about amino acid 55, from about amino acid 78 to about amino acid 98, from about amino acid 116 to about amino acid 139, from about amino acid 158 to about amino acid 187, from about amino acid 210 to about amino acid 234, and from about amino acid 260 to about amino acid 276 of SEQ ID NO:8. Fragments further include combinations of the above fragments, such as an amino terminal domain combined with one or more transmembrane segments and the attendant extra or intracellular loops or one or more transmembrane segments, and the attendant intra or extracellular loops, plus the carboxy terminal domain. Thus, any of the above fragments can be combined. Other fragments include the mature protein from about amino acid 6 to 372 of SEQ ID NO:8. Other fragments contain the various functional sites described herein, such as phosphorylation sites, glycosylation sites, and myristoylation sites and a sequence containing the GPCR signature sequence. Fragments, for example, can extend in one or both directions from the functional site to encompass 5, 10, 15, 20, 30, 40, 50, or up to 100 amino acids. Further, fragments can include sub-fragments of the specific domains mentioned above, which sub-fragments retain the function of the domain from which they are derived. Fragments also include amino acid sequences greater than 7 amino acids. Fragments also include antigenic fragments and specifically those shown to have a high antigenic index.

Accordingly, possible 15334 fragments include fragments defining a ligand-binding site, fragments defining a glycosylation site, fragments defining membrane association, fragments defining phosphorylation sites, fragments defining interaction with G proteins and signal transduction, and fragments defining myristoylation sites. By this is intended a discrete fragment that provides the relevant function or allows the relevant function to be identified. In a preferred embodiment, the fragment contains the ligand-binding site.

Biologically active 14274 fragments (peptides which are, for example, 12, 15, 20, 30, 35, 36, 37, 38, 39, 40, 50, 100 or more amino acids in length) can comprise a domain or motif, e.g., an extracellular or intracellular domain or loop, one or more transmembrane segments, G-protein binding site, or GPCR signature, glycosylation sites, protein kinase C phosphorylation sites, casein kinase II phosphorylation sites, and N-myristoylation sites.

Possible 14274 fragments include, but are not limited to: 1) soluble peptides comprising the amino terminal extracellular domain from about amino acid 1 to about amino acid 39 of SEQ ID NO:11; 2) peptides comprising the carboxy terminal intracellular domain from about amino acid 309 to about amino acid 398 of SEQ ID NO:11; 3) peptides comprising the region spanning the entire transmembrane domain from about amino acid 40 to amino acid 308 SEQ ID NO:11, or one or more of the seven transmembrane segments or the six extracellular or intracellular loops as described herein.

14274 fragments further include combinations of the above fragments, such as an amino terminal domain combined with one or more transmembrane segments and the attendant extra or intracellular loops or one or more transmembrane segments, and the attendant intra or extracellular loops, plus the carboxy terminal domain. Thus, any of the above fragments can be combined. Other fragments contain the various functional sites described herein. Fragments, for example, can extend in one or both directions from the functional site to encompass 5, 10, 15, 20, 30, 40, 50, or up to 100 amino acids. Further, fragments can include sub-fragments of the specific domains mentioned above, which sub-fragments retain the function of the domain from which they are derived. 14274 fragments also include antigenic fragments and specifically those shown to have a high antigenic index.

Further possible 14274 fragments include but are not limited to fragments defining a ligand-binding site, fragments defining membrane association, fragments defining interaction with G proteins and signal transduction. By this is intended a discrete fragment that provides the relevant function or allows the relevant function to be identified. In a preferred embodiment, the fragment contains a ligand-binding site.

Biologically active 32164 fragments (peptides which are, for example, 6, 10, 12, 15, 20, 30, 35, 36, 37, 38, 39, 40, 50, 100 or more amino acids in length) can comprise a domain or motif, e.g., an extracellular or intracellular domain or loop, one or more transmembrane segments, or parts thereof, G-protein binding site, or glycosylation sites, phosphorylation sites, and myristoylation sites. Such domains or motifs can be identified by means of routine computerized homology searching procedures.

Possible 32164 fragments include, but are not limited to: 1) soluble peptides comprising the entire amino terminal extracellular domain from amino acid 1 to about amino acid 25 of SEQ ID NO:14 or parts thereof; 2) peptides comprising the entire carboxy terminal intracellular domain from about amino acid 293 to amino acid 314 of SEQ ID NO:14 or parts thereof; 3) peptides comprising the region spanning the entire transmembrane domain from about amino acid 26 to amino acid 292 of SEQ ID NO:14; 4) any of the specific transmembrane segments, or parts thereof; 5) any of the three intracellular or three extracellular loops, or parts thereof.

32164 fragments further include combinations of the above fragments, such as an amino terminal domain combined with one or more transmembrane segments and the attendant extra or intracellular loops or one or more transmembrane segments, and the attendant intra or extracellular loops, plus the carboxy terminal domain. Thus, any of the above fragments can be combined. Other fragments include the mature protein from about amino acid 42 to 314 of SEQ ID NO:14. Other fragments contain the various functional sites described herein. Fragments, for example, can extend in one or both directions from the functional site to encompass 5, 10, 15, 20, 30, 40, 50, or up to 100 amino acids. Further, fragments can include sub-fragments of the specific domains mentioned above, which sub-fragments retain the function of the domain from which they are derived. 32164 fragments also include antigenic fragments and specifically those shown to have a high antigenic index.

Further possible 32164 fragments include but are not limited to fragments defining a ligand-binding site, fragments defining membrane association, fragments defining interaction with G proteins and signal transduction. By this is intended a discrete fragment that provides the relevant function or allows the relevant function to be identified. In a preferred embodiment, the fragment contains a ligand-binding site.

Biologically active fragments of the 39404 protein (peptides which are, for example, 5-10, 10-15, 15-20, 20-30, 30-40, 40-50, 50-100, or more amino acids in length) can comprise a domain or motif, e.g., an extracellular or intracellular domain or loop, one or more transmembrane segments or parts thereof, G-protein binding site, GPCR signature, glycosylation site, or phosphorylation site. In one embodiment, fragments are greater than eleven amino acids. Such domains or motifs can be identified by means of routine computerized homology searching procedures.

Possible 39404 fragments include, but are not limited to: 1) soluble peptides comprising the entire amino terminal extracellular domain or parts thereof; 2) peptides comprising the entire carboxy terminal intracellular domain or parts thereof; 3) peptides comprising the region spanning the entire transmembrane domain or parts thereof; 4) any of the specific transmembrane segments, or parts thereof; 5) any of the three intracellular or three extracellular loops, or parts thereof.

39404 fragments further include combinations of the above fragments, such as an amino terminal domain combined with one or more transmembrane segments and the attendant extra or intracellular loops or one or more transmembrane segments, and the attendant intra or extracellular loops, plus the carboxy terminal domain. Thus, any of the above fragments can be combined. Other fragments include the mature protein from about amino acid 6 to the last amino acid. Other fragments contain the various functional sites described herein, such as phosphorylation sites or glycosylation sites, and a sequence containing the GPCR signature sequence. Fragments, for example, can extend in one or both directions from the functional site to encompass 5, 10, 15, 20, 30, 40, 50, or up to 100 amino acids.

Further, 39404 fragments can include sub-fragments of the specific domains mentioned above, which sub-fragments retain the function of the domain from which they are derived. Fragments also include but are not limited to amino acid sequences greater than 5 amino acids, except for SILTLT (SEQ ID NO:24), SILFLTC (SEQ ID NO:25), or NLYSSILFLTC (SEQ ID NO:26) (however, it is understood that with regard to uses and methods of the invention, even these fragments and any other fragments that may be known prior to the invention are encompassed). In no way however are such fragments to be construed as encompassing fragments that may be found in the art, except as above indicated. 39404 fragments also include antigenic fragments and specifically in regions shown to have a high antigenic index.

Accordingly, possible 39404 fragments include fragments defining a ligand-binding site, fragments defining a glycosylation site, fragments defining membrane association, fragments defining phosphorylation sites, and fragments defining interaction with G proteins and signal transduction. By this is intended a discrete fragment that provides the relevant function or allows the relevant function to be identified. In a preferred embodiment, the fragment contains the ligand-binding site.

Biologically active fragments of 38911 protein (peptides which are, for example, 5-10, 10-15, 15-20, 20-30, 30-40, 40-50, 50-100, or more amino acids in length) can comprise a domain or motif, e.g., an extracellular or intracellular domain or loop, one or more transmembrane segments, or parts thereof, G-protein binding site, glycosylation sites, and cAMP- and cGMP-dependent, protein kinase C, and casein kinase II phosphorylation sites, and N-myristoylation sites. Such domains or motifs can be identified by means of routine computerized homology searching procedures.

Possible 38911 fragments include, but are not limited to: 1) soluble peptides comprising the entire amino terminal extracellular domain about amino acid 1 to about amino acid 40 of SEQ ID NO:18, or parts thereof; 2) peptides comprising the entire carboxy terminal intracellular domain from about amino acid 259 to amino acid 337 of SEQ ID NO:18, or parts thereof; 3) peptides comprising the region spanning the entire transmembrane domain from about amino acid 41 to about amino acid 294 of SEQ ID NO:18, or parts thereof; 4) any of the specific transmembrane segments, or parts thereof; 5) any of the three intracellular or three extracellular loops, or parts thereof.

38911 fragments further include combinations of the above fragments, such as an amino terminal domain combined with one or more transmembrane segments and the attendant extra or intracellular loops or one or more transmembrane segments, and the attendant intra or extracellular loops, plus the carboxy terminal domain. Thus, any of the above fragments can be combined. Other fragments include the mature protein from about amino acid 6 to 337 of SEQ ID NO:18. Other fragments contain the various functional sites described herein, such as phosphorylation sites, glycosylation sites, or myristoylation sites. Fragments, for example, can extend in one or both directions from the functional site to encompass 5, 10, 15, 20, 30, 40, 50, or up to 100 amino acids. Further, fragments can include sub-fragments of the specific domains mentioned above, which sub-fragments retain the function of the domain from which they are derived. These regions can be identified by well-known methods involving computerized homology analysis.

38911 fragments also include amino acid sequences greater than 5 amino acids except for LAVADLL (SEQ ID NO:27), LALLLT (SEQ ID NO:28), LRRSLP (SEQ ID NO:29), FLVGDPGNA (SEQ ID NO:30), GNAMV (SEQ ID NO:31), LAVAD (SEQ ID NO:32), FLVGVPGNA (SEQ ID NO:33), ALLLT (SEQ ID NO:34), ADLLCCLSLP (SEQ ID NO:35) (it is understood however that these fragments and any others that may have been disclosed prior to the invention may be encompassed in specific uses and methods disclosed herein relating to tissues/disorders with which the expression is associated). In no way however are such fragments to be construed as encompassing fragments that may be found in the art except as just indicated. 38911 fragments also include antigenic fragments and specifically from regions shown to have a high antigenic index.

Accordingly, possible 38911 fragments include fragments defining a ligand-binding site, fragments defining a glycosylation site, fragments defining membrane association, fragments defining a phosphorylation site, fragments defining interaction with G proteins and signal transduction, and fragments defining a myristoylation site. By this is intended a discrete fragment that provides the relevant function or allows the relevant function to be identified. In a preferred embodiment, the fragment contains the ligand-binding site.

Biologically active fragments of the 26904 protein (peptides which are, for example, 5-10, 10-15, 15-20, 20-30, 30-40, 40-50, 50-100, or more amino acids in length) can comprise a domain or motif, e.g., an extracellular or intracellular domain or loop, one or more transmembrane segments, or parts thereof, G-protein binding site, glycosylation site, cAMP, cGMP, protein kinase C, and casein kinase II phosphorylation site, N-myristoylation site, amidation, or ATP/GTP binding site. Such domains or motifs can be identified by means of routine computerized homology searching procedures.

Possible 26904 fragments include, but are not limited to: 1) soluble peptides comprising the entire amino terminal extracellular domain, or parts thereof; 2) peptides comprising the entire carboxy terminal intracellular domain, or parts thereof; 3) peptides comprising the region spanning the entire transmembrane domain, or parts thereof; 4) any of the specific transmembrane segments, or parts thereof; 5) any of the three intracellular or three extracellular loops, or parts thereof.

26904 fragments further include combinations of the above fragments, such as an amino terminal domain combined with one or more transmembrane segments and the attendant extra or intracellular loops or one or more transmembrane segments, and the attendant intra or extracellular loops, plus the carboxy terminal domain. Thus, any of the above fragments can be combined. Other fragments include the mature protein from about amino acid 6 to 450 of SEQ ID NO:20. Other fragments contain the various functional sites described herein, such as phosphorylation sites, glycosylation site, or myristoylation sites. Fragments, for example, can extend in one or both directions from the functional site to encompass 5, 10, 15, 20, 30, 40, 50, or up to 100 amino acids. Further, fragments can include sub-fragments of the specific domains mentioned above, which sub-fragments retain the function of the domain from which they are derived.

26904 fragments also include amino acid sequences greater than four amino acids except for YVGAAHG (SEQ ID NO:36), LVHWCHGAPGVI (SEQ ID NO:37), QAYKVF (SEQ ID NO:38), EEKYL (SEQ ID NO:39), SLFEGMAG (SEQ ID NO:40), RFPAFEL (SEQ ID NO:41), LLQQME (SEQ ID NO:42), TFLCGDAGPLAV (SEQ ID NO:43), AGIYY (SEQ ID NO:44), SGNYP (SEQ ID NO:45), QAYKVFKEE (SEQ ID NO:46), DVIWQ (SEQ ID NO:47), KYLYRACKFAEWCLDYG (SEQ ID NO:48), ELLYGR (SEQ ID NO:49), PYSLFEG (SEQ ID NO:50), and VTFLCG (SEQ ID NO:51) (it is understood however that these fragments and any others that may have been disclosed prior to the invention are in fact encompassed by the invention in methods and uses disclosed herein relevant to specific tissues or disorders with which the gene is associated). In no way however are such fragments to be construed as encompassing fragments that may be found in the art, except as just indicated. Fragments also include antigenic fragments and specifically from sites shown to have a high antigenic index.

Accordingly, possible 26904 fragments include but are not limited to fragments defining a ligand-binding site, fragments defining a glycosylation site, fragments defining membrane association, fragments defining phosphorylation sites, fragments defining interaction with G proteins and signal transduction, and fragments defining myristoylation sites. By this is intended a discrete fragment that provides the relevant function or allows the relevant function to be identified. In a preferred embodiment, the fragment contains the ligand-binding site.

Biologically active fragments of 31237 protein (peptides which are, for example, 5-10, 10-15, 15-20, 20-30,-30-40, 40-50, 50-100, or more amino acids in length) can comprise a domain or motif, e.g., an extracellular or intracellular domain or loop, one, or more transmembrane segments, or parts thereof, G-protein binding site, glycosylation sites, protein kinase C, tyrosine kinase, cAMP and cGMP-dependent kinase, and casein kinase II phosphorylation sites, N-myristoylation and glycosaminoglycan attachment sites. In one embodiment, fragments are greater than eleven amino acids. Such domains or motifs can be identified by means of routine computerized homology searching procedures.

Possible 31237 fragments include, but are not limited to: 1) soluble peptides comprising the entire amino terminal extracellular domain or parts thereof; 2) peptides comprising the entire carboxy terminal intracellular domain or parts thereof; 3),peptides comprising the region spanning the entire transmembrane domain or parts thereof; 4) any of the specific transmembrane segments, or parts thereof; 5) any of the three intracellular or three extracellular loops, or parts thereof.

31237 fragments further include combinations of the above fragments, such as an amino terminal domain combined with one or more transmembrane segments and the attendant extra or intracellular loops or one or more transmembrane segments, and the attendant intra or extracellular loops, plus the carboxy terminal domain. Thus, any of the above fragments can be combined. Other fragments include the mature protein from about amino acid 6 to the last amino acid. Other fragments contain the various functional sites described herein, such as phosphorylation sites or glycosylation sites. Fragments, for example, can extend in one or both directions from the functional site to encompass 5, 10, 15, 20, 30, 40, 50, or up to 100 amino acids. Further, fragments can include sub-fragments of the specific domains mentioned above, which sub-fragments retain the function of the domain from which they are derived. Fragments also include amino acid sequences greater than 5 amino acids, except for DPTLAI (SEQ ID NO:75), AWGIVLE (SEQ ID NO:76), FLLGTLGLF (SEQ ID NO:77), ICFSCL (SEQ ID NO:78), VYQPTEMA (SEQ ID NO:79), EAVAGAG (SEQ ID NO:80), MDFVMALIY (SEQ ID NO:81), ENKAFSMDE (SEQ ID NO:82), and a fragment beginning with amino acid 307 and ending at amino acid 365 of SEQ ID NO:22 (MYT . . . PTR) (SEQ ID NO:83). In no way however are such fragments to be construed as encompassing fragments that may be found in the art (these fragments and others may however be encompassed in specific methods and uses relating to tissues/disorders in which the gene expression is relevant). 31237 fragments also include antigenic fragments and specifically at those sites shown to have a high antigenic index.

Accordingly, possible 31237 fragments include fragments defining a ligand-binding site, fragments defining a glycosaminoglycan attachment site, fragments defining N-myristoylation sites, fragments defining immunoglobulin and major histocompatibility complex protein signature, fragments defining a glycosylation site, fragments defining membrane association, fragments defining phosphorylation sites, and fragments defining interaction with G proteins and signal transduction. By this is intended a discrete fragment that provides the relevant function or allows the relevant function to be identified. In a preferred embodiment, the fragment contains the ligand-binding site.

Biologically active 18057 fragments (peptides which are, for example, around 5-10, 10-15, 15-20, 30, 35, 36, 37, 38, 39, 40, 50, 100, 200, 300, 400, or 469 amino acids in length) can comprise a domain or motif, e.g., an extracellular or intracellular domain or loop, one or more transmembrane segments, or parts thereof, G-protein binding site, or glycosylation sites, phosphorylation sites, and myristoylation sites. Such domains or motifs can be identified by means of routine computerized homology searching procedures. As used herein, a 18057 fragment comprises at least 6 contiguous amino acids, especially from around nucleotide 700 to around nucleotide 1624 of SEQ ID NO:53 and greater than 15 contiguous amino acids from around nucleotide 218 to around nucleotide 700 of SEQ ID NO:53. Fragments can retain one or more of the biological activities of the protein, for example the ability to bind to a G-protein or ligand, as well as fragments that can be used as an immunogen to generate antibodies.

Possible 18057 fragments include, but are not limited to: 1) soluble peptides comprising the entire amino terminal extracellular domain or parts thereof; 2) peptides comprising the entire carboxy terminal intracellular domain or parts thereof; 3) peptides comprising the region spanning the entire transmembrane domain or parts thereof; 4) any of the specific transmembrane segments, or parts thereof; 5) any of the three intracellular or three extracellular loops, or parts thereof.

18057 fragments further include combinations of the above fragments, such as an amino terminal domain combined with one or more transmembrane segments and the attendant extra or intracellular loops or one or more transmembrane segments, and the attendant intra or extracellular loops, plus the carboxy terminal domain. Thus, any of the above fragments can be combined. Other fragments include the mature protein from about amino acid 14 to 469 of SEQ ID NO:52. Other fragments contain the various functional sites described herein. Fragments, for example, can extend in one or both directions from the functional site to encompass 5, 10, 15, 20, 30, 40, 50, or up to 100 amino acids. Further, fragments can include sub-fragments of the specific domains mentioned above, which sub-fragments retain the function of the domain from which they are derived.

Further possible 18057 fragments include but are not limited to fragments defining a ligand-binding site, fragments defining membrane association, fragments defining interaction with G proteins and signal transduction. By this is intended a discrete fragment that provides the relevant function or allows the relevant function to be identified. In a preferred embodiment, the fragment contains a ligand-binding site.

Biologically active 16405 fragments (peptides which are, for example, 7, 10, 12, 15, 20, 30, 35, 36, 37, 38, 39, 40, 50, 100 or more amino acids in length) can comprise a domain or motif, e.g., an extracellular or intracellular domain or loop, one or more transmembrane segments, or parts thereof, G-protein binding site, or GPCR signature, glycosylation sites, phosphorylation sites, amidation sites, and N-myristorylation sites. Such domains or motifs can be identified by means of routine computerized homology searching procedures. As used herein, a 16405 fragment comprises at least 7 contiguous amino acids from amino acid 1 to about amino acid 356 of SEQ ID NO:56. Fragments retain one or more of the biological activities of the protein, for example the ability to bind to a G-protein or ligand, as well as fragments that can be used as an immunogen to generate receptor antibodies.

Possible 16405 fragments include, but are not limited to: 1) soluble peptides comprising the entire amino terminal extracellular domain, or parts thereof; 2) peptides comprising the entire carboxy terminal intracellular domain, or parts thereof; 3) peptides comprising the region spanning the entire transmembrane domain; 4) any of the specific transmembrane segments, or parts thereof; 5) any of the three intracellular or three extracellular loops, or parts thereof.

16405 fragments further include combinations of the above fragments, such as an amino terminal domain combined with one or more transmembrane segments and the attendant extra or intracellular loops or one or more transmembrane segments, and the attendant intra or extracellular loops, plus the carboxy terminal domain. Thus, any of the above fragments can be combined. Other fragments include the mature protein. Other fragments contain the various functional sites described herein. Fragments, for example, can extend in one or both directions from the functional site to encompass 5, 10, 15, 20, 30, 40, 50, or up to 100 amino acids. Further, fragments can include sub-fragments of the specific domains mentioned above, which sub-fragments retain the function of the domain from which they are derived. These regions can be identified by well-known methods involving computerized homology analysis. 16405 fragments also include antigenic fragments and specifically those shown to have a high antigenic index.

Further, possible 16405 fragments include but are not limited to fragments defining a ligand-binding site, fragments defining membrane association, and fragments defining interaction with G proteins and signal transduction. By this is intended a discrete fragment that provides the relevant function or allows the relevant function to be identified. In a preferred embodiment, the fragment contains the ligand-binding site.

Biologically active 32705, 23224, 27423, 32700 and 32712 fragments (peptides which are about, for example, 5-10, 10-15, 15-20, 25-30, 35-40, 50, 100 or more amino acids in length) can comprise a domain or motif, e.g., a GTP or GDP binding site, a regulatory site for interaction with any of the regulatory proteins affecting GTPase activity, membrane anchoring site, site interacting with protein kinase regulatory regions, or glycosylation sites, phosphorylation sites, and myristoylation sites. Such domains or motifs can be identified by means of routine computerized homology searching procedures. Domains/motifs include, but are not limited to, those identified herein. As used herein, a 32705, 23224, 27423, 32700 or 32712 fragment comprises at least 5 contiguous amino acids. 32705 fragments can retain one or more of the biological activities of the protein, for example the ability to bind, to GTP or GDP, as well as fragments that can be used as an immunogen to generate antibodies.

32705, 23224, 27423, 32700 or 32712 fragments also include combinations of domains or motifs including, but not limited to, those mentioned above. 32705, 23224, 27423, 32700 or 32712 fragments, for example, can extend in one or both directions from the functional site to encompass 5, 10, 15, 20, 30, 40, 50, or up to 100 amino acids. Further, 32705, 23224, 27423, 32700 or 32712 fragments can include sub-fragments of the specific domains mentioned above, which sub-fragments retain the function of the domain from which they are derived. 32705, 23224, 27423, 32700 or 32712 fragments also include antigenic fragments and specifically those shown to have a high antigenic index.

Further possible 32705, 23224, 27423, 32700 or 32712 fragments include but are not limited to fragments defining a GTP or GDP binding site, regulatory protein binding site, or binding site for interacting with the regulatory region of a p21-activated protein kinase such as MAPK or JNK, fragments defining membrane association, fragments defining interaction with G protein-coupled receptors and signal transduction. By this is intended a discrete fragment that provides the relevant function or allows the relevant function to be identified. In a preferred embodiment, the fragment contains a GTP-binding site.

Biologically active 12216 fragments (peptides which are, for example, 6, 10, 12, 15, 20, 30, 35, 36, 37, 38, 39, 40, 50, 100 or more amino acids in length) can comprise a domain or motif, e.g., an extracellular or intracellular domain or loop, one or more transmembrane segments, or parts thereof, G-protein binding site, or GPCR signature, glycosylation sites, cAMP and cGMP-dependent, protein kinase C, and casein kinase II phosphorylation sites, N-myristoylation, and prenylation sites. Such domains or motifs can be identified by computerized homology searching procedures.

As used herein, a 12216 fragment comprises at least 6 contiguous amino acids, such as from amino acids 1-35, 36-65, 65-109, 108-128, 128-234, 240-291, and 295-373 of SEQ ID NO:71. The invention encompasses other fragments, however, such as any fragment in the protein greater than 16 amino acids. The fragments to which the invention pertains, however, are not to be construed as encompassing fragments that may be disclosed prior to the present invention and include all unique non-disclosed fragments. Fragments retain one or more of the biological activities of the protein, for example the ability to bind to a G-protein or ligand, as well as fragments that can be used as an immunogen to generate receptor antibodies.

Possible 12216 fragments include, but are not limited to: 1) soluble peptides comprising the entire amino terminal extracellular domain about amino acid 1 to about amino acid 25 of SEQ ID NO:71 or parts thereof; 2) peptides comprising the entire carboxy terminal intracellular domain from about amino acid 344 to amino acid 373 of SEQ ID NO:71 or parts thereof; 3) peptides comprising the region spanning the entire transmembrane domain from about amino acid 26 to amino acid 343 of SEQ ID NO:71; 4) any of the specific transmembrane segments, or parts thereof; 5) any of the three intracellular or three extracellular loops, or parts thereof.

Possible 12216 fragments include, but are not limited to: 1) soluble peptides comprising the entire amino terminal extracellular domain about amino acid 1 to about amino acid 25 of SEQ ID NO:71, or parts thereof; 2) peptides comprising the entire carboxy terminal intracellular domain from about amino acid 344 to amino acid 373 of SEQ ID NO:71, or parts thereof; 3) peptides comprising the region spanning the entire transmembrane domain from about amino acid 26 to about amino acid 343 of SEQ ID NO:71, or parts thereof; 4) any of the specific transmembrane segments, or parts thereof, from about amino acid 26 to about amino acid 48, from about amino acid 59 to about amino acid 83, from about amino acid 98 to about amino acid 119, from about amino acid 137 to about amino acid 156, from about amino acid 187 to about amino acid 204, from about amino acid 287 to about amino acid 308, and from about amino acid 321 to about amino acid 343 of SEQ ID NO:71; 5) any of the three intracellular or three extracellular loops, or parts thereof, from about amino acid 49 to about amino acid 58, from about amino acid 120 to about amino acid 136, from about amino acid 205 to about amino acid 286, from about amino acid 84 to about amino acid 97, from about amino acid 157 to about amino acid 186, and from about amino acid 309 to about amino acid 320 of SEQ ID NO:71. Fragments further include combinations of the above fragments, such as an amino terminal domain combined with one or more transmembrane segments and the attendant extra or intracellular loops or one or more transmembrane segments, and the attendant intra or extracellular loops, plus the carboxy terminal domain. Thus, any of the above fragments can be combined. Other fragments include the mature protein from about amino acid 6 to 373 of SEQ ID NO:71. Other fragments contain the various functional sites described herein, such as N-glycosylation, cAMP and cGMP-dependent, protein kinase C, and casein kinase II phosphorylation sites, N-myristoylation sites, prenylation sites, and a sequence containing the GPCR signature sequence. Fragments, for example, can extend in one or both directions from the functional site to encompass 5, 10, 15, 20, 30, 40, 50, or up to 100 amino acids. Further, fragments can include sub-fragments of the specific domains mentioned above, which sub-fragments retain the function of the domain from which they are derived. These regions can be identified by well-known methods involving computerized analysis. Fragments also include antigenic fragments and specifically those shown to have a high antigenic index.

Accordingly, possible 12216 fragments include fragments defining a ligand-binding site, fragments defining a glycosylation site, fragments defining membrane association, fragments defining N-myristoylation and prenylation sites, fragments defining interaction with G proteins and signal transduction, and fragments defining cAMP and cGMP-dependent, casein kinase II, and protein kinase C phosphorylation sites. By this is intended a discrete fragment that provides the relevant function or allows the relevant function to be identified. In a preferred embodiment, the fragment contains the ligand-binding site.

The invention also provides 14400 fragments with immunogenic properties. These contain an epitope-bearing portion of the 14400 receptor protein and variants. The invention also provides 2838 receptor fragments with immunogenic properties. These contain an epitope-bearing portion of the 2838 receptor protein and variants. The invention also provides 14618 receptor fragments with immunogenic properties. These contain an epitope-bearing portion of the 14618 receptor protein and variants. The invention also provides 15334 receptor fragments with immunogenic properties. These contain an epitope-bearing portion of the 15334 receptor protein and variants. The invention also provides 14274 fragments with immunogenic properties. These contain an epitope-bearing portion of the 14274 receptor protein and variants. The invention also provides 32164 fragments with immunogenic properties. These contain an epitope-bearing portion of the 32164 protein and variants. The invention also provides 39404 protein fragments with immunogenic properties. These contain an epitope-bearing portion of the 39404 protein and variants. The invention also provides 38911 protein fragments with immunogenic properties. These contain an epitope-bearing portion of the 38911 protein and variants. The invention also provides 26904 protein fragments with immunogenic properties. These contain an epitope-bearing portion of the 26904 protein and variants. The invention also provides 31237 protein fragments with immunogenic properties. These contain an epitope-bearing portion of the 31237 protein and variants. The invention also 18057 provides fragments with immunogenic properties. These contain an epitope-bearing portion of the 18057 protein and variants. The invention also provides 16405 fragments with immunogenic properties. These contain an epitope-bearing portion of the 16405 receptor protein and variants. The invention also provides 32705 fragments with immunogenic properties. These contain an epitope-bearing portion of the 32705 protein of the invention and variants. The invention also provides 23224 fragments with immunogenic properties. These contain an epitope-bearing portion of the 23224 protein of the invention and variants. The invention also provides 27423 fragments with immunogenic properties. These contain an epitope-bearing portion of the 27423 protein of the invention and variants. The invention also provides 32700 fragments with immunogenic properties. These contain an epitope-bearing portion of the 32700 protein of the invention and variants. The invention also provides 32712 fragments with immunogenic properties. These contain an epitope-bearing portion of the 32712 protein of the invention and variants The invention also provides 12216 fragments with immunogenic properties. These contain an epitope-bearing portion of the 12216 receptor protein and variants. These peptides can contain at least 5-10, 11, 12, 13, at least 14, or between at least about 15 to about 30 amino acids.

Non-limiting examples of antigenic polypeptides that can be used to generate antibodies include peptides derived from the amino terminal extracellular domain or any of the extracellular loops.

The epitope-bearing receptor and polypeptides may be produced by any conventional means (Houghten, R. A., Proc. Natl. Acad. Sci. USA 82:5131-5135 (1985)). Simultaneous multiple peptide synthesis is described in U.S. Pat. No. 4,631,211.

Fragments can be discrete (not fused to other amino acids or polypeptides) or can be within a larger polypeptide. Further, several fragments can be comprised within a single larger polypeptide. In one embodiment a fragment designed for expression in a host can have heterologous pre- and pro-polypeptide regions fused to the amino terminus of the receptor fragment and an additional region fused to the carboxyl terminus of the fragment.

The invention thus provides chimeric or fusion proteins. These comprise a receptor protein operatively linked to a heterologous protein having an amino acid sequence not substantially homologous to the receptor protein. “Operatively linked” indicates that the receptor protein and the heterologous protein are fused in-frame. The heterologous protein can be fused to the N-terminus or C-terminus of the receptor protein.

In one embodiment the fusion protein does not affect receptor function per se. For example, the fusion protein can be a GST-fusion protein in which the receptor sequences are fused to the C-terminus of the GST sequences. Other types of fusion proteins include, but are not limited to, enzymatic fusion proteins, for example beta-galactosidase fusions, yeast two-hybrid GAL fusions, poly-His fusions and Ig fusions. Such fusion proteins, particularly poly-His fusions, can facilitate the purification of recombinant receptor protein. In certain host cells (e.g., mammalian host cells), expression and/or secretion of a protein can be increased by using a heterologous signal sequence. Therefore, in another embodiment, the fusion protein contains a heterologous signal sequence at its N-terminus.

EP-A-O 464 533 discloses fusion proteins comprising various portions of immunoglobulin constant regions. The Fc is useful in therapy and diagnosis and thus results, for example, in improved pharmacokinetic properties (EP-A 0232 262). In drug discovery, for example, human proteins have been fused with Fc portions for the purpose of high-throughput screening assays to identify antagonists. Bennett et al. (J. Mol. Recog. 8:52-58 (1995)) and Johanson et al. (J. Biol. Chem. 270, 16:9459-9471 (1995)). Thus, this invention also encompasses soluble fusion proteins containing a receptor polypeptide and various portions of the constant regions of heavy or light chains of immunoglobulins of various subclass (IgG, IgM, IgA, IgE). Preferred as immunoglobulin is the constant part of the heavy chain of human IgG, particularly IgG1, where fusion takes place at the hinge region. For some uses it is desirable to remove the Fc after the fusion protein has been used for its intended purpose, for example when the fusion protein is to be used as antigen for immunizations. In a particular embodiment, the Fc part can be removed in a simple way by a cleavage sequence which is also incorporated and can be cleaved with factor Xa.

A chimeric or fusion protein can be produced by standard recombinant DNA techniques. For example, DNA fragments coding for the different protein sequences are ligated together in-frame in accordance with conventional techniques. In another embodiment, the fusion gene can be synthesized by conventional techniques including automated DNA synthesizers. Alternatively, PCR amplification of gene fragments can be carried out using anchor primers which give rise to complementary overhangs between two consecutive gene fragments which can subsequently be annealed and re-amplified to generate a chimeric gene sequence (see Ausubel et al., Current Protocols in Molecular Biology, 1992). Moreover, many expression vectors are commercially available that already encode a fusion moiety (e.g., a GST protein). A receptor protein-encoding nucleic acid can be cloned into such an expression vector such that the fusion moiety is linked in-frame to the receptor protein.

Another form of fusion protein is one that directly affects receptor functions.

Accordingly, a receptor polypeptide is encompassed by the present invention in which one or more of the receptor domains (or parts thereof) has been replaced by homologous domains (or parts thereof) from another G-protein coupled receptor or other type of receptor. Accordingly, various permutations are possible. The amino terminal extracellular domain, or subregion thereof, (for example, ligand-binding) can be replaced with the domain or subregion from another ligand-binding receptor protein. Alternatively, the entire transmembrane domain, or any of the seven segments or loops, or parts thereof, for example, G-protein-binding/signal transduction, can be replaced. Finally, the carboxy terminal intracellular domain or subregion can be replaced. Thus, chimeric receptors can be formed in which one or more of the native domains or subregions has been replaced.

The isolated 14400 receptor protein can be purified from cells that naturally express it, such as from spleen, thymus, prostate, testes, uterus, small intestine, colon, peripheral blood lymphocytes, heart, brain, placenta, lung, liver, skeletal muscle, kidney, and pancreas, purified from cells that have been altered to express it (recombinant), or synthesized using known protein synthesis methods.

The isolated 2838 receptor protein can be purified from cells that naturally express it, such as from lymph node, thymus, spleen, testes, colon, and peripheral blood lymphocytes, and from those cells in which it is significantly expressed such as activated T-helper cells (1 and 2), hypoxic Hep 3B cells, CD3 cells (both CD4 and CD8), activated B cells, Jurkat cells, among others, purified from cells that have been altered to express it (recombinant), or synthesized using known protein synthesis methods.

The isolated 14618 receptor protein can be purified from cells that naturally express it, such as from breast, skeletal muscle, lymph node, spleen and blood peripheral lymphocytes, as well as CD34⁺ cells and megakaryocytes, purified from cells that have been altered to express it (recombinant), or synthesized using known protein synthesis methods.

The isolated 15334 receptor protein can be purified from cells that naturally express it, such as from colon, placenta, pancreas, tonsil, lymph node, spleen, peripheral blood cells, thymus, adrenal gland and heart, as well as K562 cells, erythroblasts, and megakaryocytes, purified from cells that have been altered to express it (recombinant), or synthesized using known protein synthesis methods.

The isolated 14274 receptor protein can be purified from cells that naturally express it, such as from CD34⁻ bone marrow cells, peripheral blood cells, such as CD3 and CD8 T-cells, brain, spleen, lung, lung carcinoma, colon carcinoma, and placenta, purified from cells that have been altered to express it (recombinant), or synthesized using known protein synthesis methods.

The isolated 32164 protein can be purified from cells that naturally express it, including but not limited to, those described herein above, and particularly fetal liver and erythroblasts, purified from cells that have been altered to express it (recombinant), or synthesized using known protein synthesis methods.

The isolated 39404 protein can be purified from cells that naturally express it, such as from breast, brain, kidney, vein, fetal kidney and fetal liver, as well as aortic intimal proliferations and internal mammary artery, purified from cells that have been altered to express it (recombinant), or synthesized using known protein synthesis methods.

The isolated 38911 protein can be purified from cells that naturally express it, and especially osteoclasts, spleen, tonsils, liver, kidney, and testis, purified from cells that have been altered to express it (recombinant), or synthesized using known protein synthesis methods.

The isolated 26904 protein can be purified from cells that naturally express it, such as from brain, purified from cells that have been altered to express it (recombinant), or synthesized using known protein synthesis methods.

The isolated 31237 protein can be purified from cells that naturally express it, such as from colon, purified from cells that have been altered to express it (recombinant), or synthesized using known protein synthesis methods.

The isolated 18057 protein can be purified from cells that naturally express it, including but not limited to, those described herein above, and particularly fetal liver and erythroblasts, purified from cells that have been altered to express it (recombinant), or synthesized using known protein synthesis methods.

The isolated 16405 receptor protein can be purified from cells that naturally express it, such as from spleen, glioblastoma, and sclerotic lesions, purified from cells that have been altered to express it (recombinant), or synthesized using known protein synthesis methods.

The isolated 32705 receptor protein can be purified from cells that naturally express it, such as from brain or virus infected hepatocytes, purified from cells that have been altered to express it (recombinant), or synthesized using known protein synthesis methods.

The isolated 23224 receptor protein can be purified from cells that naturally express it, such as from kidney, pancreas, spinal cord, brain cortex, brain hypothalamus, and dorsal root ganglia, purified from cells that have been altered to express it (recombinant), or synthesized using known protein synthesis methods.

The isolated 32700 receptor protein can be purified from cells that naturally express it, such as from HUVEC (Human Umbilical Vein Endothelial Cells), hemangioma, skeletal muscle, brain cortex (normal), brain hypothalamus (normal), DRG (Dorsal Root Ganglion), ovary (tumor) and erythroid cells, purified from cells that have been altered to express it (recombinant), or synthesized using known protein synthesis methods.

The isolated 32712 receptor protein can be purified from cells that naturally express it, such as from kidney, primary osteoblasts, spinal cord (normal), brain cortex (normal), brain hypothalamus (normal), DRG (Dorsal Root Ganglion), prostate (normal), prostate (tumor), liver (normal), liver fibrosis, spleen (normal), tonsil (normal), lymph node (normal), BM-MNC (Bone Marrow Mononuclear Cells), neutrophils, megakaryocytes and erythroid cells, purified from cells that have been altered to express it (recombinant), or synthesized using known protein synthesis methods.

The isolated 12216 receptor protein can be purified from cells that naturally express it, such as from brain, skeletal muscle, colon, mobilized peripheral blood CD34⁺ cells, human embryonic kidney cell lines, aorta, kidney, and monkey coronary, femoral, and renal arterial tissue, purified from cells that have been altered to express it (recombinant), or synthesized using known protein synthesis methods.

In one embodiment, the protein is produced by recombinant DNA techniques. For example, a nucleic acid molecule encoding the receptor polypeptide is cloned into an expression vector, the expression vector introduced into a host cell and the protein expressed in the host cell. The protein can then be isolated from the cells by an appropriate purification scheme using standard protein purification techniques. Polypeptides often contain amino acids other than the 20 amino acids commonly referred to as the 20 naturally-occurring amino acids. Further, many amino acids, including the terminal amino acids, may be modified by natural processes, such as processing and other post-translational modifications, or by chemical modification techniques well known in the art. Common modifications that occur naturally in polypeptides are described in basic texts, detailed monographs, and the research literature, and they are well known to those of skill in the art.

Accordingly, the polypeptides also encompass derivatives or analogs in which a substituted amino acid residue is not one encoded by the genetic code, in which a substituent group is included, in which the mature polypeptide is fused with another compound, such as a compound to increase the half-life of the polypeptide (for example, polyethylene glycol), or in which the additional amino acids are fused to the mature polypeptide, such as a leader or secretory sequence or a sequence for purification of the mature polypeptide or a pro-protein sequence.

Known modifications include, but are not limited to, acetylation, acylation, ADP-ribosylation, amidation, covalent attachment of flavin, covalent attachment of a heme moiety, covalent attachment of a nucleotide or nucleotide derivative, covalent attachment of a lipid or lipid derivative, covalent attachment of phosphotidylinositol, cross-linking, cyclization, disulfide bond formation, demethylation, formation of covalent crosslinks, formation of cystine, formation of pyroglutamate, formylation, gamma carboxylation, glycosylation, GPI anchor formation, hydroxylation, iodination, methylation, myristoylation, oxidation, proteolytic processing, phosphorylation, prenylation, racemization, selenoylation, sulfation, transfer-RNA mediated addition of amino acids to proteins such as arginylation, and ubiquitination.

Such modifications are well-known to those of skill in the art and have been described in great detail in the scientific literature. Several particularly common modifications, glycosylation, lipid attachment, sulfation, gamma-carboxylation of glutamic acid residues, hydroxylation and ADP-ribosylation, for instance, are described in most basic texts, such as Proteins—Structure and Molecular Properties, 2nd Ed., T. E. Creighton, W. H. Freeman and Company, New York (1993). Many detailed reviews are available on this subject, such as by Wold, F., Posttranslational Covalent Modification of Proteins, B. C. Johnson, Ed., Academic Press, New York 1-12 (1983); Seifter et al. (Meth. Enzymol. 182: 626-646 (1990)) and Rattan et al. (Ann. N.Y. Acad. Sci. 663:48-62 (1992)).

As is also well known, polypeptides are not always entirely linear. For instance, polypeptides may be branched as a result of ubiquitination, and they may be circular, with or without branching, generally as a result of post-translation events, including natural processing event and events brought about by human manipulation which do not occur naturally. Circular, branched and branched circular polypeptides may be synthesized by non-translational natural processes and by synthetic methods.

Modifications can occur anywhere in a polypeptide, including the peptide backbone, the amino acid side-chains and the amino or carboxyl termini. Blockage of the amino or carboxyl group in a polypeptide, or both, by a covalent modification, is common in naturally-occurring and synthetic polypeptides. For instance, the amino terminal residue of polypeptides made in E. coli, prior to proteolytic processing, almost invariably will be N-formylmethionine.

The modifications can be a function of how the protein is made. For recombinant polypeptides, for example, the modifications will be determined by the host cell posttranslational modification capacity and the modification signals in the polypeptide amino acid sequence. Accordingly, when glycosylation is desired, a polypeptide should be expressed in a glycosylating host, generally a eukaryotic cell. Insect cells often carry out the same posttranslational glycosylations as mammalian cells and, for this reason, insect cell expression systems have been developed to efficiently express mammalian proteins having native patterns of glycosylation. Similar considerations apply to other modifications.

The same type of modification may be present in the same or varying degree at several sites in a given polypeptide. Also, a given polypeptide may contain more than one type of modification.

### Polypeptide Uses

The receptor polypeptides are useful for producing antibodies specific for the 14400, 2838, 14618, 15334, 14274, 32164, 39404, 38911, 26904, 31237, 18057, 16405, 32705, 23224, 27423, 32700, 32712 or 12216 receptor protein, regions, or fragments.

“Misexpression, altered, or aberrant expression”, as used herein, refers to a non-wild type pattern of gene expression, at the RNA or protein level. It includes: expression at non-wild type levels, i.e., over or under expression; a pattern of expression that differs from wild type in terms of the time or stage at which the gene is expressed, e.g., increased or decreased expression (as compared with wild type) at a predetermined developmental period or stage; a pattern of expression that differs from wild type in terms of decreased expression (as compared with wild type) in a predetermined cell type or tissue type; a pattern of expression that differs from wild type in terms of the splicing size, amino acid sequence, post-transitional modification, or biological activity of the expressed polypeptide; a pattern of expression that differs from wild type in terms of the effect of an environmental stimulus or extracellular stimulus on expression of the gene, e.g., a pattern of increased or decreased expression (as compared with wild type) in the presence of an increase or decrease in the strength of the stimulus.

Treatment is defined as the application or administration of a therapeutic agent to a patient, or application or administration of a therapeutic agent to an isolated tissue or cell line from a patient, who has a disease, a symptom of disease or a predisposition toward a disease, with the purpose to cure, heal, alleviate, relieve, alter, remedy, ameliorate, improve or affect the disease, the symptoms of disease or the predisposition toward disease. “Subject,” as used herein, can refer to a mammal, e.g. a human, or to an experimental or animal or disease model. The subject can also be a non-human animal, e.g. a horse, cow, goat, or other domestic animal. A therapeutic agent includes, but is not limited to, small molecules, peptides, antibodies, ribozymes and antisense oligonucleotides.

The receptor polypeptides, variants, and fragments (including those which may have been disclosed prior to the present invention) are useful for biological assays related to GPCRs. Such assays involve any of the known GPCR functions or activities or properties useful for diagnosis and treatment of GPCR-related conditions.

The receptor polypeptides are useful in drug screening assays, in cell-based or cell-free systems. Cell-based systems can be native, i.e., cells that normally express the receptor protein, as a biopsy or expanded in cell culture. In one embodiment, however, cell-based assays involve recombinant host cells expressing the receptor protein.

The polypeptides can be used to identify compounds that modulate receptor activity. Both 14400, 2838, 14618, 15334, 14274, 32164, 39404, 38911, 26904, 31237, 18057, 16405, 32705, 23224, 27423, 32700, 32712 or 12216 protein and appropriate variants and fragments can be used in high-throughput screens to assay candidate compounds for the ability to bind to the receptor. These compounds can be further screened against a functional receptor to determine the effect of the compound on the receptor activity. Compounds can be identified that activate (agonist) or inactivate (antagonist) the receptor to a desired degree.

The receptor polypeptides can be used to screen a compound for the ability to stimulate or inhibit interaction between the receptor protein and a target molecule that normally interacts with the receptor protein. The target can be ligand or a component of the signal pathway with which the receptor protein normally interacts (for example, a G-protein or other interactor involved in cAMP or phosphatidylinositol turnover and/or adenylate cyclase, or phospholipase C activation). The assay includes the steps of combining the receptor protein with a candidate compound under conditions that allow the receptor protein or fragment to interact with the target molecule, and to detect the formation of a complex between the protein and the target or to detect the biochemical consequence of the interaction with the receptor protein and the target, such as any of the associated effects of signal transduction such as G-protein phosphorylation, cyclic AMP or phosphatidylinositol turnover, and adenylate cyclase or phospholipase C activation.

Candidate compounds include, for example, 1) peptides such as soluble peptides, including Ig-tailed fusion peptides and members of random peptide libraries (see, e.g., Lam et al., Nature 354:82-84 (1991); Houghten et al., Nature 354:84-86 (1991)) and combinatorial chemistry-derived molecular libraries made of D- and/or L- configuration amino acids; 2) phosphopeptides (e.g., members of random and partially degenerate, directed phosphopeptide libraries, see, e.g., Songyang et al., Cell 72:767-778 (1993)); 3) antibodies (e.g., polyclonal, monoclonal, humanized, anti-idiotypic, chimeric, and single chain antibodies as well as Fab, F(ab′)₂, Fab expression library fragments, and epitope-binding fragments of antibodies); and 4) small organic and inorganic molecules (e.g., molecules obtained from combinatorial and natural product libraries).

One candidate compound is a soluble full-length receptor or fragment that competes for ligand binding. Other candidate compounds include mutant receptors or appropriate fragments containing mutations that affect receptor function and thus compete for ligand. Accordingly, a fragment that competes for ligand, for example with a higher affinity, or a fragment that binds ligand but does not allow release, is encompassed by the invention.

The invention-provides other end points to identify compounds that modulate (stimulate or inhibit) receptor activity. The assays typically involve an assay of events in the signal transduction pathway that indicate receptor activity. Thus, the expression of genes that are up- or down-regulated in response to the receptor protein dependent signal cascade can be assayed. In one embodiment, the regulatory region of such genes can be operably linked to a marker that is easily detectable, such as luciferase. Alternatively, phosphorylation of the receptor protein, or a receptor protein target, could also be measured.

Binding and/or activating compounds can also be screened by using chimeric receptor proteins in which the amino terminal extracellular domain, or parts thereof, the entire transmembrane domain or subregions, such as any of the seven transmembrane segments or any of the intracellular or extracellular loops and the carboxy terminal intracellular domain, or parts thereof, can be replaced by heterologous domains or subregions. For example, a G-protein-binding region can be used that interacts with a different G-protein then that which is recognized by the native receptor. Accordingly, a different set of signal transduction components is available as an end-point assay for activation. Alternatively, the entire transmembrane portion or subregions (such as transmembrane segments or intracellular or extracellular loops) can be replaced with the entire transmembrane portion or subregions specific to a host cell that is different from the host cell from which the amino terminal extracellular domain and/or the G-protein-binding region are derived. This allows for assays to be performed in other than the specific host cell from which the receptor is derived. Alternatively, the amino terminal extracellular domain (and/or other ligand-binding regions) could be replaced by a domain (and/or other binding region) binding a different ligand, thus, providing an assay for test compounds that interact with the heterologous amino terminal extracellular domain (or region) but still cause signal transduction. Finally, activation can be detected by a reporter gene containing an easily detectable coding region operably linked to a transcriptional regulatory sequence that is part of the native signal transduction pathway.

The receptor polypeptides are also useful in competition binding assays in methods designed to discover compounds that interact with the receptor. Thus, a compound is exposed to a receptor polypeptide under conditions that allow the compound to bind or to otherwise interact with the polypeptide. Soluble receptor polypeptide is also added to the mixture. If the test compound interacts with the soluble receptor polypeptide, it decreases the amount of complex formed or activity from the receptor target. This type of assay is particularly useful in cases in which compounds are sought that interact with specific regions of the receptor. Thus, the soluble polypeptide that competes with the target receptor region is designed to contain peptide sequences corresponding to the region of interest.

The polypeptides of the invention (including variants and fragments which may have been disclosed prior to the present invention) are useful for biological assays related to seven transmembrane protein and especially GPCRs. Such assays involve any of the known seven transmembrane protein or GPCR functions or activities or properties useful for diagnosis and treatment of seven transmembrane protein-related and especially GPCR-related conditions, especially diseases or disorders involving the tissues in which the protein is expressed as disclosed herein.

A polypeptide of the invention (including variants and fragments which may have been disclosed prior to the present invention) are also useful for biological assays related to GTPases, especially GTPases of the Ras family. Such assays involve any of the known GTPase functions or activities or properties useful for diagnosis and treatment of G-protein-related, and especially GTPase-related, conditions, especially diseases involving the tissues in which a protein of the invention is expressed as disclosed herein. For GTPase activity, assays include but are not limited to those disclosed herein, including those in references cited in the background herein, which are incorporated herein by reference for teaching these assays. Such assays include but are not included to GTP/GDP binding, binding to or activation by any of the regulatory proteins, activation of protein kinases, including the control of MAPK and JNK, interaction with protein kinase regulatory regions, including PAK2, hydrolysis of GTP, complex formation with any of the regulatory proteins, biological effects such as reorganization the actin cytoskeleton, transformation, growth, effects on differentiation, membrane ruffling induced by growth factors, formation of actin stress fibers, and generation of superoxide in phagocytes.

To perform cell free drug screening assays, it is desirable to immobilize either the receptor protein, or fragment, or its target molecule to facilitate separation of complexes from uncomplexed forms of one or both of the proteins, as well as to accommodate automation of the assay.

Techniques for immobilizing proteins on matrices can be used in the drug screening assays. In one embodiment, a fusion protein can be provided which adds a domain that allows the protein to be bound to a matrix. For example, glutathione-S-transferase/14400 fusion proteins can be adsorbed onto glutathione sepharose beads (Sigma Chemical, St. Louis, Mo.) or glutathione derivatized microtitre plates, which are then combined with the cell lysates (e.g., ³⁵S-labeled) and the candidate compound, and the mixture incubated under conditions conducive to complex formation (e.g., at physiological conditions for salt and pH). Following incubation, the beads are washed to remove any unbound label, and the matrix immobilized and radiolabeled determined directly, or in the supernatant after the complexes are dissociated. Alternatively, the complexes can be dissociated from the matrix, separated by SDS-PAGE, and the level of receptor-binding protein found in the bead fraction quantitated from the gel using standard electrophoretic techniques. For example, either the polypeptide or its target molecule can be immobilized utilizing conjugation of biotin and streptavidin using techniques well known in the art. Alternatively, antibodies reactive with the protein but which do not interfere with binding of the protein to its target molecule can be derivatized to the wells of the plate, and the protein trapped in the wells by antibody conjugation. Preparations of a receptor-binding protein and a candidate compound are incubated in the receptor protein-presenting wells and the amount of complex trapped in the well can be quantitated. Methods for detecting such complexes, in addition to those described above for the GST-immobilized complexes, include immunodetection of complexes using antibodies reactive with the receptor protein target molecule, or which are reactive with receptor protein and compete with the target molecule; as well as enzyme-linked assays which rely on detecting an enzymatic activity associated with the target molecule.

Modulators of 14400 receptor protein activity identified according to these drug screening assays can be used to treat a subject with a disorder mediated by the receptor pathway, by treating cells that express the 14400 protein, such as in spleen, thymus, prostate, testes, uterus, small intestine, colon, peripheral blood lymphocytes, heart, brain, placenta, lung, liver, skeletal muscle, kidney, and pancreas.

Modulators of 2838 receptor protein activity identified according to these drug screening assays can be used to treat a subject with a disorder mediated by the receptor pathway, by treating cells that express the 2838 protein, such as in lymph node, thymus, spleen, testes, colon, and peripheral blood lymphocytes including, but not limited to, T-helper cells (1 and 2), CD3⁺ (CD4 and CD8) cells, B cells and granulocytes.

TaqMan analyses demonstrated that high levels of 2838 expression are shown in lymph node and thymus. Accordingly, expression of 2838 is especially relevant to disorders involving these tissues. Extremely high 2838 expression is found in CD8 cells and activated B cells. High 2838 expression also occurs in activated T-helper cells (1 and 2), CD4 cells and Jurkat cells (a T-cell line). 2838 expression is differential in activated B cells and activated T-helper cells. 2838 expression increases upon activation in both of these cell types. Accordingly, expression of 2838 is relevant to disorders involving immune function and inflammation. 2838 is also significantly expressed in granulocytes. Accordingly, expression of 2838 is relevant to disorders involving these cells.

Modulators of 14618 receptor protein activity identified according to these drug screening assays can be used to treat a subject with a disorder mediated by the receptor pathway, by treating cells that express the 14618 protein, such as in breast, skeletal muscle, spleen and peripheral blood lymphocytes as well as CD34⁺ cells and megakaryocytes.

TaqMan analysis performed on the 14618 receptor showed expression of the 14618 receptor in a variety of normal human tissues. 14618 is highly expressed in breast and skeletal muscle. Significant 14618 expression also occurs in the thyroid, placenta, fetal kidney, fetal heart, and lymph node. Furthermore, the TaqMan analyses demonstrated lower levels of expression in a variety of other tissues. Accordingly, expression of 14618 is relevant in disorders involving these tissues.

The 14618 receptor is also expressed in various hematopoietic cells with and without activation. 14618 is highly expressed in CD34⁺ bone marrow cells. Accordingly, expression of 14618 is relevant in a variety of blood cell progenitors. Expression of 14618 is therefore relevant to disorders involving deficiencies in any of the major blood cell types, i.e. neutropenia, thrombocytopenia or anemia. 14618 is also highly expressed in mobilized peripheral blood cell megakaryocytes (mobilized with G-CSF). Accordingly, expression of 14618 is relevant to disorders involving platelet function, such as thrombocytopenia. Significant expression of 14618 is also seen in mobilized peripheral blood cell leukocytes, mobilized bone marrow CD34⁻ cells, and cord blood CD434⁺ cells. Accordingly, expression of 14618 is relevant to function of these cells, and therefore relevant to disorders involving immune function or inflammation. Further, expression of 14618 occurs in activated peripheral blood mononuclear cells. Activated B cells differentially express 14618. Accordingly, expression of 14618 is relevant to disorders involving immune function and/or inflammation.

Modulators of 15334 receptor protein activity identified according to these drug screening assays can be used to treat a subject with a disorder mediated by the receptor pathway, by treating cells that express the 15334 protein, such as in lymph node, tonsil, pancreas, colon, spleen, peripheral blood cells, thymus, adrenal gland and heart as well as megakaryocytes and erythroblasts.

TaqMan analyses demonstrate that 15334 is highly expressed in lymph node, tonsil, and pancreas. Expression of 15334 is also high in colon, testis, placenta, fetal heart, and spleen. In addition, the experiments show low levels of 15334 expression in several other tissues. Accordingly, expression of 15334 may be relevant to disorders involving these tissues. Expression of the 15334 receptor has also been studied in various hematopoietic cells. Extremely high 15334 expression occurs in primary megakaryocytes and erythroblasts. Accordingly, expression of 15334 is relevant to erythrocyte differentiation and megakaryoctye differentiation and thus is relevant to treating anemia and thrombocytopenia. Further, expression of 15334 is significantly increased in resting B cells compared to activated B cells. Accordingly, expression of 15334 is relevant to B cell immune function. Further, lower levels of 15334 expression are found in various other cells of the hematopoietic lineage. The expression of 15334 in hematopoietic cells in a lineage-restricted manner indicates that 15334 expression is relevant in regulating the development of the lineage cells, erythrocytes/red blood cells, or megakaryoctyes/platelets.

Modulators of 14274 receptor protein activity identified according to these drug screening assays can be used to treat a subject with a disorder mediated by the receptor pathway, by treating cells that express the 14274 receptor protein, such as in brain, spleen, lung, CD34⁻ bone marrow cells, peripheral blood cells, such as CD3 and CD8 T-cells, lung and colon carcinoma, liver metastases from colon, GCSF-treated mPB leukocytes, placenta and breast carcinoma, among others.

Modulators of 32164 protein activity identified according to these drug screening assays can be used to treat a subject with a disorder mediated by the 32164 protein pathway, by treating cells that express the 32164 protein, such as those disclosed herein (for example, an erythroid cell line). Preferred disorders include anemia.

Expression of 32164 is highly specific for hematopoietic cells. Hematopoietic progenitor CD34+ cells show significant expression of 32164 message. High level expression was also detected in fetal liver containing hematopoietic islands, and in erythroid lineage cells. Expression was regulated during both in vivo and in vitro generation of erythroid cells. Megakaryotes generated in vitro from CD34+ cells treated with Steel factor and thrombopoietin (which has previously been shown to induce the expression of erythroid-specific genes) showed high level expression of 32164.

Modulators of 39404 protein activity identified according to these drug screening assays can be used to treat a subject with a disorder mediated by the protein pathway, by treating cells that express the 39404 protein, such as in breast, brain, kidney, vein, fetal kidney, fetal liver, aortic intimal proliferations, internal mammary artery, and cells involved in congestive heart failure, ischemia, and myopathy, for example, cardiomyocytes.

Since the 39404 gene is expressed at high levels in brain, kidney, fetal kidney, fetal liver, aortic intimal proliferations and internal mammary artery, and in moderate levels in breast, vein, fetal kidney and fetal liver, assays are particularly useful in cells derived from these tissue types, and particularly the tissues in which the gene is highly expressed, such as brain, kidney, fetal kidney, fetal liver, internal mammary artery, and aortic intimal proliferations. Furthermore, since 39404 is expressed in these tissues, assays involving the protein in pathological tissue/disorders, particularly applies to disorders involving these tissues and especially the tissues in which the gene is highly expressed. Moreover, since 39404 is expressed in aortic intimal proliferations (atheroplaques), and heart tissue from patients with congestive heart failure, ischemia, and myopathy, the assays and methods involving pathology/disorders are particularly relevant in these disorders.

Modulators of 38911 protein activity identified according to these drug screening assays can be used to treat a subject with a disorder mediated by the protein pathway, by treating cells that express the 38911 protein, such as osteoclasts, spleen, tonsils, liver, kidney, and testis.

Since the 38911 gene is expressed in osteoclasts, spleen, tonsils, liver, kidney, and testis, the assays are particularly useful in cells derived from these tissue types, and particularly the cells and tissues in which the gene is highly expressed, such as spleen, tonsils, kidney, testis, liver, and osteoclasts. Furthermore, since 38911 is expressed in these tissues, assays involving the protein in pathological tissue/disorders, particularly applies to disorders involving these tissues and especially the tissues in which the gene is highly expressed. Since 38911 is highly expressed in osteoclasts, assays and methods involving pathology/disorders are particularly relevant to disorders involving osteoclast function. These disorders include but are not limited to those involved in bone growth and development, particularly disorders involving bone mass, such as osteoporosis. In addition, since relatively high 38911 expression occurs in fibrotic livers, liver fibrosis is a disorder relevant to expression of the 38911 receptor. Further, expression of the 38911 receptor is relevant to inflammation, in view of homology to the C5a receptor.

Modulators of 29604 protein activity identified according to these drug screening assays can be used to treat a subject with a disorder mediated by the protein pathway, by treating cells that express the 26904 protein, such as in brain.

Modulators of 31237 protein activity identified according to these drug screening assays can be used to treat a subject with a disorder mediated by the protein pathway, by treating cells that express the 31237 protein, such as in colon.

Modulators of 18057 protein activity identified according to these drug screening assays can be used to treat a subject with a disorder mediated by the 18057 protein pathway, by treating cells that express the 18057 protein, such as those involving the lung, liver, brain, kidney, breast, testes, and ovary, including, but not limited to, oncological disorders.

Modulators of 16405 receptor protein activity identified according to these drug screening assays can be used to treat a subject with a disorder mediated by the receptor pathway, by treating cells that express the 16405 protein, such as those disclosed herein. Since 16405 is expressed in tissues including, but not limited to, spleen, brain, including glioblastoma, and in sclerotic lesions, expression of the receptor and alteration of expression is important in diagnosing and treating disorders involving these tissues.

Modulators of 32705, 23224, 27423, 32700 or 32712 receptor protein activity identified according to these drug screening assays can be used to treat a subject with a disorder mediated by a protein of the invention, by treating cells that express a protein of the invention, such as those disclosed herein.

As assessed by TaqMan analysis, 32705 is highly expressed in tissues or cells that include, but are not limited to lung, brain, ganglia and virus-infected hepatocytes. Expression of 32705 is particularly high in brain. Differential expression of 32705 is shown in hepatitis B virus-infected HepG2 cells. Preferred disorders for 32705 include viral hepatitis, virus-infected liver, brain disorders, and liver fibrosis, especially from virus infection. Viruses include but are not limited to HBV. 23224 is expressed in tissues and cells that include, but are not limited to kidney, pancreas, spinal cord, brain cortex, brain hypothalamus, and dorsal root ganglia. 32700 is expressed in tissues and cells that include, but are not limited to, HUVEC (Human Umbilical Vein Endothelial Cells), hemangioma, skeletal muscle, brain cortex (normal), brain hypothalamus (normal), DRG (Dorsal Root Ganglion), ovary (tumor) and erythroid cells. 32712 is expressed in tissues and cell types including, but not limited to, kidney, primary osteoblasts, spinal cord (normal), brain cortex (normal), brain hypothalamus (normal), DRG (Dorsal Root Ganglion), prostate (normal), prostate (tumor), liver (normal), liver fibrosis, spleen (normal), tonsil (normal), lymph node (normal), BM-MNC (Bone Marrow Mononuclear Cells), neutrophils, megakaryocytes and erythroid cells.

Modulators of 12216 receptor protein activity identified according to these drug screening assays can be used to treat a subject with a disorder mediated by the receptor pathway, by treating cells that express the 12216 protein, such as those from brain, skeletal muscle, colon, heart CHF samples, mobilized peripheral blood CD34⁺ cells, human embryonic kidney cell lines, aorta, kidney, and monkey coronary, femoral, and renal arterial tissue and particularly in cells differentially expressing the protein or highly expressing the protein. Modulation is particularly relevant accordingly in brain, skeletal muscle, colon, CD34⁺ progenitor cells, aorta, and kidney. Particularly relevant disorders include, but are not limited to, congestive heart failure, ischemia and myopathy. In view of the fact that 12216 is highly expressed in CD34⁺ progenitor cells, detection/modulation is particularly relevant for treating neutropenia, thrombocytopenia or anemia. In view of the fact that 12216 is expressed in several atherogenic cell types, such as smooth muscle and macrophage, as well as endothelial cells, detection/modulation is particularly relevant for diagnosing and treating diseases involving atherogenesis, including atherosclerosis.

These methods of treatment include the steps of administering the modulators of protein activity in a pharmaceutical composition as described herein, to a subject in need of such treatment.

Disorders involving the spleen include, but are not limited to, splenomegaly, including nonspecific acute splenitis, congestive spenomegaly, and spenic infarcts; neoplasms, congenital anomalies, and rupture. Disorders associated with splenomegaly include infections, such as nonspecific splenitis, infectious mononucleosis, tuberculosis, typhoid fever, brucellosis, cytomegalovirus, syphilis, malaria, histoplasmosis, toxoplasmosis, kala-azar, trypanosomiasis, schistosomiasis, leishmaniasis, and echinococcosis; congestive states related to partial hypertension, such as cirrhosis of the liver, portal or splenic vein thrombosis, and cardiac failure; lymphohematogenous disorders, such as Hodgkin disease, non-Hodgkin lymphomas/leukemia, multiple myeloma, myeloproliferative disorders, hemolytic anemias, and thrombocytopenic purpura; immunologic-inflammatory conditions, such as rheumatoid arthritis and systemic lupus erythematosus; storage diseases such as Gaucher disease, Niemann-Pick disease, and mucopolysaccharidoses; and other conditions, such as amyloidosis, primary neoplasms and cysts, and secondary neoplasms.

Disorders involving the lung include, but are not limited to, congenital anomalies; atelectasis; diseases of vascular origin, such as pulmonary congestion and edema, including hemodynamic pulmonary edema and edema caused by microvascular injury, adult respiratory distress syndrome (diffuse alveolar damage), pulmonary embolism, hemorrhage, and infarction, and pulmonary hypertension and vascular sclerosis; chronic obstructive pulmonary disease, such as emphysema, chronic bronchitis, bronchial asthma, and bronchiectasis; diffuse interstitial (infiltrative, restrictive) diseases, such as pneumoconioses, sarcoidosis, idiopathic pulmonary fibrosis, desquamative interstitial pneumonitis, hypersensitivity pneumonitis, pulmonary eosinophilia (pulmonary infiltration with eosinophilia), Bronchiolitis obliterans-organizing pneumonia, diffuse pulmonary hemorrhage syndromes, including Goodpasture syndrome, idiopathic pulmonary hemosiderosis and other hemorrhagic syndromes, pulmonary involvement in collagen vascular disorders, and pulmonary alveolar proteinosis; complications of therapies, such as drug-induced lung disease, radiation-induced lung disease, and lung transplantation; tumors, such as bronchogenic carcinoma, including paraneoplastic syndromes, bronchioloalveolar carcinoma, neuroendocrine tumors, such as bronchial carcinoid, miscellaneous tumors, and metastatic tumors; pathologies of the pleura, including inflammatory pleural effusions, noninflammatory pleural effusions, pneumothorax, and pleural tumors, including solitary fibrous tumors (pleural fibroma) and malignant mesothelioma.

Disorders involving the colon include, but are not limited to, congenital anomalies, such as atresia and stenosis, Meckel diverticulum, congenital aganglionic megacolon-Hirschsprung disease; enterocolitis, such as diarrhea and dysentery, infectious enterocolitis, including viral gastroenteritis, bacterial enterocolitis, necrotizing enterocolitis, antibiotic-associated colitis (pseudomembranous colitis), and collagenous and lymphocytic colitis, miscellaneous intestinal inflammatory disorders, including parasites and protozoa, acquired immunodeficiency syndrome, transplantation, drug-induced intestinal injury, radiation enterocolitis, neutropenic colitis (typhlitis), and diversion colitis; idiopathic inflammatory bowel disease, such as Crohn disease and ulcerative colitis; tumors of the colon, such as non-neoplastic polyps, adenomas, familial syndromes, colorectal carcinogenesis, colorectal carcinoma, and carcinoid tumors.

Disorders involving the liver include, but are not limited to, hepatic injury; jaundice and cholestasis, such as bilirubin and bile formation; hepatic failure and cirrhosis, such as cirrhosis, portal hypertension, including ascites, portosystemic shunts, and splenomegaly; infectious disorders, such as viral hepatitis, including hepatitis A-E infection and infection by other hepatitis viruses, clinicopathologic syndromes, such as the carrier state, asymptomatic infection, acute viral hepatitis, chronic viral hepatitis, and fulminant hepatitis; autoimmune hepatitis; drug- and toxin-induced liver disease, such as alcoholic liver disease; inborn errors of metabolism and pediatric liver disease, such as hemochromatosis, Wilson disease, α₁-antitrypsin deficiency, and neonatal hepatitis; intrahepatic biliary tract disease, such as secondary biliary cirrhosis, primary biliary cirrhosis, primary sclerosing cholangitis, and anomalies of the biliary tree; circulatory disorders, such as impaired blood flow into the liver, including hepatic artery compromise and portal vein obstruction and thrombosis, impaired blood flow through the liver, including passive congestion and centrilobular necrosis and peliosis hepatis, hepatic vein outflow obstruction, including hepatic vein thrombosis (Budd-Chiari syndrome) and veno-occlusive disease; hepatic disease associated with pregnancy, such as preeclampsia and eclampsia, acute fatty liver of pregnancy, and intrehepatic cholestasis of pregnancy; hepatic complications of organ or bone marrow transplantation, such as drug toxicity after bone marrow transplantation, graft-versus-host disease and liver rejection, and nonimmunologic damage to liver allografts; tumors and tumorous conditions, such as nodular hyperplasias, adenomas, and malignant tumors, including primary carcinoma of the liver and metastatic tumors.

Disorders involving the uterus and endometrium include, but are not limited to, endometrial histology in the menstrual cycle; functional endometrial disorders, such as anovulatory cycle, inadequate luteal phase, oral contraceptives and induced endometrial changes, and menopausal and postmenopausal changes; inflammations, such as chronic endometritis; adenomyosis; endometriosis; endometrial polyps; endometrial hyperplasia; malignant tumors, such as carcinoma of the endometrium; mixed Müllerian and mesenchymal tumors, such as malignant mixed Müllerian tumors; tumors of the myometrium, including leiomyomas, leiomyosarcomas, and endometrial stromal tumors.

Disorders involving the brain include, but are not limited to, disorders involving neurons, and disorders involving glia, such as astrocytes, oligodendrocytes, ependymal cells, and microglia; cerebral edema, raised intracranial pressure and herniation, and hydrocephalus; malformations and developmental diseases, such as neural tube defects, forebrain anomalies, posterior fossa anomalies, and syringomyelia and hydromyelia; perinatal brain injury; cerebrovascular diseases, such as those related to hypoxia, ischemia, and infarction, including hypotension, hypoperfusion, and low-flow states—global cerebral ischemia and focal cerebral ischemia—infarction from obstruction of local blood supply, intracranial hemorrhage, including intracerebral (intraparenchymal) hemorrhage, subarachnoid hemorrhage and ruptured berry aneurysms, and vascular malformations, hypertensive cerebrovascular disease, including lacunar infarcts, slit hemorrhages, and hypertensive encephalopathy; infections, such as acute meningitis, including acute pyogenic (bacterial) meningitis and acute aseptic (viral) meningitis, acute focal suppurative infections, including brain abscess, subdural empyema, and extradural abscess, chronic bacterial meningoencephalitis, including tuberculosis and mycobacterioses, neurosyphilis, and neuroborreliosis (Lyme disease), viral meningoencephalitis, including arthropod-borne (Arbo) viral encephalitis, Herpes simplex virus Type 1, Herpes simplex virus Type 2, Varicalla-zoster virus (Herpes zoster), cytomegalovirus, poliomyelitis, rabies, and human immunodeficiency virus 1, including HIV-1 meningoencephalitis (subacute encephalitis), vacuolar myelopathy, AIDS-associated myopathy, peripheral neuropathy, and AIDS in children, progressive multifocal leukoencephalopathy, subacute sclerosing panencephalitis, fungal meningoencephalitis, other infectious diseases of the nervous system; transmissible spongiform encephalopathies (prion diseases); demyelinating diseases, including multiple sclerosis, multiple sclerosis variants, acute disseminated encephalomyelitis and acute necrotizing hemorrhagic encephalomyelitis, and other diseases with demyelination; degenerative diseases, such as degenerative diseases affecting the cerebral cortex, including Alzheimer disease and Pick disease, degenerative diseases of basal ganglia and brain stem, including Parkinsonism, idiopathic Parkinson disease (paralysis agitans), progressive supranuclear palsy, corticobasal degeneration, multiple system atrophy, including striatonigral degeneration, Shy-Drager syndrome, and olivopontocerebellar atrophy, and Huntington disease; spinocerebellar degenerations, including spinocerebellar ataxias, including Friedreich ataxia, and ataxia-telanglectasia, degenerative diseases affecting motor neurons, including amyotrophic lateral sclerosis (motor neuron disease), bulbospinal atrophy (Kennedy syndrome), and spinal muscular atrophy; inborn errors of metabolism, such as leukodystrophies, including Krabbe disease, metachromatic leukodystrophy, adrenoleukodystrophy, Pelizaeus-Merzbacher disease, and Canavan disease, mitochondrial encephalomyopathies, including Leigh disease and other mitochondrial encephalomyopathies; toxic and acquired metabolic diseases, including vitamin deficiencies such as thiamine (vitamin B₁) deficiency and vitamin B₁₂ deficiency, neurologic sequelae of metabolic disturbances, including hypoglycemia, hyperglycemia, and hepatic encephatopathy, toxic disorders, including carbon monoxide, methanol, ethanol, and radiation, including combined methotrexate and radiation-induced injury; tumors, such as gliomas, including astrocytoma, including fibrillary (diffuse) astrocytoma and glioblastoma multiforme, pilocytic astrocytoma, pleomorphic xanthoastrocytoma, and brain stem glioma, oligodendroglioma, and ependymoma and related paraventricular mass lesions, neuronal tumors, poorly differentiated neoplasms, including medulloblastoma, other parenchymal tumors, including primary brain lymphoma, germ cell tumors, and pineal parenchymal tumors, meningiomas, metastatic tumors, paraneoplastic syndromes, peripheral nerve sheath tumors, including schwannoma, neurofibroma, and malignant peripheral nerve sheath tumor (malignant schwannoma), and neurocutaneous syndromes (phakomatoses), including neurofibromotosis, including Type 1 neurofibromatosis (NF1) and TYPE 2 neurofibromatosis (NF2), tuberous sclerosis, and Von Hippel-Lindau disease.

Disorders involving T-cells include, but are not limited to, cell-mediated hypersensitivity, such as delayed type hypersensitivity and T-cell-mediated cytotoxicity, and transplant rejection; autoimmune diseases, such as systemic lupus erythematosus, Sjögren syndrome, systemic sclerosis, inflammatory myopathies, mixed connective tissue disease, and polyarteritis nodosa and other vasculitides; immunologic deficiency syndromes, including but not limited to, primary immunodeficiencies, such as thymic hypoplasia, severe combined immunodeficiency diseases, and AIDS; leukopenia; reactive (inflammatory) proliferations of white cells, including but not limited to, leukocytosis, acute nonspecific lymphadenitis, and chronic nonspecific lymphadenitis; neoplastic proliferations of white cells, including but not limited to lymphoid neoplasms, such as precursor T-cell neoplasms, such as acute lymphoblastic leukemia/lymphoma, peripheral T-cell and natural killer cell neoplasms that include peripheral T-cell lymphoma, unspecified, adult T-cell leukemia/lymphoma, mycosis fungoides and Sézary syndrome, and Hodgkin disease.

Diseases of the skin, include but are not limited to, disorders of pigmentation and melanocytes, including but not limited to, vitiligo, freckle, melasma, lentigo, nevocellular nevus, dysplastic nevi, and malignant melanoma; benign epithelial tumors, including but not limited to, seborrheic keratoses, acanthosis nigricans, fibroepithelial polyp, epithelial cyst, keratoacanthoma, and adnexal (appendage) tumors; premalignant and malignant epidermal tumors, including but not limited to, actinic keratosis, squamous cell carcinoma, basal cell carcinoma, and merkel cell carcinoma; tumors of the dermis, including but not limited to, benign fibrous histiocytoma, dermatofibrosarcoma protuberans, xanthomas, and dermal vascular tumors; tumors of cellular immigrants to the skin, including but not limited to, histiocytosis X, mycosis fungoides (cutaneous T-cell lymphoma), and mastocytosis; disorders of epidermal maturation, including but not limited to, ichthyosis; acute inflammatory dermatoses, including but not limited to, urticaria, acute eczematous dermatitis, and erythema multiforme; chronic inflammatory dermatoses, including but not limited to, psoriasis, lichen planus, and lupus erythematosus; blistering (bullous) diseases, including but not limited to, pemphigus, bullous pemphigoid, dermatitis herpetiformis, and noninflammatory blistering diseases: epidermolysis bullosa and porphyria; disorders of epidermal appendages, including but not limited to, acne vulgaris; panniculitis, including but not limited to, erythema nodosum and erythema induratum; and infection and infestation, such as verrucae, molluscum contagiosum, impetigo, superficial fungal infections, and arthropod bites, stings, and infestations.

In normal bone marrow, the myelocytic series (polymorphoneuclear cells) make up approximately 60% of the cellular elements, and the erythrocytic series, 20-30%. Lymphocytes, monocytes, reticular cells, plasma cells and megakaryocytes together constitute 10-20%. Lymphocytes make up 5-15% of normal adult marrow. In the bone marrow, cell types are add mixed so that precursors of red blood cells (erythroblasts), macrophages (monoblasts), platelets (megakaryocytes), polymorphoneuclear leucocytes (myeloblasts), and lymphocytes (lymphoblasts) can be visible in one microscopic field. In addition, stem cells exist for the different cell lineages, as well as a precursor stem cell for the committed progenitor cells of the different lineages. The various types of cells and stages of each would be known to the person of ordinary skill in the art and are found, for example, on page 42 (FIGS. 2-8) of Immunology, Imunopathology and Immunity, Fifth Edition, Sell et al. Simon and Schuster (1996), incorporated by reference for its teaching of cell types found in the bone marrow. According, the invention is directed to disorders arising from these cells. These disorders include but are not limited to the following: diseases involving hematopoetic stem cells; committed lymphoid progenitor cells; lymphoid cells including B and T-cells; committed myeloid progenitors, including monocytes, granulocytes, and megakaryocytes; and committed erythroid progenitors. These include but are not limited to the leukemias, including B-lymphoid leukemias, T-lymphoid leukemias, undifferentiated leukemias; erythroleukemia, megakaryoblastic leukemia, monocytic; [leukemias are encompassed with and without differentiation]; chronic and acute lymphoblastic leukemia, chronic and acute lymphocytic leukemia, chronic and acute myelogenous leukemia, lymphoma, myelo dysplastic syndrome, chronic and acute myeloid leukemia, myelomonocytic leukemia; chronic and acute myeloblastic leukemia, chronic and acute myelogenous leukemia, chronic and acute promyelocytic leukemia, chronic and acute myelocytic leukemia, hematologic malignancies of monocyte-macrophage lineage, such as juvenile chronic myelogenous leukemia; secondary AML, antecedent hematological disorder; refractory anemia; aplastic anemia; reactive cutaneous angioendotheliomatosis; fibrosing disorders involving altered expression in dendritic cells, disorders including systemic sclerosis, E-M syndrome, epidemic toxic oil syndrome, eosinophilic fasciitis localized forms of scleroderma, keloid, and fibrosing colonopathy; angiomatoid malignant fibrous histiocytoma; carcinoma, including primary head and neck squamous cell carcinoma; sarcoma, including kaposi's sarcoma; fibroadenoma and phyllodes tumors, including mammary fibroadenoma; stromal tumors; phyllodes tumors, including histiocytoma; erythroblastosis; neurofibromatosis; diseases of the vascular endothelium; demyelinating, particularly in old lesions; gliosis, vasogenic edema, vascular disease, Alzheimer's and Parkinson's disease; T-cell lymphomas; B cell lymphomas.

Disorders involving the heart, include but are not limited to, heart failure, including but not limited to, cardiac hypertrophy, left-sided heart failure, and right-sided heart failure; ischemic heart disease, including but not limited to angina pectoris, myocardial infarction, chronic ischemic heart disease, and sudden cardiac death; hypertensive heart disease, including but not limited to, systemic (left-sided) hypertensive heart disease and pulmonary (right-sided) hypertensive heart disease; valvular heart disease, including but not limited to, valvular degeneration caused by calcification, such as calcific aortic stenosis, calcification of a congenitally bicuspid aortic valve, and mitral annular calcification, and myxomatous degeneration of the mitral valve (mitral valve prolapse), rheumatic fever and rheumatic heart disease, infective endocarditis, and noninfected vegetations, such as nonbacterial thrombotic endocarditis and endocarditis of systemic lupus erythematosus (Libman-Sacks disease), carcinoid heart disease, and complications of artificial valves; myocardial disease, including but not limited to dilated cardiomyopathy, hypertrophic cardiomyopathy, restrictive cardiomyopathy, and myocarditis; pericardial disease, including but not limited to, pericardial effusion and hemopericardium and pericarditis, including acute pericarditis and healed pericarditis, and rheumatoid heart disease; neoplastic heart disease, including but not limited to, primary cardiac tumors, such as myxoma, lipoma, papillary fibroelastoma, rhabdomyoma, and sarcoma, and cardiac effects of noncardiac neoplasms; congenital heart disease, including but not limited to, left-to-right shunts—late cyanosis, such as atrial septal defect, ventricular septal defect, patent ductus arteriosus, and atrioventricular septal defect, right-to-left shunts—early cyanosis, such as tetralogy of fallot, transposition of great arteries, truncus arteriosus, tricuspid atresia, and total anomalous pulmonary venous connection, obstructive congenital anomalies, such as coarctation of aorta, pulmonary stenosis and atresia, and aortic stenosis and atresia, and disorders involving cardiac transplantation.

Disorders involving blood vessels include, but are not limited to, responses of vascular cell walls to injury, such as endothelial dysfunction and endothelial activation and intimal thickening; vascular diseases including, but not limited to, congenital anomalies, such as arteriovenous fistula, atherosclerosis, and hypertensive vascular disease, such as hypertension; inflammatory disease—the vasculitides, such as giant cell (temporal) arteritis, Takayasu arteritis, polyarteritis nodosa (classic), Kawasaki syndrome (mucocutaneous lymph node syndrome), microscopic polyanglitis (microscopic polyarteritis, hypersensitivity or leukocytoclastic anglitis), Wegener granulomatosis, thromboanglitis obliterans (Buerger disease), vasculitis associated with other disorders, and infectious arteritis; Raynaud disease; aneurysms and dissection, such as abdominal aortic aneurysms, syphilitic (luetic) aneurysms, and aortic dissection (dissecting hematoma); disorders of veins and lymphatics, such as varicose veins, thrombophlebitis and phlebothrombosis, obstruction of superior vena cava (superior vena cava syndrome), obstruction of inferior vena cava (inferior vena cava syndrome), and lymphangitis and lymphedema; tumors, including benign tumors and tumor-like conditions, such as hemangioma, lymphangioma, glomus tumor (glomangioma), vascular ectasias, and bacillary angiomatosis, and intermediate-grade (borderline low-grade malignant) tumors, such as Kaposi sarcoma and hemangloendothelioma, and malignant tumors, such as angiosarcoma and hemangiopericytoma; and pathology of therapeutic interventions in vascular disease, such as balloon angioplasty and related techniques and vascular replacement, such as coronary artery bypass graft surgery.

Disorders involving red cells include, but are not limited to, anemias, such as hemolytic anemias, including hereditary spherocytosis, hemolytic disease due to erythrocyte enzyme defects: glucose-6-phosphate dehydrogenase deficiency, sickle cell disease, thalassemia syndromes, paroxysmal nocturnal hemoglobinuria, immunohemolytic anemia, and hemolytic anemia resulting from trauma to red cells; and anemias of diminished erythropoiesis, including megaloblastic anemias, such as anemias of vitamin B12 deficiency: pernicious anemia, and anemia of folate deficiency, iron deficiency anemia, anemia of chronic disease, aplastic anemia, pure red cell aplasia, and other forms of marrow failure.

Disorders involving the thymus include developmental disorders, such as DiGeorge syndrome with thymic hypoplasia or aplasia; thymic cysts; thymic hypoplasia, which involves the appearance of lymphoid follicles within the thymus, creating thymic follicular hyperplasia; and thymomas, including germ cell tumors, lynphomas, Hodgkin disease, and carcinoids. Thymomas can include benign or encapsulated thymoma, and malignant thymoma Type I (invasive thymoma) or Type II, designated thymic carcinoma.

Disorders involving B cells include, but are not limited to precursor B cell neoplasms, such as lymphoblastic leukemia/lymphoma. Peripheral B cell neoplasms include, but are not limited to, chronic lymphocytic leukemia/small lymphocytic lymphoma, follicular lymphoma, diffuse large B cell lymphoma, Burkitt lymphoma, plasma cell neoplasms, multiple myeloma, and related entities, lymphoplasmacytic lymphoma (Waldenstr\overline{o}m macroglobulinemia), mantle cell lymphoma, marginal zone lymphoma (MALToma), and hairy cell leukemia.

Disorders involving the kidney include, but are not limited to, congenital anomalies including, but not limited to, cystic diseases of the kidney, that include but are not limited to, cystic renal dysplasia, autosomal dominant (adult) polycystic kidney disease, autosomal recessive (childhood) polycystic kidney disease, and cystic diseases of renal medulla, which include, but are not limited to, medullary sponge kidney, and nephronophthisis-uremic medullary cystic disease complex, acquired (dialysis-associated) cystic disease, such as simple cysts; glomerular diseases including pathologies of glomerular injury that include, but are not limited to, in situ immune complex deposition, that includes, but is not limited to, anti-GBM nephritis, Heymann nephritis, and antibodies against planted antigens, circulating immune complex nephritis, antibodies to glomerular cells, cell-mediated immunity in glomerulonephritis, activation of alternative complement pathway, epithelial cell injury, and pathologies involving mediators of glomerular injury including cellular and soluble mediators, acute glomerulonephritis, such as acute proliferative (poststreptococcal, postinfectious) glomerulonephritis, including but not limited to, poststreptococcal glomerulonephritis and nonstreptococcal acute glomerulonephritis, rapidly progressive (crescentic) glomerulonephritis, nephrotic syndrome, membranous glomerulonephritis (membranous nephropathy), minimal change disease (lipoid nephrosis), focal segmental glomerulosclerosis, membranoproliferative glomerulonephritis, IgA nephropathy (Berger disease), focal proliferative and necrotizing , glomerulonephritis (focal glomerulonephritis), hereditary nephritis, including but not limited to, Alport syndrome and thin membrane disease (benign familial hematuria), chronic glomerulonephritis, glomerular lesions associated with systemic disease, including but not limited to, systemic lupus erythematosus, Henoch-Schönlein purpura, bacterial endocarditis, diabetic glomerulosclerosis, amyloidosis, fibrillary and immunotactoid glomerulonephritis, and other systemic disorders; diseases affecting tubules and interstitium, including acute tubular necrosis and tubulointerstitial nephritis, including but not limited to, pyelonephritis and urinary tract infection, acute pyelonephritis, chronic pyelonephritis and reflux nephropathy, and tubulointerstitial nephritis induced by drugs and toxins, including but not limited to, acute drug-induced interstitial nephritis, analgesic abuse nephropathy, nephropathy associated with nonsteroidal anti-inflammatory drugs, and other tubulointerstitial diseases including, but not limited to, urate nephropathy, hypercalcemia and nephrocalcinosis, and multiple myeloma; diseases of blood vessels including benign nephrosclerosis, malignant hypertension and accelerated nephrosclerosis, renal artery stenosis, and thrombotic microangiopathies including, but not limited to, classic (childhood) hemolytic-uremic syndrome, adult hemolytic-uremic syndrome/thrombotic thrombocytopenic purpura, idiopathic HUS/TTP, and other vascular disorders including, but not limited to, atherosclerotic ischemic renal disease, atheroembolic renal disease, sickle cell disease nephropathy, diffuse cortical necrosis, and renal infarcts; urinary tract obstruction (obstructive uropathy); urolithiasis (renal calculi, stones); and tumors of the kidney including, but not limited to, benign tumors, such as renal papillary adenoma, renal fibroma or hamartoma (renomedullary interstitial cell tumor), angiomyolipoma, and oncocytoma, and malignant tumors, including renal cell carcinoma (hypernephroma, adenocarcinoma of kidney), which includes urothelial carcinomas of renal pelvis.

Disorders of the breast include, but are not limited to, disorders of development; inflammations, including but not limited to, acute mastitis, periductal mastitis, periductal mastitis (recurrent subareolar abscess, squamous metaplasia of lactiferous ducts), mammary duct ectasia, fat necrosis, granulomatous mastitis, and pathologies associated with silicone breast implants; fibrocystic changes; proliferative breast disease including, but not limited to, epithelial hyperplasia, sclerosing adenosis, and small duct papillomas; tumors including, but not limited to, stromal tumors such as fibroadenoma, phyllodes tumor, and sarcomas, and epithelial tumors such as large duct papilloma; carcinoma of the breast including in situ (noninvasive) carcinoma that includes ductal carcinoma in situ (including Paget's disease) and lobular carcinoma in situ, and invasive (infiltrating) carcinoma including, but not limited to, invasive ductal carcinoma, no special type, invasive lobular carcinoma, medullary carcinoma, colloid (mucinous) carcinoma, tubular carcinoma, and invasive papillary carcinoma, and miscellaneous malignant neoplasms.

Disorders in the male breast include, but are not limited to, gynecomastia and carcinoma.

Disorders involving the testis and epididymis include, but are not limited to, congenital anomalies such as cryptorchidism, regressive changes such as atrophy, inflammations such as nonspecific epididymitis and orchitis, granulomatous (autoimmune) orchitis, and specific inflammations including, but not limited to, gonorrhea, mumps, tuberculosis, and syphilis, vascular disturbances including torsion, testicular tumors including germ cell tumors that include, but are not limited to, seminoma, spermatocytic seminoma, embryonal carcinoma, yolk sac tumor choriocarcinoma, teratoma, and mixed tumors, tumore of sex cord-gonadal stroma including, but not limited to, leydig (interstitial) cell tumors and sertoli cell tumors (androblastoma), and testicular lymphoma, and miscellaneous lesions of tunica vaginalis.

Disorders involving the prostate include, but are not limited to, inflammations, benign enlargement, for example, nodular hyperplasia (benign prostatic hypertrophy or hyperplasia), and tumors such as carcinoma.

Disorders involving the thyroid include, but are not limited to, hyperthyroidism; hypothyroidism including, but not limited to, cretinism and myxedema; thyroiditis including, but not limited to, hashimoto thyroiditis, subacute (granulomatous) thyroiditis, and subacute lymphocytic (painless) thyroiditis; Graves disease; diffuse and multinodular goiter including, but not limited to, diffuse nontoxic (simple) goiter and multinodular goiter; neoplasms of the thyroid including, but not limited to, adenomas, other benign tumors, and carcinomas, which include, but are not limited to, papillary carcinoma, follicular carcinoma, medullary carcinoma, and anaplastic carcinoma; and cogenital anomalies.

Disorders involving the skeletal muscle include tumors such as rhabdomyosarcoma.

Disorders involving the pancreas include those of the exocrine pancreas such as congenital anomalies, including but not limited to, ectopic pancreas; pancreatitis, including but not limited to, acute pancreatitis; cysts, including but not limited to, pseudocysts; tumors, including but not limited to, cystic tumors and carcinoma of the pancreas; and disorders of the endocrine pancreas such as, diabetes mellitus; islet cell tumors, including but not limited to, insulinomas, gastrinomas, and other rare islet cell tumors.

Disorders involving the small intestine include the malabsorption syndromes such as, celiac sprue, tropical sprue (postinfectious sprue), whipple disease, disaccharidase (lactase) deficiency, abetalipoproteinemia, and tumors of the small intestine including adenomas and adenocarcinoma.

Disorders related to reduced platelet number, thrombocytopenia, include idiopathic thrombocytopenic purpura, including acute idiopathic thrombocytopenic purpura, drug-induced thrombocytopenia, HIV associated thrombocytopenia, and thrombotic microangiopathies: thrombotic thrombocytopenic purpura and hemolytic-uremic syndrome.

Disorders involving precursor T-cell neoplasms include precursor T lymphoblastic leukemia/lymphoma. Disorders involving peripheral T-cell and natural killer cell neoplasms include T-cell chronic lymphocytic leukemia, large granular lymphocytic leukemia, mycosis fungoides and Sézary syndrome, peripheral T-cell lymphoma, unspecified, angioimmunoblastic T-cell lymphoma, angiocentric lymphoma (NK/T-cell lymphoma^{4a}), intestinal T-cell lymphoma, adult T-cell leukemia/lymphoma, and anaplastic large cell lymphoma.

Disorders involving the ovary include, for example, polycystic ovarian disease, Stein-leventhal syndrome, Pseudomyxoma peritonei and stromal hyperthecosis; ovarian tumors such as, tumors of coelomic epithelium, serous tumors, mucinous tumors, endometeriod tumors, clear cell adenocarcinoma, cystadenofibroma, brenner tumor, surface epithelial tumors; germ cell tumors such as mature (benign) teratomas, monodermal teratomas, immature malignant teratomas, dysgerminoma, endodermal sinus tumor, choriocarcinoma; sex cord-stomal tumors such as, granulosa-theca cell tumors, thecomafibromas, androblastomas, hill cell tumors, and gonadoblastoma; and metastatic tumors such as Krukenberg tumors.

Bone-forming cells include the osteoprogenitor cells, osteoblasts, and osteocytes. The disorders of the bone are complex because they may have an impact on the skeleton during any of its stages of development. Hence, the disorders may have variable manifestations and may involve one, multiple or all bones of the body. Such disorders include, congenital malformations, achondroplasia and thanatophoric dwarfism, diseases associated with abnormal matix such as type 1 collagen disease, osteoporois, paget disease, rickets, osteomalacia, high-turnover osteodystrophy, low-turnover of aplastic disease, osteonecrosis, pyogenic osteomyelitis, tuberculous osteomyelitism, osteoma, osteoid osteoma, osteoblastoma, osteosarcoma, osteochondroma, chondromas, chondroblastoma, chondromyxoid fibroma, chondrosarcoma, fibrous cortical defects, fibrous dysplasia, fibrosarcoma, malignant fibrous histiocytoma, ewing saracoma, primitive neuroectodermal tumor, giant cell tumor, and metastatic tumors.

The receptor polypeptides also are useful to provide a target for diagnosing a disease or predisposition to disease mediated by the 14400, 2838, 14618, 15334, 14274, 32164, 39404, 38911, 26904, 31237, 18057, 16405, 32705, 23224, 27423, 32700, 32712 or 12216 receptor protein, especially in spleen, lung, colon, liver, uterus, brain, T-cells, skin, bone marrow, heart, blood vessels, red cells, thymus, B-cells, kidney, breast, testis, prostate, thyroid, skeletal muscle, pancreas, small intestine, platelet, ovary, bone, placenta, lymph nodes and tonsil as disclosed herein. Accordingly, methods are provided for detecting the presence, or levels of, the receptor protein in a cell, tissue, or organism. The method involves contacting a biological sample with a compound capable of interacting with the receptor protein such that the interaction can be detected.

One agent for detecting receptor protein is an antibody capable of selectively binding to receptor protein. A biological sample includes tissues, cells and biological fluids isolated from a subject, as well as tissues, cells and fluids present within a subject.

The receptor protein also provides a target for diagnosing active disease, or predisposition to disease, in a patient having a variant receptor protein. Thus, receptor protein can be isolated from a biological sample, assayed for the presence of a genetic mutation that results in aberrant receptor protein. This includes amino acid substitution, deletion, insertion, rearrangement, (as the result of aberrant splicing events), and inappropriate post-translational modification. Analytic methods include altered electrophoretic mobility, altered tryptic peptide digest, altered receptor activity in cell-based or cell-free assay, alteration in ligand or antibody-binding pattern, altered isoelectric point, direct amino acid sequencing, and any other of the known assay techniques useful for detecting mutations in a protein.

In vitro techniques for detection of receptor protein include enzyme linked immunosorbent assays (ELISAs), Western blots, immunoprecipitations and immunofluorescence. Alternatively, the protein can be detected in vivo in a subject by introducing into the subject a labeled anti-receptor antibody. For example, the antibody can be labeled with a radioactive marker whose presence and location in a subject can be detected by standard imaging techniques. Particularly useful are methods which detect the allelic variant of a receptor protein expressed in a subject and methods which detect fragments of a receptor protein in a sample.

The receptor polypeptides are also useful in pharmacogenomic analysis. Pharmacogenomics deal with clinically significant hereditary variations in the response to drugs due to altered drug disposition and abnormal action in affected persons. See, e.g., Eichelbaum, M., Clin. Exp. Pharmacol. Physiol. 23(10-11) :983-985 (1996), and Linder, M. W., Clin. Chem. 43(2):254-266 (1997). The clinical outcomes of these variations result in severe toxicity of therapeutic drugs in certain individuals or therapeutic failure of drugs in certain individuals as a result of individual variation in metabolism. Thus, the genotype of the individual can determine the way a therapeutic compound acts on the body or the way the body metabolizes the compound. Further, the activity of drug metabolizing enzymes affects both the intensity and duration of drug action. Thus, the pharmacogenomics of the individual permit the selection of effective compounds and effective dosages of such compounds for prophylactic or therapeutic treatment based on the individual's genotype. The discovery of genetic polymorphisms in some drug metabolizing enzymes has explained why some patients do not obtain the expected drug effects, show an exaggerated drug effect, or experience serious toxicity from standard drug dosages. Polymorphisms can be expressed in the phenotype of the extensive metabolizer and the phenotype of the poor metabolizer. Accordingly, genetic polymorphism may lead to allelic protein variants of the receptor protein in which one or more of the receptor functions in one population is different from those in another population. The polypeptides thus allow a target to ascertain a genetic predisposition that can affect treatment modality. Thus, in a ligand-based treatment, polymorphism may give rise to amino terminal extracellular domains and/or other ligand-binding regions that are more or less active in ligand binding, and receptor activation. Accordingly, ligand dosage would necessarily be modified to maximize the therapeutic effect within a given population containing a polymorphism. As an alternative to genotyping, specific polymorphic polypeptides could be identified.

The receptor polypeptides are also useful for monitoring therapeutic effects during clinical trials and other treatment. Thus, the therapeutic effectiveness of an agent that is designed to increase or decrease gene expression, protein levels or receptor activity can be monitored over the course of treatment using the receptor polypeptides as an end-point target. The monitoring can be, for example, as follows: (i) obtaining a pre-administration sample from a subject prior to administration of the agent; (ii) detecting the level of expression or activity of a specified protein in the pre-administration sample; (iii) obtaining one or more post-administration samples from the subject; (iv) detecting the level of expression or activity of the protein in the post-administration samples; (v) comparing the level of expression or activity of the protein in the pre-administration sample with the protein in the post-administration sample or samples; and (vi) increasing or decreasing the administration of the agent to the subject accordingly.

The receptor polypeptides are also useful for treating a receptor-associated disorder. Accordingly, methods for treatment include the use of soluble receptor or fragments of the receptor protein that compete for ligand binding. These receptors or fragments can have a higher affinity for the ligand so as to provide effective competition.

### Antibodies

The invention also provides antibodies that selectively bind to the 14400, 2838, 14618, 15334, 14274, 32164, 39404, 38911, 26904, 31237, 18057, 16405, 32705, 23224, 27423, 32700, 32712 or 12216 receptor protein and its variants and fragments. An antibody is considered to selectively bind, even if it also binds to other proteins that are not substantially homologous with the receptor protein. These other proteins share homology with a fragment or domain of the receptor protein. This conservation in specific regions gives rise to antibodies that bind to both proteins by virtue of the homologous sequence. In this case, it would be understood that antibody binding to the receptor protein is still selective.

To generate antibodies, an isolated receptor polypeptide is used as an immunogen to generate antibodies using standard techniques for polyclonal and monoclonal antibody preparation. Either the full-length protein or antigenic peptide fragment can be used. Antibodies are preferably prepared from these regions or from discrete fragments in these regions. However, antibodies can be prepared from any region of the peptide as described herein. A preferred fragment produces an antibody that diminishes or completely prevents ligand-binding. Antibodies can be developed against the entire receptor or portions of the receptor, for example, the intracellular carboxy terminal domain, the amino terminal extracellular domain, the entire transmembrane domain or specific segments, any of the intra or extracellular loops, or any portions of the above. Antibodies may also be developed against specific functional sites, such as the site of ligand-binding, the site of G protein coupling, or sites that are phosphorylated, glycosylated, or myristoylated.

An antigenic 14400 fragment will typically comprise at least 7 contiguous amino acid residues. The antigenic peptide can comprise, however, at least 12 amino acid residues, at least 14 amino acid residues, at least 15 amino acid residues, at least 20 amino acid residues, or at least 30 amino acid residues.

An antigenic 2838 fragment will typically comprise at least 8 contiguous amino acid residues. The antigenic peptide can comprise, however, a contiguous sequence of at least 12, 14 amino acid residues, at least 15 amino acid residues, at least 20 amino acid residues, or at least 30 amino acid residues.

An antigenic 14618 fragment will typically comprise at least 9 contiguous amino acid residues. The antigenic peptide can comprise, however, a contiguous sequence of at least 12, 14 amino acid residues, at least 15 amino acid residues, at least 20 amino acid residues, or at least 30 amino acid residues.

An antigenic 15334 fragment will typically comprise at least 8 contiguous amino acid residues. The antigenic peptide can comprise, however, a contiguous sequence of at least 12, 14 amino acid residues, at least 15 amino acid residues, at least 20 amino acid residues, or at least 30 amino acid residues.

An antigenic 14274 fragment will typically comprise at least 12 contiguous amino acid residues. The antigenic peptide can comprise, however, at least 14 amino acid residues, at least 15 amino acid residues, at least 20 amino acid residues, or at least 30 amino acid residues.

An antigenic 32164 fragment will typically comprise at least 6 contiguous amino acid residues. The antigenic peptide can comprise a contiguous sequence of at least 12, at least 14 amino acid residues, at least 15 amino acid residues, at least 20 amino acid residues, or at least 30 amino acid residues.

An antigenic 39404, 38911, 26904 or 31237 fragment will typically comprise at least 8-10 contiguous amino acid residues. The antigenic peptide can comprise, however, a contiguous sequence of at least 12, 14 amino acid residues, at least 15 amino acid residues, at least 20 amino acid residues, or at least 30 amino acid residues.

An antigenic 18057 fragment will typically comprise at least 6 contiguous amino acid residues. The antigenic peptide can comprise a contiguous sequence of at least 12, at least 14 amino acid residues, at least 15 amino acid residues, at least 20 amino acid residues, or at least 30 amino acid residues.

An antigenic 16405 fragment will typically comprise at least 7 contiguous amino acid residues. The antigenic peptide can comprise a contiguous sequence, however, at least 12, at least 14 amino acid residues, at least 15 amino acid residues, at least 20 amino acid residues, or at least 30 amino acid residues.

An antigenic 32705, 23224, 27423, 32700 or 32712 fragment will typically comprise at least 6 contiguous amino acid residues. The antigenic peptide can comprise a contiguous sequence of at least 12, at least 14 amino acid residues, at least 15 amino acid residues, at least 20 amino acid residues, or at least 30 amino acid residues.

In one embodiment, fragments correspond to regions that are located on the surface of the protein, e.g., hydrophilic regions. These fragments are not to be construed, however, as encompassing any fragments which may be disclosed prior to the invention.

Antibodies can be polyclonal or monoclonal. An intact antibody, or a fragment thereof (e.g. Fab or F(ab′)₂) can be used.

Detection can be facilitated by coupling (i.e., physically linking) the antibody to a detectable substance. Examples of detectable substances include various enzymes, prosthetic groups, fluorescent materials, luminescent materials, bioluminescent materials, and radioactive materials. Examples of suitable enzymes include horseradish peroxidase, alkaline phosphatase, β-galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; examples of bioluminescent materials include luciferase, luciferin, and aequorin, and examples of suitable radioactive material include ¹²⁵I, ¹³¹I, ³⁵S or ³H.

An appropriate immunogenic preparation can be derived from native, recombinantly expressed, protein or chemically synthesized peptides.

### Antibody Uses

The antibodies can be used to isolate a receptor protein by standard techniques, such as affinity chromatography or immunoprecipitation. The antibodies can facilitate the purification of the natural receptor protein from cells and recombinantly produced receptor protein expressed in host cells.

The antibodies are useful to detect the presence of receptor protein in cells or tissues to determine the pattern of expression of the receptor among various tissues in an organism and over the course of normal development.

The antibodies can be used to detect receptor protein in situ, in vitro, or in a cell lysate or supernatant in order to evaluate the abundance and pattern of expression.

The antibodies can be used to assess abnormal tissue distribution or abnormal expression during development.

Antibody detection of circulating fragments of the full length receptor protein can be used to identify receptor turnover.

The antibodies are also useful for inhibiting protein function, for example, blocking GTP, GDP, or regulatory protein binding.

Further, the antibodies can be used to assess receptor expression in disease states such as in active stages of the disease or in an individual with a predisposition toward disease related to receptor function. When a disorder is caused by an inappropriate tissue distribution, developmental expression, or level of expression of the receptor protein, the antibody can be prepared against the normal receptor protein. If a disorder is characterized by a specific mutation in the receptor protein, antibodies specific for this mutant protein can be used to assay for the presence of the specific mutant receptor protein.

The antibodies can also be used to assess normal and aberrant subcellular localization of cells in the various tissues in an organism. Antibodies can be developed against the whole receptor or portions of the receptor, for example, portions of the amino terminal extracellular domain or extracellular loops.

The diagnostic uses can be applied, not only in genetic testing, but also in monitoring a treatment modality. Accordingly, where treatment is ultimately aimed at correcting receptor expression level or the presence of aberrant receptors and aberrant tissue distribution or developmental expression, antibodies directed against the receptor or relevant fragments can be used to monitor therapeutic efficacy.

Additionally, antibodies are useful in pharmacogenomic analysis. Thus, antibodies prepared against polymorphic receptor proteins can be used to identify individuals that require modified treatment modalities.

The antibodies are also useful as diagnostic tools as an immunological marker for aberrant receptor protein analyzed by electrophoretic mobility, isoelectric point, tryptic peptide digest, and other physical assays known to those in the art.

The antibodies are also useful for tissue typing. Thus, where a specific receptor protein has been correlated with expression in a specific tissue, antibodies that are specific for this receptor protein can be used to identify a tissue type.

The antibodies are also useful in forensic identification. Accordingly, where an individual has been correlated with a specific genetic polymorphism resulting in a specific polymorphic protein, an antibody specific for the polymorphic protein can be used as an aid in identification.

The antibodies are also useful for inhibiting receptor function, for example, blocking ligand binding.

These uses can also be applied in a therapeutic context in which treatment involves inhibiting receptor function. An antibody can be used, for example, to block ligand binding. Antibodies can be prepared against specific fragments containing sites required for function or against intact receptor associated with a cell.

Completely human antibodies are particularly desirable for therapeutic treatment of human patients. For an overview of this technology for producing human antibodies, see Lonberg and Huszar (1995, Int. Rev. Immunol. 13:65-93). For a detailed discussion of this technology for producing human antibodies and human monoclonal antibodies and protocols for producing such antibodies, see, e.g., U.S. Pat. No. 5,625,126; U.S. Pat. No. 5,633,425; U.S. Pat. No. 5,569,825; U.S. Pat. No. 5,661,016; and U.S. Pat. No. 5,545,806.

The invention also encompasses kits for using antibodies to detect the presence of a receptor protein in a biological sample. The kit can comprise antibodies such as a labeled or labelable antibody and a compound or agent for detecting receptor protein in a biological sample; means for determining the amount of receptor protein in the sample; and means for comparing the amount of receptor protein in the sample with a standard. The compound or agent can be packaged in a suitable container. The kit can further comprise instructions for using the kit to detect receptor protein.

### Polynucleotides

The nucleotide sequence in SEQ ID NO:2, 5, 7, 9, 12, 15, 17, 19, 21, 23, 53, 57, 60, 62, 64, 66, 68 or 72 was obtained by sequencing the deposited human full length cDNA. Accordingly, the sequence of the deposited clone is controlling as to any discrepancies between the two and any reference to the sequence of SEQ ID NO:2, 5, 7, 9, 12, 15, 17, 19, 21, 23, 53, 57, 60, 62, 64, 66, 68 or 72 includes reference to the sequence of the deposited cDNA.

The specifically disclosed cDNA comprises the coding region and 5′ and 3′ untranslated sequences (SEQ ID NO:2, 5, 7, 9, 12, 15, 17, 19, 21, 23, 53, 57, 60, 62, 64, 66, 68 or 72). In one embodiment, the receptor nucleic acid comprises only the coding region.

The human 14400 receptor cDNA is approximately 1955 nucleotides in length (SEQ ID NO:2) and encodes a full length protein that is approximately 359 amino acid residues in length (SEQ ID NO:1). The nucleic acid is expressed in spleen, thymus, prostate, testes, uterus, small intestine, colon, peripheral blood lymphocytes, heart, brain, placenta, lung, liver, skeletal muscle, kidney, and pancreas. Structural analysis of the amino acid sequence of SEQ ID NO:1 was performed which demonstrated the putative structure of the seven transmembrane segments, the amino terminal extracellular domain and the carboxy terminal intracellular domain, as well as the hydrophobic and hydrophilic areas of the molecule as discussed herein.

The human 2838 receptor cDNA is approximately 1617 nucleotides in length (SEQ ID NO:5) and encodes a full length protein that is approximately 319 amino acid residues in length (SEQ ID NO:4). The nucleic acid is expressed in 2838 receptor protein is expressed in lymph node, thymus, spleen, testes, colon, and peripheral blood lymphocytes, and in activated T-helper cells (1 and 2), hypoxic Hep 3B cells, CD3 cells (both CD4 and CD8), activated B cells, Jurkat cells, granulocytes, among others. Structural analysis of the amino acid sequence of SEQ ID NO:4 was performed which demonstrated the putative structure of the seven transmembrane segments, the amino terminal extracellular domain and the carboxy terminal intracellular domain, as well as the hydrophobic and hydrophilic areas of the molecule as discussed herein.

The human 14618 receptor cDNA is approximately 1358 nucleotides in length (SEQ ID NO:7) and encodes a full length protein that is approximately 337 amino acid residues in length (SEQ ID NO:6). The nucleic acid is expressed in breast, skeletal muscle, lymph node, spleen and blood peripheral lymphocytes, as well as CD34⁺ cells and megakaryocytes. Structural analysis of the amino acid sequence of SEQ ID NO:6 was performed which demonstrated the putative structure of the seven transmembrane segments, the amino terminal extracellular domain and the carboxy terminal intracellular domain, as well as the hydrophobic and hydrophilic areas of the molecule as discussed herein.

The human 15334 receptor cDNA is approximately 2559 nucleotides in length (SEQ ID NO:9) and encodes a full length protein that is approximately 372 amino acid residues in length (SEQ ID NO:8). The nucleic acid is expressed in colon, placenta, pancreas, tonsil, lymph node, spleen, peripheral blood cells, thymus, adrenal gland and heart, as well as K562 cells, erythroblasts, and megakaryocytes. Structural analysis of the amino acid sequence of SEQ ID NO:8 was performed which demonstrated the putative structure of the seven transmembrane segments, the amino terminal extracellular domain and the carboxy terminal intracellular domain, as well as the hydrophobic and hydrophilic areas of the molecule as discussed herein.

The human 14274 receptor cDNA is approximately 1901 nucleotides in length (SEQ ID NO:12) and encodes a full length protein that is approximately 398 amino acid residues in length (SEQ ID NO:11). The nucleic acid is expressed in CD34⁻ bone marrow cells, peripheral blood cells, such as CD3 and CD8 T-cells, brain, spleen, lung, lung carcinoma, colon carcinoma, liver metastases from colon, GCSF-treated mPB leukocytes, and placenta, among others. Structural analysis of the amino acid sequence of SEQ ID NO:11 was performed which demonstrated the putative structure of the seven transmembrane segments, the amino terminal extracellular domain and the carboxy terminal intracellular domain, as well as the hydrophobic and hydrophilic areas of the molecule as discussed herein.

The human 32164 cDNA is approximately 1629 nucleotides in length (SEQ ID NO:15) and encodes a full length protein that is approximately 314 amino acid residues in length (SEQ ID NO:14). The nucleic acid is expressed at elevated levels in hematopoietic cells such as hematopoietic progenitor CD34+ cells. High level expression was also detected in fetal liver containing hematopoietic islands, and in erythroid lineage cells. Expression was regulated during both in vivo and in vitro generation of erythroid cells. Megakaryotes generated in vitro from CD34+ cells treated with Steel factor and thrombopoietin (which has previously been shown to induce the expression of erythroid-specific genes) showed high level expression of 32164. Structural analysis of the amino acid sequence of SEQ ID NO:14 was performed which demonstrated the putative structure of the seven transmembrane segments, the amino terminal extracellular domain and the carboxy terminal intracellular domain, as well as the hydrophobic and hydrophilic areas of the molecule as discussed herein.

The human 39404 cDNA is approximately 1729 nucleotides in length (SEQ ID NO:17) and encodes a full length protein that is approximately 337 amino acid residues in length (SEQ ID NO:16). The 39404 nucleic acid is expressed at high levels in brain, kidney, fetal kidney and fetal liver and in moderate levels in breast, vein, fetal kidney and fetal liver. High expression was also onserved in aortic intimal proliferations and internal mammary artery. Structural analysis of the amino acid sequence of SEQ ID NO:16 was performed which demonstrated the putative structure of the seven transmembrane segments, the amino terminal extracellular domain and the carboxy terminal intracellular domain, as well as the hydrophobic and hydrophilic areas of the molecule as discussed herein.

The human 38911 cDNA is approximately 1334 nucleotides in length (SEQ ID NO:19) and encodes a full length protein that is approximately 337 amino acid residues in length (SEQ ID NO:18). The nucleic acid is expressed in osteoclasts, spleen, tonsils, liver, kidney, and testis. Structural analysis of the amino acid sequence of SEQ ID NO:18 was performed which demonstrated the putative structure of the seven transmembrane segments, the amino terminal extracellular domain and the carboxy terminal intracellular domain, as well as the hydrophobic and hydrophilic areas of the molecule as discussed herein.

The human 26904 cDNA is approximately 1743 nucleotides in length (SEQ ID NO:21) and encodes a full length protein that is approximately 450 amino acid residues in length (SEQ ID NO:20). The nucleic acid is expressed in brain samples. Structural analysis of the amino acid sequence of SEQ ID NO:20 was performed which demonstrated the putative structure of the seven transmembrane segments, the amino terminal extracellular domain and the carboxy terminal intracellular domain, as well as the hydrophobic and hydrophilic areas of the molecule as discussed herein.

The human 31237 cDNA is approximately 2025 nucleotides in length (SEQ ID NO:23) and encodes a full length protein that is approximately 486 amino acid residues in length (SEQ ID NO:22). The nucleic acid is expressed in colon samples. Structural analysis of the amino acid sequence of SEQ ID NO:22 was performed which demonstrated the putative structure of the seven transmembrane segments, the amino terminal extracellular domain and the carboxy terminal intracellular domain, as well as the hydrophobic and hydrophilic areas of the molecule as discussed herein.

The human 18057 cDNA is approximately 1859 nucleotides in length (SEQ ID NO:53) and encodes a full length protein that is approximately 469 amino acid residues in length (SEQ ID NO:52). The 18057 nucleic acid is highly expressed in tissues or cells that include, but are not limited to human testes. The gene also shows expression in various other normal human tissues including, but not limited to, aorta, brain, breast, cervix, colon, esophagus, heart, kidney, liver, lung, lymph, muscle, ovary, placenta, prostate, small intestine, spleen, testes, thymus, thyroid, vein, pancreas, spinal cord, and astrocytes. Additional TaqMan analyses using oncology panels demonstrate 18057 expression in breast tumor, lung tumor, ovary tumor, colon tumor, prostate tumor, brain tumor, and metastatic liver cells. Structural analysis of the amino acid sequence of SEQ ID NO:52 was performed which demonstrated the putative structure of the seven transmembrane segments, the amino terminal extracellular domain and the carboxy terminal intracellular domain, as well as the hydrophobic and hydrophilic areas of the molecule as discussed herein.

The human 16405 receptor cDNA is approximately 2040 nucleotides in length (SEQ ID NO:57) and encodes a full length protein that is approximately 384 amino acid residues in length (SEQ ID NO:56). The 16405 nucleic acid is expressed in spleen, glioblastoma, and sclerotic lesion samples. Structural analysis of the amino acid sequence of SEQ ID NO:56 was performed which demonstrated the putative structure of the seven transmembrane segments, the amino terminal extracellular domain and the carboxy terminal intracellular domain, as well as the hydrophobic and hydrophilic areas of the molecule as discussed herein.

The human 32705 receptor cDNA is approximately 1347 nucleotides in length (SEQ ID NO:60) and encodes a full length protein that is approximately 236 amino acid residues in length (SEQ ID NO:61). The 32705 nucleic acid is highly expressed in tissues or cells that include, but are not limited to lung, brain, pancreas, skeletal muscle, nerve, normal skin, static HUVEC (Human Umbilical Vein Endothelial Cells), ganglia and virus-infected hepatocytes. Expression of 32705 is particularly high in brain. Differential expression of 32705 is shown in hepatitis B virus-infected HepG2 cells. Structural analysis of the amino acid sequence of SEQ ID NO:61 was performed which demonstrated the putative structure of the seven transmembrane segments, the amino terminal extracellular domain and the carboxy terminal intracellular domain, as well as the hydrophobic and hydrophilic areas of the molecule as discussed herein.

The human 23224 receptor cDNA is approximately 1023 nucleotides in length (SEQ ID NO:62) and encodes a full length protein that is approximately 213 amino acid residues in length (SEQ ID NO:63). The 23224 nucleic acid is expressed in tissues and cells that include, but are not limited to kidney, pancreas, spinal cord, brain cortex, brain hypothalamus, erythroid and dorsal root ganglia. Structural analysis of the amino acid sequence of SEQ ID NO:63 was performed which demonstrated the putative structure of the seven transmembrane segments, the amino terminal extracellular domain and the carboxy terminal intracellular domain, as well as the hydrophobic and hydrophilic areas of the molecule as discussed herein.

The human 27423 receptor cDNA is approximately 1161 nucleotides in length (SEQ ID NO:64) and encodes a full length protein that is approximately 207 amino acid residues in length (SEQ ID NO:65). Structural analysis of the amino acid sequence of SEQ ID NO:65 was performed which demonstrated the putative structure of the seven transmembrane segments, the amino terminal extracellular domain and the carboxy terminal intracellular domain, as well as the hydrophobic and hydrophilic areas of the molecule as discussed herein.

The human 32700 receptor cDNA is approximately 1199 nucleotides in length (SEQ ID NO:66) and encodes a full length protein that is approximately 183 amino acid residues in length (SEQ ID NO:67). The 32700 nucleic acid is expressed in tissues and cells that include, but are not limited to, HUVEC (Human Umbilical Vein Endothelial Cells), hemangioma, skeletal muscle, brain cortex (normal), brain hypothalamus (normal), DRG (Dorsal Root Ganglion), ovary (tumor) and erythroid cells. Structural analysis of the amino acid sequence of SEQ ID NO:67 was performed which demonstrated the putative structure of the seven transmembrane segments, the amino terminal extracellular domain and the carboxy terminal intracellular domain, as well as the hydrophobic and hydrophilic areas of the molecule as discussed herein.

The human 32712 receptor cDNA is approximately 1116 nucleotides in length (SEQ ID NO:68) and encodes a full length protein that is approximately 191 amino acid residues in length (SEQ ID NO:69). The 32712 nucleic acid is expressed in tissues and cell types including, but not limited to, kidney, primary osteoblasts, spinal cord (normal), brain cortex (normal), brain hypothalamus (normal), DRG (Dorsal Root Ganglion), prostate (normal), prostate (tumor), liver (normal), liver fibrosis, spleen (normal), tonsil (normal), lymph node (normal), BM-MNC (Bone Marrow Mononuclear Cells), neutrophils, megakaryocytes and erythroid cells. Structural analysis of the amino acid sequence of SEQ ID NO:69 was performed which demonstrated the putative structure of the seven transmembrane segments, the amino terminal extracellular domain and the carboxy terminal intracellular domain, as well as the hydrophobic and hydrophilic areas of the molecule as discussed herein.

The human 12216 receptor cDNA is approximately 2548 nucleotides in length (SEQ ID NO:72) and encodes a full length protein that is approximately 373 amino acid residues in length (SEQ ID NO:71). The 12216 nucleic acid is expressed in brain, skeletal muscle, colon, heart CHF samples, mobilized peripheral blood CD34⁺ cells, human embryonic kidney cell lines, aorta, kidney, and monkey coronary, femoral, and renal arterial tissue, among others. Structural analysis of the amino acid sequence of SEQ ID NO:71 was performed which demonstrated the putative structure of the seven transmembrane segments, the amino terminal extracellular domain and the carboxy terminal intracellular domain, as well as the hydrophobic and hydrophilic areas of the molecule as discussed herein.

As used herein, the term “transmembrane segment” refers to a structural amino acid motif which includes a hydrophobic helix that spans the plasma membrane.

The entire transmembrane domain of 14400 spans from about amino acid 24 to about amino acid 296 of SEQ ID NO:1. The entire transmembrane domain of 2838 spans from about amino acid 25 to about amino acid 292 of SEQ ID NO:4. The entire ransmembrane domain of 14618 spans from about amino acid 29 to about amino acid 297 of SEQ ID NO:6. The entire transmembrane domain of 15334 spans from about amino acid 26 to about amino acid 299 of SEQ ID NO:8. The entire transmembrane domain of 14274 span amino acids from about 40 to about 308 of SEQ ID NO:11. The entire transmembrane domain of 39404 spans from about amino acid 38 to about amino acid 305 of SEQ ID NO:16. The entire transmembrane domain of 38911 spans from about amino acid 41 to about amino acid 294 of SEQ ID NO:18. The entire transmembrane domain of 26904 spans from about amino acid 30 to about amino acid 430 of SEQ ID NO:20. The entire transmembrane domain of 31237 spans from about amino acid 100 to about amino acid 342 of SEQ ID NO:22. The entire transmembrane domain of 12216 spans from about amino acid 26 to about amino acid 343 of SEQ ID NO:71. Seven segments span the membrane and there are three intracellular and three extracellular loops in this domain.

The invention provides isolated polynucleotides encoding a 14400, 2838, 14618, 15334, 14274, 32164, 39404, 38911, 26904, 31237, 18057, 16405, 32705, 23224, 27423, 32700, 32712 or 12216 receptor protein. The term “14400, 2838, 14618, 15334, 14274, 32164, 39404, 38911, 26904, 31237, 18057, 16405, 32705, 23224, 27423, 32700, 32712 or 12216 polynucleotide” or “14400, 2838, 14618, 15334, 14274, 32164, 39404, 38911, 26904, 31237, 18057, 16405, 32705, 23224, 27423, 32700, 32712 or 12216 nucleic acid” refers to the sequence shown in SEQ ID NO:2, 5, 7, 9, 12, 15, 17, 19, 21, 23, 53, 57, 60, 62, 64, 66, 68 or 72 or in the deposited cDNA. The term “receptor polynucleotide” or “receptor nucleic acid” further includes variants and fragments of the 14400, 2838, 14618, 15334, 14274, 32164, 39404, 38911, 26904, 31237, 18057, 16405, 32705, 23224, 27423, 32700, 32712 or 12216 polynucleotide.

An “isolated” receptor nucleic acid is one that is separated from other nucleic acid present in the natural source of the receptor nucleic acid. Preferably, an “isolated” nucleic acid is free of sequences which naturally flank the nucleic acid (i.e., sequences located at the 5′ and 3′ ends of the nucleic acid) in the genomic DNA of the organism from which the nucleic acid is derived. However, there can be some flanking nucleotide sequences, for example up to about 5 KB. The important point is that the nucleic acid is isolated from flanking sequences such that it can be subjected to the specific manipulations described herein such as recombinant expression, preparation of probes and primers, and other uses specific to the receptor nucleic acid sequences.

Moreover, an “isolated” nucleic acid molecule, such as a cDNA molecule, can be substantially free of other cellular material, or culture medium when produced by recombinant techniques, or chemical precursors or other chemicals when chemically synthesized. However, the nucleic acid molecule can be fused to other coding or regulatory sequences and still be considered isolated.

For example, recombinant DNA molecules contained in a vector are considered isolated. Further examples of isolated DNA molecules include recombinant DNA molecules maintained in heterologous host cells or purified (partially or substantially) DNA molecules in solution. Isolated RNA molecules include in vivo or in vitro RNA transcripts of the isolated DNA molecules of the present invention. Isolated nucleic acid molecules according to the present invention further include such molecules produced synthetically.

The receptor polynucleotides can encode the mature protein plus additional amino or carboxyl-terminal amino acids, or amino acids interior to the mature polypeptide (when the mature form has more than one polypeptide chain, for instance). Such sequences may play a role in processing of a protein from precursor to a mature form, facilitate protein trafficking, prolong or shorten protein half-life or facilitate manipulation of a protein for assay or production, among other things. As generally is the case in situ, the additional amino acids may be processed away from the mature protein by cellular enzymes.

The receptor polynucleotides include, but are not limited to, the sequence encoding the mature polypeptide alone, the sequence encoding the mature polypeptide and additional coding sequences, such as a leader or secretory sequence (e.g., a pre-pro or pro-protein sequence), the sequence encoding the mature polypeptide, with or without the additional coding sequences, plus additional non-coding sequences, for example introns and non-coding 5′ and 3′ sequences such as transcribed but non-translated sequences that play a role in transcription, mRNA processing (including splicing and polyadenylation signals), ribosome binding and stability of mRNA. In addition, the polynucleotide may be fused to a marker sequence encoding, for example, a peptide that facilitates purification.

Receptor polynucleotides can be in the form of RNA, such as mRNA, or in the form DNA, including cDNA and genomic DNA obtained by cloning or produced by chemical synthetic techniques or by a combination thereof. The nucleic acid, especially DNA, can be double-stranded or single-stranded. Single-stranded nucleic acid can be the coding strand (sense strand) or the non-coding strand (anti-sense strand).

One receptor nucleic acid comprises the nucleotide sequence shown in SEQ ID NO:2, corresponding to human 14400 cDNA.

One receptor nucleic acid comprises the nucleotide sequence shown in SEQ ID NO:5, corresponding to human 2838 cDNA.

One receptor nucleic acid comprises the nucleotide sequence shown in SEQ ID NO:7, corresponding to human 14618 cDNA.

One receptor nucleic acid comprises the nucleotide sequence shown in SEQ ID NO:9, corresponding to human 15334 cDNA.

One receptor nucleic acid comprises the nucleotide sequence shown in SEQ ID NO:12, corresponding to human 14274 cDNA.

One receptor nucleic acid comprises the nucleotide sequence shown in SEQ ID NO:15, corresponding to human 32164 cDNA.

One receptor nucleic acid comprises the nucleotide sequence shown in SEQ ID NO:17, corresponding to human 39404 cDNA.

One receptor nucleic acid comprises the nucleotide sequence shown in SEQ ID NO:19, corresponding to human 38911 cDNA.

One receptor nucleic acid comprises the nucleotide sequence shown in SEQ ID NO:21, corresponding to human 26904 cDNA.

One receptor nucleic acid comprises the nucleotide sequence shown in SEQ ID NO:23, corresponding to human 31237 cDNA.

One receptor nucleic acid comprises the nucleotide sequence shown in SEQ ID NO:53, corresponding to human 18057 cDNA.

One receptor nucleic acid comprises the nucleotide sequence shown in SEQ ID NO:57, corresponding to human 16405 cDNA.

One receptor nucleic acid comprises the nucleotide sequence shown in SEQ ID NO:60, corresponding to human 32705 cDNA.

One receptor nucleic acid comprises the nucleotide sequence shown in SEQ ID NO:62, corresponding to human 23224 cDNA.

One receptor nucleic acid comprises the nucleotide sequence shown in SEQ ID NO:64, corresponding to human 27423 cDNA.

One receptor nucleic acid comprises the nucleotide sequence shown in SEQ ID NO:66, corresponding to human 32700 cDNA.

One receptor nucleic acid comprises the nucleotide sequence shown in SEQ ID NO:68, corresponding to human 32712 cDNA.

One receptor nucleic acid comprises the nucleotide sequence shown in SEQ ID NO:72, corresponding to human 12216 cDNA.

In one embodiment, the receptor nucleic acid comprises only the coding region.

The invention further provides variant receptor polynucleotides, and fragments thereof, that differ from the nucleotide sequence shown in SEQ ID NO:2, 5, 7, 9, 12, 15, 17, 19, 21, 23, 53, 57, 60, 62, 64, 66, 68 or 72 due to degeneracy of the genetic code and thus encode the same protein as that encoded by the nucleotide sequence shown in SEQ ID NO:2, 5, 7, 9, 12, 15, 17, 19, 21, 23, 53, 57, 60, 62, 64, 66, 68 or 72.

The invention also provides receptor nucleic acid molecules encoding the variant polypeptides described herein. Such polynucleotides may be naturally occurring, such as allelic variants (same locus), homologs (different locus), and orthologs (different organism), or may be constructed by recombinant DNA methods or by chemical synthesis. Such non-naturally occurring variants may be made by mutagenesis techniques, including those applied to polynucleotides, cells, or organisms. Accordingly, as discussed above, the variants can contain nucleotide substitutions, deletions, inversions and insertions.

Variation can occur in either or both the coding and non-coding regions. The variations can produce both conservative and non-conservative amino acid substitutions.

Orthologs, homologs, and allelic variants can be identified using methods well known in the art. These variants comprise a nucleotide sequence, encoding a receptor, that is at least about 50-55%, 55-60%, 60-65%, 65-70%, typically at least about 70-75%, more typically at least about 80-85%, and most typically at least about 90-95% or more homologous to the nucleotide sequence shown in SEQ ID NO:2, 5, 7, 9, 12, 15, 17, 19, 21, 23, 53, 57, 60, 62, 64, 66, 68 or 72 or a fragment of this sequence. Such nucleic acid molecules can readily be identified as being able to hybridize under stringent conditions, to the nucleotide sequence shown in SEQ ID NO:2, 5, 7, 9, 12, 15, 17, 19, 21, 23, 53, 57, 60, 62, 64, 66, 68 or 72 or a fragment of the sequence. It is understood that stringent hybridization does not indicate substantial homology where it is due to general homology, such as poly A sequences, or sequences common to all or most proteins, all GPCRs, or all family I GPCRs. Moreover, it is understood that variants do not include any of the nucleic acid sequences that may have been disclosed prior to the invention.

As used herein, the term “hybridizes under stringent conditions” is intended to describe conditions for hybridization and washing under which nucleotide sequences encoding a receptor at least 55% homologous to each other typically remain hybridized to each other. The conditions can be such that sequences at least about 65%, at least about 70%, or at least about 75% or more homologous to each other typically remain hybridized to each other. Such stringent conditions are known to those skilled in the art and can be found in Current Protocols in Molecular Biology, John Wiley & Sons, N.Y. (1989), 6.3.1-6.3.6. One example of stringent hybridization conditions are hybridization in 6×sodium chloride/sodium citrate (SSC) at about 45° C., followed by one or more washes in 0.2×SSC, 0.1% SDS at 50-65° C. In one embodiment, an isolated receptor nucleic acid molecule that hybridizes under stringent conditions to the sequence of SEQ ID NO:2, 5, 7, 9, 12, 15, 17, 19, 21, 23, 53, 57, 60, 62, 64, 66, 68 or 72 corresponds to a naturally-occurring nucleic acid molecule. As used herein, a “naturally-occurring” nucleic acid molecule refers to an RNA or DNA molecule having a nucleotide sequence that occurs in nature (e.g., encodes a natural protein).

As understood by those of ordinary skill, the exact conditions can be determined empirically and depend on ionic strength, temperature and the concentration of destabilizing agents such as formamide or denaturing agents such as SDS. Other factors considered in determining the desired hybridization conditions include the length of the nucleic acid sequences, base composition, percent mismatch between the hybridizing sequences and the frequency of occurrence of subsets of the sequences within other non-identical sequences. Thus, equivalent conditions can be determined by varying one or more of these parameters while maintaining a similar degree of identity or similarity between the two nucleic acid molecules.

The present invention also provides isolated nucleic acids that contain a single or double stranded fragment or portion that hybridizes under stringent conditions to a nucleotide sequence selected from the group consisting of SEQ ID NO:2, 5, 7, 9, 12, 15, 17, 19, 21, 23, 53, 57, 60, 62, 64, 66, 68 or 72 and the complements of SEQ ID NO:2, 5, 7, 9, 12, 15, 17, 19, 21, 23, 53, 57, 60, 62, 64, 66, 68 or 72. In one embodiment, the nucleic acid consists of a portion of a nucleotide sequence selected from the group consisting of SEQ ID NO:2, 5, 7, 9, 12, 15, 17, 19, 21, 23, 53, 57, 60, 62, 64, 66, 68 or 72 and the complements. The nucleic acid fragments of the invention are at least about 15, preferably at least about 18, 20, 23 or 25 nucleotides, and can be 30, 40, 50, 100, 200 or more nucleotides in length. Longer fragments, for example, 30 or more nucleotides in length, which encode antigenic proteins or polypeptides described herein are useful.

Furthermore, the invention provides polynucleotides that comprise a fragment of the full length receptor polynucleotides. The fragment can be single or double stranded and can comprise DNA or RNA. The fragment can be derived from either the coding or the non-coding sequence.

In one embodiment, an isolated 14400 receptor nucleic acid is at least 539 nucleotides in length and hybridizes under stringent conditions to the nucleic acid molecule comprising the nucleotide sequence of SEQ ID NO:2. In another embodiment an isolated receptor nucleic acid encodes the entire coding region from amino acid 1 to amino acid 359 of SEQ ID NO:2. In another embodiment the isolated receptor nucleic acid encodes a sequence corresponding to the mature protein from about amino acid 6 to amino acid 359 of SEQ ID NO:2. Fragments further include nucleic acid sequences encoding a portion of the amino acid sequence described herein and further including flanking nucleotide sequences at the 3′ region. Other fragments include nucleotide sequences encoding the amino acid fragments described herein. Receptor nucleic acid fragments also include a fragment from around nucleotide 609 to around 1794 of SEQ ID NO:2 and subfragments thereof. Receptor nucleic acid fragments further include a nucleotide sequence from around 647 to around 1794 of SEQ ID NO:2 and subfragments thereof. A further receptor nucleic acid fragment includes nucleic acid from around 653 to around 1794 of SEQ ID NO:2 and subfragments thereof. In these embodiments, the nucleic acid can be at least 17, 20, 30, 40, 50, 100, 250, or 500 nucleotides in length or greater. Nucleic acid fragments, according to the present invention, are not to be construed as encompassing those fragments that may have been disclosed prior to the invention. However, it is understood that a receptor fragment includes any nucleic acid sequence that does not include the entire gene.

14400 receptor nucleic acid fragments further include sequences corresponding to the domains described herein, subregions also described, and specific functional sites. 14400 receptor nucleic acid fragments include nucleic acid molecules encoding a polypeptide comprising the amino terminal extracellular domain including amino acid residues from 1 to about 23 of SEQ ID NO:2, a polypeptide comprising the region spanning the transmembrane domain (amino acid residues from about 24 to about 296 of SEQ ID NO:2), a polypeptide comprising the carboxy terminal intracellular domain (amino acid residues from about 297 to about 359 of SEQ ID NO:2), and a polypeptide encoding the G-protein receptor signature (120-122 of SEQ ID NO:2 or surrounding amino acid residues from about 109 to about 125 of SEQ ID NO:2), nucleic acid molecules encoding any of the seven transmembrane segments, extracellular or intracellular loops, glycosylation sites, cAMP or a GMP phosphorylation sites, and casein kinase II phosphorylation sites and myristoylation sites. 14400 receptor nucleic acid fragments also include combinations of the domains, segments, loops, and other functional sites described above. Thus, for example, a 14400 receptor nucleic acid could include sequences corresponding to the amino terminal extracellular domain and one transmembrane fragment. A person of ordinary skill in the art would be aware of the many permutations that are possible. Where the location of the domains have been predicted by computer analysis, one of ordinary skill would appreciate that the amino acid residues constituting these domains can vary depending on the criteria used to define the domains.

In another embodiment, an isolated 2838 receptor nucleic acid from nucleotide 1 to around nucleotide 990 is at least 16 nucleotides in length and hybridizes under stringent conditions to the nucleic acid molecule comprising the nucleotide sequence of SEQ ID NO:5. In another embodiment, the nucleic acid from around nucleotide 1487-1617 is at least 20 nucleotides. In other embodiments, the nucleic acid is at least 40, 50, 100, 250 or 500 nucleotides in length or greater. In another embodiment, an isolated 2838 receptor nucleic acid encodes the entire coding region from amino acid 1 to amino acid 319 of SEQ ID NO:4. In another embodiment, the isolated 2838 receptor nucleic acid encodes a sequence corresponding to the mature protein from about amino acid 6 to about amino acid 319 of SEQ ID NO:4.

2838 receptor nucleic acid fragments further include sequences corresponding to the domains described herein, subregions also described, and specific functional sites. 2838 receptor nucleic acid fragments include nucleic acid molecules encoding a polypeptide comprising the amino terminal extracellular domain including amino acid residues from 1 to about 24 of SEQ ID NO:4, a polypeptide comprising the region spanning the transmembrane domain (amino acid residues from about 25 to about 292 of SEQ ID NO:4), a polypeptide comprising the carboxy terminal intracellular domain (amino acid residues from about 293-to about 319 of SEQ ID NO:4), and a polypeptide encoding the G-protein receptor signature (118-120 or surrounding amino acid residues from about 107 to about 123 of SEQ ID NO:4), nucleic acid molecules encoding any of the seven transmembrane segments, extracellular or intracellular loops, glycosylation sites, phosphorylation sites, myristoylation sites, and amidation site.

In another embodiment, an isolated 14168 receptor nucleic acid from around nucleotide 1 to around nucleotide 911 is at least 8 nucleotides in length and hybridizes under stringent conditions to the nucleic acid molecule comprising the nucleotide sequence of SEQ ID NO:7. In other embodiments, the nucleic acid is at least 40, 50, 100, 250, or 500 nucleotides in length or greater. In another embodiment, an isolated 14618 receptor nucleic acid encodes the entire coding region from amino acid 1 to amino acid 337 of SEQ ID NO:6. In another embodiment, the isolated 14618 receptor nucleic acid encodes a sequence corresponding to the mature protein from about amino acid 6 to about amino acid 337 of SEQ ID NO:6.

14618 receptor nucleic acid fragments include nucleic acid molecules encoding a polypeptide comprising the amino terminal extracellular domain including amino acid residues from 1 to about 28 of SEQ ID NO:6, a polypeptide comprising the region spanning the transmembrane domain (amino acid residues from about 29 to about 297 of SEQ ID NO:6), a polypeptide comprising the carboxy terminal intracellular domain (amino acid residues from about 298 to about 337 of SEQ ID NO:6), and a polypeptide encoding the G-protein receptor signature (120-122 or surrounding amino acid residues from about 110 to about 132 of SEQ ID NO:6), nucleic acid molecules encoding any of the seven transmembrane segments, extracellular or intracellular loops, glycosylation sites and phosphorylation sites.

In another embodiment, an isolated 15334 receptor nucleic acid from nucleotide 1 to around nucleotide 1355 is at least 18 nucleotides in length and hybridizes under stringent conditions to the nucleic acid molecule comprising the nucleotide sequence of SEQ ID NO:9. In another embodiment, the nucleic acid from around nucleotide 868 to around 1355 is at least 11 nucleotides. In other embodiments, the nucleic acid is at least 40, 50, 100, 250, or 500 nucleotides in length or greater. In another embodiment, an isolated 15334 receptor nucleic acid encodes the entire coding region from amino acid 1 to amino acid 372 of SEQ ID NO:8. In another embodiment, the isolated 15334 receptor nucleic acid encodes a sequence corresponding to the mature protein from about amino acid 6 to about amino acid 372 of SEQ ID NO:8.

15334 receptor nucleic acid fragments include nucleic acid molecules encoding a polypeptide comprising the amino terminal extracellular domain including amino acid residues from 1 to about 25 of SEQ ID NO:8, a polypeptide comprising the region spanning the transmembrane domain (amino acid residues from about 26 to about 299 of SEQ ID NO:8), a polypeptide comprising the carboxy terminal intracellular domain (amino acid residues from about 300 to about 372 of SEQ ID NO:8), and a polypeptide encoding the G-protein receptor signature (118-120 or surrounding amino acid residues from about 110 to about 130 of SEQ ID NO:8), nucleic acid molecules encoding any of the seven transmembrane segments, extracellular or intracellular loops, glycosylation sites, protein kinase C, cAMP, cGMP, and casein kinase II phosphorylation sites, and myristoylation sites.

In another embodiment, an isolated 14274 receptor nucleic acid is at least 36 nucleotides in length and hybridizes under stringent conditions to the nucleic acid molecule comprising the nucleotide sequence of SEQ ID NO:12. In other embodiments, the 14274 nucleic acid is at least 40, 50, 100, 250 or 500 nucleotides in length. However, it is understood that a receptor fragment includes any nucleic acid sequence that does not include the entire gene.

14274 receptor nucleic acid fragments include nucleic acid molecules encoding a polypeptide comprising the amino terminal extracellular domain including amino acid residues from 1 to about 39 of SEQ ID NO:11, a polypeptide comprising the region spanning the entire transmembrane domain (amino acid residues from about 40 to about 308 of SEQ ID NO:11), a polypeptide comprising the carboxy terminal intracellular domain (amino acid residues from about 309 to about 398 of SEQ ID NO:11), and a polypeptide encoding the G-protein receptor signature (ERS or surrounding amino acid residues from about 121 to about 137 of SEQ ID NO:11). Further fragments include the specific seven transmembrane segments as well as the six intracellular and extracellular loops. Where the location of the domains have been predicted by computer analysis, one of ordinary skill would appreciate that the amino acid residues constituting these domains can vary depending on the criteria used to define the domains.

In another embodiment, an isolated 32164 nucleic acid encodes the entire coding region from amino acid 1 to amino acid 314 of SEQ ID NO:14. In another embodiment the isolated nucleic acid encodes a sequence corresponding to the mature protein from about amino acid 42 to amino acid 314 of SEQ ID NO:14. Other fragments include nucleotide sequences encoding the amino acid fragments described herein. Further fragments can include subfragments of the specific domains or sites described herein. Fragments also include nucleic acid sequences corresponding to specific amino acid sequences described above or fragments thereof. Nucleic acid fragments, according to the present invention, are not to be construed as encompassing those fragments that may have been disclosed prior to the invention.

In another embodiment, an isolated 39404 nucleic acid is at least 23 nucleotides in length and hybridizes under stringent conditions to the nucleic acid molecule comprising the nucleotide sequence of SEQ ID NO:17. The isolated fragments can be at least between 5-10, 10-20, 20-30, 30-40, 40-50, etc. including but not limited to 50, 75, 100, 200, 250, or 500 nucleotides in length or greater. In another embodiment, an isolated 39404 nucleic acid encodes the entire coding region from amino acid 1 to amino acid 337 of SEQ ID NO:16. In another embodiment, the isolated 39404 nucleic acid encodes a sequence corresponding to the mature protein from about amino acid 6 to about amino acid 337 of SEQ ID NO:16.

39404 nucleic acid fragments further include sequences corresponding to the domains described herein, subregions also described, and specific functional sites. 39404 nucleic acid fragments include but are not limited to nucleic acid molecules encoding a polypeptide comprising the amino terminal extracellular domain, comprising the region spanning the transmembrane domain, a polypeptide comprising the carboxy terminal intracellular domain, and a polypeptide encoding the G-protein receptor signature (130-132 or surrounding amino acid residues from about 120 to about 140 of SEQ ID NO16), nucleic acid molecules encoding any of the seven transmembrane segments, extracellular or intracellular loops, glycosylation sites or phosphorylation sites.

In another embodiment, an isolated 38911 nucleic acid from around nucleotide 1 to around nucleotide 200 is at least 5 nucleotides in length and hybridizes under stringent conditions to the nucleic acid molecule comprising the nucleotide sequence of SEQ ID NO:19. In other embodiments, the isolated nucleic acid is from around nucleotide 950 to nucleotide 1080 and is at least five nucleotides in length, hybridizing under stringent conditions. In other embodiments, from about nucleotide 190 to about nucleotide 950, fragments can be at least 5-10 nucleotides, at least 10-15 nucleotides, at least 15-20 nucleotides, at least 20-25 nucleotides, at least 25-30 nucleotides, at least 30-35 nucleotides, at least 35-40 nucleotides, for example, greater than 13 nucleotides, greater than 14 nucleotides, and greater than 18 nucleotides. In other embodiments, the nucleic acid is at least 40, 50, 100, 250, or 500 nucleotides in length or greater. In another embodiment, an isolated 38911 nucleic acid encodes the entire coding region from amino acid 1 to amino acid 337 of SEQ ID NO:18. In another embodiment, the isolated 38911 nucleic acid encodes a sequence corresponding to the mature protein from about amino acid 6 to about amino acid 337 of SEQ ID NO:18.

38911 nucleic acid fragments include but are not limited to nucleic acid molecules encoding a polypeptide comprising the amino terminal extracellular domain, the region spanning the transmembrane domain, and/or the carboxy terminal intracellular domain, and nucleic acid molecules encoding any of the seven transmembrane segments, extracellular or intracellular loops, glycosylation sites and phosphorylation sites.

In another embodiment, an isolated 26904 nucleic acid from nucleotide 1 to around nucleotide 498 is at least 14 nucleotides in length and hybridizes under stringent conditions to the nucleic acid molecule comprising the nucleotide sequence of SEQ ID NO:21. In another embodiment, the nucleic acid from around nucleotide 691 to around 1014 is at least 14 nucleotides. In other embodiments, the nucleic acid is at least 40, 50, 100, 250, or 500 nucleotides in length or greater. In another embodiment, an isolated 26904 nucleic acid encodes the entire coding region from amino acid 1 to amino acid 450 of SEQ ID NO:20. In another embodiment, the isolated 26904 nucleic acid encodes a sequence corresponding to the mature protein from about amino acid 6 to about amino acid 450 of SEQ ID NO:20.

26904 nucleic acid fragments include but are not limited to nucleic acid molecules encoding a polypeptide comprising the amino terminal extracellular domain, a polypeptide comprising the region spanning the transmembrane domain, and/or the carboxy terminal intracellular domain, and nucleic acid molecules encoding any of the seven transmembrane segments, extracellular or intracellular loops, glycosylation sites, protein kinase C, cAMP, cGMP, and casein kinase II phosphorylation sites, and myristoylation sites.

In another embodiment, an isolated 31237 nucleic acid encodes the entire coding region from amino acid 1 to amino acid 486 of SEQ ID NO:22. In another embodiment, the isolated 31237 nucleic acid encodes a sequence corresponding to the mature protein from about amino acid 6 to about amino acid 486 of SEQ ID NO:22.

31237 nucleic acid fragments include but are not limited to nucleic acid molecules encoding a polypeptide comprising the amino terminal extracellular domain, the region spanning the transmembrane domain, and/or a polypeptide comprising the carboxy terminal intracellular domain, and nucleic acid molecules encoding any of the seven transmembrane segments, extracellular or intracellular loops, glycosylation sites, protein kinase C, cAMP, cGMP, and casein kinase II phosphorylation sites, N-myristoylation sites, a glycosaminoglycan attachment site and immunoglobulin and major histocompatibility complex protein signature site.

In one embodiment, a 18057 fragment includes a contiguous stretch of nucleotides of 5-10 or 10-15 from around nucleotide 1 to around nucleotide 218 of SEQ ID NO:53, a contiguous stretch of 5-10, 10-20, 20-30, 30-40, or greater than 40 contiguous nucleotides from around nucleotide 218 to around nucleotide 700 of SEQ ID NO:53, a contiguous stretch of 5-10 or 10-15 nucleotides from around nucleotide 700 to around nucleotide 1200 of SEQ ID NO:53, and a contiguous stretch of 5-10, 10-20, or greater than 20 nucleotides from around nucleotide 1200 to around nucleotide 1859 of SEQ ID NO:53.

In another embodiment an isolated 18057 nucleic acid encodes the entire coding region from amino acid 1 to amino acid 469 of SEQ ID NO:52. In another embodiment the isolated nucleic acid encodes a sequence corresponding to the mature protein from about amino acid 14 to amino acid 469 of SEQ ID NO:52. Other fragments include nucleotide sequences encoding the amino acid fragments described herein. Further fragments can include subfragments of the specific domains or sites described herein. Fragments also include nucleic acid sequences corresponding to specific amino acid sequences described above or fragments thereof. Nucleic acid fragments, according to the present invention, are not to be construed as encompassing those fragments that may have been disclosed prior to the invention.

In another embodiment, an isolated 16405 receptor nucleic acid fragment is from nucleotide 1 to about nucleotide 1237, and from about nucleotide 1754 to about nucleotide 2040 is at least 5 nucleotides in length and hybridizes under stringent conditions to the nucleic acid molecule comprising the nucleotide sequence of SEQ ID NO:57. In other embodiments, the nucleic acid is at least about 10, 15, 20, 30, 40, 50, 75, 100, 150, 200, 250, 300, 400, or 500 nucleotides in length or greater.

In another embodiment, an isolated 16405 receptor nucleic acid encodes the entire coding region from amino acid 1 to amino acid 383 of SEQ ID NO:56. In another embodiment the isolated receptor nucleic acid encodes a sequence corresponding to the mature protein from about amino acid 6 to amino acid 383 of SEQ ID NO:56. Other fragments include nucleotide sequences encoding the amino acid fragments described herein. Further fragments can include subfragments of the specific domains or sites described herein. Fragments also include nucleic acid sequences corresponding to specific amino acid sequences described above or fragments thereof.

The 32705 nucleic acid fragments of the invention are at least about 10, 15, preferably at least about 20 or 25 nucleotides, and can be 30, 40, 50, 75, 100, 150, 200, 250, 300, 400, 500, 600, 700, 800, 900, 1000, 1100, 1200, 1300, or 1347 nucleotides in length. Alternatively, a nucleic acid molecule that is a fragment of a 32705-like nucleotide sequence of the present invention comprises a nucleotide sequence consisting of nucleotides 1-100, 100-200, 200-300, 300-400, 400-500, 500-600, 600-700, 700-800, 800-900, 900-1000, 1000-1100, 1100-1200, 1200-1360, or 1300-1347 of SEQ ID NO:60.

The 23224 nucleic acid fragments of the invention are at least about 10, 15, preferably at least about 20 or 25 nucleotides, and can be 30, 40, 50, 75, 100, 150, 200, 250, 300, 400, 500, 600, 700, 800, 900, 1000, or 1023 nucleotides in length. Alternatively, a nucleic acid molecule that is a fragment of a 23224-like nucleotide sequence of the present invention comprises a nucleotide sequence consisting of nucleotides 1-100, 100-200, 200-300, 300-400, 400-500, 500-600, 600-700, 700-800, 800-900, 900-1000, or 1000-1023 of SEQ ID NO:62.

The 27423 nucleic acid fragments of the invention are at least about 10, 15, preferably at least about 20 or 25 nucleotides, and can be 30, 40, 50, 75, 100, 150, 200, 250, 300, 400, 500, 600, 700, 800, 900, 1000, or 1161 nucleotides in length. Alternatively, a nucleic acid molecule that is a fragment of a 27423-like nucleotide sequence of the present invention comprises a nucleotide sequence consisting of nucleotides 1-100, 100-200, 200-300, 300-400, 400-500, 500-600, 600-700, 700-800, 800-900, 900-1000, 1000-1100, or 1100-1161 of SEQ ID NO:64.

The 32700 nucleic acid fragments of the invention are at least about 10, 15, preferably at least about 20 or 25 nucleotides, and can be 30, 40, 50, 75, 100, 150, 200, 250, 300, 400, 500, 600, 700, 800, 900, 1000, 1100, or 1199 nucleotides in length. Alternatively, a nucleic acid molecule that is a fragment of a 32700-like nucleotide sequence of the present invention comprises a nucleotide sequence consisting of nucleotides 1-100, 100-200, 200-300, 300-400, 400-500, 500-600, 600-700, 700-800, 800-900, 900-1000, 1000-1100, or 1100-1199 of SEQ ID NO:66.

The 32712 nucleic acid fragments of the invention are at least about 10, 15, preferably at least about 20 or 25 nucleotides, and can be 30, 40, 50, 75, 100, 150, 200, 250, 300, 400, 500, 600, 700, 800, 900, 1000, or 1116 nucleotides in length. Alternatively, a nucleic acid molecule that is a fragment of a 32712-like nucleotide sequence of the present invention comprises a nucleotide sequence consisting of nucleotides 1-100, 100-200, 200-300, 300-400, 400-500, 500-600, 600-700, 700-800, 800-900, 900-1000, 1000-1100, or 1100-1116 of SEQ ID NO:68.

In another embodiment, the 12216 nucleic acid is at least 40, 50, 100, 250 or 500 nucleotides in length. For example, nucleotide sequences 1 to about 360, about 475 to about 800, about 1109 to about 1269, and about 2167 to about 2548 of SEQ ID NO:72 are not disclosed prior to the present invention. Other regions of the nucleotide sequence may comprise fragments of various sizes, depending upon potential homology with previous disclosed sequences. For example, the nucleotide sequence from about 360 to about 475 of SEQ ID NO:72 encompasses fragments greater than 81 nucleotides, the nucleotide sequence from about 800 to about 1109 of SEQ ID NO:72 encompasses fragments greater than 15 nucleotides, the nucleotide sequence from about 1269 to about 1498 of SEQ ID NO:72 encompasses fragments greater than 131 nucleotides, the nucleotide sequence from about 1498 to about 1577 of SEQ ID NO:72 encompasses fragments greater than 35 nucleotides, the nucleotide sequence from about 1577 to about 1950 of SEQ ID NO:72 encompasses nucleotide fragments greater than 12, the nucleotide sequence from about 1950 to about 2112 of SEQ ID NO:72 encompasses nucleotide fragments greater than 88, and the nucleotide sequence from about 2108 to about 2167 of SEQ ID NO:72 encompasses nucleotide fragments greater than 32. In these embodiments, depending on the region, the nucleic acid can be at least 15, 20, 30, 40, 50, 100, 250, or 500 nucleotides in length or greater. Nucleic acid fragments also include those encoding the receptor polypeptide but extending into the 5′ and/or 3′ noncoding regions. Further, fragments include parts of the receptor coding region with extensions in the 5′ or 3′ noncoding sequences.

In another embodiment an isolated 12216 receptor nucleic acid encodes the entire coding region from amino acid 1 to amino acid 373 of SEQ ID NO:71. In another embodiment the isolated receptor nucleic acid encodes a sequence corresponding to the mature protein from about amino acid 6 to amino acid 373 of SEQ ID NO:71. Other fragments include nucleotide sequences encoding the amino acid fragments described herein. Further fragments can include subfragments of the specific domains or sites described herein. Fragments also include nucleic acid sequences corresponding to specific amino acid sequences described above or fragments thereof. Nucleic acid fragments, according to the present invention, are not to be construed as encompassing those fragments that may have been disclosed prior to the invention and include all non-disclosed fragments.

12216 receptor nucleic acid fragments include nucleic acid molecules encoding a polypeptide comprising the amino terminal extracellular domain including amino acid residues from 1 to about 25 of SEQ ID NO:71, a polypeptide comprising the region spanning the transmembrane domain (amino acid residues from about 26 to about 343 of SEQ ID NO:71), a polypeptide comprising the carboxy terminal intracellular domain (amino acid residues from about 344 to about 373 of SEQ ID NO:71), and a polypeptide encoding the G-protein receptor signature (120-122 or surrounding amino acid residues from about 110 to about 130 of SEQ ID NO:71), nucleic acid molecules encoding any of the seven transmembrane segments, extracellular or intracellular loops, glycosylation, phosphorylation, myristoylation, and prenylation sites. Where the location of the domains have been predicted by computer analysis, one of ordinary skill would appreciate that the amino acid residues constituting these domains can vary depending on the criteria used to define the domains.

The invention also provides receptor nucleic acid fragments that encode epitope bearing regions of the receptor proteins described herein.

The isolated receptor polynucleotide sequences, and especially fragments, are useful as DNA probes and primers.

For example, the coding region of a receptor gene can be isolated using the known nucleotide sequence to synthesize an oligonucleotide probe. A labeled probe can then be used to screen a cDNA library, genomic DNA library, or mRNA to isolate nucleic acid corresponding to the coding region. Further, primers can be used in PCR reactions to clone specific regions of receptor genes.

A probe/primer typically comprises substantially purified oligonucleotide. The oligonucleotide typically comprises a region of nucleotide sequence that hybridizes under stringent conditions to at least about 12, typically about 25, more typically about 40, 50 or 75 consecutive nucleotides of SEQ ID NO:2, 5, 7, 9, 12, 15, 17, 19, 21, 23, 53, 57, 60, 62, 64, 66, 68 or 72 sense or anti-sense strand or other receptor polynucleotides. A probe further comprises a label, e.g., radioisotope, fluorescent compound, enzyme, or enzyme co-factor.

### Polynucleotide Uses

The nucleic acid sequences of the present invention can be used as a “query sequence” to perform a search against public databases to, for example, identify other family members or related sequences. Such searches can be performed using the NBLAST and XBLAST programs (version 2.0) of Altschul et al. (1990) J. Mol. Biol. 215:403-10. BLAST nucleotide searches can be performed with the NBLAST program, score=100, wordlength =12 to obtain nucleotide sequences homologous to the nucleic acid molecules of the invention. To obtain gapped alignments for comparison purposes, Gapped BLAST can be utilized as described in Altschul et al. (1997) Nucleic Acids Res. 25(17):3389-3402. When utilizing BLAST and Gapped BLAST programs, the default parameters of the respective programs (e.g., XBLAST and NBLAST) can be used.

The nucleic acid fragments of the invention provide probes or primers in assays such as those described below. “Probes” are oligonucleotides that hybridize in a base-specific manner to a complementary strand of nucleic acid. Such probes include polypeptide nucleic acids, as described in Nielsen et al. (1991) Science 254:1497-1500. Typically, a probe comprises a region of nucleotide sequence that hybridizes under highly stringent conditions to at least about 15, typically about 20-25, and more typically about 40, 50 or 75 consecutive nucleotides of the nucleic acid of SEQ ID NO:2, 5, 7, 9, 12, 15, 17, 19, 21, 23, 53, 57, 60, 62, 64, 66, 68 or 72 and the complements thereof. More typically, the probe further comprises a label, e.g., radioisotope, fluorescent compound, enzyme, or enzyme co-factor.

As used herein, the term “primer” refers to a single-stranded oligonucleotide which acts as a point of initiation of template-directed DNA synthesis using well-known methods (e.g., PCR, LCR) including, but not limited to those described herein. The appropriate length of the primer depends on the particular use, but typically ranges from about 15 to 30 nucleotides. The term “primer site” refers to the area of the target DNA to which a primer hybridizes. The term “primer pair” refers to a set of primers including a 5′ (upstream) primer that hybridizes with the 5′ end of the nucleic acid sequence to be amplified and a 3′ (downstream) primer that hybridizes with the complement of the sequence to be amplified.

The polynucleotides are useful for probes, primers, and in biological assays, including, but not limited to, methods using the cells and tissues in which the gene is expressed, particularly in which the gene is significantly expressed, and involving disorders including, but not limited to, those also discussed herein above with respect to biological methods and assays involving the 14400, 2838, 14618, 15334, 14274, 32164, 39404, 38911, 26904, 31237, 18057, 16405, 32705, 23224, 27423, 32700, 32712 or 12216 polypeptides.

Where the polynucleotides are used to assess seven transmembrane protein properties, and especially GPCR properties or functions, such as in the assays described herein, all or less than all of the entire cDNA can be useful. In this case, even fragments that may have been known prior to the invention are encompassed. Thus, for example, assays specifically directed to seven transmembrane proteins, and especially GPCR functions, such as assessing agonist or antagonist activity, encompass the use of known fragments. Further, diagnostic methods for assessing function can also be practiced with any fragment, including those fragments that may have been known prior to the invention. Similarly, in methods involving modulation or treatment of 14400, 2838, 14618, 15334, 14274, 32164, 39404, 38911, 26904, 31237, 18057, 16405, 32705, 23224, 27423, 32700, 32712 or 12216-related dysfunction, all fragments are encompassed including those which may have been known in the art.

The polynucleotides are useful as a hybridization probe for cDNA and genomic DNA to isolate a full-length cDNA and genomic clones encoding the polypeptide described in SEQ ID NO:1, 4, 6, 8, 11, 14, 16, 18, 20, 22, 52, 56, 61, 63, 65, 67, 69 or 71 and to isolate cDNA and genomic clones that correspond to variants producing the same polypeptide shown in SEQ ID NO:1, 4, 6, 8, 11, 14, 16, 18, 20, 22, 52, 56, 61, 63, 65, 67, 69 or 71 or the other variants described herein. Variants can be isolated from the same tissue and organism from which the polypeptide shown in SEQ ID NO:1, 4, 6, 8, 11, 14, 16, 18, 20, 22, 52, 56, 61, 63, 65, 67, 69 or 71 was isolated, different tissues from the same organism, or from different organisms. This method is useful for isolating genes and cDNA that are developmentally-controlled and therefore may be expressed in the same tissue or different tissues at different points in the development of an organism.

The probe can correspond to any sequence along the entire length of the gene encoding the 14400, 2838, 14618, 15334, 14274, 32164, 39404, 38911, 26904, 31237, 18057, 16405, 32705, 23224, 27423, 32700, 32712 or 12216 protein. Accordingly, it could be derived from 5′ noncoding regions, the coding region, and 3′ noncoding regions. It is understood, however, as discussed herein, that fragments corresponding to the probe do not include those fragments that may have been disclosed prior to the present invention.

The nucleic acid probe can be, for example, the full-length cDNA of SEQ ID NO:1, 4, 6, 8, 11, 14, 16, 18, 20, 22, 52, 56, 61, 63, 65, 67, 69 or 71, or a fragment thereof, such as an oligonucleotide of at least 5, 10, 12, 15, 30, 50, 100, 250 or 500 nucleotides in length and sufficient to specifically hybridize under stringent conditions to mRNA or DNA.

Fragments of the polynucleotides described herein are also useful to synthesize larger fragments or full-length polynucleotides described herein. For example, a fragment can be hybridized to any portion of an mRNA and a larger or full-length cDNA can be produced.

The fragments are also useful to synthesize antisense molecules of desired length and sequence.

Antisense nucleic acids of the invention can be designed using the nucleotide sequences of SEQ ID NO:2, 5, 7, 9, 12, 15, 17, 19, 21, 23, 53, 57, 60, 62, 64, 66, 68 or 72, and constructed using chemical synthesis and enzymatic ligation reactions using procedures known in the art. For example, an antisense nucleic acid (e.g., an antisense oligonucleotide) can be chemically synthesized using naturally occurring nucleotides or variously modified nucleotides designed to increase the biological stability of the molecules or to increase the physical stability of the duplex formed between the antisense and sense nucleic acids, e.g., phosphorothioate derivatives and acridine substituted nucleotides can be used. Examples of modified nucleotides which can be used to generate the antisense nucleic acid include 5-fluorouracil, 5-bromouracil, 5-chlorouracil, 5-iodouracil, hypoxanthine, xanthine, 4-acetylcytosine, 5-(carboxyhydroxylmethyl) uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl) uracil, (acp3)w, and 2,6-diaminopurine. Alternatively, the antisense nucleic acid can be produced biologically using an expression vector into which a nucleic acid has been subcloned in an antisense orientation (i.e., RNA transcribed from the inserted nucleic acid will be of an antisense orientation to a target nucleic acid of interest.

Additionally, the nucleic acid molecules of the invention can be modified at the base moiety, sugar moiety or phosphate backbone to improve, e.g., the stability, hybridization, or solubility of the molecule. For example, the deoxyribose phosphate backbone of the nucleic acids can be modified to generate peptide nucleic acids (see Hyrup et al. (1996) Bioorganic & Medicinal Chemistry 4:5). As used herein, the terms “peptide nucleic acids” or “PNAs” refer to nucleic acid mimics, e.g., DNA mimics, in which the deoxyribose phosphate backbone is replaced by a pseudopeptide backbone and only the four natural nucleobases are retained. The neutral backbone of PNAs has been shown to allow for specific hybridization to DNA and RNA under conditions of low ionic strength. The synthesis of PNA oligomers can be performed using standard solid phase peptide synthesis protocols as described in Hyrup et al. (1996), supra; Perry-O'Keefe et al. (1996) Proc. Natl. Acad. Sci. USA 93:14670. PNAs can be further modified, e.g., to enhance their stability, specificity or cellular uptake, by attaching lipophilic or other helper groups to PNA, by the formation of PNA-DNA chimeras, or by the use of liposomes or other techniques of drug delivery known in the art. The synthesis of PNA-DNA chimeras can be performed as described in Hyrup (1996), supra, Finn et al. (1996) Nucleic Acids Res. 24(17):3357-63, Mag et al. (1989) Nucleic Acids Res. 17:5973, and Peterser et al. (1975) Bioorganic Med. Chem. Lett. 5:1119.

The nucleic acid molecules and fragments of the invention can also include other appended groups such as peptides (e.g., for targeting host cell 14400, 2838, 14618, 15334, 14274, 32164, 39404, 38911, 26904, 31237, 18057, 16405, 32705, 23224, 27423, 32700, 32712 or 12216 proteins in vivo), or agents facilitating transport across the cell membrane (see, e.g., Letsinger et al. (1989) Proc. Natl. Acad. Sci. USA 86:6553-6556; Lemaitre et al. (1987) Proc. Natl. Acad. Sci. USA 84:648-652; PCT Publication No. WO 88/0918) or the blood brain barrier (see, e.g., PCT Publication No. WO 89/10134). In addition, oligonucleotides can be modified with hybridization-triggered cleavage agents (see, e.g., Krol et al. (1988) Bio-Techniques 6:958-976) or intercalating agents (see, e.g., Zon (1988) Pharm Res. 5:539-549).

The polynucleotides are also useful as primers for PCR to amplify any given region of a 14400, 2838, 14618, 15334, 14274, 32164, 39404, 38911, 26904, 31237, 18057, 16405, 32705, 23224, 27423, 32700, 32712 or 12216 polynucleotide.

The polynucleotides are also useful for constructing recombinant vectors. Such vectors include expression vectors that express a portion of, or all of, the 14400, 2838, 14618, 15334, 14274, 32164, 39404, 38911, 26904, 31237, 18057, 16405, 32705, 23224, 27423, 32700, 32712 or 12216 polypeptides. Vectors also include insertion vectors, used to integrate into another polynucleotide sequence, such as into the cellular genome, to alter in situ expression of 14400, 2838, 14618, 15334, 14274, 32164, 39404, 38911, 26904, 31237, 18057, 16405, 32705, 23224, 27423, 32700, 32712 or 12216 genes and gene products. For example, an endogenous 14400, 2838, 14618, 15334, 14274, 32164, 39404, 38911, 26904, 31237, 18057, 16405, 32705, 23224, 27423, 32700, 32712 or 12216 coding sequence can be replaced via homologous recombination with all or part of the coding region containing one or more specifically introduced mutations.

The polynucleotides are also useful for expressing antigenic portions of the 14400, 2838, 14618, 15334, 14274, 32164, 39404, 38911, 26904, 31237, 18057, 16405, 32705, 23224, 27423, 32700, 32712 or 12216 proteins.

The polynucleotides are also useful as probes for determining the chromosomal positions of the 14400, 2838, 14618, 15334, 14274, 32164, 39404, 38911, 26904, 31237, 18057, 16405, 32705, 23224, 27423, 32700, 32712 or 12216 polynucleotides by means of in situ hybridization methods, such as FISH (For a review of this technique, see Verma et al. (1988) Human Chromosomes: A Manual of Basic Techniques (Pergamon Press, New York)), and PCR mapping of somatic cell hybrids. The mapping of the sequences to chromosomes is an important first step in correlating these sequences with genes associated with disease.

Reagents for chromosome mapping can be used individually to mark a single chromosome or a single site on that chromosome, or panels of reagents can be used for marking multiple sites and/or multiple chromosomes. Reagents corresponding to noncoding regions of the genes actually are preferred for mapping purposes. Coding sequences are more likely to be conserved within gene families, thus increasing the chance of cross hybridizations during chromosomal mapping.

Once a sequence has been mapped to a precise chromosomal location, the physical position of the sequence on the chromosome can be correlated with genetic map data. (Such data are found, for example, in Mendelian Inheritance in Man, V. McKusick, available on-line through Johns Hopkins University Welch Medical Library). The relationship between a gene and a disease, mapped to the same chromosomal region, can then be identified through linkage analysis (co-inheritance of physically adjacent genes), described in, for example, Egeland et al. (1987) Nature 325:783-787.

Moreover, differences in the DNA sequences between individuals affected and unaffected with a disease associated with a specified gene, can be determined. If a mutation is observed in some or all of the affected individuals but not in any unaffected individuals, then the mutation is likely to be the causative agent of the particular disease. Comparison of affected and unaffected individuals generally involves first looking for structural alterations in the chromosomes, such as deletions or translocations that are visible form chromosome spreads or detectable using PCR based on that DNA sequence. Ultimately, complete sequencing of genes from several individuals can be performed to confirm the presence of a mutation and to distinguish mutations from polymorphisms.

The polynucleotide probes are also useful to determine patterns of the presence of the gene encoding the proteins and their variants with respect to tissue distribution, for example, whether gene duplication has occurred and whether the duplication occurs in all or only a subset of tissues. The genes can be naturally occurring or can have been introduced into a cell, tissue, or organism exogenously.

The polynucleotides are also useful for designing ribozymes corresponding to all, or a part, of the mRNA produced from genes encoding the polynucleotides described herein.

The polynucleotides are also useful for constructing host cells expressing a part, or all, of the polynucleotides and polypeptides.

The polynucleotides are also useful for constructing transgenic animals expressing all, or a part, of the polynucleotides and polypeptides.

The polynucleotides are also useful for making vectors that express part, or all, of the polypeptides.

The polynucleotides are also useful as hybridization probes for determining the level of 14400, 2838, 14618, 15334, 14274, 32164, 39404, 38911, 26904, 31237, 18057, 16405, 32705, 23224, 27423, 32700, 32712 or 12216 nucleic acid expression. Accordingly, the probes can be used to detect the presence of, or to determine levels of, 14400, 2838, 14618, 15334, 14274, 32164, 39404, 38911, 26904, 31237, 18057, 16405, 32705, 23224, 27423, 32700, 32712 or 12216 nucleic acid in cells, tissues, and in organisms. The nucleic acid whose level is determined can be DNA or RNA. Accordingly, probes corresponding to the polypeptides described herein can be used to assess gene copy number in a given cell, tissue, or organism. This is particularly relevant in cases in which there has been an amplification of the 14400, 2838, 14618, 15334, 14274, 32164, 39404, 38911, 26904, 31237, 18057, 16405, 32705, 23224, 27423, 32700, 32712 or 12216 genes.

Alternatively, the probe can be used in an in situ hybridization context to assess the position of extra copies of the 14400, 2838, 14618, 15334, 14274, 32164, 39404, 38911, 26904, 31237, 18057, 16405, 32705, 23224, 27423, 32700, 32712 or 12216 genes, as on extrachromosomal elements or as integrated into chromosomes in which the gene is not normally found, for example as a homogeneously staining region.

These uses are relevant for diagnosis of disorders involving an increase or decrease in expression relative to normal, such as a proliferative disorder, a differentiative or developmental disorder, or a hematopoietic disorder, especially as disclosed hereinabove.

Thus, the present invention provides a method for identifying a disease or disorder associated with aberrant expression or activity of 14400, 2838, 14618, 15334, 14274, 32164, 39404, 38911, 26904, 31237, 18057, 16405, 32705, 23224, 27423, 32700, 32712 or 122164 nucleic acid, in which a test sample is obtained from a subject and nucleic acid (e.g., mRNA, genomic DNA) is detected, wherein the presence of the nucleic acid is diagnostic for a subject having or at risk of developing a disease or disorder associated with aberrant expression or activity of the nucleic acid.

One aspect of the invention relates to diagnostic assays for determining nucleic acid expression as well as activity in the context of a biological sample (e.g., blood, serum, cells, tissue) to determine whether an individual has a disease or disorder, or is at risk of developing a disease or disorder, associated with aberrant nucleic acid expression or activity. Such assays can be used for prognostic or predictive purpose to thereby prophylactically treat an individual prior to the onset of a disorder characterized by or associated with expression or activity of the nucleic acid molecules.

In vitro techniques for detection of mRNA include Northern hybridizations and in situ hybridizations. In vitro techniques for detecting DNA includes Southern hybridizations and in situ hybridization.

Probes can be used as a part of a diagnostic test kit for identifying cells or tissues that express a 14400, 2838, 14618, 15334, 14274, 32164, 39404, 38911, 26904, 31237, 18057, 16405, 32705, 23224, 27423, 32700, 32712 or 12216 protein, such as by measuring a level of a 14400, 2838, 14618, 15334, 14274, 32164, 39404, 38911, 26904, 31237, 18057, 16405, 32705, 23224, 27423, 32700, 32712 or 12216 protein-encoding nucleic acid in a sample of cells from a subject e.g., mRNA or genomic DNA, or determining if a 14400, 2838, 14618, 15334, 14274, 32164, 39404, 38911, 26904, 31237, 18057, 16405, 32705, 23224, 27423, 32700, 32712 or 12216 gene has been mutated.

Nucleic acid expression assays are useful for drug screening to identify compounds that modulate 14400, 2838, 14618, 15334, 14274, 32164, 39404, 38911, 26904, 31237, 18057, 16405, 32705, 23224, 27423, 32700, 32712 or 12216 nucleic acid expression (e.g., antisense, polypeptides, peptidomimetics, small molecules or other drugs). A cell is contacted with a candidate compound and the expression of mRNA determined. The level of expression of 14400, 2838, 14618, 15334, 14274, 32164, 39404, 38911, 26904, 31237, 18057, 16405, 32705, 23224, 27423, 32700, 32712 or 12216 mRNA in the presence of the candidate compound is compared to the level of expression of 14400, 2838, 14618, 15334, 14274, 32164, 39404, 38911, 26904, 31237, 18057, 16405, 32705, 23224, 27423, 32700, 32712 or 12216 mRNA in the absence of the candidate compound. The candidate compound can then be identified as a modulator of nucleic acid expression based on this comparison and be used, for example to treat a disorder characterized by aberrant nucleic acid expression. The modulator can bind to the nucleic acid or indirectly modulate expression, such as by interacting with other cellular components that affect nucleic acid expression.

Modulatory methods can be performed in vitro (e.g., by culturing the cell with the agent) or, alternatively, in vivo (e.g., by administering the agent to a subject) in patients or in transgenic animals.

The invention thus provides a method for identifying a compound that can be used to treat a disorder associated with nucleic acid expression of the 14400, 2838, 14618, 15334, 14274, 32164, 39404, 38911, 26904, 31237, 18057, 16405, 32705, 23224, 27423, 32700, 32712 or 12216 gene. The method typically includes assaying the ability of the compound to modulate the expression of the 14400, 2838, 14618, 15334, 14274, 32164, 39404, 38911, 26904, 31237, 18057, 16405, 32705, 23224, 27423, 32700, 32712 or 12216 nucleic acid and thus identifying a compound that can be used to treat a disorder characterized by undesired 14400, 2838, 14618, 15334, 14274, 32164, 39404, 38911, 26904, 31237, 18057, 16405, 32705, 23224, 27423, 32700, 32712 or 12216 nucleic acid expression.

The assays can be performed in cell-based and cell-free systems. Cell-based assays include cells naturally expressing the 14400, 2838, 14618, 15334, 14274, 32164, 39404, 38911, 26904, 31237, 18057, 16405, 32705, 23224, 27423, 32700, 32712 or 12216 nucleic acid, such as discussed hereinabove, or recombinant cells genetically engineered to express specific nucleic acid sequences.

Alternatively, candidate compounds can be assayed in vivo in patients or in transgenic animals.

The assay for 14400, 2838, 14618, 15334, 14274, 32164, 39404, 38911, 26904, 31237, 18057, 16405, 32705, 23224, 27423, 32700, 32712 or 12216 nucleic acid expression can involve direct assay of nucleic acid levels, such as mRNA levels, or on collateral compounds involved in the signal pathway (such as cyclic AMP or phosphatidylinositol turnover). Further, the expression of genes that are up- or down-regulated in response to the receptor protein signal pathway can also be assayed. In this embodiment the regulatory regions of these genes can be operably linked to a reporter gene such as luciferase.

Thus, modulators of 14400, 2838, 14618, 15334, 14274, 32164, 39404, 38911, 26904, 31237, 18057, 16405, 32705, 23224, 27423, 32700, 32712 or 12216 gene expression can be identified in a method wherein a cell is contacted with a candidate compound and the expression of mRNA determined. The level of expression of mRNA in the presence of the candidate compound is compared to the level of expression of mRNA in the absence of the candidate compound. The candidate compound can then be identified as a modulator of nucleic acid expression based on this comparison and be used, for example to treat a disorder characterized by aberrant nucleic acid expression. When expression of mRNA is statistically significantly greater in the presence of the candidate compound than in its absence, the candidate compound is identified as a stimulator of nucleic acid expression. When nucleic acid expression is statistically significantly less in the presence of the candidate compound than in its absence, the candidate compound is identified as an inhibitor of nucleic acid expression.

Accordingly, the invention provides methods of treatment, with the nucleic acid as a target, using a compound identified through drug screening as a gene modulator to modulate nucleic acid expression. Modulation includes both up-regulation (i.e. activation or agonization) or down-regulation (suppression or antagonization) or effects on nucleic acid activity (e.g. when nucleic acid is mutated or improperly modified).

Alternatively, a modulator for nucleic acid expression can be a small molecule or drug identified using the screening assays described herein as long as the drug or small molecule inhibits the nucleic acid expression.

The polynucleotides are also useful for monitoring the effectiveness of modulating compounds on the expression or activity of the gene in clinical trials or in a treatment regimen. Thus, the gene expression pattern can serve as a barometer for the continuing effectiveness of treatment with the compound, particularly with compounds to which a patient can develop resistance. The gene expression pattern can also serve as a marker indicative of a physiological response of the affected cells to the compound. Accordingly, such monitoring would allow either increased administration of the compound or the administration of alternative compounds to which the patient has not become resistant. Similarly, if the level of nucleic acid expression falls below a desirable level, administration of the compound could be commensurately decreased.

Monitoring can be, for example, as follows: (i) obtaining a pre-administration sample from a subject prior to administration of the agent; (ii) detecting the level of expression of a specified mRNA or genomic DNA of the invention in the pre-administration sample; (iii) obtaining one or more post-administration samples from the subject; (iv). detecting the level of expression or activity of the mRNA or genomic DNA in the post-administration samples; (v) comparing the level of expression or activity of the mRNA or genomic DNA in the pre-administration sample with the mRNA or genomic DNA in the post-administration sample or samples; and (vi) increasing or decreasing the administration of the agent to the subject accordingly.

The polynucleotides are also useful in diagnostic assays for qualitative changes in 14400, 2838, 14618, 15334, 14274, 32164, 39404, 38911, 26904, 31237, 18057, 16405, 32705, 23224, 27423, 32700, 32712 or 12216 nucleic acid, and particularly in qualitative changes that lead to pathology. The polynucleotides can be used to detect mutations in 14400, 2838, 14618, 15334, 14274, 32164, 39404, 38911, 26904, 31237, 18057, 16405, 32705, 23224, 27423, 32700, 32712 or 12216 genes and gene expression products such as mRNA. The polynucleotides can be used as hybridization probes to detect naturally-occurring genetic mutations in the 14400, 2838, 14618, 15334, 14274, 32164, 39404, 38911, 26904, 31237, 18057, 16405, 32705, 23224, 27423, 32700, 32712 or 12216 gene and thereby to determine whether a subject with the mutation is at risk for a disorder caused by the mutation. Mutations include deletion, addition, or substitution of one or more nucleotides in the gene, chromosomal rearrangement, such as inversion or transposition, modification of genomic DNA, such as aberrant methylation patterns or changes in gene copy number, such as amplification. Detection of a mutated form of the gene associated with a dysfunction provides a diagnostic tool for an active disease or susceptibility to disease when the disease results from overexpression, underexpression, or altered expression of a 14400, 2838, 14618, 15334, 14274, 32164, 39404, 38911, 26904, 31237, 18057, 16405, 32705, 23224, 27423, 32700, 32712 or 12216 protein.

Mutations in the gene can be detected at the nucleic acid level by a variety of techniques. Genomic DNA can be analyzed directly or can be amplified by using PCR prior to analysis. RNA or cDNA can be used in the same way.

In certain embodiments, detection of the mutation involves the use of a probe/primer in a polymerase chain reaction (PCR) (see, e.g. U.S. Pat. Nos. 4,683,195 and 4,683,202), such as anchor PCR or RACE PCR, or, alternatively, in a ligation chain reaction (LCR) (see, e.g., Landegran et al., Science 241:1077-1080 (1988); and Nakazawa et al., PNAS 91:360-364 (1994)), the latter of which can be particularly useful for detecting point mutations in the gene (see Abravaya et al., Nucleic Acids Res. 23:675-682 (1995)). This method can include the steps of collecting a sample of cells from a patient, isolating nucleic acid (e.g., genomic, mRNA or both) from the cells of the sample, contacting the nucleic acid sample with one or more primers which specifically hybridize to a gene under conditions such that hybridization and amplification of the gene (if present) occurs, and detecting the presence or absence of an amplification product, or detecting the size of the amplification product and comparing the length to a control sample. Deletions and insertions can be detected by a change in size of the amplified product compared to the normal genotype. Point mutations can be identified by hybridizing amplified DNA to normal RNA or antisense DNA sequences.

It is anticipated that PCR and/or LCR may be desirable to use as a preliminary amplification step in conjunction with any of the techniques used for detecting mutations described herein.

Alternative amplification methods include: self sustained sequence replication (Guatelli et al. (1990) Proc. Natl. Acad. Sci. USA 87:1874-1878), transcriptional amplification system (Kwoh et al. (1989) Proc. Natl. Acad. Sci. USA 86:1173-1177), Q-Beta Replicase (Lizardi et al. (1988) Bio/Technology 6:1197), or any other nucleic acid amplification method, followed by the detection of the amplified molecules using techniques well-known to those of skill in the art. These detection schemes are especially useful for the detection of nucleic acid molecules if such molecules are present in very low numbers.

Alternatively, mutations in a 14400, 2838, 14618, 15334, 14274, 32164, 39404, 38911, 26904, 31237, 18057, 16405, 32705, 23224, 27423, 32700, 32712 or 12216 gene can be directly identified, for example, by alterations in restriction enzyme digestion patterns determined by gel electrophoresis.

Further, sequence-specific ribozymes (U.S. Pat. No. 5,498,531) can be used to score for the presence of specific mutations by development or loss of a ribozyme cleavage site.

Perfectly matched sequences can be distinguished from mismatched sequences by nuclease cleavage digestion assays or by differences in melting temperature.

Sequence changes at specific locations can also be assessed by nuclease protection assays such as RNase and SI protection or the chemical cleavage method.

Furthermore, sequence differences between a mutant 14400, 2838, 14618, 15334, 14274, 32164, 39404, 38911, 26904, 31237, 18057, 16405, 32705, 23224, 27423, 32700, 32712 or 12216 gene and a wild-type gene can be determined by direct DNA sequencing. A variety of automated sequencing procedures can be utilized when performing the diagnostic assays ((1995) Biotechniques 19:448), including sequencing by mass spectrometry (see, e.g., PCT International Publication No. WO 94/16101; Cohen et al., Adv. Chromatogr. 36:127-162 (1996); and Griffin et al., Appl. Biochem. Biotechnol. 38:147-159 (1993)).

Other methods for detecting mutations in the gene include methods in which protection from cleavage agents is used to detect mismatched bases in RNA/RNA or RNA/DNA duplexes (Myers et al., Science 230:1242 (1985)); Cotton et al., PNAS 85:4397 (1988); Saleeba et al., Meth. Enzymol. 217:286-295 (1992)), electrophoretic mobility of mutant and wild type nucleic acid is compared (Orita et al., PNAS 86:2766 (1989); Cotton et al., Mutat. Res. 285:125-144 (1993); and Hayashi et al., Genet. Anal. Tech. Appl. 9:73-79 (1992)), and movement of mutant or wild-type fragments in polyacrylamide gels containing a gradient of denaturant is assayed using denaturing gradient gel electrophoresis (Myers et al., Nature 313:495 (1985)). The sensitivity of the assay may be enhanced by using RNA (rather than DNA), in which the secondary structure is more sensitive to a change in sequence. In one embodiment, the subject method utilizes heteroduplex analysis to separate double stranded heteroduplex molecules on the basis of changes in electrophoretic mobility (Keen et al. (1991) Trends Genet. 7:5). Examples of other techniques for detecting point mutations include, selective oligonucleotide hybridization, selective amplification, and selective primer extension.

In other embodiments, genetic mutations can be identified by hybridizing a sample and control nucleic acids, e.g., DNA or RNA, to high density arrays containing hundreds or thousands of oligonucleotide probes (Cronin et al. (1996) Human Mutation 7:244-255; Kozal et al. (1996) Nature Medicine 2:753-759). For example, genetic mutations can be identified in two dimensional arrays containing light-generated DNA probes as described in Cronin et al. supra. Briefly, a first hybridization array of probes can be used to scan through long stretches of DNA in a sample and control to identify base changes between the sequences by making linear arrays of sequential overlapping probes. This step allows the identification of point mutations. This step is followed by a second hybridization array that allows the characterization of specific mutations by using smaller, specialized probe arrays complementary to all variants or mutations detected. Each mutation array is composed of parallel probe sets, one complementary to the wild-type gene and the other complementary to the mutant gene.

The polynucleotides are also useful for testing an individual for a genotype that while not necessarily causing the disease, nevertheless affects the treatment modality. Thus, the polynucleotides can be used to study the relationship between an individual's genotype and the individual's response to a compound used for treatment (pharmacogenomic relationship). In the present case, for example, a mutation in the gene that results in altered affinity for ligand could result in an excessive or decreased drug effect with standard concentrations of ligand that activates the protein. Accordingly, the polynucleotides described herein can be used to assess the mutation content of the gene in an individual in order to select an appropriate compound or dosage regimen for treatment.

Thus polynucleotides displaying genetic variations that affect treatment provide a diagnostic target that can be used to tailor treatment in an individual. Accordingly, the production of recombinant cells and animals containing these polymorphisms allow effective clinical design of treatment compounds and dosage regimens.

The methods can involve obtaining a control biological sample from a control subject, contacting the control sample with a compound or agent capable of detecting mRNA, or genomic DNA, such that the presence of mRNA or genomic DNA is detected in the biological sample, and comparing the presence of mRNA or genomic DNA in the control sample with the presence of mRNA or genomic DNA in the test sample.

The polynucleotides are also useful for chromosome identification when the sequence is identified with an individual chromosome and to a particular location on the chromosome. First, the DNA sequence is matched to the chromosome by in situ or other chromosome-specific hybridization. Sequences can also be correlated to specific chromosomes by preparing PCR primers that can be used for PCR screening of somatic cell hybrids containing individual chromosomes from the desired species. Only hybrids containing the chromosome containing the gene homologous to the primer will yield an amplified fragment. Sublocalization can be achieved using chromosomal fragments. Other strategies include prescreening with labeled flow-sorted chromosomes and preselection by hybridization to chromosome-specific libraries. Further mapping strategies include fluorescence in situ hybridization which allows hybridization with probes shorter than those traditionally used. Reagents for chromosome mapping can be used individually to mark a single chromosome or a single site on the chromosome, or panels of reagents can be used for marking multiple sites and/or multiple chromosomes. Reagents corresponding to noncoding regions of the genes actually are preferred for mapping purposes. Coding sequences are more likely to be conserved within gene families, thus increasing the chance of cross hybridizations during chromosomal mapping.

The polynucleotides can also be used to identify individuals from small biological samples. This can be done for example using restriction fragment-length polymorphism (RFLP) to identify an individual. Thus, the polynucleotides described herein are useful as DNA markers for RFLP (See U.S. Pat. No. 5,272,057).

Furthermore, the sequence can be used to provide an alternative technique which determines the actual DNA sequence of selected fragments in the genome of an individual. Thus, the sequences described herein can be used to prepare two PCR primers from the 5′ and 3′ ends of the sequences. These primers can then be used to amplify DNA from an individual for subsequent sequencing.

Panels of corresponding DNA sequences from individuals prepared in this manner can provide unique individual identifications, as each individual will have a unique set of such DNA sequences. It is estimated that allelic variation in humans occurs with a frequency of about once per each 500 bases. Allelic variation occurs to some degree in the coding regions of these sequences, and to a greater degree in the noncoding regions. The sequences can be used to obtain such identification sequences from individuals and from tissue. The sequences represent unique fragments of the human genome. Each of the sequences described herein can, to some degree, be used as a standard against which DNA from an individual can be compared for identification purposes.

If a panel of reagents from the sequences is used to generate a unique identification database for an individual, those same reagents can later be used to identify tissue from that individual. Using the unique identification database, positive identification of the individual, living or dead, can be made from extremely small tissue samples.

The polynucleotides can also be used in forensic identification procedures. PCR technology can be used to amplify DNA sequences taken from very small biological samples, such as a single hair follicle, body fluids (eg. blood, saliva, or semen). The amplified sequence can then be compared to a standard allowing identification of the origin of the sample.

The polynucleotides can thus be used to provide polynucleotide reagents, e.g., PCR primers, targeted to specific loci in the human genome, which can enhance the reliability of DNA-based forensic identifications by, for example, providing another “identification marker” (i.e. another DNA sequence that is unique to a particular individual). As described above, actual base sequence information can be used for identification as an accurate alternative to patterns formed by restriction enzyme generated fragments. Sequences targeted to the noncoding region are particularly useful since greater polymorphism occurs in the noncoding regions, making it easier to differentiate individuals using this technique.

The polynucleotides can further be used to provide polynucleotide reagents, e.g., labeled or labelable probes which can be used in, for example, an in situ hybridization technique, to identify a specific tissue. This is useful in cases in which a forensic pathologist is presented with a tissue of unknown origin. Panels of probes can be used to identify tissue by species and/or by organ type.

In a similar fashion, these primers and probes can be used to screen tissue culture for contamination (i.e. screen for the presence of a mixture of different types of cells in a culture).

Alternatively, the polynucleotides can be used directly to block transcription or translation of 14400, 2838, 14618, 15334, 14274, 32164, 39404, 38911, 26904, 31237, 18057, 16405, 32705, 23224, 27423, 32700, 32712 or 12216 gene sequences by means of antisense or ribozyme constructs. Thus, in a disorder characterized by abnormally high or undesirable 14400, 2838, 14618, 15334, 14274, 32164, 39404, 38911, 26904, 31237, 18057, 16405, 32705, 23224, 27423, 32700, 32712 or 12216 gene expression, nucleic acids can be directly used for treatment.

The polynucleotides are thus useful as antisense constructs to control 14400, 2838, 14618, 15334, 14274, 32164, 39404, 38911, 26904, 31237, 18057, 16405, 32705, 23224, 27423, 32700, 32712 or 12216 gene expression in cells, tissues, and organisms. A DNA antisense polynucleotide is designed to be complementary to a region of the gene involved in transcription, preventing transcription and hence production of protein. An antisense RNA or DNA polynucleotide would hybridize to the mRNA and thus block translation of mRNA into protein.

Examples of antisense molecules useful to inhibit nucleic acid expression include antisense molecules complementary to a fragment of the 5′ untranslated region of SEQ ID NO:2, 5, 7, 9, 12, 15, 17, 19, 21, 23, 53, 57, 60, 62, 64, 66, 68 or 72 which also includes the start codon and antisense molecules which are complementary to a fragment of the 3′ untranslated region of SEQ ID NO:2, 5, 7, 9, 12, 15, 17, 19, 21, 23, 53, 57, 60, 62, 64, 66, 68 or 72.

Alternatively, a class of antisense molecules can be used to inactivate mRNA in order to decrease expression of 14400, 2838, 14618, 15334, 14274, 32164, 39404, 38911, 26904, 31237, 18057, 16405, 32705, 23224, 27423, 32700, 32712 or 12216 nucleic acid. Accordingly, these molecules can treat a disorder characterized by abnormal or undesired 14400, 2838, 14618, 15334, 14274, 32164, 39404, 38911, 26904, 31237, 18057, 16405, 32705, 23224, 27423, 32700, 32712 or 12216 nucleic acid expression. This technique involves cleavage by means of ribozymes containing nucleotide sequences complementary to one or more regions in the mRNA that attenuate the ability of the mRNA to be translated. Possible regions include coding regions and particularly coding regions corresponding to the catalytic and other functional activities of the protein, such as ligand binding. It is understood that these regions include any of those specific domains, sites, segments, loops, and the like that are disclosed as specific regions or sites herein.

The polynucleotides also provide vectors for gene therapy in patients containing cells that are aberrant in 14400, 2838, 14618, 15334, 14274, 32164, 39404, 38911, 26904, 31237, 18057, 16405, 32705, 23224, 27423, 32700, 32712 or 12216 gene expression. Thus, recombinant cells, which include the patient's cells that have been engineered ex vivo and returned to the patient, are introduced into an individual where the cells produce the desired protein to treat the individual.

The invention also encompasses kits for detecting the presence of a 14400, 2838, 14618, 15334, 14274, 32164, 39404, 38911, 26904, 31237, 18057, 16405, 32705, 23224, 27423, 32700, 32712 or 12216 nucleic acid in a biological sample. For example, the kit can comprise reagents such as a labeled or labelable nucleic acid or agent capable of detecting 14400, 2838, 14618, 15334, 14274, 32164, 39404, 38911, 26904, 31237, 18057, 16405, 32705, 23224, 27423, 32700, 32712 or 12216 nucleic acid in a biological sample; means for determining the amount of 14400, 2838, 14618, 15334, 14274, 32164, 39404, 38911, 26904, 31237, 18057, 16405, 32705, 23224, 27423, 32700, 32712 or 12216 nucleic acid in the sample; and means for comparing the amount of 14400, 2838, 14618, 15334, 14274, 32164, 39404, 38911, 26904, 31237, 18057, 16405, 32705, 23224, 27423, 32700, 32712 or 12216 nucleic acid in the sample with a standard. The compound or agent can be packaged in a suitable container. The kit can further comprise instructions for using the kit to detect 14400, 2838, 14618, 15334, 14274, 32164, 39404, 38911, 26904, 31237, 18057, 16405, 32705, 23224, 27423, 32700, 32712 or 12216 mRNA or DNA.

### Computer Readable Means

The nucleotide or amino acid sequences of the invention are also provided in a variety of mediums to facilitate use thereof. As used herein, “provided” refers to a manufacture, other than an isolated nucleic acid or amino acid molecule, which contains a nucleotide or amino acid sequence of the present invention. Such a manufacture provides the nucleotide or amino acid sequences, or a subset thereof (e.g., a subset of open reading frames (ORFs)) in a form which allows a skilled artisan to examine the manufacture using means not directly applicable to examining the nucleotide or amino acid sequences, or a subset thereof, as they exists in nature or in purified form.

In one application of this embodiment, a nucleotide or amino acid sequence of the present invention can be recorded on computer readable media. As used herein, “computer readable media” refers to any medium that can be read and accessed directly by a computer. Such media include, but are not limited to: magnetic storage media, such as floppy discs, hard disc storage medium, and magnetic tape; optical storage media such as CD-ROM; electrical storage media such as RAM and ROM; and hybrids of these categories such as magnetic/optical storage media. The skilled artisan will readily appreciate how any of the presently known computer readable mediums can be used to create a manufacture comprising computer readable medium having recorded thereon a nucleotide or amino acid sequence of the present invention.

As used herein, “recorded” refers to a process for storing information on computer readable medium. The skilled artisan can readily adopt any of the presently known methods for recording information on computer readable medium to generate manufactures comprising the nucleotide or amino acid sequence information of the present invention.

A variety of data storage structures are available to a skilled artisan for creating a computer readable medium having recorded thereon a nucleotide or amino acid sequence of the present invention. The choice of the data storage structure will generally be based on the means chosen to access the stored information. In addition, a variety of data processor programs and formats can be used to store the nucleotide sequence information of the present invention on computer readable medium. The sequence information can be represented in a word processing text file, formatted in commercially-available software such as WordPerfect and MicroSoft Word, or represented in the form of an ASCII file, stored in a database application, such as DB2, Sybase, Oracle, or the like. The skilled artisan can readily adapt any number of dataprocessor structuring formats (e.g., text file or database) in order to obtain computer readable medium having recorded thereon the nucleotide sequence information of the present invention.

By providing the nucleotide or amino acid sequences of the invention in computer readable form, the skilled artisan can routinely access the sequence information for a variety of purposes. For example, one skilled in the art can use the nucleotide or amino acid sequences of the invention in computer readable form to compare a target sequence or target structural motif with the sequence information stored within the data storage means. Search means are used to identify fragments or regions of the sequences of the invention which match a particular target sequence or target motif.

As used herein, a “target sequence” can be any DNA or amino acid sequence of six or more nucleotides or two or more amino acids. A skilled artisan can readily recognize that the longer a target sequence is, the less likely a target sequence will be present as a random occurrence in the database. The most preferred sequence length of a target sequence is from about 10 to 100 amino acids or from about 30 to 300 nucleotide residues. However, it is well recognized that commercially important fragments, such as sequence fragments involved in gene expression and protein processing, may be of shorter length.

As used herein, “a target structural motif,” or “target motif,” refers to any rationally selected sequence or combination of sequences in which the sequence(s) are chosen based on a three-dimensional configuration which is formed upon the folding of the target motif. There are a variety of target motifs known in the art. Protein target motifs include, but are not limited to, enzyme active sites and signal sequences. Nucleic acid target motifs include, but are not limited to, promoter sequences, hairpin structures and inducible expression elements (protein binding sequences).

Computer software is publicly available which allows a skilled artisan to access sequence information provided in a computer readable medium for analysis and comparison to other sequences. A variety of known algorithms are disclosed publicly and a variety of commercially available software for conducting search means are and can be used in the computer-based systems of the present invention. Examples of such software includes, but is not limited to, MacPattern (EMBL), BLASTN and BLASTX (NCBIA).

For example, software which implements the BLAST (Altschul et al. (1990) J. Mol. Biol. 215:403-410) and BLAZE (Brutlag et al. (1993) Comp. Chem. 17:203-207) search algorithms on a Sybase system can be used to identify open reading frames (ORFs) of the sequences of the invention which contain homology to ORFs or proteins from other libraries. Such ORFs are protein encoding fragments and are useful in producing commercially important proteins such as enzymes used in various reactions and in the production of commercially useful metabolites.

### Vectors/Host Cells

The invention also provides vectors containing the receptor polynucleotides. The term “vector” refers to a vehicle, preferably a nucleic acid molecule, that can transport the receptor polynucleotides. When the vector is a nucleic acid molecule, the receptor polynucleotides are covalently linked to the vector nucleic acid. With this aspect of the invention, the vector includes a plasmid, single or double stranded phage, a single or double stranded RNA or DNA viral vector, or artificial chromosome, such as a BAC, PAC, YAC, or MAC.

A vector can be maintained in the host cell as an extrachromosomal element where it replicates and produces additional copies of the receptor polynucleotides. Alternatively, the vector may integrate into the host cell genome and produce additional copies of the receptor polynucleotides when the host cell replicates.

The invention provides vectors for the maintenance (cloning vectors) or vectors for expression (expression vectors) of the receptor polynucleotides. The vectors can function in procaryotic or eukaryotic cells or in both (shuttle vectors).

Expression vectors contain cis-acting regulatory regions that are operably linked in the vector to the receptor polynucleotides such that transcription of the polynucleotides is allowed in a host cell. The polynucleotides can be introduced into the host cell with a separate polynucleotide capable of affecting transcription. Thus, the second polynucleotide may provide a trans-acting factor interacting with the cis-regulatory control region to allow transcription of the receptor polynucleotides from the vector. Alternatively, a trans-acting factor may be supplied by the host cell. Finally, a trans-acting factor can be produced from the vector itself.

It is understood, however, that in some embodiments, transcription and/or translation of the receptor polynucleotides can occur in a cell-free system.

The regulatory sequence to which the polynucleotides described herein can be operably linked include promoters for directing mRNA transcription. These include, but are not limited to, the left promoter from bacteriophage λ, the lac, TRP, and TAC promoters from E. coli, the early and late promoters from SV40, the CMV immediate early promoter, the adenovirus early and late promoters, and retrovirus long-terminal repeats.

In addition to control regions that promote transcription, expression vectors may also include regions that modulate transcription, such as repressor binding sites and enhancers. Examples include the SV40 enhancer, the cytomegalovirus immediate early enhancer, polyoma enhancer, adenovirus enhancers, and retrovirus LTR enhancers.

In addition to containing sites for transcription initiation and control, expression vectors can also contain sequences necessary for transcription termination and, in the transcribed region a ribosome binding site for translation. Other regulatory control elements for expression include initiation and termination codons as well as polyadenylation signals. The person of ordinary skill in the art would be aware of the numerous regulatory sequences that are useful in expression vectors. Such regulatory sequences are described, for example, in Sambrook et al., Molecular Cloning: A Laboratory Manual. 2nd. ed., Cold Spring Harbor Laboratory Press, Cold Spring Harbor, N.Y., (1989).

A variety of expression vectors can be used to express a receptor polynucleotide. Such vectors include chromosomal, episomal, and virus-derived vectors, for example vectors derived from bacterial plasmids, from bacteriophage, from yeast episomes, from yeast chromosomal elements, including yeast artificial chromosomes, from viruses such as baculoviruses, papovaviruses such as SV40, Vaccinia viruses, adenoviruses, poxviruses, pseudorabies viruses, and retroviruses. Vectors may also be derived from combinations of these sources such as those derived from plasmid and bacteriophage genetic elements, eg. cosmids and phagemids. Appropriate cloning and expression vectors for prokaryotic and eukaryotic hosts are described in Sambrook et al., Molecular Cloning: A Laboratory Manual. 2nd. ed., Cold Spring Harbor Laboratory Press, Cold Spring Harbor, N.Y., (1989).

The regulatory sequence may provide constitutive expression in one or more host cells (i.e. tissue specific) or may provide for inducible expression in one or more cell types such as by temperature, nutrient additive, or exogenous factor such as a hormone or other ligand. A variety of vectors providing for constitutive and inducible expression in prokaryotic and eukaryotic hosts are well known to those of ordinary skill in the art.

The receptor polynucleotides can be inserted into the vector nucleic acid by well-known methodology. Generally, the DNA sequence that will ultimately be expressed is joined to an expression vector by cleaving the DNA sequence and the expression vector with one or more restriction enzymes and then ligating the fragments together. Procedures for restriction enzyme digestion and ligation are well known to those of ordinary skill in the art.

The vector containing the appropriate polynucleotide can be introduced into an appropriate host cell for propagation or expression using well-known techniques. Bacterial cells include, but are not limited to, E. coli, Streptomyces, and Salmonella typhimurium. Eukaryotic cells include, but are not limited to, yeast, insect cells such as Drosophila, animal cells such as COS and CHO cells, and plant cells.

As described herein, it may be desirable to express the polypeptide as a fusion protein. Accordingly, the invention provides fusion vectors that allow for the production of the receptor polypeptides. Fusion vectors can increase the expression of a recombinant protein, increase the solubility of the recombinant protein, and aid in the purification of the protein by acting for example as a ligand for affinity purification. A proteolytic cleavage site may be introduced at the junction of the fusion moiety so that the desired polypeptide can ultimately be separated from the fusion moiety. Proteolytic enzymes include, but are not limited to, factor Xa, thrombin, and enterokinase. Typical fusion expression vectors include pGEX (Smith et al., Gene 67:31-40 (1988)), pMAL (New England Biolabs, Beverly, Mass.) and pRIT5 (Pharmacia, Piscataway, N.J.) which fuse glutathione S-transferase (GST), maltose E binding protein, or protein A, respectively, to the target recombinant protein. Examples of suitable inducible non-fusion E. coli expression vectors include pTrc (Amann et al., Gene 69:301-315 (1988)) and pET 11d (Studier et al., Gene Expression Technology: Methods in Enzymology 185:60-89 (1990)).

Recombinant protein expression can be maximized in a host bacteria by providing a genetic background wherein the host cell has an impaired capacity to proteolytically cleave the recombinant protein. (Gottesman, S., Gene Expression Technology: Methods in Enzymology 185, Academic Press, San Diego, Calif. (1990) 119-128). Alternatively, the sequence of the polynucleotide of interest can be altered to provide preferential codon usage for a specific host cell, for example E. coli. (Wada et al., Nucleic Acids Res. 20:2111-2118 (1992)).

The receptor polynucleotides can also be expressed by expression vectors that are operative in yeast. Examples of vectors for expression in yeast e.g., S. cerevisiae include pYepSec1 (Baldari, et al., EMBO J. 6:229-234 (1987)), pMFa (Kurjan et al., Cell 30:933-943(1982)), pJRY88 (Schultz et al., Gene 54:113-123 (1987)), and pYES2 (Invitrogen Corporation, San Diego, Calif.).

The receptor polynucleotides can also be expressed in insect cells using, for example, baculovirus expression vectors. Baculovirus vectors available for expression of proteins in cultured insect cells (e.g., Sf 9 cells) include the pAc series (Smith et al., Mol. Cell Biol. 3:2156-2165 (1983)) and the pVL series (Lucklow et al., Virology 170:31-39 (1989)).

In certain embodiments of the invention, the polynucleotides described herein are expressed in mammalian cells using mammalian expression vectors. Examples of mammalian expression vectors include pCDM8 (Seed, B. Nature 329:840(1987)) and pMT2PC (Kaufman et al., EMBO J. 6:187-195 (1987)).

The expression vectors listed herein are provided by way of example only of the well-known vectors available to those of ordinary skill in the art that would be useful to express the receptor polynucleotides. The person of ordinary skill in the art would be aware of other vectors suitable for maintenance propagation or expression of the polynucleotides described herein. These are found for example in Sambrook, J., Fritsh, E. F., and Maniatis, T. Molecular Cloning: A Laboratory Manual. 2nd, ed., Cold Spring Harbor Laboratory, Cold Spring Harbor Laboratory Press, Cold Spring Harbor, N.Y., 1989.

The invention also encompasses vectors in which the nucleic acid sequences described herein are cloned into the vector in reverse orientation, but operably linked to a regulatory sequence that permits transcription of antisense RNA. Thus, an antisense transcript can be produced to all, or to a portion, of the polynucleotide sequences described herein, including both coding and non-coding regions. Expression of this antisense RNA is subject to each of the parameters described above in relation to expression of the sense RNA (regulatory sequences, constitutive or inducible expression, tissue-specific expression).

The invention also relates to recombinant host cells containing the vectors described herein. Host cells therefore include prokaryotic cells, lower eukaryotic cells such as yeast, other eukaryotic cells such as insect cells, and higher eukaryotic cells such as mammalian cells.

The recombinant host cells are prepared by introducing the vector constructs described herein into the cells by techniques readily available to the person of ordinary skill in the art. These include, but are not limited to, calcium phosphate transfection, DEAE-dextran-mediated transfection, cationic lipid-mediated transfection, electroporation, transduction, infection, lipofection, and other techniques such as those found in Sambrook, et al. (Molecular Cloning: A Laboratory Manual. 2nd, ed., Cold Spring Harbor Laboratory, Cold Spring Harbor Laboratory Press, Cold Spring Harbor, N.Y., 1989).

Host cells can contain more than one vector. Thus, different nucleotide sequences can be introduced on different vectors of the same cell. Similarly, the receptor polynucleotides can be introduced either alone or with other polynucleotides that are not related to the receptor polynucleotides such as those providing trans-acting factors for expression vectors. When more than one vector is introduced into a cell, the vectors can be introduced independently, co-introduced or joined to the receptor polynucleotide vector.

In the case of bacteriophage and viral vectors, these can be introduced into cells as packaged or encapsulated virus by standard procedures for infection and transduction. Viral vectors can be replication-competent or replication-defective. In the case in which viral replication is defective, replication will occur in host cells providing functions that complement the defects.

Vectors generally include selectable markers that enable the selection of the subpopulation of cells that contain the recombinant vector constructs. The marker can be contained in the same vector that contains the polynucleotides described herein or may be on a separate vector. Markers include tetracycline or ampicillin-resistance genes for prokaryotic host cells and dihydrofolate reductase or neomycin resistance for eukaryotic host cells. However, any marker that provides selection for a phenotypic trait will be effective.

While the mature proteins can be produced in bacteria, yeast, mammalian cells, and other cells under the control of the appropriate regulatory sequences, cell-free transcription and translation systems can also be used to produce these proteins using RNA derived from the DNA constructs described herein.

Where secretion of the polypeptide is desired, appropriate secretion signals are incorporated into the vector. The signal sequence can be endogenous to the receptor polypeptides or heterologous to these polypeptides.

Where the polypeptide is not secreted into the medium, the protein can be isolated from the host cell by standard disruption procedures, including freeze thaw, sonication, mechanical disruption, use of lysing agents and the like. The polypeptide can then be recovered and purified by well-known purification methods including ammonium sulfate precipitation, acid extraction, anion or cationic exchange chromatography, phosphocellulose chromatography, hydrophobic-interaction chromatography, affinity chromatography, hydroxylapatite chromatography, lectin chromatography, or high performance liquid chromatography.

It is also understood that depending upon the host cell in recombinant production of the polypeptides described herein, the polypeptides can have various glycosylation patterns, depending upon the cell, or maybe non-glycosylated as when produced in bacteria. In addition, the polypeptides may include an initial modified methionine in some cases as a result of a host-mediated process.

### Uses of Vectors and Host Cells

The host cells expressing the polypeptides described herein, and particularly recombinant host cells, have a variety of uses. First, the cells are useful for producing receptor proteins or polypeptides that can be further purified to produce desired amounts of receptor protein or fragments. Thus, host cells containing expression vectors are useful for polypeptide production.

Host cells are also useful for conducting cell-based assays involving the receptor or receptor fragments. Thus, a recombinant host cell expressing a native receptor is useful to assay for compounds that stimulate or inhibit receptor function. This includes ligand binding, gene expression at the level of transcription or translation, G-protein interaction, and components of the signal transduction pathway.

Host cells are also useful for identifying receptor mutants in which these functions are affected. If the mutants naturally occur and give rise to a pathology, host cells containing the mutations are useful to assay compounds that have a desired effect on the mutant receptor (for example, stimulating or inhibiting function) which may not be indicated by their effect on the native receptor.

Recombinant host cells are also useful for expressing the chimeric polypeptides described herein to assess compounds that activate or suppress activation by means of a heterologous amino terminal extracellular domain (or other binding region). Alternatively, a heterologous region spanning the entire transmembrane domain (or parts thereof) can be used to assess the effect of a desired amino terminal extracellular domain (or other binding region) on any given host cell. In this embodiment, a region spanning the entire transmembrane domain (or parts thereof) compatible with the specific host cell is used to make the chimeric vector. Alternatively, a heterologous carboxy terminal intracellular, e.g., signal transduction, domain can be introduced into the host cell.

Further, mutant receptors can be designed in which one or more of the various functions is engineered to be increased or decreased (e.g., ligand binding or G-protein binding) and used to augment or replace receptor proteins in an individual. Thus, host cells can provide a therapeutic benefit by replacing an aberrant receptor or providing an aberrant receptor that provides a therapeutic result. In one embodiment, the cells provide receptors that are abnormally active.

In another embodiment, the cells provide receptors that are abnormally inactive. These receptors can compete with endogenous receptors in the individual. In another embodiment, cells expressing receptors that cannot be activated, are introduced into an individual in order to compete with endogenous receptors for ligand. For example, in the case in which excessive ligand is part of a treatment modality, it may be necessary to inactivate this ligand at a specific point in treatment. Providing cells that compete for the ligand, but which cannot be affected by receptor activation would be beneficial.

Homologously recombinant host cells can also be produced that allow the in situ alteration of endogenous receptor polynucleotide sequences in a host cell genome. This technology is more fully described in WO 93/09222, WO 91/12650 and U.S. Pat. No. 5,641,670. Briefly, specific polynucleotide sequences corresponding to the receptor polynucleotides or sequences proximal or distal to a receptor gene are allowed to integrate into a host cell genome by homologous recombination where expression of the gene can be affected. In one embodiment, regulatory sequences are introduced that either increase or decrease expression of an endogenous sequence. Accordingly, a receptor protein can be produced in a cell not normally producing it, or increased expression of receptor protein can result in a cell normally producing the protein at a specific level. Alternatively, the entire gene can be deleted. Still further, specific mutations can be introduced into any desired region of the gene to produce mutant receptor proteins. Such mutations could be introduced, for example, into the specific functional regions such as the ligand-binding site or the G-protein binding site.

In one embodiment, the host cell can be a fertilized oocyte or embryonic stem cell that can be used to produce a transgenic animal containing the altered receptor gene. Alternatively, the host cell can be a stem cell or other early tissue precursor that gives rise to a specific subset of cells and can be used to produce transgenic tissues in an animal. See also Thomas et al., Cell 51:503 (1987) for a description of homologous recombination vectors. The vector is introduced into an embryonic stem cell line (e.g., by electroporation) and cells in which the introduced gene has homologously recombined with the endogenous receptor gene is selected (see e.g., Li, E. et al., Cell 69:915 (1992)). The selected cells are then injected into a blastocyst of an animal (e.g., a mouse) to form aggregation chimeras (see e.g., Bradley, A. in Teratocarcinomas and Embryonic Stem Cells: A Practical Approach, E. J. Robertson, ed. (IRL, Oxford, 1987) pp. 113-152). A chimeric embryo can then be implanted into a suitable pseudopregnant female foster animal and the embryo brought to term. Progeny harboring the homologously recombined DNA in their germ cells can be used to breed animals in which all cells of the animal contain the homologously recombined DNA by germline transmission of the transgene. Methods for constructing homologous recombination vectors and homologous recombinant animals are described further in Bradley, A. (1991) Current Opinion

in Biotechnology 2:823-829 and in PCT International Publication Nos. WO 90/11354; WO 91/01140; and WO 93/04169.

The genetically engineered host cells can be used to produce non-human transgenic animals. A transgenic animal is preferably a mammal, for example a rodent, such as a rat or mouse, in which one or more of the cells of the animal include a transgene. A transgene is exogenous DNA which is integrated into the genome of a cell from which a transgenic animal develops and which remains in the genome of the mature animal in one or more cell types or tissues of the transgenic animal. These animals are useful for studying the function of a receptor protein and identifying and evaluating modulators of receptor protein activity.

Other examples of transgenic animals include non-human primates, sheep, dogs, cows, goats, chickens, and amphibians.

In one embodiment, a host cell is a fertilized oocyte or an embryonic stem cell into which receptor polynucleotide sequences have been introduced.

A transgenic animal can be produced by introducing nucleic acid into the male pronuclei of a fertilized oocyte, e.g., by microinjection, retroviral infection, and allowing the oocyte to develop in a pseudopregnant female foster animal. Any of the receptor nucleotide sequences can be introduced as a transgene into the genome of a non-human animal, such as a mouse.

Any of the regulatory or other sequences useful in expression vectors can form part of the transgenic sequence. This includes intronic sequences and polyadenylation signals, if not already included. A tissue-specific regulatory sequence(s) can be operably linked to the transgene to direct expression of the receptor protein to particular cells.

Methods for generating transgenic animals via embryo manipulation and microinjection, particularly animals such as mice, have become conventional in the art and are described, for example, in U.S. Pat. Nos. 4,736,866 and 4,870,009, both by Leder et al., U.S. Pat. No. 4,873,191 by Wagner et al. and in Hogan, B., Manipulating the Mouse Embryo, (Cold Spring Harbor Laboratory Press, Cold Spring Harbor, N.Y., 1986). Similar methods are used for production of other transgenic animals. A transgenic founder animal can be identified based upon the presence of the transgene in its genome and/or expression of transgenic mRNA in tissues or cells of the animals. A transgenic founder animal can then be used to breed additional animals carrying the transgene. Moreover, transgenic animals carrying a transgene can further be bred to other transgenic animals carrying other transgenes. A transgenic animal also includes animals in which the entire animal or tissues in the animal have been produced using the homologously recombinant host cells described herein.

In another embodiment, transgenic non-human animals can be produced which contain selected systems which allow for regulated expression of the transgene. One example of such a system is the cre/loxP recombinase system of bacteriophage P_{1.} For a description of the cre/loxP recombinase system, see, e.g., Lakso et al. PNAS 89:6232-6236 (1992). Another example of a recombinase system is the FLP recombinase system of S. cerevisiae (O'Gorman et al. Science 251:1351-1355 (1991). If a cre/loxP recombinase system is used to regulate expression of the transgene, animals containing transgenes encoding both the Cre recombinase and a selected protein is required. Such animals can be provided through the construction of “double” transgenic animals, e.g., by mating two transgenic animals, one containing a transgene encoding a selected protein and the other containing a transgene encoding a recombinase.

Clones of the non-human transgenic animals described herein can also be produced according to the methods described in Wilmut, I. et al. Nature 385:810-813 (1997) and PCT International Publication Nos. WO 97/07668 and WO 97/07669. In brief, a cell, e.g., a somatic cell, from the transgenic animal can be isolated and induced to exit the growth cycle and enter Gₒ phase. The quiescent cell can then be fused, e.g., through the use of electrical pulses, to an enucleated oocyte from an animal of the same species from which the quiescent cell is isolated. The reconstructed oocyte is then cultured such that it develops to morula or blastocyst and then transferred to pseudopregnant female foster animal. The offspring borne of this female foster animal will be a clone of the animal from which the cell, e.g., the somatic cell, is isolated.

Transgenic animals containing recombinant cells that express the polypeptides described herein are useful to conduct the assays described herein in an in vivo context. Accordingly, the various physiological factors that are present in vivo and that could effect ligand binding, receptor activation, and signal transduction, may not be evident from in vitro cell-free or cell-based assays. Accordingly, it is useful to provide non-human transgenic animals to assay in vivo receptor function, including ligand interaction, the effect of specific mutant receptors on receptor function and ligand interaction, and the effect of chimeric receptors. It is also possible to assess the effect of null mutations, that is mutations that substantially or completely eliminate one or more receptor functions.

### Pharmaceutical Compositions

The receptor nucleic acid molecules, protein (particularly fragments such as the amino terminal extracellular domain), modulators of the protein, and antibodies (also referred to herein as “active compounds”) can be incorporated into pharmaceutical compositions suitable for administration to a subject, e.g., a human. Such compositions typically comprise the nucleic acid molecule, protein, modulator, or antibody and a pharmaceutically acceptable carrier.

As used herein the language “pharmaceutically acceptable carrier” is intended to include any and all solvents, dispersion media, coatings, antibacterial and antifungal agents, isotonic and absorption delaying agents, and the like, compatible with pharmaceutical administration. The use of such media and agents for pharmaceutically active substances is well known in the art. Except insofar as any conventional media or agent is incompatible with the active compound, such media can be used in the compositions of the invention. Supplementary active compounds can also be incorporated into the compositions. A pharmaceutical composition of the invention is formulated to be-compatible with its intended route of administration. Examples of routes of administration include parenteral, e.g., intravenous, intradermal, subcutaneous, oral (e.g., inhalation), transdermal (topical), transmucosal, and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. PH can be adjusted with acids or bases, such as hydrochloric acid or sodium hydroxide. The parenteral preparation can be enclosed in ampules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy syringability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as manitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound (e.g., a receptor protein or anti-receptor antibody) in the required amount in an appropriate solvent with one or a combination of ingredients enumerated above, as required, followed by filtered sterilization. Generally, dispersions are prepared by incorporating the active compound into a sterile vehicle which contains a basic dispersion medium and the required other ingredients from those enumerated above. In the case of sterile powders for the preparation of sterile injectable solutions, the preferred methods of preparation are vacuum drying and freeze-drying which yields a powder of the active ingredient plus any additional desired ingredient from a previously sterile-filtered solution thereof.

Oral compositions generally include an inert diluent or an edible carrier. They can be enclosed in gelatin capsules or compressed into tablets. For oral administration, the agent can be contained in enteric forms to survive the stomach or further coated or mixed to be released in a particular region of the GI tract by known methods. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds are delivered in the form of an aerosol spray from pressured container or dispenser which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The compounds can also be prepared in the form of suppositories (e.g., with conventional suppository bases such as cocoa butter and other glycerides) or retention enemas for rectal delivery.

In one embodiment, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

It is especially advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the invention are dictated by and directly dependent on the unique characteristics of the active compound and the particular therapeutic effect to be achieved, and the limitations inherent in the art of compounding such an active compound for the treatment of individuals.

The nucleic acid molecules of the invention can be inserted into vectors and used as gene therapy vectors. Gene therapy vectors can be delivered to a subject by, for example, intravenous injection, local administration (U.S. Pat. No. 5,328,470) or by stereotactic injection (see e.g., Chen et al., PNAS 91:3054-3057 (1994)). The pharmaceutical preparation of the gene therapy vector can include the gene therapy vector in an acceptable diluent, or can comprise a slow release matrix in which the gene delivery vehicle is imbedded. Alternatively, where the complete gene delivery vector can be produced intact from recombinant cells, e.g. retroviral vectors, the pharmaceutical preparation can include one or more cells which produce the gene delivery system.

The pharmaceutical compositions can be included in a container, pack, or dispenser together with instructions for administration.

This invention may be embodied in many different forms and should not be construed as limited to the embodiments set forth herein; rather, these embodiments are provided so that this disclosure will fully convey the invention to those skilled in the art. Many modifications and other embodiments of the invention will come to mind in one skilled in the art to which this invention pertains having the benefit of the teachings presented in the foregoing description. Although specific terms are employed, they are used as in the ail unless otherwise indicated.

### EQUIVALENTS

Those skilled in the art will recognize, or be able to ascertain using no more than routine experimentation, many equivalents to the specific embodiments of the invention described herein.

This invention is further illustrated by the following examples which should not be construed as limiting. The contents of all references, patents and published patent applications cited throughout this application are incorporated herein by reference.

### EXAMPLES

### Example 1

### Identification and Characterization of Human 18057 cDNAs

The human 18057 sequence (SEQ ID NO:53), which is approximately 1859 nucleotides long including untranslated regions, contains a predicted methionine-initiated coding sequence of about 1410 nucleotides (nucleotides 218-1627 of SEQ ID NO:53). The coding sequence encodes a 469 amino acid protein (SEQ ID NO:52). The originally cloned human 18057 cDNA corresponds to SEQ ID NO:54, it is approximately 1536 nucleotides long including untranslated regions, it contains a predicted methionine-initiated coding sequence of about 1071 nucleotides (nucleotides 229-1299 of SEQ ID NO:54) and it encodes a 356 amino acid protein (SEQ ID NO:55).

In one embodiment, a 18057-like protein includes at least one transmembrane domain. As used herein, the term “transmembrane domain” includes an amino acid sequence of about 15 amino acid residues in length that spans a phospholipid membrane. More preferably, a transmembrane domain includes about at least 18, 20, 22, 24, 25, or 30 amino acid residues and spans a phospholipid membrane. Transmembrane domains are rich in hydrophobic residues, and typically have an α-helical structure. In a preferred embodiment, at least 50%, 60%, 70%, 80%, 90%, 95% or more of the amino acids of a transmembrane domain are hydrophobic, e.g., leucines, isoleucines, tyrosines, or tryptophans. Transmembrane domains are described in, for example Zagotta W. N. et al. (1996) Annual Rev. Neuronsci. 19:235-63, the contents of which are incorporated herein by reference.

In a preferred embodiment, a 18057-like polypeptide or protein has at least one transmembrane domain or a region which includes at least 18, 20, 22, 24, 25, 30 amino acid residues and has at least about 60%, 70% 80% 90% 95%, 99%, or 100% sequence identity with a “transmembrane domain,” e.g., at least one transmembrane domain of human 18057-like protein (e.g., about amino acid residue 7 to about amino acid residue 25 of SEQ ID NO:52; about amino acid residue 38 to about amino acid residue 61 of SEQ ID NO:52; about amino acid residue 72 to about amino acid residue 93 of SEQ ]ID NO:52; about amino acid residue 106 to about amino acid residue 127 of SEQ ID NO:52; about amino acid residue 136 to about amino acid residue 158 of SEQ ID NO:52; about amino acid residue 221 to about amino acid residue 241 of SEQ ID NO:52; about amino acid residue 292 to about amino acid residue 310 of SEQ ID NO:52; about amino acid residue 332 to about amino acid residue 351 of SEQ ID NO:52; about amino acid residue 360 to about amino acid residue 383 of SEQ ID NO:52; about amino acid residue 397 to about amino acid residue 421 of SEQ ID NO:52; or about amino acid residue 428 to about amino acid residue 451 of SEQ ID NO:52).

In another embodiment, a 18057-like protein includes at least one “non-transmembrane domain.” As used herein, “non-transmembrane domains” are domains that reside outside of the membrane. When referring to plasma membranes, non-transmembrane domains include extracellular domains (i.e., outside of the cell) and intracellular domains (i.e., within the cell). When referring to membrane-bound proteins found in intracellular organelles (e.g., mitochondria, endoplasmic reticulum, peroxisomes and microsomes), non-transmembrane domains include those domains of the protein that reside in the cytosol (i.e., the cytoplasm), the lumen of the organelle, or the matrix or the intermembrane space (the latter two relate specifically to mitochondria organelles). The C-terminal amino acid residue of a non-transmembrane domain is adjacent to an N-terminal amino acid residue of a transmembrane domain in a naturally occurring 18057-like polypeptide, or 18057-like protein.

In a preferred embodiment, a 18057-like polypeptide or protein has a “non-transmembrane domain” or a region which includes at least about 1-50, preferably about 5-40, more preferably about 5-25, and even more preferably about 5 to 10 amino acid residues, and has at least about 60%, 70% 80% 90% 95%, 99% or 100% sequence identity with a “non-transmembrane domain”, e.g., a non-transmembrane domain of human 18057-like polypeptide (e.g.,,about amino acid residue 25 to about amino acid residue 38 of SEQ ID NO:52; about amino acid residue 61 to about amino acid residue 72 of SEQ ID NO:52; about amino acid residue 93 to about amino acid residue 106 of SEQ ID NO:52; about amino acid residue 127 to about amino acid residue 136 of SEQ ID NO:52; about amino acid residue 158 to about amino acid residue 221 of SEQ ID NO:52; about amino acid residue 241 to about amino acid residue 292 of SEQ ID NO:52; about amino acid residue 310 to about amino acid residue 332 of SEQ ID NO:52; about amino acid residue 351 to about amino acid residue 360 of SEQ ID NO:52; about amino acid residue 383 to about amino acid -residue 397 of SEQ ID NO:52; or about amino acid residue 421 to about amino acid residue 428 of SEQ ID NO:52).

A non-transmembrane domain located at the N-terminus of a 18057-like protein or polypeptide is referred to herein as an “N-terminal non-transmembrane domain.” As used herein, an “N-terminal non-transmembrane domain” includes an amino acid sequence having about 1-25, preferably about 2-10 amino acid residues in length and is located outside the boundaries of a membrane. For example, an N-terminal non-transmembrane domain in the 18057-like presumed mature peptide is located at about amino acid residues 14-38 of SEQ ID NO:52.

Similarly, a non-transmembrane domain located at the C-terminus of a 18057-like protein or polypeptide is referred to herein as a “C-terminal non-transmembrane domain.” As used herein, an “C-terminal non-transmembrane domain” includes an amino acid sequence having about 1-18, preferably about 2-15, preferably about 5-10 amino acid residues in length and is located outside the boundaries of a membrane. For example, an C-terminal non-transmembrane domain is located at about amino acid residues 451-469 of SEQ ID NO:52.

### Example 2

### Tissue Distribution of 18057 mRNA

In normal human tissues tested, significant expression of 18057 was observed in brain, heart, kidney, and testesIn comparisons of normal and tumor tissue, increased 18057 expression was detected in breast, ovary, and lung tumor tissue. Metastatic liver tissue showed higher relative expression of 18057,than normal liver tissue. Expression levels were determined by quantitative PCR (Taqman® brand quantitative PCR kit, Applied Biosystems). The quantitative PCR reactions were performed according to the kit manufacturer's instructions.

### Example 3

### Identification and Characterization of Human 32705 cDNAs

The human 32705 sequence (SEQ ID NO:60), which is approximately 1347 nucleotides long including untranslated regions, contains a predicted methionine-initiated coding sequence of about 711 nucleotides (nucleotides 176-886 of SEQ ID NO:60). The coding sequence encodes a 236 amino acid protein (SEQ ID NO:61).

32705 has homology with G-proteins. For example, PFAM analysis indicates that the 32705 polypeptide shares a high degree of sequence similarity with the ras-like family. For general information regarding PFAM identifiers, PS prefix and PF prefix domain identification numbers, refer to Sonnhammer et al. (1997) Protein 28:405-420.

As used herein, the term “ras domain” includes an amino acid sequence of about 80-198 amino acid residues in length and having a bit score for the alignment of the sequence to the ras domain (HMM) of at least 8. Preferably, a ras domain includes at least about 100-175 amino acids, more preferably about 125 -150 amino acid residues, and has a bit score for the alignment of the sequence to the ras domain (HMM) of at least 16 or greater. The ras domain (HMM) has been assigned the PFAM Accession number PF00071 (SEQ ID NO:70).

In a preferred embodiment 32705-like polypeptide or protein has a “ras domain” or a region which includes at least about 80-195, more preferably about 100-175 or 125-160 amino acid residues and has at least about 60%, 70%, 80%, 90%, 95%, 99%, or 100% sequence identity with a “ras domain,” e.g., the ras domain of human 32705-like polypeptide (e.g., amino acid residues 33-228 of SEQ ID NO:61).

To identify the presence of a “ras” domain in a 32705-like protein sequence, and make the determination that a polypeptide or protein of interest has a particular profile, the amino acid sequence of the protein can be searched against a database of HMMs (e.g., the Pfam database, release 2.1) using the default parameters). For example, the hmmsf program, which is available as part of the HMMER package of search programs, is a family specific default program for MILPAT0063 and a score of 15 is the default threshold score for determining a hit. Alternatively, the threshold score for determining a hit can be lowered (e.g., to 8 bits). A description of the Pfam database can be found in Sonhammer et al. (1997) Proteins 28(3):405-420 and a detailed description of HMMs can be found, for example, in Gribskov et al. (1990) Meth. Enzymol. 183:146-159; Gribskov et al. (1987) Proc. Natl. Acad. Sci. USA 84:4355-4358; Krogh et al. (1994) J. Mol. Biol. 235:1501-1531; and Stultz et al. (1993) Protein Sci. 2:305-314, the contents of which are incorporated herein by reference.

### Example 4

### Tissue Distribution of 32705 mRNA

Expression of 32705 was detected in normal human tissue, especially brain, as well as in the hepatitis B-infected cell line, HepG2. Expression was also detected in hepatitis C infected liver samples, HepG2 and HuH7 cells. 32705 was also widely expressed in various normal and tumor human tissue, with particularly high levels of expression detected in nerve tissue.

### Example 5

### Identification and Characterization of Human 23224 cDNAs

The human 23224 sequence (SEQ ID NO:62), which is approximately 1023 nucleotides long including untranslated regions, contains a predicted methionine-initiated coding sequence of about 642 nucleotides (nucleotides 245-886 of SEQ ID NO:64). The coding sequence encodes a 213 amino acid protein (SEQ ID NO:65).

23224 has homology with G-proteins. For example, PFAM analysis indicates that the 23224 polypeptide shares a high degree of sequence similarity with the ras-like family and, particularly, the Rab subgroup. See Example 3 for more information regarding the ras domain.

In a preferred embodiment 23224-like polypeptide or protein has a “ras domain” or a region which includes at least about 80-195, more preferably about 100-175 or 125-160 amino acid residues and has at least about 60%, 70%, 80%, 90%, 95%, 99%, or 100% sequence identity with a “ras domain,” e.g., the ras domain of human 23224-like polypeptide (e.g., amino acid residues 10 to 213 of SEQ ID NO:63).

### Example 6

### Tissue Distribution of 23224 mRNA

Expression of 23224 was detected in the following human tissues: Kidney, pancreas, normal spinal cord, normal brain cortex, hypothalamus, dorsal root ganglion, prostate tumor, lung tumor, normal tonsil, normal lymph node, activated peripheral blood mononuclear cells, megakaryocytes, and erythroid tissue.

### Example 7

### Identification and Characterization of Human 27423 cDNAs

The human 27423 sequence (SEQ ID NO:64), which is approximately 1161 nucleotides long including untranslated regions, contains a predicted methionine-initiated coding sequence of about 624 nucleotides (nucleotides 18-641 of SEQ ID NO:64). The coding sequence encodes a 207 amino acid protein (SEQ ID NO:65).

27423 has homology with G-proteins. For example, PFAM analysis indicates that the 27423 polypeptide shares a high degree of sequence similarity with the ras-like family and, particularly, the Rab subgroup. See Example 3 for more information regarding the ras domain.

In a preferred embodiment 23224-like polypeptide or protein has a “ras domain” or a region which includes at least about 80-195, more preferably about 100-175 or 125-160 amino acid residues and has at least about 60%, 70%, 80%, 90%, 95%, 99%, or 100% sequence identity with a “ras domain,” e.g., the ras domain of human 27423-like polypeptide (e.g., amino acid residues 10 to 207 of SEQ ID NO:65).

### Example 8

### Tissue Distribution of 27423 mRNA

Northern blot hybridizations with various RNA samples are performed under standard conditions and washed under stringent conditions, i.e., 0.2×SSC at 65° C. A DNA probe corresponding to all or a portion of the 27423 cDNA (SEQ ID NO:64) can be used. The DNA is radioactively labeled with ³²P-dCTP using the Prime-It Kit (Stratagene, La Jolla, Calif.) according to the instructions of the supplier. Filters containing mRNA from mouse hematopoietic and endocrine tissues, and cancer cell lines (Clontech, Palo Alto, Calif.) are probed in ExpressHyb hybridization solution (Clontech) and washed at high stringency according to manufacturer's recommendations.

### Example 9

### Identification and Characterization of Human 32700 cDNAs

The human 32700 sequence (SEQ ID NO:66), which is approximately 1199 nucleotides long including untranslated regions, contains a predicted methionine-initiated coding sequence of about 552 nucleotides (nucleotides 193-744 of SEQ ID NO:66). The coding sequence encodes a 183 amino acid protein (SEQ ID NO:67).

32700 has homology with G-proteins. For example, PFAM analysis indicates that the 32700 polypeptide shares a high degree of sequence similarity with the ras-like family. See Example 3 for more information regarding the ras domain.

In a preferred embodiment 32700-like polypeptide or protein has a “ras domain” or a region which includes at least about 80-195, more preferably about 100-175 or 125-160 amino acid residues and has at least about 60%, 70%, 80%, 90%, 95%, 99%, or 100% sequence identity with a “ras domain,” e.g., the ras domain of human 32700-like polypeptide (e.g., amino acid residues 8 to 183 of SEQ ID NO:67).

### Example 10

### Tissue Distribution of 32700 mRNA

32700 is widely expressed in various normal and tumor human tissue, with particularly high levels of expression detected in human umbilical vein epithelial cells, normal brain cortex, dorsal root ganglion, lung tumor, and erythroid tissue.

### Example 11

### Identification and Characterization of Human 32712 cDNAs

The human 32712 sequence (SEQ ID NO:68), which is approximately 1116 nucleotides long including untranslated regions, contains a predicted methionine-initiated coding sequence of about 576 nucleotides (nucleotides 124-699 of SEQ ID NO:68). The coding sequence encodes a 191 amino acid protein (SEQ ID NO:69).

32712 has homology with G-proteins. For example, PFAM analysis indicates that the 32712 polypeptide shares a high degree of sequence similarity with the ras-like family and, particularly, the Rab subgroup. See Example 3 for more information regarding the ras domain.

In a preferred embodiment 32712-like polypeptide or protein has a “ras domain” or a region which includes at least about 80-195, more preferably about 100-175 or 125-160 amino acid residues and has at least about 60%, 70%, 80%, 90%, 95%, 99%, or 100% sequence identity with a “ras domain,” e.g., the ras domain of human 32712-like polypeptide (e.g., amino acid residues 2 to 191 of SEQ ID NO:69).

### Example 12

### Tissue Distribution of 32712 mRNA

32712 was widely expressed in various normal and tumor human tissue.

### Example 13

### Recombinant Expression of 14400, 2838, 14618, 15334, 14274, 32164, 39404, 38911, 26904, 31237, 18057, 16405, 32705, 23224, 27423, 32700, 32712 or 12216 in Bacterial Cells

In this example, 14400, 2838, 14618, 15334, 14274, 32164, 39404, 38911, 26904, 31237, 18057, 16405, 32705, 23224, 27423, 32700, 32712 or 12216 is expressed as a recombinant glutathione-S-transferase (GST) fusion polypeptide in E. coli and the fusion polypeptide is isolated and characterized. Specifically, 14400, 2838, 14618, 15334, 14274, 32164, 39404, 38911, 26904, 31237, 18057, 16405, 32705, 23224, 27423, 32700, 32712 or 12216 is fused to GST and this fusion polypeptide is expressed in E. coli, e.g., strain PEB199. Expression of the GST-14400, 2838, 14618, 15334, 14274, 32164, 39404, 38911, 26904, 31237, 18057, 16405, 32705, 23224, 27423, 32700, 32712 or 12216 fusion protein in PEB199 is induced with IPTG. The recombinant fusion polypeptide is purified from crude bacterial lysates of the induced PEB199 strain by affinity chromatography on glutathione beads. Using polyacrylamide gel electrophoretic analysis of the polypeptide purified from the bacterial lysates, the molecular weight of the resultant fusion polypeptide is determined.

### Example 14

### Expression of Recombinant 14400, 2838, 14618, 15334, 14274, 32164, 39404, 38911, 26904, 31237, 18057, 16405, 32705, 23224, 27423, 32700, 32712 or 12216 Protein in COS Cells

To express the 14400, 2838, 14618, 15334, 14274, 32164, 39404, 38911, 26904, 31237, 18057, 16405, 32705, 23224, 27423, 32700, 32712 or 12216 gene in COS cells, the pcDNA/Amp vector by Invitrogen Corporation (San Diego, Calif.) is used. This vector contains an SV40 origin of replication, an ampicillin resistance gene, an E. coli replication origin, a CMV promoter followed by a polylinker region, and an SV40 intron and polyadenylation site. A DNA fragment encoding the entire 14400, 2838, 14618, 15334, 14274, 32164, 39404, 38911, 26904, 31237, 18057, 16405, 32705, 23224, 27423, 32700, 32712 or 12216 protein and an HA tag (Wilson et al. (1984) Cell 37:767) or a FLAG tag fused in-frame to its 3′ end of the fragment is cloned into the polylinker region of the vector, thereby placing the expression of the recombinant protein under the control of the CMV promoter.

To construct the plasmid, the 14400, 2838, 14618, 15334, 14274, 32164, 39404, 38911, 26904, 31237, 18057, 16405, 32705, 23224, 27423, 32700, 32712 or 12216 DNA sequence is amplified by PCR using two primers. The 5′ primer contains the restriction site of interest followed by approximately twenty nucleotides of the 14400, 2838, 14618, 15334, 14274, 32164, 39404, 38911, 26904, 31237, 18057, 16405, 32705, 23224, 27423, 32700, 32712 or 12216 coding sequence starting from the initiation codon; the 3′ end sequence contains complementary sequences to the other restriction site of interest, a translation stop codon, the HA tag or FLAG tag and the last 20 nucleotides of the 14400, 2838, 14618, 15334, 14274, 32164, 39404, 38911, 26904, 31237, 18057, 16405, 32705, 23224, 27423, 32700, 32712 or 12216 coding sequence. The PCR amplified fragment and the pcDNA/Amp vector are digested with the appropriate restriction enzymes and the vector is dephosphorylated using the CIAP enzyme (New England Biolabs, Beverly, Mass.). Preferably the two restriction sites chosen are different so that the 14400, 2838, 14618, 15334, 14274, 32164, 39404, 38911, 26904, 31237, 18057, 16405, 32705, 23224, 27423, 32700, 32712 or 12216 gene is inserted in the correct orientation. The ligation mixture is transformed into E. coli cells (strains HB101, DH5α, SURE, available from Stratagene Cloning Systems, La Jolla, Calif., can be used), the transformed culture is plated on ampicillin media plates, and resistant colonies are selected. Plasmid DNA is isolated from transformants and examined by restriction analysis for the presence of the correct fragment.

COS cells are subsequently transfected with the 14400, 2838, 14618, 15334, 14274, 32164, 39404, 38911, 26904, 31237, 18057, 16405, 32705, 23224, 27423, 32700, 32712 or 12216-pcDNA/Amp plasmid DNA using the calcium phosphate or calcium chloride co-precipitation methods, DEAE-dextran-mediated transfection, lipofection, or electroporation. Other suitable methods for transfecting host cells can be found in Sambrook, J., Fritsh, E. F., and Maniatis, T. Molecular Cloning: A Laboratory Manual. 2nd, ed., Cold Spring Harbor Laboratory, Cold Spring Harbor Laboratory Press, Cold Spring Harbor, N.Y., 1989. The expression of the 14400, 2838, 14618, 15334, 14274, 32164, 39404, 38911, 26904, 31237, 18057, 16405, 32705, 23224, 27423, 32700, 32712 or 12216 polypeptide is detected by radiolabelling (³⁵S-methionine or ³⁵S-cysteine available from NEN, Boston, Mass., can be used) and immunoprecipitation (Harlow, E. and Lane, D. Antibodies: A Laboratory Manual, Cold Spring Harbor Laboratory Press, Cold Spring Harbor, N.Y., 1988) using an HA specific monoclonal antibody. Briefly, the cells are labeled for 8 hours with ³⁵S-methionine (or ³⁵S-cysteine). The culture media are then collected and the cells are lysed using detergents (RIPA buffer, 150 mM NaCl, 1% NP-40, 0.1% SDS, 0.5% DOC, 50 mM Tris, pH 7.5). Both the cell lysate and the culture media are precipitated with an HA specific monoclonal antibody. Precipitated polypeptides are then analyzed by SDS-PAGE.

Alternatively, DNA containing the 14400, 2838, 14618, 15334, 14274, 32164, 39404, 38911, 26904, 31237, 18057, 16405, 32705, 23224, 27423, 32700, 32712 or 12216 coding sequence is cloned directly into the polylinker of the pcDNA/Amp vector using the appropriate restriction sites. The resulting plasmid is transfected into COS cells in the manner described above, and the expression of the 14400, 2838, 14618, 15334, 14274, 32164, 39404, 38911, 26904, 31237, 18057, 16405, 32705, 23224, 27423, 32700, 32712 or 12216 polypeptide is detected by radiolabelling and immunoprecipitation using a 14400, 2838, 14618, 15334, 14274, 32164, 39404, 38911, 26904, 31237, 18057, 16405, 32705, 23224, 27423, 32700, 32712 or 12216 specific monoclonal antibody.

This invention may be embodied in many different forms and should not be construed as limited to the embodiments set forth herein; rather, these embodiments are provided so that this disclosure will fully convey the invention to those skilled in the art. Many modifications and other embodiments of the invention will come to mind in one skilled in the art to which this invention pertains having the benefit of the teachings presented in the foregoing description. Although specific terms are employed, they are used as in the art unless otherwise indicated.

What is claimed is:

1. An isolated 14400, 2838, 14618, 15334, 14274, 32164, 39404, 38911, 26904, 31237, 18057, 16405, 32705, 23224, 27423, 32700, 32712 or 12216 nucleic acid molecule selected from the group consisting of:
a) a nucleic acid molecule comprising a nucleotide sequence which is at least 60% identical to the nucleotide sequence of SEQ ID NO:2, 5, 7, 9, 12, 15, 17, 19, 21, 23, 53, 57, 60, 62, 64, 66, 68 or 72, or the nucleotide sequence of the DNA insert of the plasmid deposited with ATCC Accession Number______;
b) a nucleic acid molecule comprising a fragment of at least 15 nucleotides of the nucleotide sequence of SEQ ID NO:2, 5, 7, 9, 12, 15, 17, 19, 21, 23, 53, 57, 60, 62, 64, 66, 68 or 72, or the nucleotide sequence of the DNA insert of the plasmid deposited with ATCC Accession Number______;
c) a nucleic acid molecule which encodes a polypeptide comprising the amino acid sequence of SEQ ID NO:1, 4, 6, 8, 11, 14, 16, 18, 20, 22, 52, 56, 61, 63, 65, 67, 69 or 71, or the amino acid sequence encoded by the cDNA insert of the plasmid deposited with the ATCC Accession Number______;
d) a nucleic acid molecule which encodes a fragment of a polypeptide comprising the amino acid sequence of SEQ ID NO:1, 4, 6, 8, 11, 14, 16, 18, 20, 22, 52, 56, 61, 63, 65, 67, 69 or 71, or the amino acid sequence encoded by the cDNA insert of the plasmid deposited with the ATCC Accession Number______, wherein the fragment comprises at least 15 contiguous amino acids of SEQ ID NO:1, 4, 6, 8, 11, 14, 16, 18, 20, 22, 52, 56, 61, 63, 65, 67, 69 or 71, or the amino acid sequence encoded by the cDNA insert of the plasmid deposited with the ATCC Accession Number______;
e) a nucleic acid molecule which encodes a naturally occurring allelic variant of a polypeptide comprising the amino acid sequence of SEQ ID NO:1, 4, 6, 8, 11, 14, 16, 18, 20, 22, 52, 56, 61, 63, 65, 67, 69 or 71, or the amino acid sequence encoded by the cDNA insert of the plasmid deposited with the ATCC Accession Number ______, wherein the nucleic acid molecule hybridizes to a nucleic acid molecule comprising SEQ ID NO:2, 5, 7, 9, 12, 15, 17, 19, 21, 23, 53, 57, 60, 62, 64, 66, 68 or 72, or a complement thereof, under stringent conditions;
f) a nucleic acid molecule comprising the nucleotide sequence of SEQ ID NO:2, 5, 7, 9, 12, 15, 17, 19, 21, 23, 53, 57, 60, 62, 64, 66, 68 or 72, or the nucleotide sequence of the DNA insert of the plasmid deposited with ATCC Accession Number______; and
g) a nucleic acid molecule which encodes a polypeptide comprising the amino acid sequence of SEQ ID NO:1, 4, 6, 8, 11, 14, 16, 18, 20, 22, 52, 56, 61, 63, 65, 67, 69 or 71, or the amino acid sequence encoded by the cDNA insert of the plasmid deposited with the ATCC Accession Number______.

2. The isolated nucleic acid molecule of claim 1, which is the nucleotide sequence SEQ ID NO:2, 5, 7, 9, 12, 15, 17, 19, 21, 23, 53, 57, 60, 62, 64, 66, 68 or 72.

3. A host cell which contains the nucleic acid molecule of claim 1.

4. An isolated 14400, 2838, 14618, 15334, 14274, 32164, 39404, 38911, 26904, 31237, 18057, 16405, 32705, 23224, 27423, 32700, 32712 or 12216 polypeptide selected from the group consisting of:
a) a polypeptide which is encoded by a nucleic acid molecule comprising a nucleotide sequence which is at least 60% identical to a nucleic acid comprising the nucleotide sequence of SEQ ID NO:2, 5, 7, 9, 12, 15, 17, 19, 21, 23, 53, 57, 60, 62, 64, 66, 68 or 72, or the nucleotide sequence of the DNA insert of the plasmid deposited with ATCC Accession Number______, or a complement thereof;
b) a naturally occurring allelic variant of a polypeptide comprising the amino acid sequence of SEQ ID NO:1, 4, 6, 8, 11, 14, 16, 18, 20, 22, 52, 56, 61, 63, 65, 67, 69 or 71, or the amino acid sequence encoded by the cDNA insert of the plasmid deposited with the ATCC Accession Number______, wherein the polypeptide is encoded by a nucleic acid molecule which hybridizes to a nucleic acid molecule comprising SEQ ID NO:2, 5, 7, 9, 12, 15, 17, 19, 21, 23, 53, 57, 60, 62, 64, 66, 68 or 72, or a complement thereof under stringent conditions;
c) a fragment of a polypeptide comprising the amino acid sequence of SEQ ID NO:1, 4, 6, 8, 11, 14, 16, 18, 20, 22, 52, 56, 61, 63, 65, 67, 69 or 71, or the amino acid sequence encoded by the cDNA insert of the plasmid deposited with the ATCC Accession Number______, wherein the fragment comprises at least 15 contiguous amino acids of SEQ ID NO:1, 4, 6, 8, 11, 14, 16, 18, 20, 22, 52, 56, 61, 63, 65, 67, 69 or 71; and
d) the amino acid sequence of SEQ ID NO:1, 4, 6, 8, 11, 14, 16, 18, 20, 22, 52, 56, 61, 63, 65, 67, 69 or 71.

5. An antibody which selectively binds to a polypeptide of claim 4.

6. The polypeptide of claim 4, further comprising heterologous amino acid sequences.

7. A method for producing a polypeptide selected from the group consisting of:
a) a polypeptide comprising the amino acid sequence of SEQ ID NO:1, 4, 6, 8, 11, 14, 16, 18, 20, 22, 52, 56, 61, 63, 65, 67, 69 or 71, or the amino acid sequence encoded by the cDNA insert of the plasmid deposited with the ATCC Accession Number______;
b) a polypeptide comprising a fragment of the amino acid sequence of SEQ ID NO:1, 4, 6, 8, 11, 14, 16, 18, 20, 22, 52, 56, 61, 63, 65, 67, 69 or 71, or the amino acid sequence encoded by the cDNA insert of the plasmid deposited with the ATCC Accession Number______, wherein the fragment comprises at least 15 contiguous amino acids of SEQ ID NO:1, 4, 6, 8, 11, 14, 16, 18, 20, 22, 52, 56, 61, 63, 65, 67, 69 or 71, or the amino acid sequence encoded by the cDNA insert of the plasmid deposited with the ATCC Accession Number______;
c) a naturally occurring allelic variant of a polypeptide comprising the amino acid sequence of SEQ ID NO:1, 4, 6, 8, 11, 14, 16, 18, 20, 22, 52, 56, 61, 63, 65, 67, 69 or 71, or the amino acid sequence encoded by the cDNA insert of the plasmid deposited with the ATCC Accession Number______, wherein the polypeptide is encoded by a nucleic acid molecule which hybridizes to a nucleic acid molecule comprising SEQ ID NO:2, 5, 7, 9, 12, 15, 17, 19, 21, 23, 53, 57, 60, 62, 64, 66, 68 or 72; and
d) the amino acid sequence of SEQ ID NO:1, 4, 6, 8, 11, 14, 16, 18, 20, 22, 52, 56, 61, 63, 65, 67, 69 or 71;
comprising culturing the host cell of claim 3 under conditions in which the nucleic acid molecule is expressed.

8. A method for detecting the presence of a nucleic acid molecule of claim 1 or a polypeptide encoded by the nucleic acid molecule in a sample, comprising:
a) contacting the sample with a compound which selectively hybridizes to the nucleic acid molecule of claim 1 or binds to the polypeptide encoded by the nucleic acid molecule; and
b) determining whether the compound hybridizes to the nucleic acid or binds to the polypeptide in the sample.

9. A kit comprising a compound which selectively hybridizes to a nucleic acid molecule of claim 1 or binds to a polypeptide encoded by the nucleic acid molecule and instructions for use.

10. A method for identifying a compound which binds to a polypeptide or modulates the activity of the polypeptide of claim 4 comprising the steps of:
a) contacting a polypeptide, or a cell expressing a polypeptide of claim 4 with a test compound; and
b) determining whether the polypeptide binds to the test compound or determining the effect of the test compound on the activity of the polypeptide.

11. A method for modulating the activity of a polypeptide of claim 4 comprising contacting the polypeptide or a cell expressing the polypeptide with a compound which binds to the polypeptide in a sufficient concentration to modulate the activity of the polypeptide.

12. A method for identifying a compound capable of treating a disorder characterized by aberrant 14400, 2838, 14618, 15334, 14274, 32164, 39404, 38911, 26904, 31237, 18057, 16405, 32705, 23224, 27423, 32700, 32712 or 12216 activity, comprising assaying the ability of the compound to modulate 14400, 2838, 14618, 15334, 14274, 32164, 39404, 38911, 26904, 31237, 18057, 16405, 32705, 23224, 27423, 32700, 32712 or 12216 nucleic acid expression or 14400, 2838, 14618, 15334, 14274, 32164, 39404, 38911, 26904, 31237, 18057, 16405, 32705, 23224, 27423, 32700, 32712 or 12216 polypeptide activity, thereby identifying a compound capable of treating a disorder characterized by aberrant 14400, 2838, 14618, 15334, 14274, 32164, 39404, 38911, 26904, 31237, 18057, 16405, 32705, 23224, 27423, 32700, 32712 or 12216 activity.

13. A method of identifying a nucleic acid molecule associated with a disorder characterized by aberrant 14400, 2838, 14618, 15334, 14274, 32164, 39404, 38911, 26904, 31237, 18057, 16405, 32705, 23224, 27423, 32700, 32712 or 12216 activity, comprising:
a) contacting a sample from a subject with a disorder characterized by aberrant 14400, 2838, 14618, 15334, 14274, 32164, 39404, 38911, 26904, 31237, 18057, 16405, 32705, 23224, 27423, 32700, 32712 or 12216 activity, comprising nucleic acid molecules with a hybridization probe comprising at least 25 contiguous nucleotides of SEQ ID NO:2, 5, 7, 9, 12, 15, 17, 19, 21, 23, 53, 57, 60, 62, 64, 66, 68 or 72; and
b) detecting the presence of a nucleic acid molecule in the sample that hybridizes to the probe, thereby identifying a nucleic acid molecule associated with a disorder characterized by aberrant 14400, 2838, 14618, 15334, 14274, 32164, 39404, 38911, 26904, 31237, 18057, 16405, 32705, 23224, 27423, 32700, 32712 or 12216 activity.

14. A method of identifying a polypeptide associated with a disorder characterized by aberrant 14400, 2838, 14618, 15334, 14274, 32164, 39404, 38911, 26904, 31237, 18057, 16405, 32705, 23224, 27423, 32700, 32712 or 12216 activity, comprising:
a) contacting a sample comprising polypeptides with a 14400, 2838, 14618, 15334, 14274, 32164, 39404, 38911, 26904, 31237, 18057, 16405, 32705, 23224, 27423, 32700, 32712 or 12216 polypeptide defined in claim 4; and
b) detecting the presence of a polypeptide in the sample that binds to the 14400, 2838, 14618, 15334, 14274, 32164, 39404, 38911, 26904, 31237, 18057, 16405, 32705, 23224, 27423, 32700, 32712 or 12216 binding partner, thereby identifying the polypeptide associated with a disorder characterized by aberrant 14400, 2838, 14618, 15334, 14274, 32164, 39404, 38911, 26904, 31237, 18057, 16405, 32705, 23224, 27423, 32700, 32712 or 12216 activity.

15. A method of identifying a subject having a disorder characterized by aberrant 14400, 2838, 14618, 15334, 14274, 32164, 39404, 38911, 26904, 31237, 18057, 16405, 32705, 23224, 27423, 32700, 32712 or 12216 activity, comprising:
a) contacting a sample obtained from the subject comprising nucleic acid molecules with a hybridization probe comprising at least 25 contiguous nucleotides of SEQ ID NO:2, 5, 7, 9, 12, 15, 17, 19, 21, 23, 53, 57, 60, 62, 64, 66, 68 or 72 defined in claim 2; and
b) detecting the presence of a nucleic acid molecule in the sample that hybridizes to the probe, thereby identifying a subject having a disorder characterized by aberrant 14400, 2838, 14618, 15334, 14274, 32164, 39404, 38911, 26904, 31237, 18057, 16405, 32705, 23224, 27423, 32700, 32712 or 12216 activity.

16. A method for treating a subject having a disorder characterized by aberrant 14400, 2838, 14618, 15334, 14274, 32164, 39404, 38911, 26904, 31237, 18057, 16405, 32705, 23224, 27423, 32700, 32712 or 12216 activity, or a subject at risk of developing a disorder characterized by aberrant 14400, 2838, 14618, 15334, 14274, 32164, 39404, 38911, 26904, 31237, 18057, 16405, 32705, 23224, 27423, 32700, 32712 or 12216 activity, comprising administering to the subject a 14400, 2838, 14618, 15334, 14274, 32164, 39404, 38911, 26904, 31237, 18057, 16405, 32705, 23224, 27423, 32700, 32712 or 12216 modulator of the nucleic acid molecule defined in claim 1 or the polypeptide encoded by the nucleic acid molecule or contacting a cell with a 14400, 2838, 14618, 15334, 14274, 32164, 39404, 38911, 26904, 31237, 18057, 16405, 32705, 23224, 27423, 32700, 32712 or 12216 modulator.

17. The method defined in claim 16 wherein said disorder is a cellular proliferative and/or differentiative disorder, spleen disorder, lung disorder, colon disorder, liver disorder, uterus disorder, brain disorder, T-cell disorder, skin disorder, bone marrow disorder, heart disorder, blood vessel disorder, red cell disorder, thymus disorder, B-cell disorder, kidney disorder, breast disorder, testis disorder, prostate disorder, thyroid disorder, skeletal muscle disorder, pancreas disorder, small intestine disorder, platelet disorder, ovary disorder, bone disorder, placenta disorder, lymph node disorder and tonsil disorder.

18. The method of claim 16, wherein the 14400, 2838, 14618, 15334, 14274, 32164, 39404, 38911, 26904, 31237, 18057, 16405, 32705, 23224, 27423, 32700, 32712 or 12216 modulator is
a) a small molecule;
b) peptide;
c) phosphopeptide;
d) anti-14400, 2838, 14618, 15334, 14274, 32164, 39404, 38911, 26904, 31237, 18057, 16405, 32705, 23224, 27423, 32700, 32712 or 12216 antibody;
e) a 14400, 2838, 14618, 15334, 14274, 32164, 39404, 38911, 26904, 31237, 18057, 16405, 32705, 23224, 27423, 32700, 32712 or 12216 polypeptide comprising the amino acid sequence of SEQ ID NO:1, 4, 6, 8, 11, 14, 16, 18, 20, 22, 52, 56, 61, 63, 65, 67, 69 or 71, or a fragment thereof;
f) a 14400, 2838, 14618, 15334, 14274, 32164, 39404, 38911, 26904, 31237, 18057, 16405, 32705, 23224, 27423, 32700, 32712 or 12216 polypeptide comprising an amino acid sequence which is at least 90 percent identical to the amino acid sequence of SEQ ID NO:1, 4, 6, 8, 11, 14, 16, 18, 20, 22, 52, 56, 61, 63, 65, 67, 69 or 71, wherein the percent identity is calculated using the ALIGN program for comparing amino acid sequences, a PAM120 weight residue table, a gap length penalty of 12, and a gap penalty of 4; or
g) an isolated naturally occurring allelic variant of a polypeptide consisting of the amino acid sequence of SEQ ID NO:1, 4, 6, 8, 11, 14, 16, 18, 20, 22, 52, 56, 61, 63, 65, 67, 69 or 71, wherein the polypeptide is encoded by a nucleic acid molecule which hybridizes to a complement of a nucleic acid molecule consisting of SEQ ID NO:2, 5, 7, 9, 12, 15, 17, 19, 21, 23, 53, 57, 60, 62, 64, 66, 68 or 72 at 6×SSC at 45° C., followed by one or more washes in 0.2×SSC, 0.1% SDS at 65° C.

19. The method of claim 16, wherein the 14400, 2838, 14618, 15334, 14274, 32164, 39404, 38911, 26904, 31237, 18057, 16405, 32705, 23224, 27423, 32700, 32712 or 12216 modulator is
a) an antisense 14400, 2838, 14618, 15334, 14274, 32164, 39404, 38911, 26904, 31237, 18057, 16405, 32705, 23224, 27423, 32700, 32712 or 12216 nucleic acid molecule;
b) is a ribozyme;
c) the nucleotide sequence of SEQ ID NO:2, 5, 7, 9, 12, 15, 17, 19, 21, 23, 53, 57, 60, 62, 64, 66, 68 or 72 or a fragment thereof;
d) a nucleic acid molecule encoding a polypeptide comprising an amino acid sequence which is at least 90 percent identical to the amino acid sequence of SEQ ID NO:1, 4, 6, 8, 11, 14, 16, 18, 20, 22, 52, 56, 61, 63, 65, 67, 69 or 71,wherein the percent identity is calculated using the ALIGN program for comparing amino acid sequences, a PAM120 weight residue table, a gap length penalty of 12, and a gap penalty of 4;
e) a nucleic acid molecule encoding a naturally occurring allelic variant of a polypeptide comprising the amino acid sequence of SEQ ID NO:1, 4, 6, 8, 11, 14, 16, 18, 20, 22, 52, 56, 61, 63, 65, 67, 69 or 71, wherein the nucleic acid molecule which hybridizes to a complement of a nucleic acid molecule consisting of SEQ ID NO:2, 5, 7, 9, 12, 15, 17, 19, 21, 23, 53, 57, 60, 62, 64, 66, 68 or 72 at 6×SSC at 45° C., followed by one or more washes in 0.2×SSC, 0.1% SDS at 65° C.; or
f) a gene therapy vector.
